(19) United States
(12) Patent Application Publication
(10) Pub. No.: US 2025/0034196 A1
(43) Pub. Date: Jan. 30, 2025

(54) ESTER DERIVATIVES OF N4-HYDROXYCYTIDINE AND USE THEREOF

(71) Applicant: SUZHOU SPRING-SEA BIO-PHARMACEUTICALS CO., LTD., Suzhou (CN)

(72) Inventors: Zuchun ZHAO, Suzhou (CN); Kai XU, Suzhou (CN); Yan XIE, Suzhou (CN)

(21) Appl. No.: 18/709,201

(22) PCT Filed: Nov. 11, 2022
(86) PCT No.: PCT/CN2022/131334

§ 371 Date: May 10, 2024

(30) Foreign Application Priority Data
Nov. 12, 2021 (WO) PCT/CN2021/130348

(51) Int. Cl. C07H 19/067; A61K 31/403; A61K 31/4439; A61K 31/7068; A61P 31/14

(52) U.S. Cl. CPC C07H 19/067; A61K 31/403; A61K 31/4439; A61K 31/7068; A61P 31/14

(57) ABSTRACT

The present disclosure relates to ester derivatives of N⁴-hydroxycytidine (NHC), to pharmaceutical compositions comprising thereof, to the preparation method thereof, and to the use and method of the ester derivatives of N⁴-hydroxycytidine to treat viral infections.

### TECHNICAL FIELD

The present disclosure relates to ester derivatives of N⁴-hydroxycytidine (NHC), to pharmaceutical compositions comprising thereof, and to the use of the ester derivatives of N⁴-hydroxycytidine for treating viral infections. The compounds can be administered orally to provide N⁴-hydroxycytidine.

### BACKGROUND

Currently, SARS-CoV-2, the virus that causes COVID-19 has infected over 240 million people worldwide and caused about five million deaths without sign of slowing down. The world economy and human activities has been negatively impacted very significantly. Even though vaccines are being introduced recently but treatment of infected patients with oral medicine is still highly desired and can complement vaccine use. N⁴-hydroxycytidine (NHC) is a ribonucleoside analog with broad-spectrum antiviral activity against various unrelated RNA viruses including influenza, Ebola, CoV, and Venezuelan equine encephalitis virus (VEEV) and most importantly the human SARS-CoV-2 virus. Although the exact molecular mechanism of action of NHC remained undefined, it was proposed that viral error catastrophe is the basis for anti-virus activity [“Characterization of orally efficacious influenza drug with high resistance barrier in ferrets and human airway epithelia”, Sci Transl Med. 2019 Oct. 23; 11(515): eaax5866], this comes from the tautomerization property of NHC:

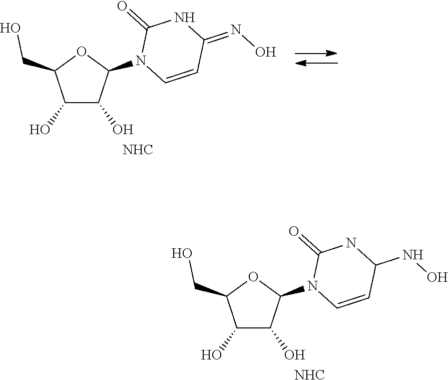

The oxime form of NHC mimics uridine, matching up with adenosine (left structure below), while the other tautomer mimics cytidine and matches up with guanosine (right structure below). Such mismatching might cause virus error catastrophe.

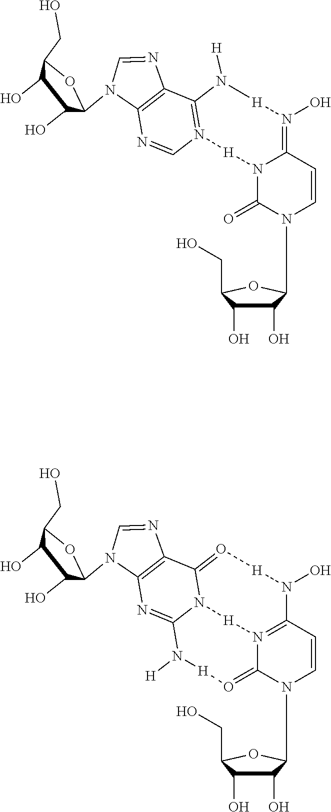

One prodrug of N⁴-hydroxycytidine (NHC) Molnupiravire/EIDD2801/MK4486 has just finished clinical trials for treating SARS-CoV-2, the virus that causes COVID-19. The reported phase III clinical trial for treating early infected patients by SARS-CoV-2 with dose of 800 mg twice a day for 5 days showed 50% reduction in patients progressing into hospitalizations. Both the high dose and BID dosing are required for sustained efficacious concentration of NHC in human body that can induce viral error catastrophe. Thus, more and potentially better prodrugs (i.e. smaller pills, less frequent dosing and more efficacious) are still needed to treat viral infections, especially urgently treat the current human calamity worldwide.

### SUMMARY OF THE INVENTION

The inventors have found a series of ester derivatives of N⁴-hydroxycytidine (NHC) that can deliver NHC in animal blood stream with improved bioavailability and extended exposure compared to the parent molecule NHC.

The present disclosure relates to certain ester prodrugs of NHC, combinations, pharmaceutical compositions, use and methods related thereto.

The present disclosure provides a compound of formula (I):

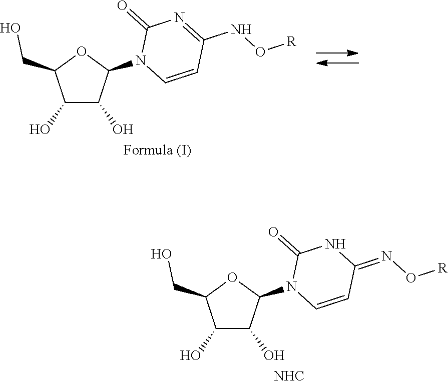

or a tautomer, stereoisomer or racemate thereof or a pharmaceutically acceptable salt thereof, wherein
R is Ra—(C═O)—;
wherein Ra is selected from the group consisting of C₁₋₇ alkyl, C₂₋₆ alkenyl, C₂₋₆ alkynyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl, wherein each of said alkyl, cycloalkyl, aryl, heteroaryl and heterocycloalkyl is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, aryloxy, heteroaryloxy, halo-C₁₋₇ alkyl, halo-C₁₋₇ alkoxy, halo-C₂₋₆ alkenyl, halo-C₂₋₆ alkynyl, hydroxy-C₁₋₇ alkyl, C₁₋₇ alkoxy-C₁₋₇ alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₈ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

In a preferred embodiment, Ra is selected from the group consisting of C₁₋₇ alkyl, C₃₋₈ cycloalkyl, C₆₋₁₀ aryl, 5 to 10 membered heteroaryl, and 3 to 12 membered heterocycloalkyl, each of which is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, halo-C₁₋₇ alkyl, halo-C₁₋₇ alkoxy, aryloxy, heteroaryloxy, halo-C₂₋₆alkenyl, halo-C₂₋₆ alkynyl, hydroxy-C₁₋₇ alkyl, C₁₋₇ alkoxy-C₁₋₇ alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₈ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

In a further preferred embodiment, Ra is selected from the group consisting of C₁₋₇ alkyl, C₃₋₈ cycloalkyl, C₆₋₁₀ aryl, 5 to 10 membered heteroaryl, 3 to 12 membered heterocycloalkyl, halo-C₁₋₇ alkyl, C₁₋₇alkyl-O—C₁₋₇alkyl, C₁₋₇alkyl-O-aryl, C₁₋₇alkyl-O-heteroaryl, halo-C₃₋₈cycloalkyl, C₁₋₆alkyl-O—(CH₂)ₙ—, C₁₋₆alkyl-O—C₁₋₆alkyl-O—(CH₂)ₙ—, halo-C₁₋₆alkyl-O—(CH₂)ₙ—, C₃₋₆cycloalkyl-O—(CH₂)ₙ—, halo-C₃₋₆cycloalkyl-O—(CH₂)ₙ—, 3 to 12 membered heterocycloalkyl-O—(CH₂)ₙ— and 3 to 12 membered haloheterocycloalkyl-O—(CH₂)ₙ—.

The compounds above as well as the compounds disclosed in the context of the present disclosure below (including the compounds of formula (I), and the specific compounds, especially the Example compounds) or their tautomer, stereoisomer, enantiomers, diastereomers, racemate, geometric isomer, hydrate, or solvates, or pharmaceutically acceptable salt thereof are collectively called “the compound of the present invention” or “the compound of the present disclosure”.

The present disclosure also provides the compound of the present invention for use as a medicament.

The present disclosure also provides the compound of the present invention for use in treating or preventing a RNA viral infection.

The present disclosure also provides a pharmaceutical composition, comprising the compound of the present invention and optionally comprising a pharmaceutically acceptable excipient.

The present disclosure also provides a kit for treating or preventing a RNA viral infection, comprising a pharmaceutical composition of the present disclosure and an instruction for use.

The present disclosure also provides use of the compound of the present invention in the manufacture of a medicament for treating or preventing a RNA viral infection.

The present disclosure also provides use of the compound of the present invention for treating or preventing a RNA viral infection.

The present disclosure also provides a method of treating or preventing a RNA viral infection in a subject, comprising administering to the subject in need thereof an effective amount of the compound of the present invention.

The present disclosure also provides a method for increasing bioavailability of N⁴-hydroxycytidine for treating or preventing a RNA viral infection comprising administering to the subject in need thereof an effective amount of the compound of the present invention.

The present disclosure also provides a pharmaceutical combination, comprising the compound of the present invention, and at least one additional therapeutic agent.

The present disclosure also provides a process for the preparation of the compound of the present invention, and intermediates for preparing the compound of the present invention.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The drawings described herein are for illustration purposes only. The drawings are not intended to limit the scope of the present disclosure.

FIG. 1 shows Mean±SD plasma concentration-time data of the Example EX-2, Molnupiravir and NHC after a p.o. dose of EX-2 and Molnupiravir in beagle dogs (EX-2=CH2101, Molnupiravir=CH2017, NHC=CH2018).

FIG. 2 shows the curves of inhibitory activity of test compounds against SARS-CoV-2 Omicron B.1.1.529 variant.

FIG. 3 shows changes of animal body weights in the study p26262-15.

FIG. 4 shows clinical scores in the study P26262-15.

FIG. 5 shows survival proportions in the study P26262-15.

FIG. 6 shows lung viral titer in the study p26262-15.

FIG. 7 shows individual plasma concentration-time data of CH2101 after a PO dose of CH2101 at 10 mg/kg in Beagle Dogs.

FIG. 8 shows individual plasma concentration-time data of EX-1/NHC/CH2018 after a PO dose of CH2101 at 10 mg/kg in Beagle Dogs.

FIG. 9 shows individual plasma concentration-time data of CH2101 after a PO dose of CH2101 at 20 mg/kg in Beagle Dogs.

FIG. 10 shows individual plasma concentration-time data of EX-1/NHC/CH2018 after a PO dose of CH2101 at 20 mg/kg in Beagle Dogs.

FIG. 11 shows individual plasma concentration-time data of CH2107 (Molnupiravir) after a PO dose of CH2107 (Molnupiravir) at 22 mg/kg in Beagle Dogs.

FIG. 12 shows individual plasma concentration-time data of EX-1/NHC/CH2018 after a PO dose of CH2107 (Molnupiravir) at 22 mg/kg in Beagle Dogs.

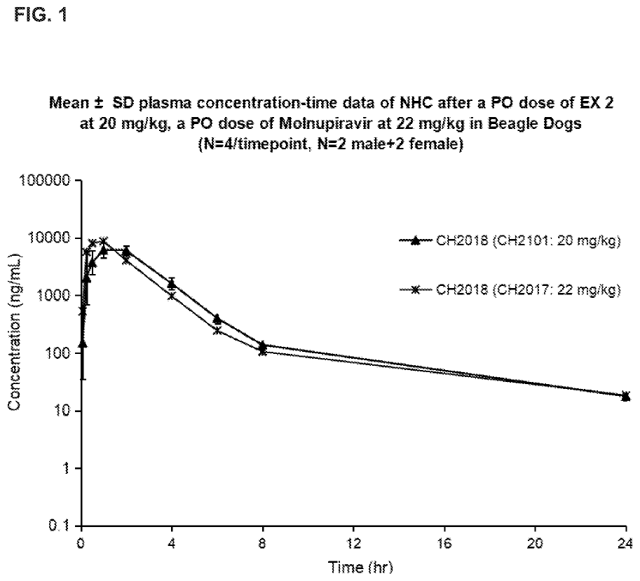

### DETAILED DESCRIPTION OF THE INVENTION

### Definitions

As used herein, the words, phrases and symbols are generally intended to have the meanings as set forth below, except to the extent that the context in which they are used indicates otherwise.

As used herein, the singular forms “a”, “an” and “the” are intended to include the plural forms as well, unless the context clearly indicates otherwise.

The compound of the present invention may be identified either by their chemical structure and/or chemical name. When the chemical structure and chemical name conflict with each other, the chemical structure is determinative of the identity of the compound.

The symbol “ [symbol] ” or “⇄” herein means that the relevant structures are tautomers, which exist in equilibrium and are readily converted from one isomeric form to another. The compound of the present invention may exist in oxime form and the other form. Accordingly, the chemical structures depicted herein encompass all possible tautomeric forms of the illustrated compounds, especially the tautomer in oxime form and the tautomer in the other form. Regardless of the tautomer that is shown, and regardless of the nature of the equilibrium between tautomers, the compound of the present invention is understood by those skilled in the art to encompass both oxime form and the other form.

“Bioavailability” refers to the rate and amount of a drug that reaches the systemic circulation of a subject following administration of the drug or prodrug thereof to the subject and can be determined by evaluating, for example, the plasma or blood concentration-versus-time profile for a drug. Parameters useful in characterizing a plasma or blood concentration-versus-time curve include the area under the curve (AUC), the time to maximum concentration (Tmax), and the maximum drug concentration (Cmax), where Cmax is the maximum concentration of a drug in the plasma or blood of a subject following administration of a dose of the drug or form of drug to the subject, and Tmax is the time to the maximum concentration (Cmax) of a drug in the plasma or blood of a subject following administration of a dose of the drug or form of drug to the subject.

Prodrugs are derivatized forms of drugs that following administration are converted or metabolized to an active form of the parent drug in vivo. Prodrugs are used to modify one or more aspects of the pharmacokinetics of a drug in a manner that enhances the therapeutic efficacy of a parent drug. For example, prodrugs are often used to enhance the oral bioavailability of a drug. To be therapeutically effective, drugs exhibiting poor oral bioavailability may require frequent dosing, large administered doses, or may need to be administered by other than oral routes, such as intravenously. Examples of prodrugs that can be used to improve bioavailability include esters, optionally substituted esters, branched esters, optionally substituted branched esters.

“Metabolic intermediate” refers to a compound that is formed in vivo by metabolism of a parent compound and that further undergoes reaction in vivo to release an active agent. Compounds of Formula (I) are protected ester prodrugs that are metabolized in vivo to provide the corresponding metabolic intermediates such as N4-hydroxycytidine (NHC). It is desirable that the reaction products or metabolites thereof not be toxic.

“Subject” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids and one or more protonable functional groups such as hydroxylamine within the parent compound. Examples of inorganic acids include hydrochloric acid, hydrobromic acid, sulfuric acid, nitric acid, phosphoric acid, and the like. A salt can be formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Also, “pharmaceutically acceptable salt” includes base addition salts formed by the compound of the present invention carrying an acidic moiety with pharmaceutically acceptable cations, for example, sodium, potassium, calcium, aluminum, lithium, and ammonium.

“Pharmaceutical combination” as used herein means a product that results from the mixing or combining of more than one therapeutic agents and includes both fixed and non-fixed combinations of the therapeutic agents. The term “fixed combination” means that the therapeutic agents, e.g. the compound of the present invention and said at least one additional therapeutic agent, are both administered to a subject simultaneously in the form of a single entity or dosage. The term “non-fixed combination” means that the therapeutic agents, e.g. the compound of the present invention and said at least one additional therapeutic agent, are both administered to a subject as separate entities either simultaneously, concurrently or sequentially with no specific time limits, wherein such administration provides therapeutically effective levels of the agents in the body of the subject.

“Preventing” or “prevention” refers to a reduction in risk of acquiring a disease or disorder, e.g. virus infection, (i.e., causing at least one of the clinical symptoms of the disease not to develop in a subject that may be exposed to or predisposed to the disease but does not yet experience or display symptoms of the disease). In some embodiments, “preventing” or “prevention” refers to reducing symptoms of the disease by taking the compound in a preventative fashion. The application of a therapeutic for preventing or prevention of a disease of disorder is known as prophylaxis. Compounds provided by the present disclosure can provide superior prophylaxis because of antiviral activities.

“Treating” or “treatment” of a disease or disorder, e.g. virus infection, refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the subject. “Treating” or “treatment” also refers to delaying the onset of the disease, e.g. virus infection, or at least one or more symptoms thereof in a subject who may be exposed to or predisposed to a disease or disorder even though that subject does not yet experience or display symptoms of the disease.

The term “effective amount” as used herein refers to an amount of the compound of the present invention effective for “treating” or “preventing”, as defined above, virus infection in a subject. The effective amount may cause any changes observable or measurable in a subject as described in the definition of “treating”, “treatment”, “preventing”, or “prevention” above. The “effective amount” may vary depending, for example, on the compound, the disease and/or symptoms of the disease, severity of the disease and/or symptoms of the disease or disorder, the age, weight, and/or health of the subject to be treated, and the judgment of the prescribing physician. An appropriate amount in any given instance may be ascertained by those skilled in the art or capable of determination by routine experimentation.

As used herein, “alkyl” means a straight or branched chain saturated hydrocarbon moieties, such as those containing from 1 to 7 carbon atoms (C₁₋₇), preferably 1 to 6 carbon atoms (C₁₋₆), 1-4 carbon atoms (C₁₋₄) or 1-3 carbon atoms (C₁₋₃). For example, “C₁₋₇alkyl” refers to the alkyl having 1-7 (including 1, 2, 3, 4, 5, 6 or 7) carbon atoms. Representative C₁₋₇alkyl groups include methyl, ethyl, n-propyl, isopropyl, n-butyl, sec-butyl, isobutyl, tert-butyl, n-pentyl, isopentyl, n-hexyl, n-heptyl and the like.

As used herein, “alkenyl” means a straight or branched chain saturated hydrocarbon moieties comprising at least one double bond, such as those containing from 2 to 7 carbon atoms (C₂₋₇), 2 to 6 carbon atoms (C₂₋₄), 2-4 carbon atoms (C₂₋₄) or 2-3 carbon atoms (C₂₋₃). For example, “C₂₋₆ alkenyl” refers to the alkenyl having 2-6 (including 2, 3, 4, 5, or 6) carbon atoms. Representative C₂₋₆ alkenyl groups include ethenyl, propenyl, allyl, butenyl, pentenyl and the like.

As used herein, “alkynyl” means a straight or branched chain saturated hydrocarbon moieties comprising at least one triple bond, such as those containing from 2 to 7 carbon atoms (C₂₋₇), 2 to 6 carbon atoms (C₂₋₄), 2-4 carbon atoms (C₂₋₄) or 2-3 carbon atoms (C₂₋₃). For example, “C₂₋₆ alkynyl” refers to the alkynyl having 2-6 (including 2, 3, 4, 5, or 6) carbon atoms. Representative C₂₋₆ alkynyl groups include ethynyl, propynyl, propargyl, butynyl, and the like.

As used herein, “alkoxy” refers to —O-alkyl wherein said alkyl has the meaning as defined above, such as alkoxy containing from 1 to 7 carbon atoms (C₁₋₇), 1 to 6 carbon atoms (C₁₋₆), 1-4 carbon atoms (C₁₋₄) or 1-3 carbon atoms (C₁₋₃). For example, “C₁₋₇ alkoxy” refers to the alkoxy having 1-7 (including 1, 2, 3, 4, 5, 6 or 7) carbon atoms. Representative C₁₋₇ alkoxy groups include methoxy, ethoxy, n-propoxy, isopropoxy, n-butoxy, sec-butoxy, isobutoxy, tert-butoxy, n-pentyloxy, isopentyloxy, n-hexyloxy and the like.

The term “cycloalkyl” as used herein are referred to saturated cyclic hydrocarbon radical having 3 to 8 ring carbon atoms (C₃₋₈), such as 3-6 ring carbon atoms (C₃₋₆) or 5-6 ring carbon atoms (C₅₋₆). Examples of the cycloalkyl include, but are not limited to, cyclopropyl, cyclobutyl, cyclopentyl, cyclohexyl, cycloheptyl or cyclooctyl, or bicyclo-system, including spiro and bridging rings, such as bicyclo[1.1.1]pentyl, bicyclo[2.2.1]heptyl, spiro[3.4]octyl, bicyclo[3.1.1]hexyl, bicyclo[3.1.1]heptyl, or bicyclo[3.2.1]octyl. The term “halo-cycloalkyl” herein refers to the cycloalkyl as defined above, in which one or more, for example 1, 2 or 3 hydrogen atoms are replaced with halogen atom.

The term “heterocycloalkyl” as used herein refers to a saturated ring having 3-12 ring atoms (3-12 membered), 3-10 ring atoms (3-10 membered), 3-6 ring atoms (3-6 membered), 4-6 ring atoms (4-6 membered) or 5-6 ring atoms (5-6 membered), with one or more of, such as 1, 2, 3 or 4, preferably 1 or 2 of the ring atoms being heteroatoms independently selected from N, O and S, preferably 0, and the remaining ring atoms being carbon. Examples of the heterocycloalkyl include, but are not limited to, morpholinyl, pyrrolidinonyl, pyrrolidinyl, piperidinyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, 1,3-dioxolane moiety and the like. Preferably, the heterocycloalkyl is tetrahydrofuranyl or tetrahydropyranyl. For example, heterocycloalkyl can be selected from the following groups:

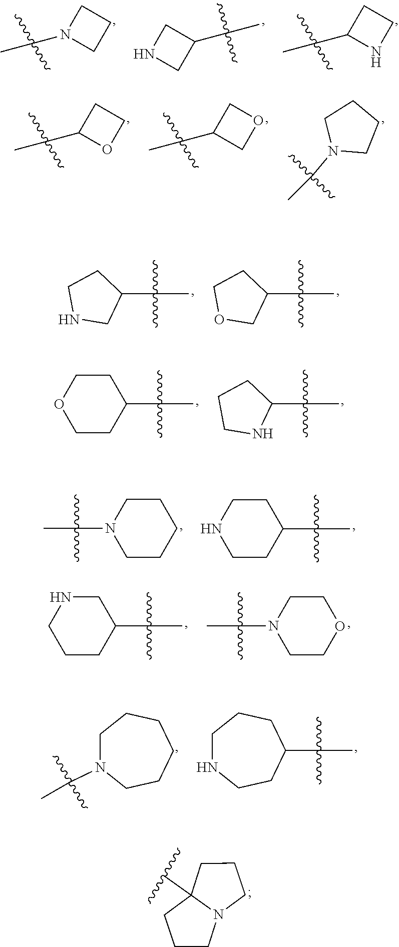

It should be understood that the structures having one or more asymmetric centers cover its racemic mixture and/or single enantiomer or mixture thereof. For instance, the structure

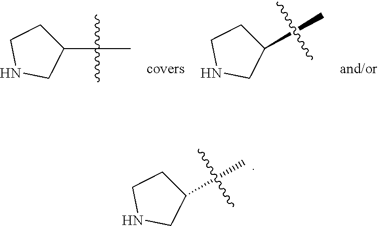

As used herein, the term “heterocycloalkyl” also includes “heterocycloalkenyl”, which refers to “heterocycloalkyl” defined herein which contains at least one (e.g., 1, 2 or 3) double bond. Examples of heterocycloalkenyl groups include, but are not limited to:

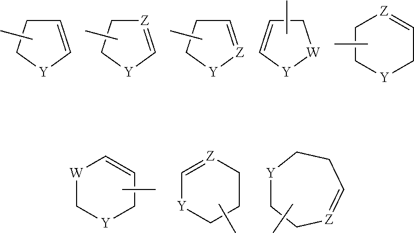

wherein each W is selected from CH₂, NH, O and S, each Y is selected from NH, O, C(═O), SO₂ and S, and each Z is selected from N and CH, provided that each ring contains at least one heteroatom selected from N, O or S. For example, said heterocycloalkenyl is pyrrolinyl (for example, 1-pyrrolinyl, 2-pyrrolinyl, 3-pyrrolinyl, 4-pyrrolinyl or 5-pyrrolinyl), dihydrofuranyl (for example, 1-, 2-, 3-, or 4-dihydrofuran), dihydrothienyl (for example, 1-, 2-, 3- or 4-dihydrothienyl), tetrahydropyridinyl (for example, 1-, 2-, 3-, 4-, 5- or 6-tetrahydropyridinyl), tetrahydropyranyl (for example, 4-tetrahydropyranyl) or tetrahydrothiopyranyl (for example, 4-tetrahydrothiopyranyl).

As used herein, the term “aryl” means a monovalent aromatic hydrocarbon group derived by removing one hydrogen atom from a single carbon atom in an aromatic ring system. Aryl refers to a monocyclic or fused polycyclic aromatic ring structure with a specified number of ring atoms. Specifically, the term includes groups containing 6 to 14, for example 6 to 10, preferably 6 ring members. Representative aryl groups include phenyl and naphthyl, preferably phenyl. The term “aryl” also includes biaryl, such as biphenyl and binaphthyl.

As used herein, the term “heteroaryl” means monocyclic or fused polycyclic aromatic ring structure including one or more (for example, 1, 2, 3 or 4) heteroatoms independently selected from O, N and S and a specified number of ring atoms, or its N-oxide, or its S-oxide or S-dioxide. Specifically, the aromatic ring structure may have 5 to 10 ring members. Typically, the heteroaryl ring will contain up to 4 heteroatoms, up to 3 heteroatoms, up to 2 heteroatoms, for example, one heteroatom independently selected from O, N and S, where N and S may be in an oxidation state such as S═O or S(O)₂. For example, heteroaryl may be a fused ring containing 1, 2, 3 or 4 heteroatoms independently selected from N, O or S, such as benzofuran, benzothiophene, indole, benzimidazole, indazole, benzotriazole, pyrrolo[2,3-b]pyridine, pyrrolo[2,3-c]pyridine, pyrazolo[4,3-c]pyridine, pyrazolo[3,4-c]pyridine, pyrazolo[3,4-b]pyridine, isoindole, purine, indolizine, imidazo[1,2-a]pyridine, imidazo[1,5-a]pyridine, 1H-pyrazolo[3,4-d]pyrimidine, 7H-pyrrolo[2,3-d]pyrimidine, quinoline, isoquinoline, cinnarine, quinazoline, quinoxaline, phthalazine, 1,6-naphthyridine, 1,7-naphthyridine, pyrido[2,3-b]pyrazine, pyrido[3,4-b]pyrazine, pyrimido[5,4-d]pyrimidine, pyrazino[2,3-b]pyrazine and pyrimido[4,5-d]pyrimidine. For example, the heteroaryl may be a 5-6 membered heteroaryl containing 1 or 2 heteroatoms independently selected from N, O or S. Examples of 5-6 membered monocyclic heteroaryl groups include, but are not limited to, pyrrolyl, furyl, thienyl, imidazolyl, furazan, oxazolyl, oxadiazole, oxatriazolyl, isoxazole, thiazolyl, isothiazolyl, pyrazolyl, triazolyl, tetrazolyl, pyridyl, pyrazinyl, pyridazinyl, pyrimidinyl and triazinyl.

The term “acyl” refers to the group Rx-(C═O)—, wherein Rx is C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl, wherein each of said alkyl, cycloalkyl, aryl, heteroaryl and heterocycloalkyl is optionally substituted with one or more substituents selected from the following groups: halogen, hydroxy, cyano, nitro, amino, —NH(C₁₋₇ alkyl), —N(C₁₋₇ alkyl)₂, —NH(acyl), —N(acyl)₂, amino-acyl, C₁₋₇ alkyl, C₁₋₆ alkoxy, halo-C₁₋₇ alkyl, or halo-C₁₋₇ alkoxy.

The terms “halogen” and “halo” refer to fluorine, chlorine, bromo or iodo.

The term “halo-alkyl” herein refers to the alkyl as defined herein, in which one or more, for example 1, 2, 3, 4, 5, or all hydrogen atoms are replaced with halogen atom.

The term “substituted” refers to a molecule wherein at least one hydrogen atom is replaced with a substituent. When substituted, one or more of the groups are “substituents.” The molecule can be multiply substituted.

The term “optionally” as used herein means that the subsequently described event or circumstance may or may not occur, and the description includes instances wherein the event or circumstance occur and instances in which it does not occur.

The term “lower aliphatic alcohol” refers to C₁-C₄ alcohol, which represents aliphatic alcohol having 1-4 carbon atoms, such as methanol, ethanol, propanol, isopropanol, n-butanol, isobutanol, tert-butyl alcohol, and the like.

All numerical ranges herein should be understood as disclosing each and every value within the range and each and every subset of values within the range, regardless of whether they are specifically disclosed otherwise. For example, when referring to any numerical range, it should be regarded as referring to each and every numerical value in the numerical range, for example, each and every integer in the numerical range. The present disclosure includes all values falling within these ranges, all smaller ranges, and the upper or lower limit of the range.

Technical and scientific terms used herein and not specifically defined have the meaning commonly understood by a person skilled in the art, to which the present disclosure pertains.

### EMBODIMENTS OF THE DISCLOSURE

Embodiment 1. A compound of Formula (I):

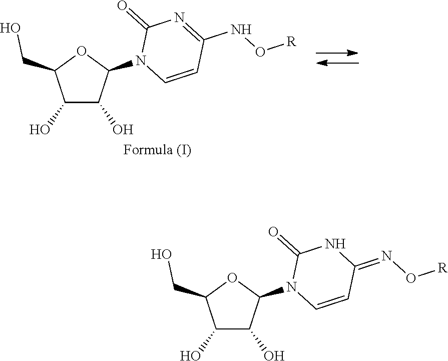

or a tautomer, stereoisomer or racemate thereof or a pharmaceutically acceptable salt thereof, wherein
R is Ra—(C═O)—;
wherein Ra is selected from the group consisting of C₁₋₇ alkyl, C₂₋₆ alkenyl, C₂₋₆ alkynyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl, wherein each of said alkyl, cycloalkyl, aryl, heteroaryl and heterocycloalkyl is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, aryloxy, heteroaryloxy, halo-C₁₋₇ alkyl, halo-C₁₋₇ alkoxy, halo-C₂₋₆ alkenyl, halo-C₂₋₆ alkynyl, hydroxy-C₁₋₇ alkyl, C₁₋₇ alkoxy-C₁₋₇ alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₈ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

Preferably, Ra is selected from the group consisting of C₁₋₇alkyl, C₃₋₈cycloalkyl, C₆₋₁₀ aryl, 5 to 10 membered heteroaryl, and 3 to 12 membered heterocycloalkyl, each of which is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, halo-C₁₋₇ alkyl, halo-C₁₋₇ alkoxy, aryloxy, heteroaryloxy, halo-C₂₋₆ alkenyl, halo-C₂₋₆ alkynyl, hydroxy-C₁₋₇alkyl, C₁₋₇ alkoxy-C₁₋₇ alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₄ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

Embodiment 2. The compound of Formula (I) according to Embodiment 1, or a tautomer, stereoisomer or racemate thereof or a pharmaceutically acceptable salt thereof, wherein R is selected from the following groups:

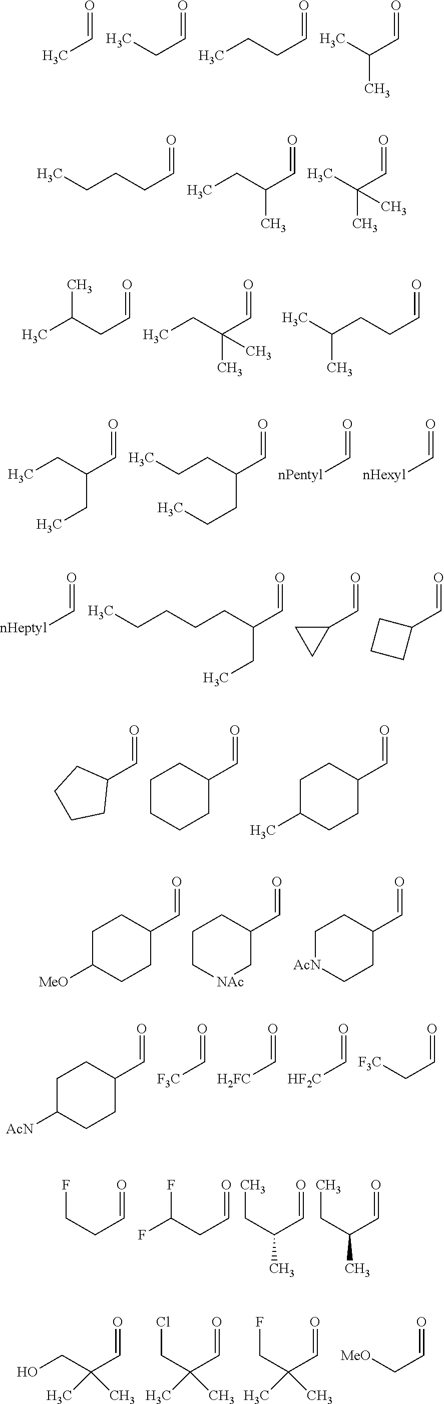

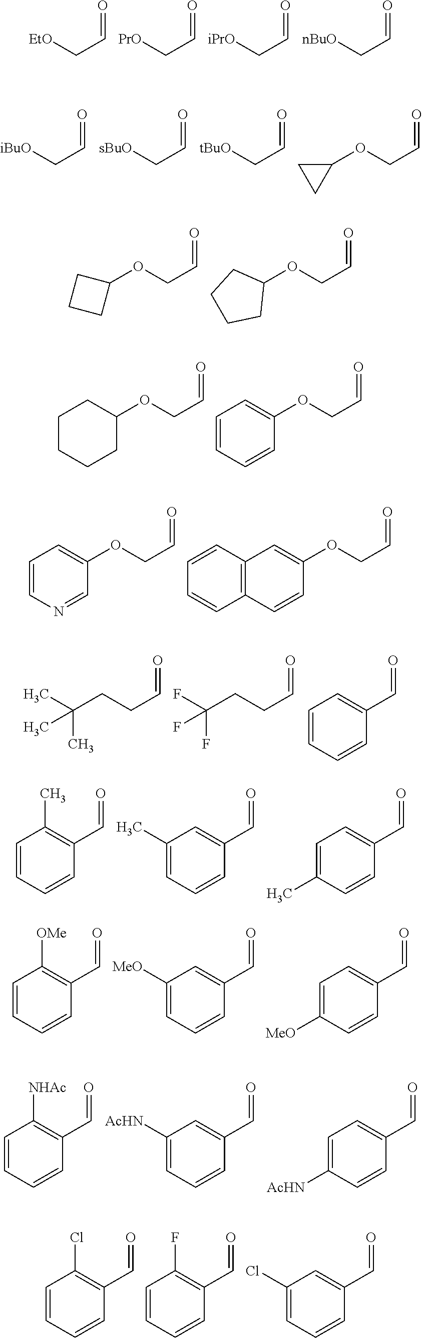

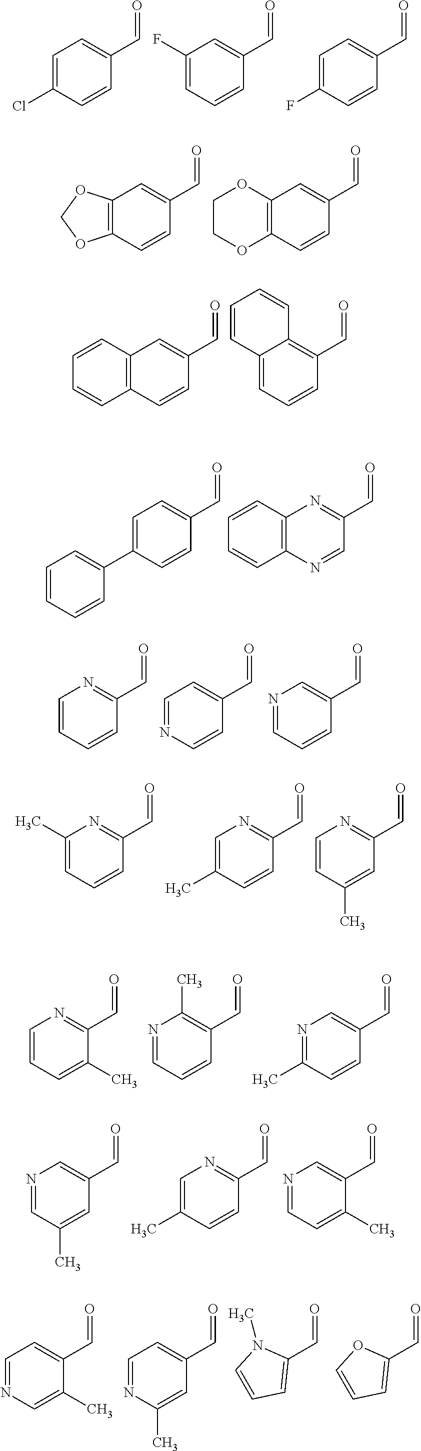

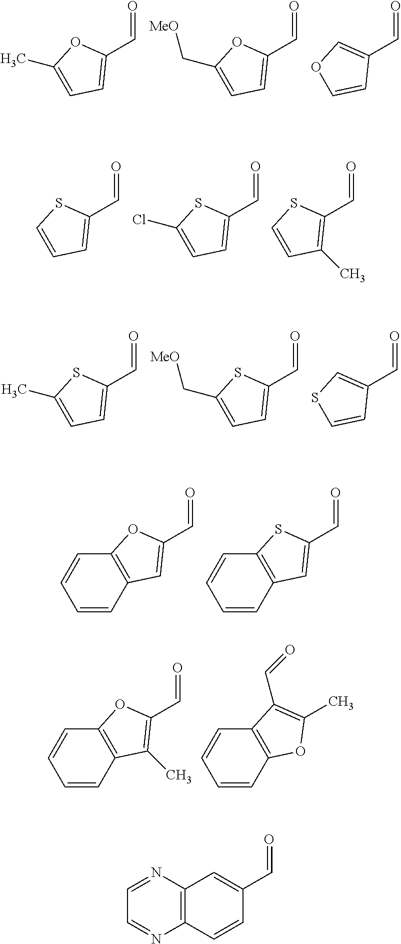

Embodiment 3. A compound according to Embodiment 1, or a tautomer, stereoisomer or racemate thereof or a pharmaceutically acceptable salt thereof, wherein
R is Ra—(C═O)—,
Ra is methyl substituted with Ra1, Ra2 and Ra3;
wherein each of Ra1, Ra2 and Ra3 is independently selected from the group consisting of H, C₁₋₆alkyl, C₁₋₆alkyl-O—C₁₋₆alkyl, C₃₋₆cycloalkyl, 3-6 membered heterocycloalkyl, C₁₋₆alkyl-O—(CH₂)ₙ—, C₁₋₇alkyl-O-aryl, C₁₋₇alkyl-O-heteroaryl, C₁₋₆alkyl-O—C₁₋₆alkyl-O—(CH₂)ₙ—, C₁₋₆haloalkyl-O—(CH₂)ₙ—, C₃₋₆cycloalkyl-O—(CH₂)ₙ— and 3-6 membered heterocycloalkyl-O—(CH₂)ₙ—, wherein each of said alkyl, cycloalkyl and heterocycloalkyl is optionally substituted with one or more substituents selected from the following groups: halogen, acyl, hydroxy, cyano, nitro, amino, —NH(C₁₋₇ alkyl), —N(C₁₋₇ alkyl)₂, C₁₋₇ alkyl, C₁₋₆ alkoxy, halo-C₁₋₇ alkyl, or halo-C₁₋₇ alkoxy; and
n is 0 or 1.

Embodiment 4. A compound according to any of the preceding Embodiments, or a tautomer, stereoisomer or racemate thereof or a pharmaceutically acceptable salt thereof, wherein
R is Ra—(C═O)—,
wherein Ra—(C═O)— is selected from the group consisting of:

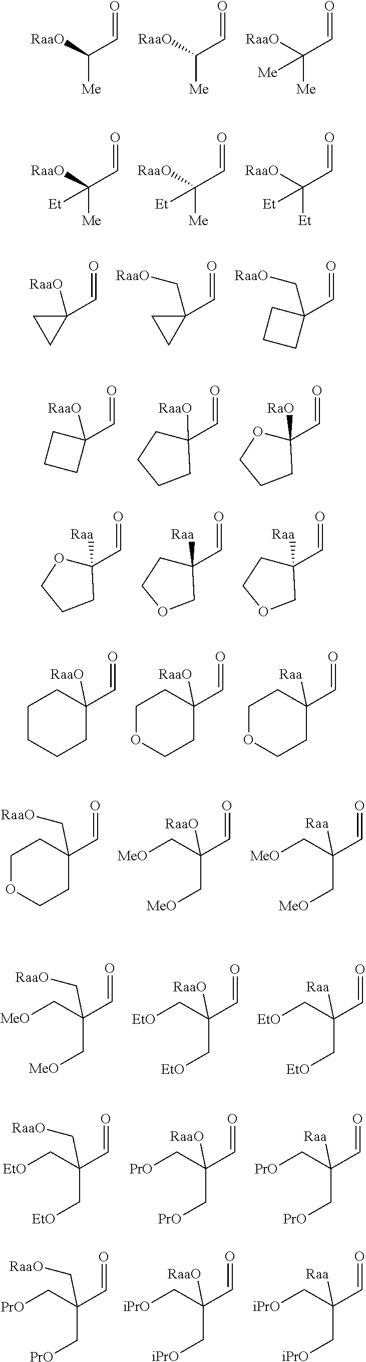

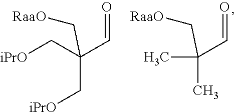

Raa is selected from the group consisting of C₁₋₆alkyl, C₁₋₆haloalkyl, C₁₋₆alkyl-O—C₁₋₆alkyl-, C₃₋₆cycloalkyl and 3-6 membered heterocycloalkyl; preferably C₁₋₆alkyl.

Embodiment 5. A compound according to Embodiment 4, or a tautomer, stereoisomer or racemate thereof or a pharmaceutically acceptable salt thereof, wherein Ra—(C═O)— is selected from the group consisting of:

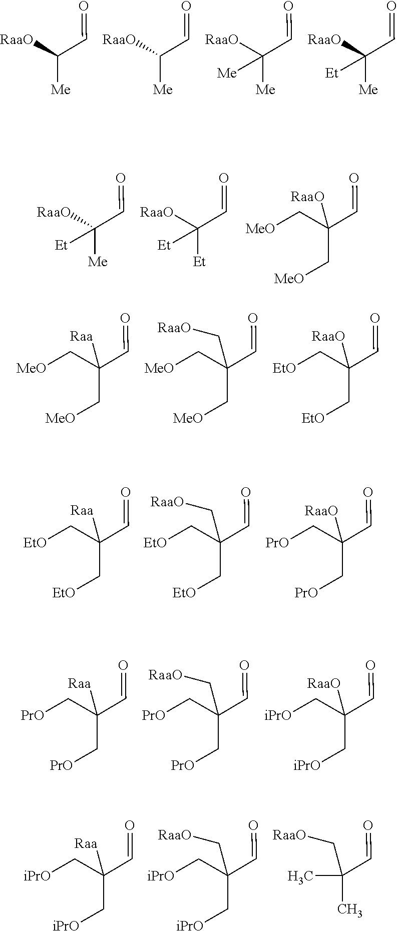

wherein Raa is defined as above.

Embodiment 6. A compound according to Embodiment 4, or a tautomer, stereoisomer or racemate thereof or a pharmaceutically acceptable salt thereof, wherein Ra—(C═O)— is selected from the group consisting of:

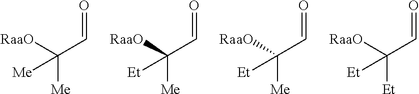

wherein Raa is defined as above.

Embodiment 7. A compound according to Embodiment 4, or a tautomer, stereoisomer or racemate thereof or a pharmaceutically acceptable salt thereof, wherein Ra—(C═O)— is selected from the group consisting of:

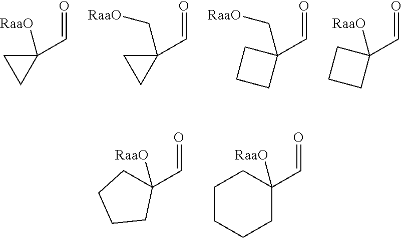

wherein Raa is defined as above.

Embodiment 8. A compound according to Embodiment 4, or a tautomer, stereoisomer or racemate thereof or a pharmaceutically acceptable salt thereof, wherein Ra—(C═O)— is selected from the group consisting of:

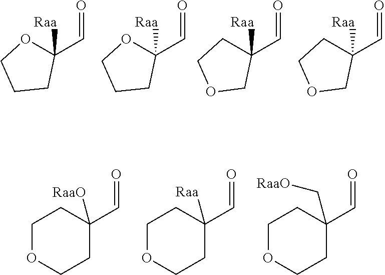

wherein Raa is defined as above.

Embodiment 9. A compound according to any of the preceding Embodiments, or a tautomer, stereoisomer or racemate thereof or a pharmaceutically acceptable salt thereof, wherein Raa is selected from the group consisting of C₁₋₄alkyl, C₁₋₄haloalkyl, C₁₋₄alkyl-O—C₁₋₄alkyl-, C₃₋₅cycloalkyl and 4-6 membered heterocycloalkyl.

Embodiment 10. A compound according to any of the preceding Embodiments, or a tautomer, stereoisomer or racemate thereof or a pharmaceutically acceptable salt thereof, wherein Raa is selected from the group consisting of methyl, ethyl, propyl, isopropyl, n-butyl, sec-butyl, 2-methoxyethyl, fluorosubstitued ethyl, flurosubstituted propyl, cyclopropyl, cyclobutyl, cyclopentyl, oxetanyl, tetrahydro-2-furanyl, tetrahydro-3-furanyl or tetrahydro-2H-pyran-4-yl; preferably methyl, ethyl, propyl, isopropyl, oxetanyl and tetrahydro-2H-pyran-4-yl.

Embodiment 11. A compound according to any of the preceding Embodiments, or a tautomer, stereoisomer or racemate thereof or a pharmaceutically acceptable salt thereof, wherein Raa is selected from the group consisting of methyl, ethyl, propyl, isopropyl, n-butyl and sec-butyl.

Embodiment 12. A compound according to any of Embodiments 1-3, or a tautomer, stereoisomer or racemate thereof or a pharmaceutically acceptable salt thereof, wherein
Ra1 and Ra3 is independently selected from the group consisting of H, C₁₋₆alkyl, C₁₋₆alkyl-O— and C₁₋₆alkyl-O—CH₂—;
Ra2 is selected from the group consisting of C₁₋₆alkyl, C₁₋₆alkyl-O— and C₁₋₆alkyl-O—CH₂—;
or Ra2 and Ra3 are taken together with the carbon they attached to form C₃₋₆cycloalkyl, or 5-6 membered haloheterocycloalkyl comprising 1 ring heteroatom selected from O).

Embodiment 13. A compound according to any of the preceding Embodiments, or a tautomer, stereoisomer or racemate thereof or a pharmaceutically acceptable salt thereof, wherein
Ra1 is selected from the group consisting of C₁₋₆alkyl, C₁₋₆alkyl-O— and C₁₋₆alkyl-O—CH₂—.

Embodiment 14. A compound according to any of the preceding Embodiments, or a tautomer, stereoisomer or racemate thereof or a pharmaceutically acceptable salt thereof, wherein
Ra3 is selected from the group consisting of C₁₋₆alkyl, C₁₋₆alkyl-O— and C₁₋₆alkyl-O—CH₂—.

Embodiment 15. A compound according to any of Embodiments 1-3, or a tautomer, stereoisomer or racemate thereof or a pharmaceutically acceptable salt thereof, wherein
Ra1 is C₁₋₆alkyl-O— or C₁₋₆alkyl-O—CH₂—,
Ra2 is selected from the group consisting of C₁₋₆alkyl, C₁₋₆alkyl-O— and C₁₋₆alkyl-O—CH₂—, and
Ra3 is selected from the group consisting of H, C₁₋₆alkyl, C₁₋₆alkyl-O— and C₁₋₆alkyl-O—CH₂—.

Embodiment 16. A compound according to any of Embodiments 1-3, or a tautomer, stereoisomer or racemate thereof or a pharmaceutically acceptable salt thereof, wherein
Ra1 is C₁₋₆alkyl-O—,
Ra2 is C₁₋₆alkyl, and
Ra3 is H or C₁₋₆alkyl.

Embodiment 17. A compound according to any of Embodiments 1-3, or a tautomer, stereoisomer or racemate thereof or a pharmaceutically acceptable salt thereof, wherein
Ra1 is C₁₋₆alkyl-O—CH₂—, and
each of Ra2 and Ra3 is C₁₋₆alkyl.

Embodiment 18. A compound according to any of the preceding Embodiments, or a tautomer, stereoisomer or racemate thereof or a pharmaceutically acceptable salt thereof, wherein
Ra1 is C₁₋₆alkyl-O— or C₁₋₆alkyl-O—CH₂—, and
each of Ra2 and Ra3 is C₁₋₆alkyl-O—CH₂—.

Embodiment 19. A compound according to any of the preceding Embodiments, or a tautomer, stereoisomer or racemate thereof or a pharmaceutically acceptable salt thereof, wherein
Ra1 is C₁₋₆alkyl-O—CH₂, and
one of Ra2 and Ra3 is C₁₋₆alkyl, and the other is C₁₋₆alkyl-O—CH₂.

Embodiment 20. A compound according to any of the preceding Embodiments, or a tautomer, stereoisomer or racemate thereof or a pharmaceutically acceptable salt thereof, wherein
Ra1 is C₁₋₆alkyl-O—, and
Ra2 and Ra3 is independently C₁₋₃alkyl, preferably Ra2 and Ra3 are the same.

Embodiment 21. A compound according to any of the preceding Embodiments, or a tautomer, stereoisomer or racemate thereof or a pharmaceutically acceptable salt thereof, wherein
Ra1 is C₁₋₆alkyl-O— or C₁₋₆alkyl-O—CH₂—, and
Ra2 and Ra3 are taken together with the carbon they attached to form a C₃₋₆cycloalkyl.

Embodiment 22. A compound according to any of the preceding Embodiments, or a tautomer, stereoisomer or racemate thereof or a pharmaceutically acceptable salt thereof, wherein
Ra1 is selected from the group consisting of H, C₁₋₆alkyl, C₁₋₆alkyl-O— and C₁₋₆alkyl-O—CH₂—; and
Ra2 and Ra3 are taken together with the carbon they attached to form a 5-6 membered haloheterocycloalkyl comprising 1 ring heteroatom selected from O;

Preferably, Ra1 is selected from the group consisting of C₁₋₆alkyl, C₁₋₆alkyl-O— and C₁₋₆alkyl-O—CH₂—.

Embodiment 23. A compound according to any of the preceding Embodiments, or a tautomer, stereoisomer or racemate thereof or a pharmaceutically acceptable salt thereof, wherein R′ is RaC═O, and each of R² and R³ is H.

Embodiment 24. A compound according to any of the preceding Embodiments, or a tautomer, stereoisomer or racemate thereof or a pharmaceutically acceptable salt thereof, wherein each of R¹, R² and R³ are RaC═O.

Embodiment 25. A compound according to any of the preceding Embodiments, or a tautomer, stereoisomer or racemate thereof or a tautomer, stereoisomer or racemate thereof or a pharmaceutically acceptable salt thereof, wherein Ra1 is C₁₋₆alkyl-O)—.

Embodiment 26. A compound according to any of the preceding Embodiments, or a tautomer, stereoisomer or racemate thereof or a tautomer, stereoisomer or racemate thereof or a pharmaceutically acceptable salt thereof, wherein Ra—(C═O)— is selected from the group consisting of:

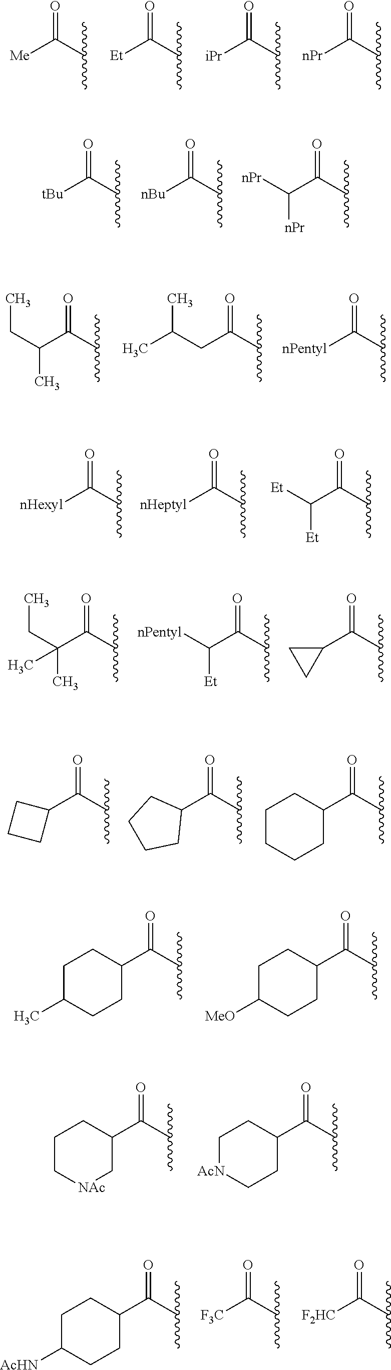

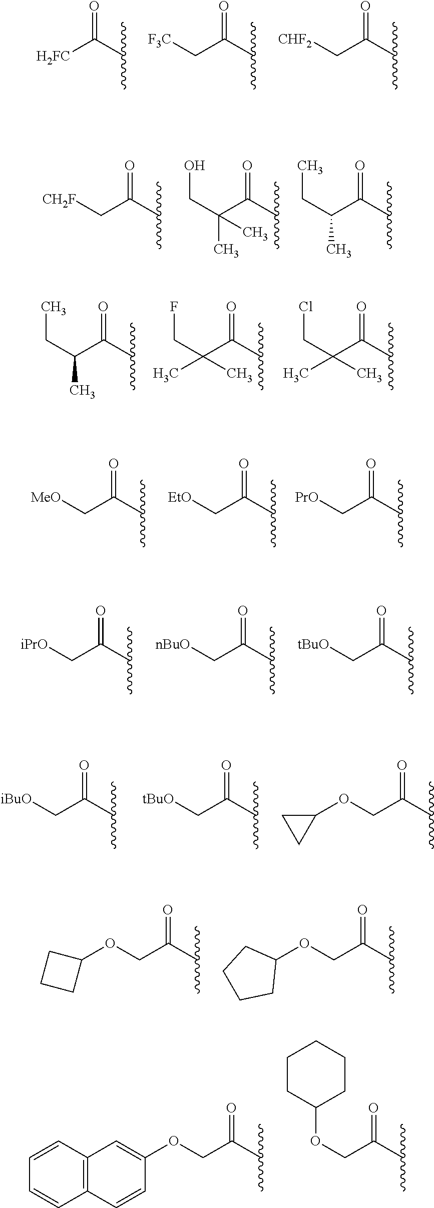

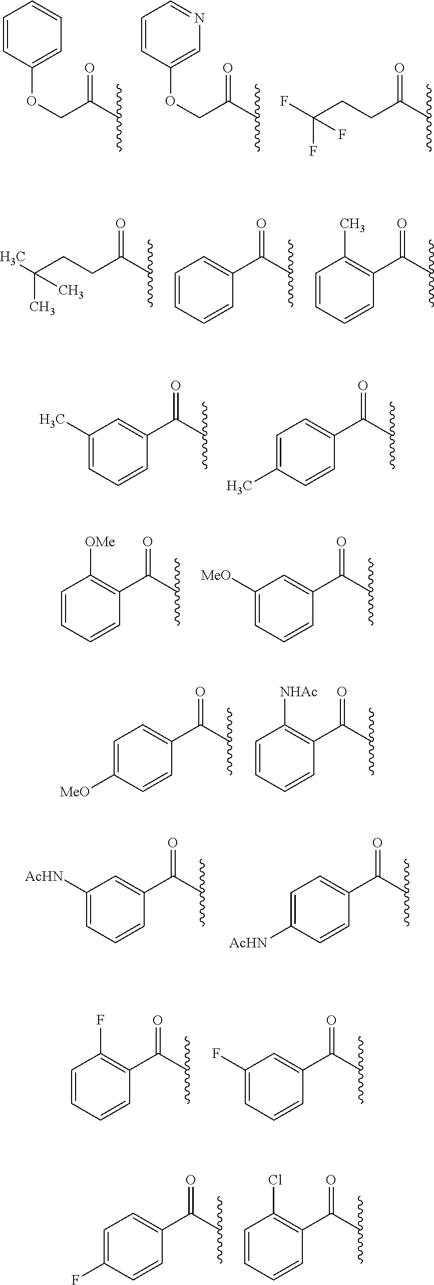

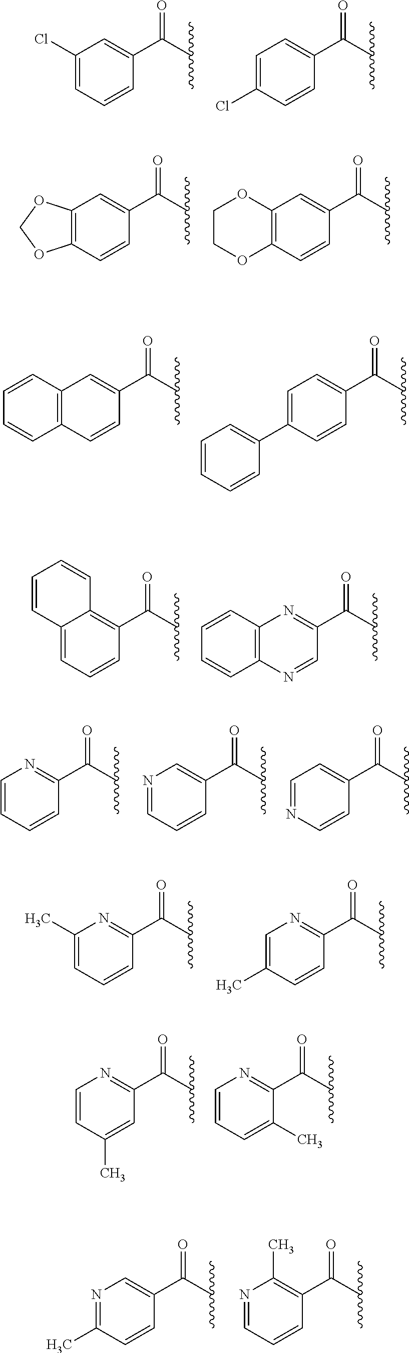

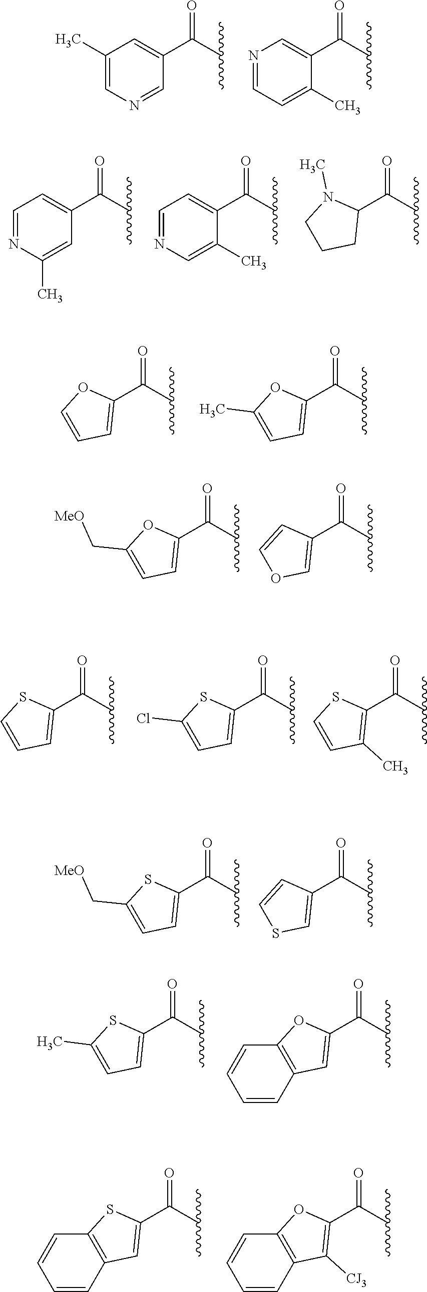

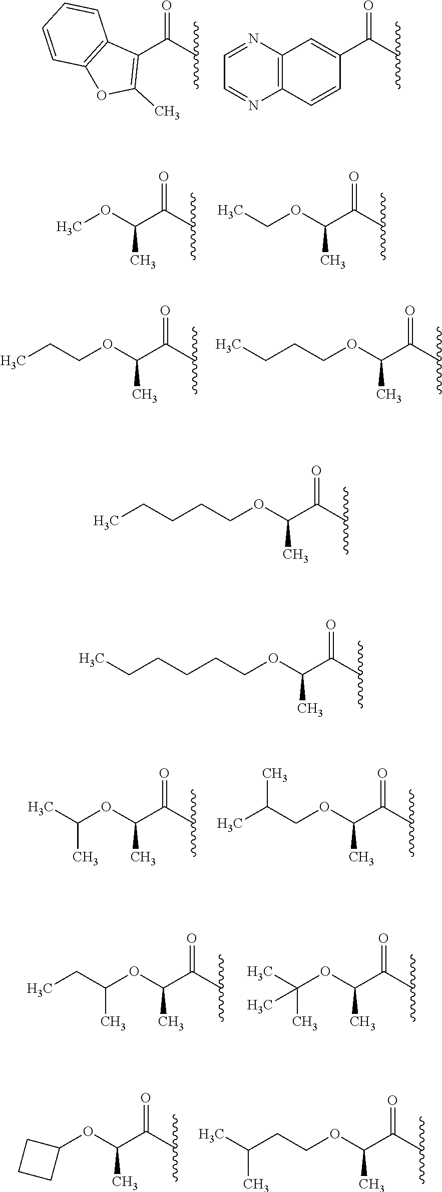

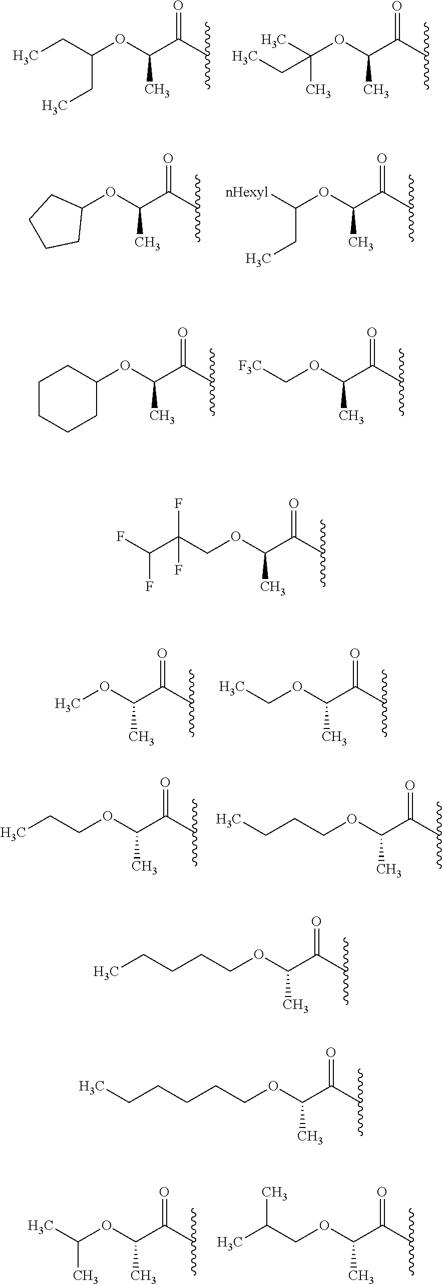

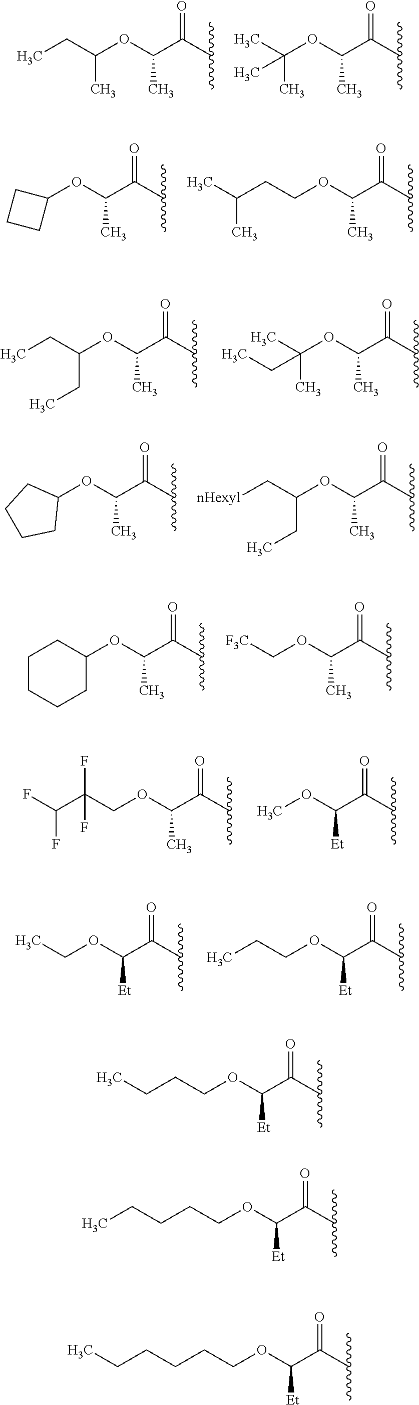

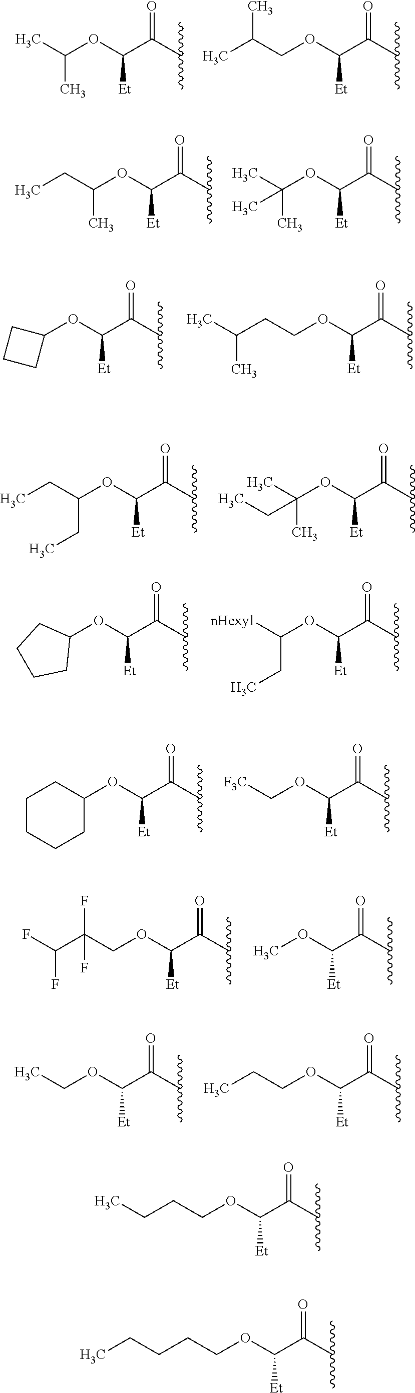

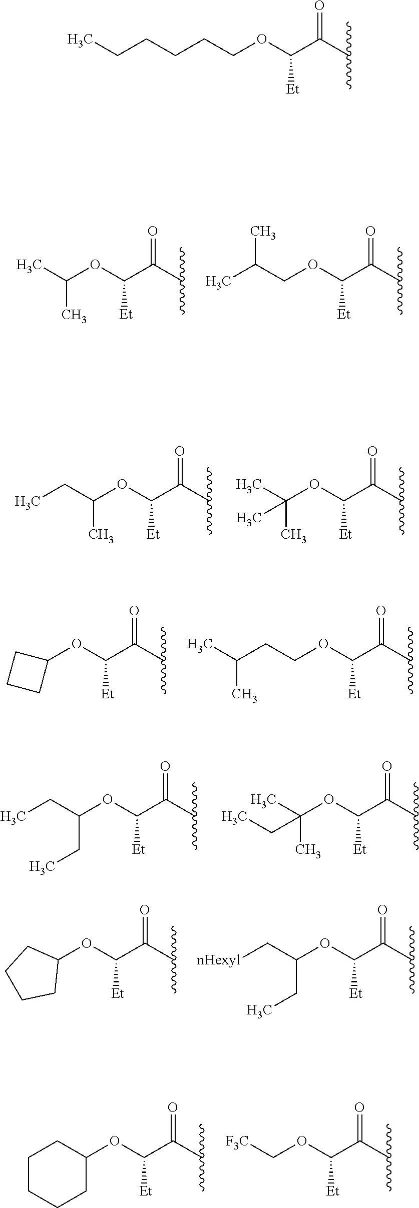

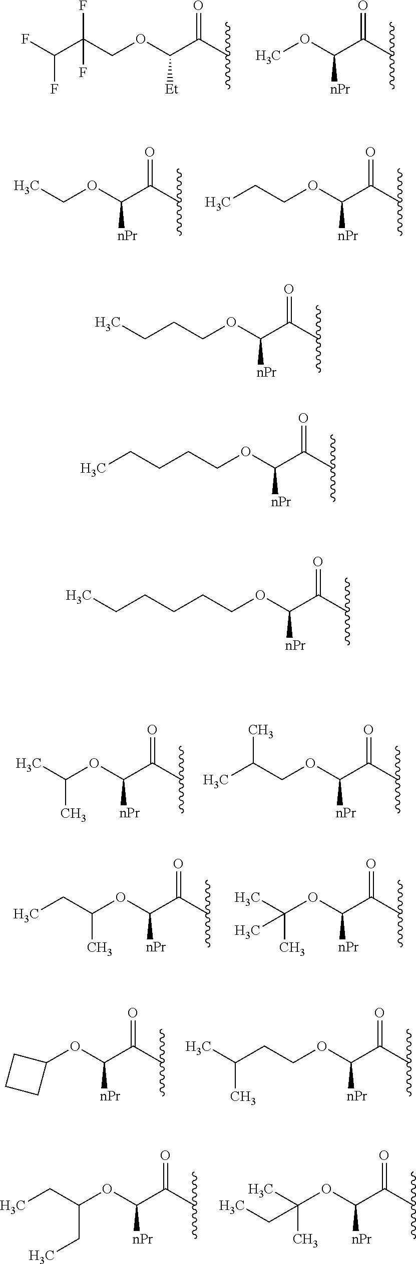

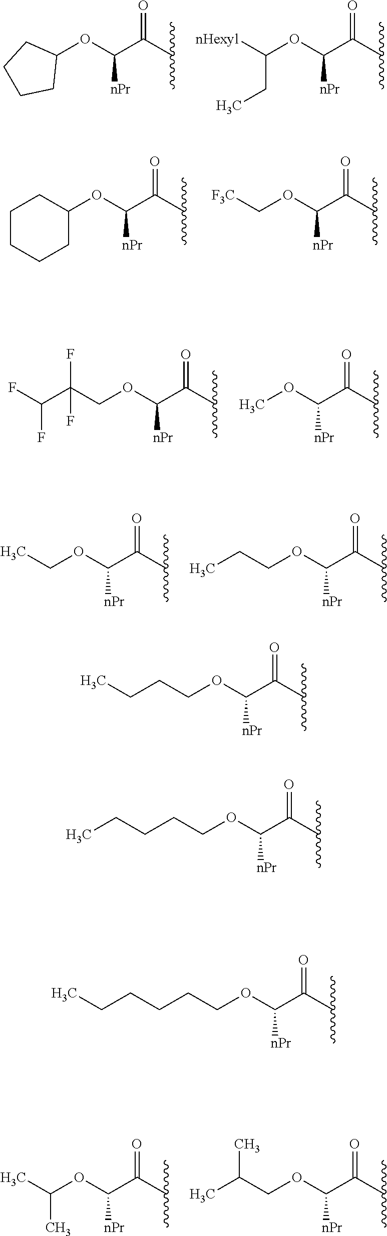

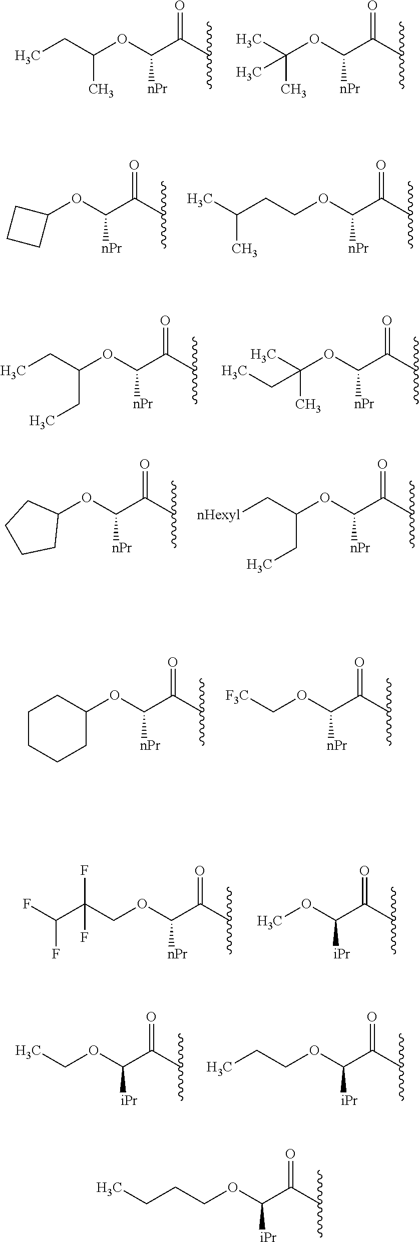

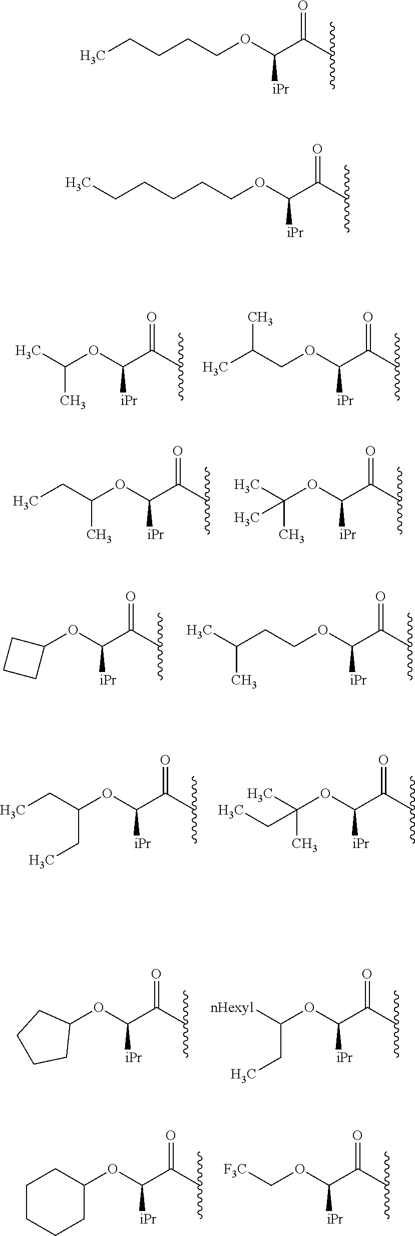

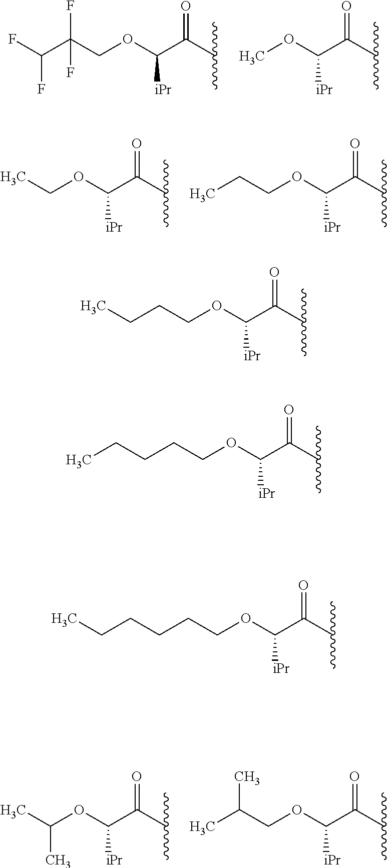

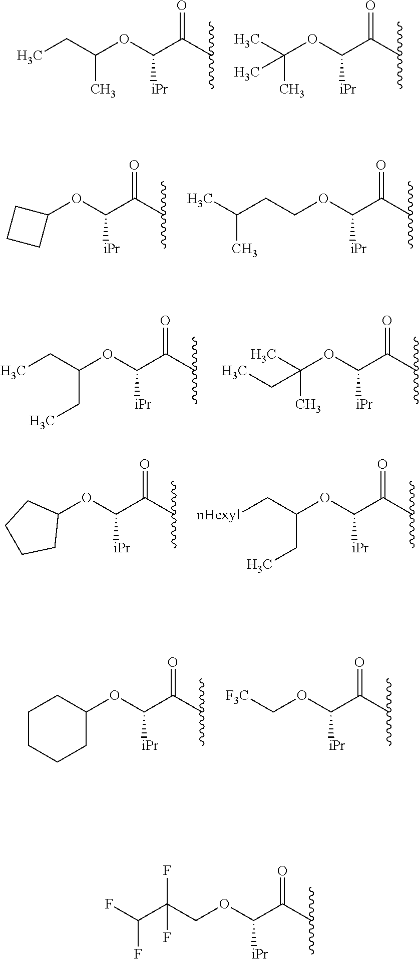

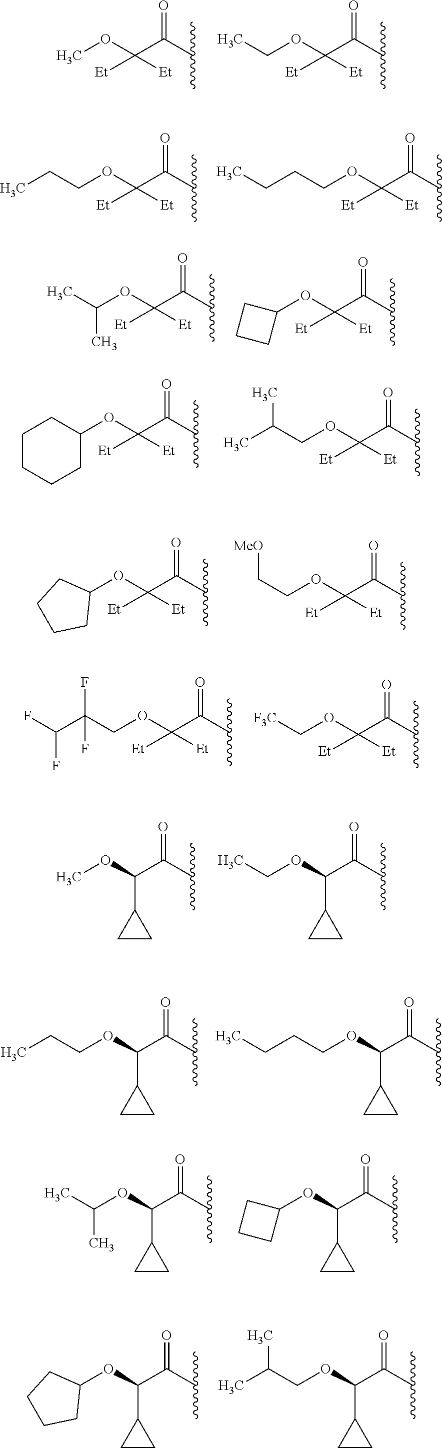

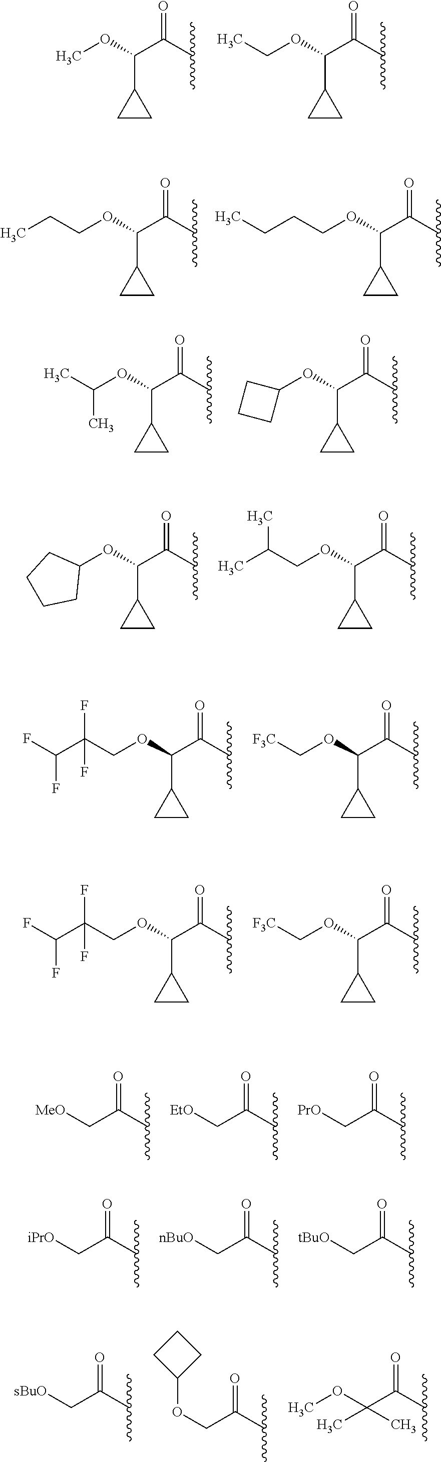

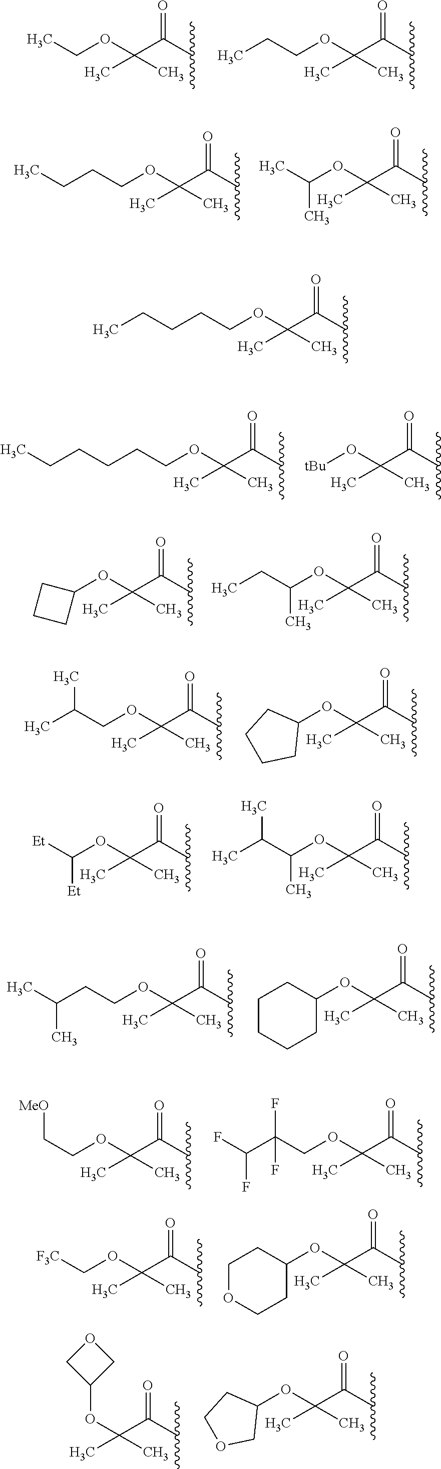

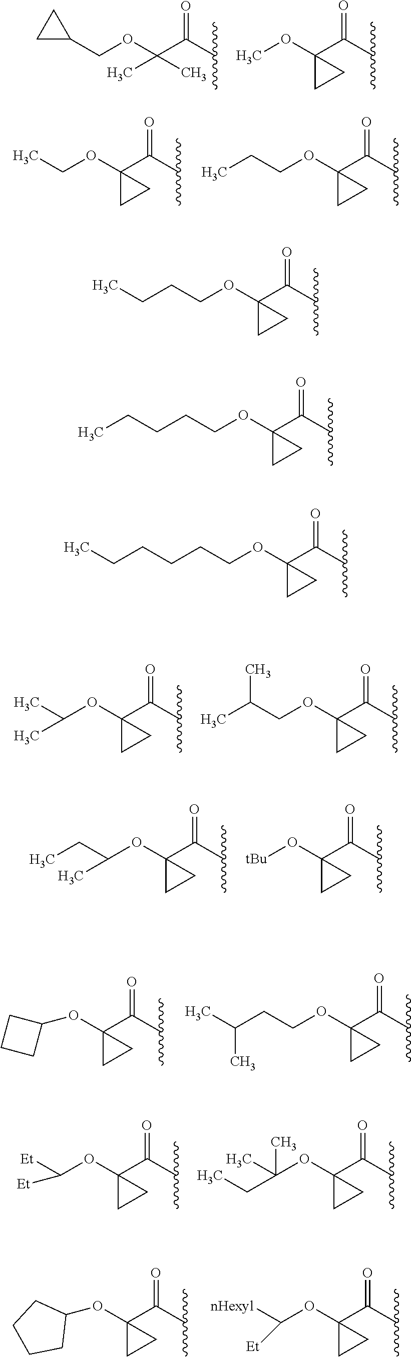

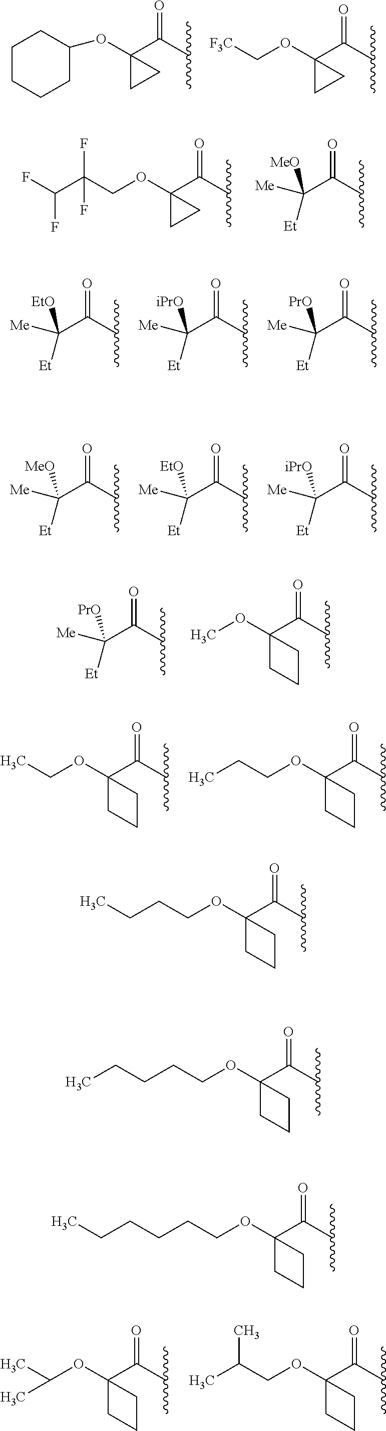

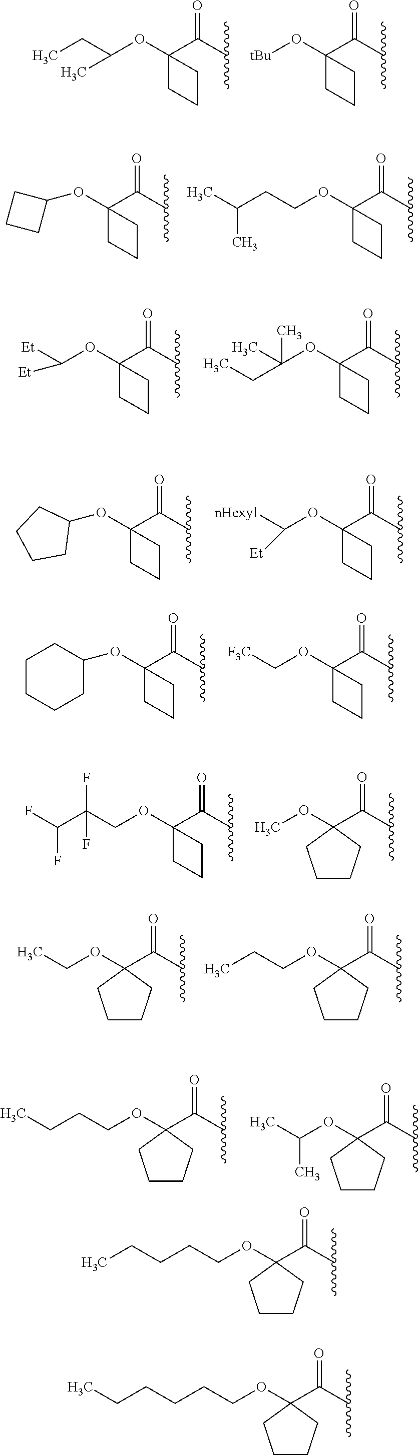

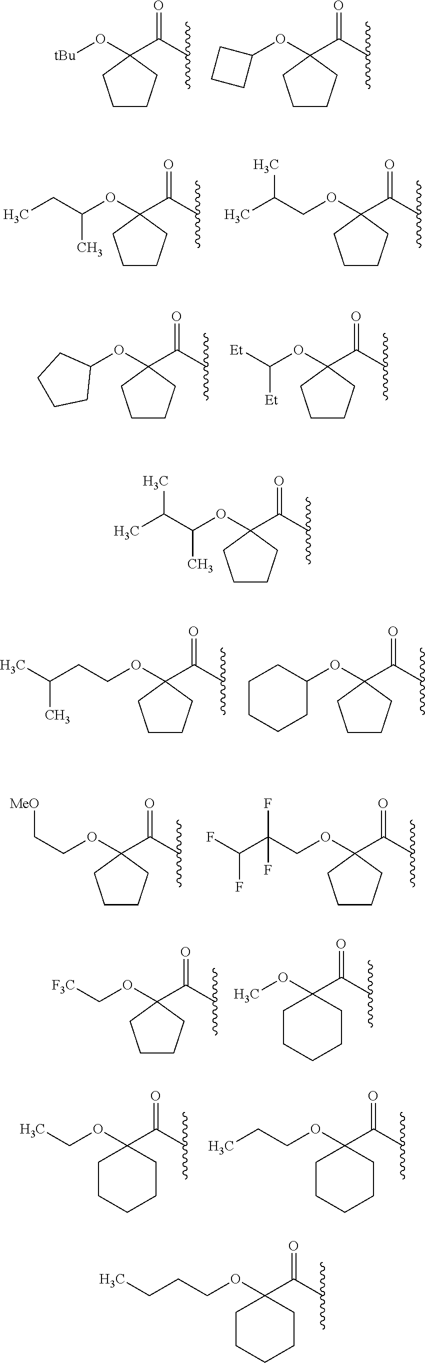

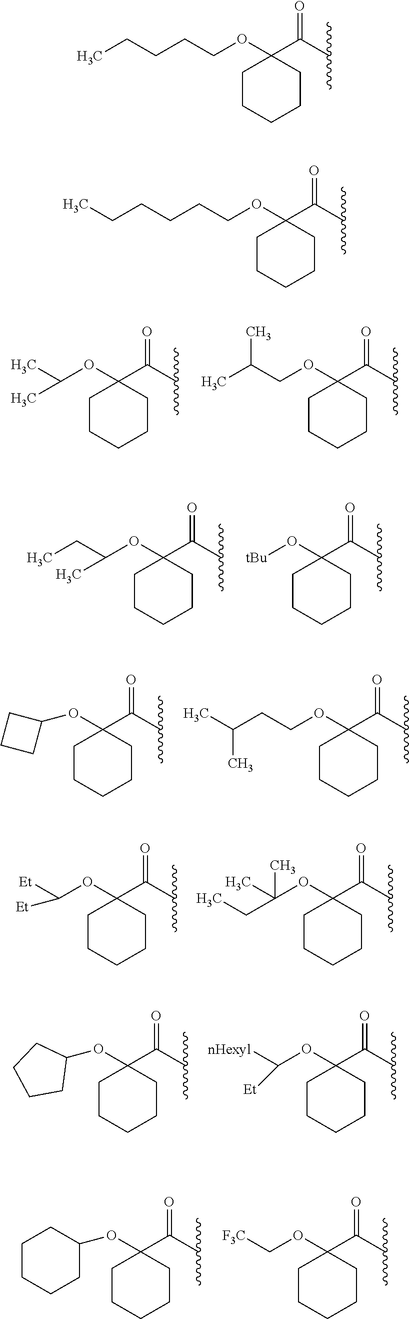

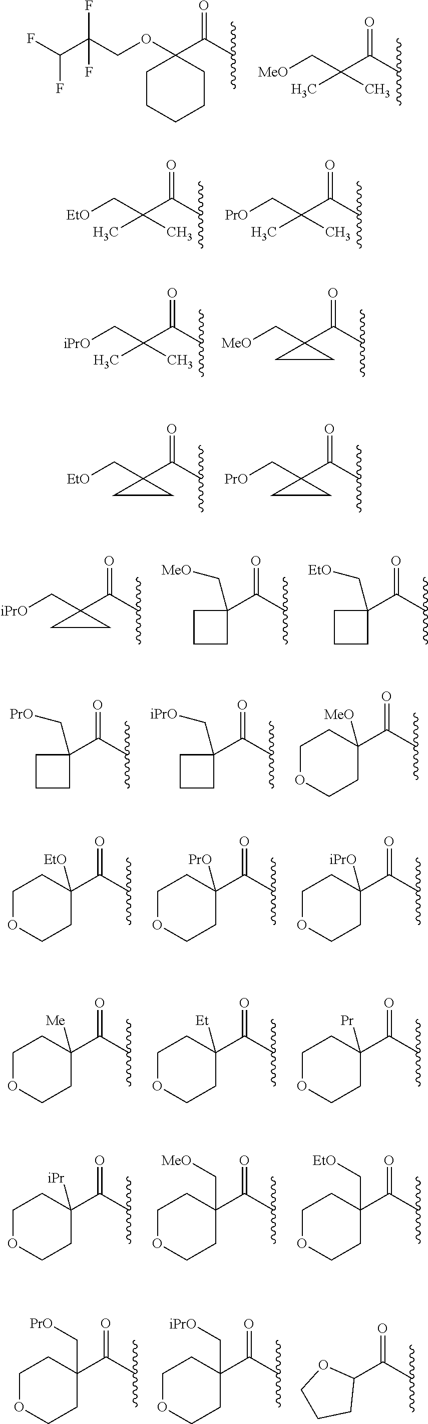

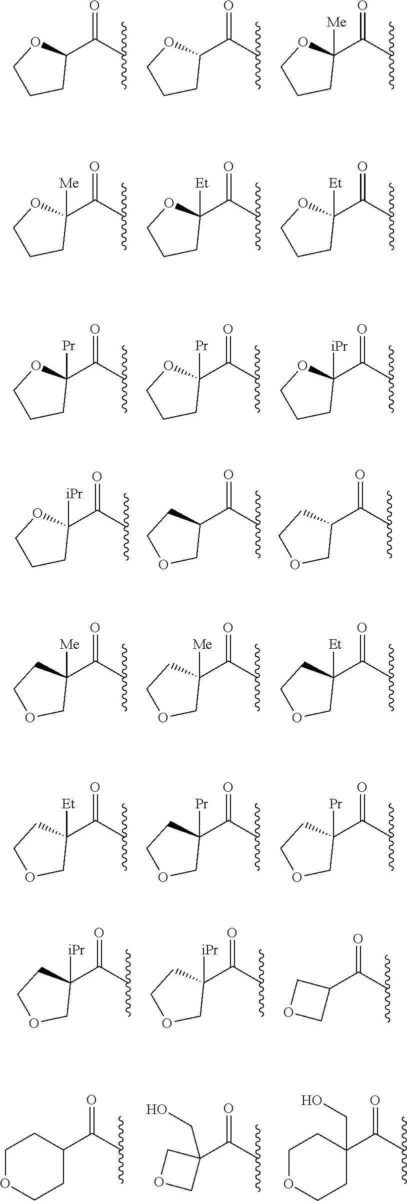

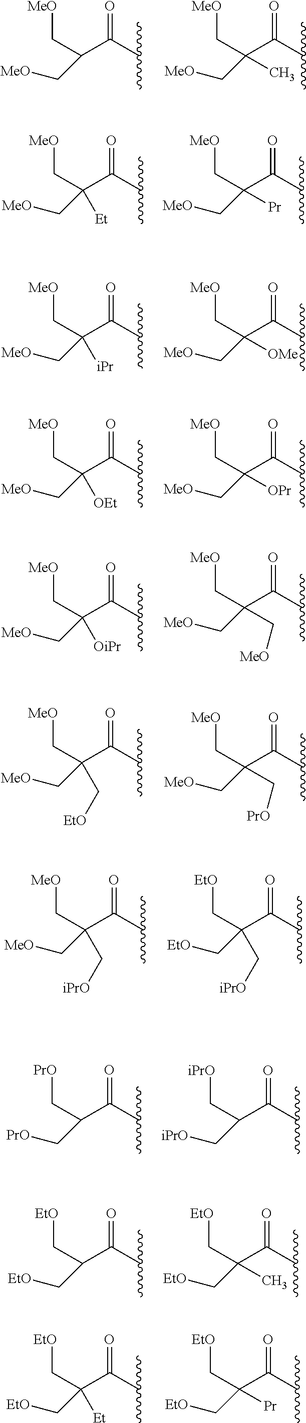

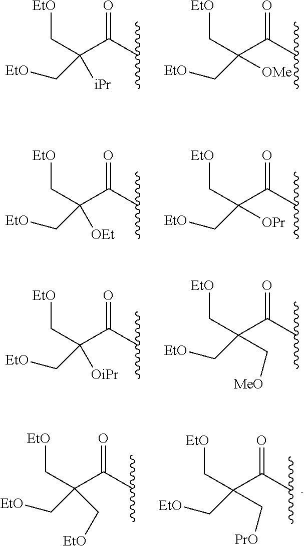

Embodiment 27. A compound according to Embodiment 1, or a tautomer, stereoisomer or racemate thereof or a tautomer, stereoisomer or racemate thereof or a pharmaceutically acceptable salt thereof, wherein the compound is selected from the group consisting of:

| Example # | Structure |
|---|---|
| EX-2 | 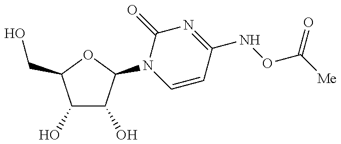 [SMILES: CC(=O)ONc1ccn([C@@H]2O[C@H](CO)[C@@H](O)[C@H]2O)c(=O)n1] |
| EX-3 | 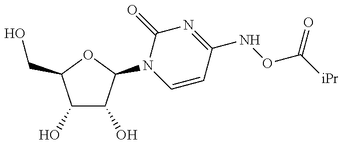 [SMILES: CC(C)C(=O)ONc1ccn([C@@H]2O[C@H](CO)[C@@H](O)[C@H]2O)c(=O)n1] |
| EX-4 | 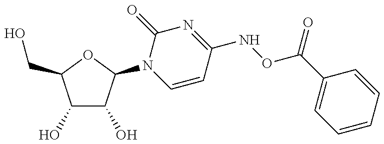 [SMILES: O=C(ONc1ccn([C@@H]2O[C@H](CO)[C@@H](O)[C@H]2O)c(=O)n1)c1ccccc1] |
| EX-5 | 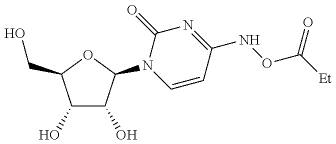 [SMILES: CCC(=O)ONc1ccn([C@@H]2O[C@H](CO)[C@@H](O)[C@H]2O)c(=O)n1] |
| EX-4a | 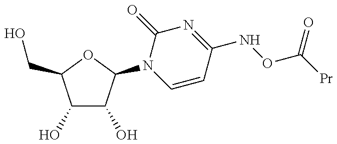 |
| EX-5a | 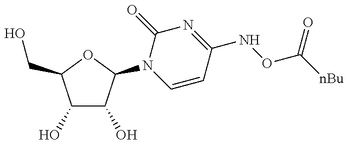 |
| EX-6 | 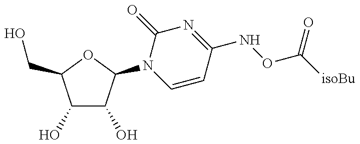 |
| EX-7 | 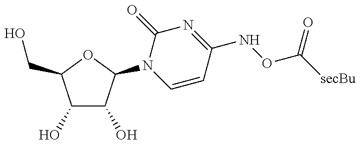 |
| EX-8 | 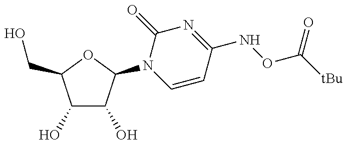 [SMILES: CC(C)(C)C(=O)ONc1ccn([C@@H]2O[C@H](CO)[C@@H](O)[C@H]2O)c(=O)n1] |
| EX-9 | 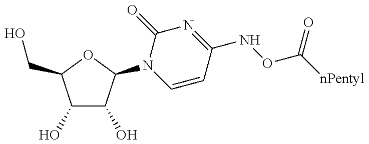 |
| EX-10 | 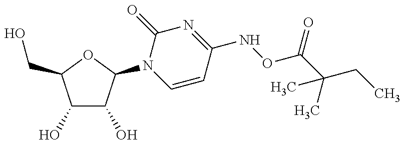 [SMILES: CCC(C)(C)C(=O)ONc1ccn([C@@H]2O[C@H](CO)[C@@H](O)[C@H]2O)c(=O)n1] |
| EX-11 | 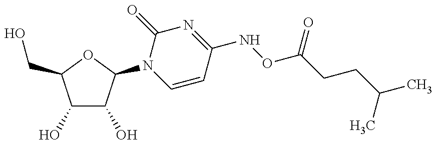 [SMILES: CC(C)CCC(=O)ONc1ccn([C@@H]2O[C@H](CO)[C@@H](O)[C@H]2O)c(=O)n1] |
| EX-12 | 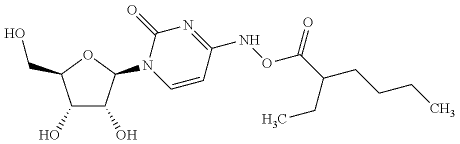 [SMILES: CCCCC(CC)C(=O)ONc1ccn([C@@H]2O[C@H](CO)[C@@H](O)[C@H]2O)c(=O)n1] |
| EX-13 | 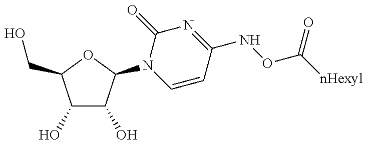 |
| EX-14 | 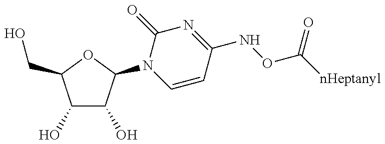 |
| EX-15 | 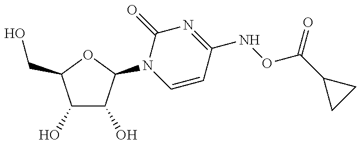 [SMILES: O=C(ONc1ccn([C@@H]2O[C@H](CO)[C@@H](O)[C@H]2O)c(=O)n1)C1CC1] |
| EX-16 | 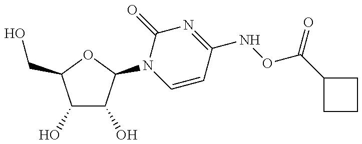 [SMILES: O=C(ONc1ccn([C@@H]2O[C@H](CO)[C@@H](O)[C@H]2O)c(=O)n1)C1CCC1] |
| EX-17 | 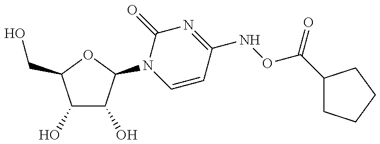 [SMILES: O=C(ONc1ccn([C@@H]2O[C@H](CO)[C@@H](O)[C@H]2O)c(=O)n1)C1CCCC1] |
| EX-18 | 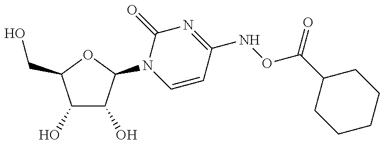 [SMILES: O=C(ONc1ccn([C@@H]2O[C@H](CO)[C@@H](O)[C@H]2O)c(=O)n1)C1CCCCC1] |
| EX-19 | 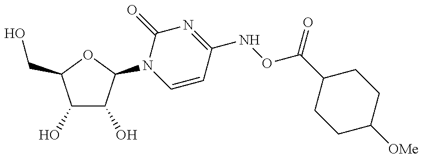 [SMILES: COC1CCC(C(=O)ONc2ccn([C@@H]3O[C@H](CO)[C@@H](O)[C@H]3O)c(=O)n2)CC1] |
| EX-20 | 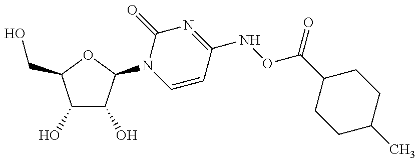 [SMILES: CC1CCC(C(=O)ONc2ccn([C@@H]3O[C@H](CO)[C@@H](O)[C@H]3O)c(=O)n2)CC1] |
| EX-21 | 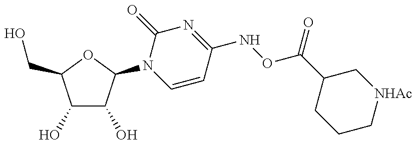 |
| EX-22 | 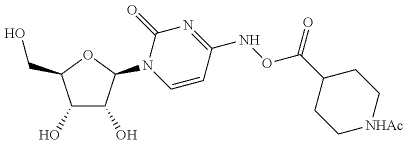 |
| EX-23 | 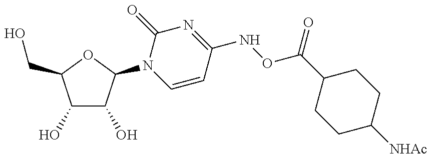 [SMILES: CC(=O)NC1CCC(C(=O)ONc2ccn([C@@H]3O[C@H](CO)[C@@H](O)[C@H]3O)c(=O)n2)CC1] |
| EX-24 | 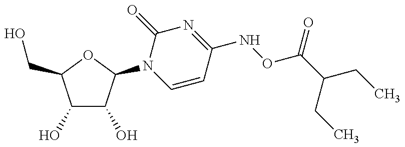 [SMILES: CCC(CC)C(=O)ONc1ccn([C@@H]2O[C@H](CO)[C@@H](O)[C@H]2O)c(=O)n1] |
| EX-25 | 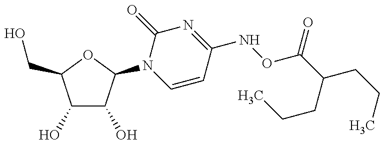 [SMILES: CCCC(CCC)C(=O)ONc1ccn([C@@H]2O[C@H](CO)[C@@H](O)[C@H]2O)c(=O)n1] |
| EX-26 | 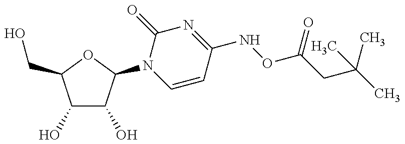 [SMILES: CC(C)(C)CC(=O)ONc1ccn([C@@H]2O[C@H](CO)[C@@H](O)[C@H]2O)c(=O)n1] |
| EX-27 | 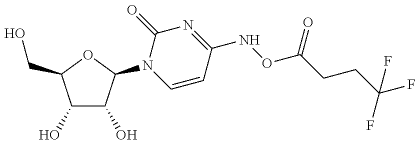 [SMILES: O=C(CCC(F)(F)F)ONc1ccn([C@@H]2O[C@H](CO)[C@@H](O)[C@H]2O)c(=O)n1] |
| EX-28 | 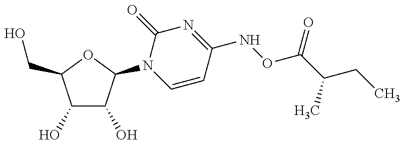 [SMILES: CC[C@H](C)C(=O)ONc1ccn([C@@H]2O[C@H](CO)[C@@H](O)[C@H]2O)c(=O)n1] |
| EX-29 | 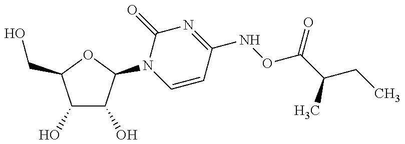 [SMILES: CC[C@@H](C)C(=O)ONc1ccn([C@@H]2O[C@H](CO)[C@@H](O)[C@H]2O)c(=O)n1] |
| EX-30 | 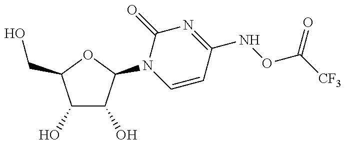 [SMILES: O=C(ONc1ccn([C@@H]2O[C@H](CO)[C@@H](O)[C@H]2O)c(=O)n1)C(F)(F)F] |
| EX-31 | 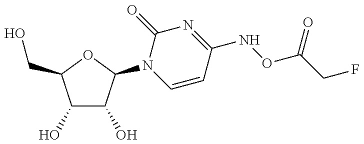 [SMILES: O=C(CF)ONc1ccn([C@@H]2O[C@H](CO)[C@@H](O)[C@H]2O)c(=O)n1] |
| EX-32 | 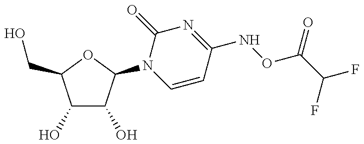 [SMILES: O=C(ONc1ccn([C@@H]2O[C@H](CO)[C@@H](O)[C@H]2O)c(=O)n1)C(F)F] |
| EX-33 | 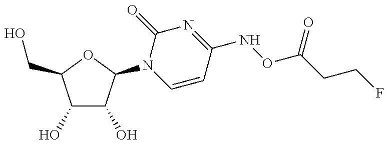 [SMILES: O=C(CCF)ONc1ccn([C@@H]2O[C@H](CO)[C@@H](O)[C@H]2O)c(=O)n1] |
| EX-34 | 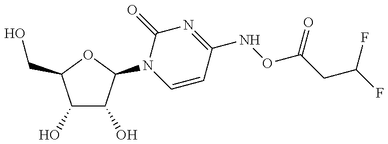 [SMILES: O=C(CC(F)F)ONc1ccn([C@@H]2O[C@H](CO)[C@@H](O)[C@H]2O)c(=O)n1] |
| EX-35 | 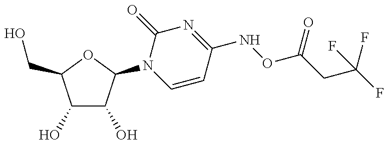 [SMILES: O=C(CC(F)(F)F)ONc1ccn([C@@H]2O[C@H](CO)[C@@H](O)[C@H]2O)c(=O)n1] |
| EX-36 | 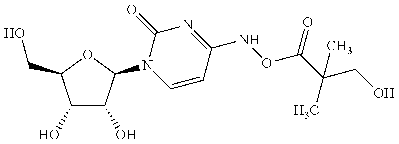 [SMILES: CC(C)(CO)C(=O)ONc1ccn([C@@H]2O[C@H](CO)[C@@H](O)[C@H]2O)c(=O)n1] |
| EX-37 | 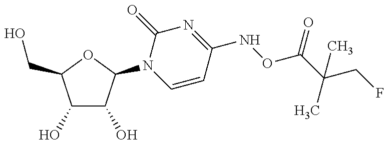 [SMILES: CC(C)(CF)C(=O)ONc1ccn([C@@H]2O[C@H](CO)[C@@H](O)[C@H]2O)c(=O)n1] |
| EX-38 | 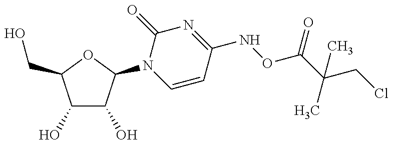 [SMILES: CC(C)(CCl)C(=O)ONc1ccn([C@@H]2O[C@H](CO)[C@@H](O)[C@H]2O)c(=O)n1] |
| EX-39 | 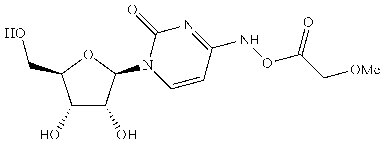 [SMILES: COCC(=O)ONc1ccn([C@@H]2O[C@H](CO)[C@@H](O)[C@H]2O)c(=O)n1] |
| EX-40 | 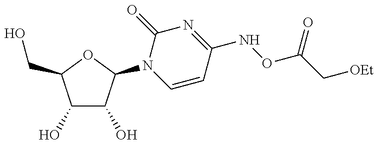 [SMILES: CCOCC(=O)ONc1ccn([C@@H]2O[C@H](CO)[C@@H](O)[C@H]2O)c(=O)n1] |
| EX-41 | 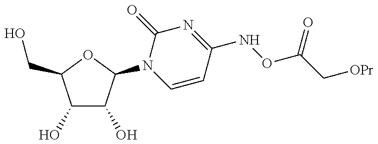 |
| EX-42 | 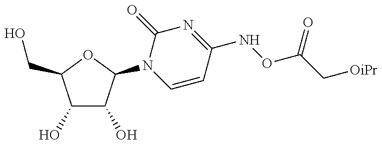 [SMILES: CC(C)OCC(=O)ONc1ccn([C@@H]2O[C@H](CO)[C@@H](O)[C@H]2O)c(=O)n1] |
| EX-43 | 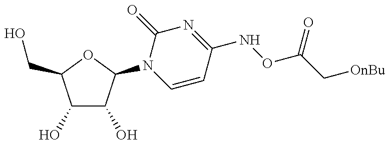 |
| EX-44 | 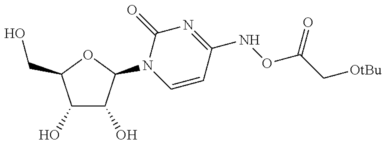 [SMILES: CC(C)(C)OCC(=O)ONc1ccn([C@@H]2O[C@H](CO)[C@@H](O)[C@H]2O)c(=O)n1] |
| EX-45 | 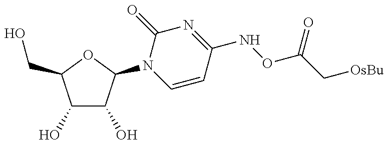 |
| EX-46 | 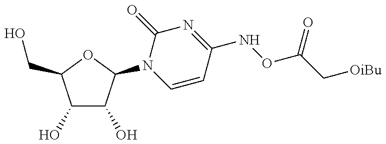 |
| EX-47 | 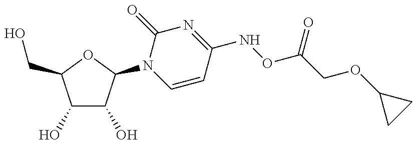 [SMILES: O=C(COC1CC1)ONc1ccn([C@@H]2O[C@H](CO)[C@@H](O)[C@H]2O)c(=O)n1] |
| EX-48 | 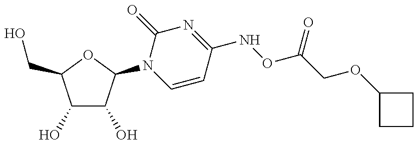 [SMILES: O=C(COC1CCC1)ONc1ccn([C@@H]2O[C@H](CO)[C@@H](O)[C@H]2O)c(=O)n1] |
| EX-49 | 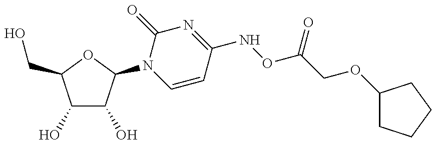 [SMILES: O=C(COC1CCCC1)ONc1ccn([C@@H]2O[C@H](CO)[C@@H](O)[C@H]2O)c(=O)n1] |
| EX-50 | 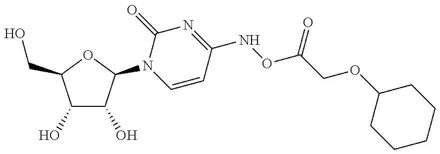 [SMILES: O=C(COC1CCCCC1)ONc1ccn([C@@H]2O[C@H](CO)[C@@H](O)[C@H]2O)c(=O)n1] |
| EX-51 | 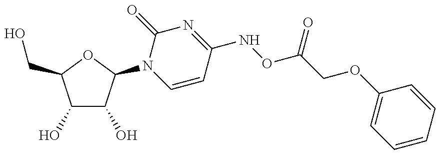 [SMILES: O=C(COc1ccccc1)ONc1ccn([C@@H]2O[C@H](CO)[C@@H](O)[C@H]2O)c(=O)n1] |
| EX-52 | 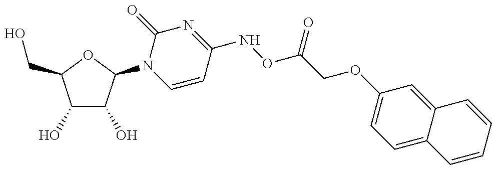 [SMILES: O=C(COc1ccc2ccccc2c1)ONc1ccn([C@@H]2O[C@H](CO)[C@@H](O)[C@H]2O)c(=O)n1] |
| EX-53 | 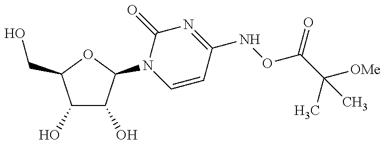 [SMILES: COC(C)(C)C(=O)ONc1ccn([C@@H]2O[C@H](CO)[C@@H](O)[C@H]2O)c(=O)n1] |
| EX-54 | 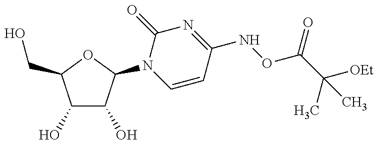 [SMILES: CCOC(C)(C)C(=O)ONc1ccn([C@@H]2O[C@H](CO)[C@@H](O)[C@H]2O)c(=O)n1] |
| EX-55 | 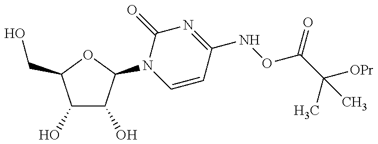 |
| EX-56 | 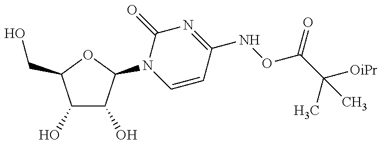 [SMILES: CC(C)OC(C)(C)C(=O)ONc1ccn([C@@H]2O[C@H](CO)[C@@H](O)[C@H]2O)c(=O)n1] |
| EX-57 | 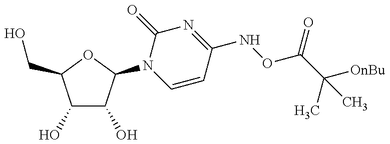 |
| EX-58 | 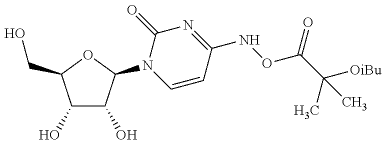 |
| EX-59 | 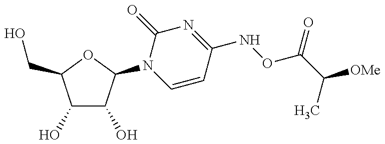 [SMILES: CO[C@@H](C)C(=O)ONc1ccn([C@@H]2O[C@H](CO)[C@@H](O)[C@H]2O)c(=O)n1] |
| EX-60 | 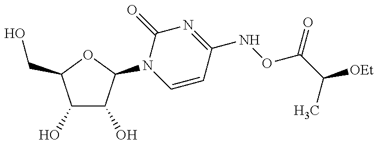 [SMILES: CCO[C@@H](C)C(=O)ONc1ccn([C@@H]2O[C@H](CO)[C@@H](O)[C@H]2O)c(=O)n1] |
| EX-61 | 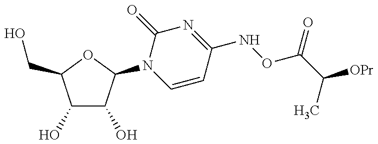 |
| EX-62 | 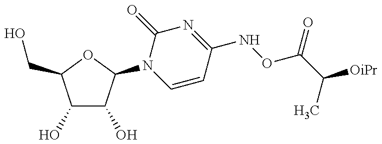 [SMILES: CC(C)O[C@@H](C)C(=O)ONc1ccn([C@@H]2O[C@H](CO)[C@@H](O)[C@H]2O)c(=O)n1] |
| EX-63 | 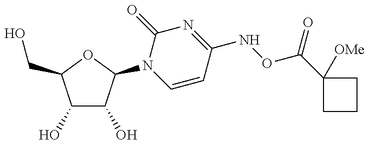 [SMILES: COC1(C(=O)ONc2ccn([C@@H]3O[C@H](CO)[C@@H](O)[C@H]3O)c(=O)n2)CCC1] |
| EX-64 | 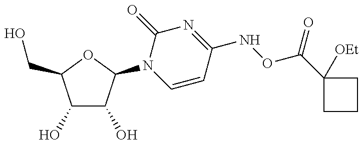 [SMILES: CCOC1(C(=O)ONc2ccn([C@@H]3O[C@H](CO)[C@@H](O)[C@H]3O)c(=O)n2)CCC1] |
| EX-65 | 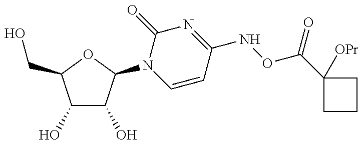 |
| EX-66 | 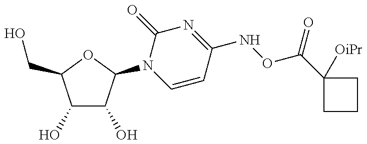 [SMILES: CC(C)OC1(C(=O)ONc2ccn([C@@H]3O[C@H](CO)[C@@H](O)[C@H]3O)c(=O)n2)CCC1] |
| EX-67 | 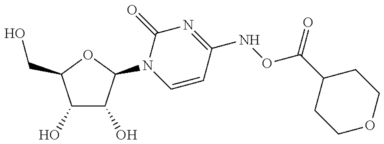 [SMILES: O=C(ONc1ccn([C@@H]2O[C@H](CO)[C@@H](O)[C@H]2O)c(=O)n1)C1CCOCC1] |
| EX-68 | 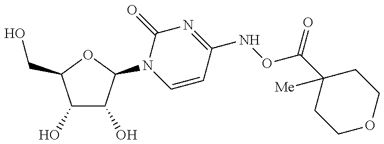 [SMILES: CC1(C(=O)ONc2ccn([C@@H]3O[C@H](CO)[C@@H](O)[C@H]3O)c(=O)n2)CCOCC1] |
| EX-69 | 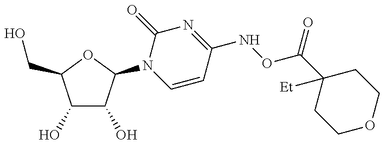 [SMILES: CCC1(C(=O)ONc2ccn([C@@H]3O[C@H](CO)[C@@H](O)[C@H]3O)c(=O)n2)CCOCC1] |
| EX-70 | 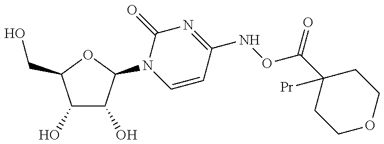 |
| EX-71 | 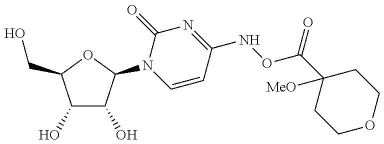 [SMILES: COC1(C(=O)ONc2ccn([C@@H]3O[C@H](CO)[C@@H](O)[C@H]3O)c(=O)n2)CCOCC1] |
| EX-72 | 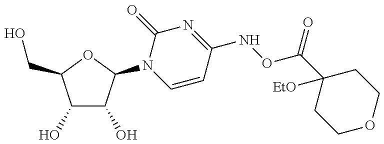 [SMILES: CCOC1(C(=O)ONc2ccn([C@@H]3O[C@H](CO)[C@@H](O)[C@H]3O)c(=O)n2)CCOCC1] |
| EX-73 | 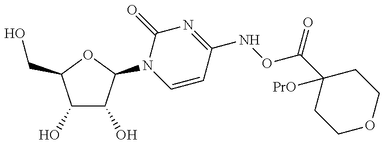 |
| EX-74 | 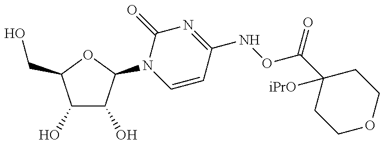 [SMILES: CC(C)OC1(C(=O)ONc2ccn([C@@H]3O[C@H](CO)[C@@H](O)[C@H]3O)c(=O)n2)CCOCC1] |
| EX-75 | 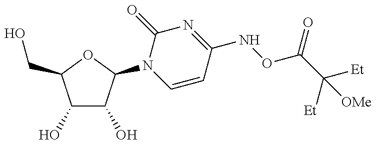 [SMILES: CCC(CC)(OC)C(=O)ONc1ccn([C@@H]2O[C@H](CO)[C@@H](O)[C@H]2O)c(=O)n1] |
| EX-76 | 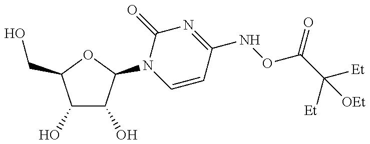 [SMILES: CCOC(CC)(CC)C(=O)ONc1ccn([C@@H]2O[C@H](CO)[C@@H](O)[C@H]2O)c(=O)n1] |
| EX-77 | 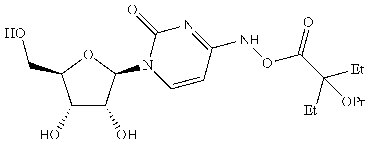 |
| EX-78 | 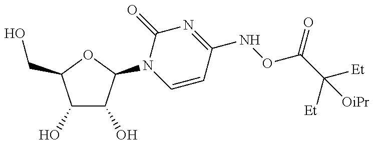 [SMILES: CCC(CC)(OC(C)C)C(=O)ONc1ccn([C@@H]2O[C@H](CO)[C@@H](O)[C@H]2O)c(=O)n1] |
| EX-79 | 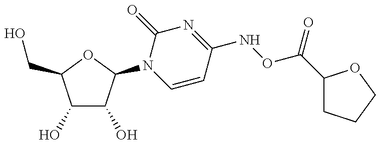 [SMILES: O=C(ONc1ccn([C@@H]2O[C@H](CO)[C@@H](O)[C@H]2O)c(=O)n1)C1CCCO1] |
| EX-80 | 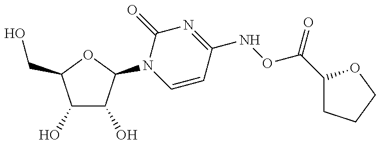 |
| EX-81 | 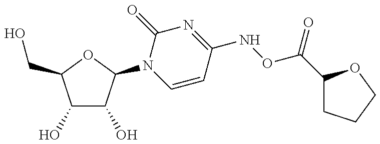 |
| EX-82 | 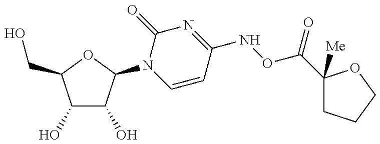 [SMILES: C[C@]1(C(=O)ONc2ccn([C@@H]3O[C@H](CO)[C@@H](O)[C@H]3O)c(=O)n2)CCCO1] |
| EX-83 | 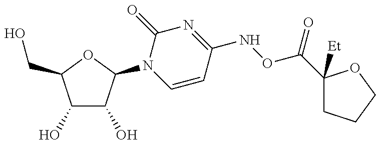 [SMILES: CC[C@]1(C(=O)ONc2ccn([C@@H]3O[C@H](CO)[C@@H](O)[C@H]3O)c(=O)n2)CCCO1] |
| EX-84 | 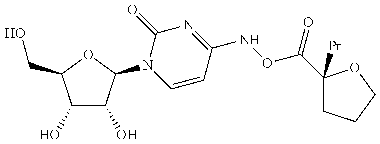 |
| EX-85 | 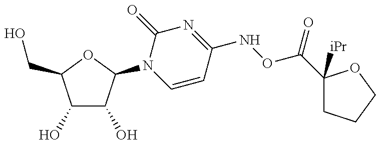 [SMILES: CC(C)[C@]1(C(=O)ONc2ccn([C@@H]3O[C@H](CO)[C@@H](O)[C@H]3O)c(=O)n2)CCCO1] |
| EX-86 | 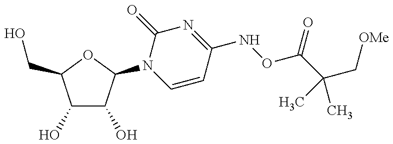 [SMILES: COCC(C)(C)C(=O)ONc1ccn([C@@H]2O[C@H](CO)[C@@H](O)[C@H]2O)c(=O)n1] |
| EX-87 | 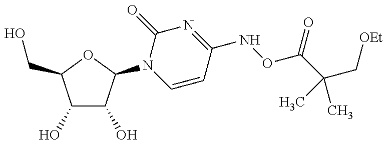 [SMILES: CCOCC(C)(C)C(=O)ONc1ccn([C@@H]2O[C@H](CO)[C@@H](O)[C@H]2O)c(=O)n1] |
| EX-88 | 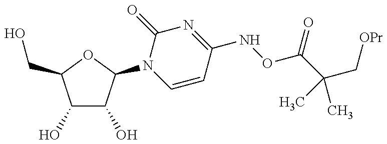 |
| EX-89 | 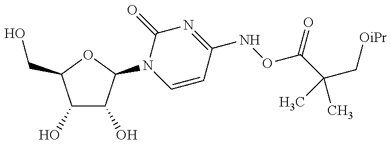 [SMILES: CC(C)OCC(C)(C)C(=O)ONc1ccn([C@@H]2O[C@H](CO)[C@@H](O)[C@H]2O)c(=O)n1] |
| EX-90 | 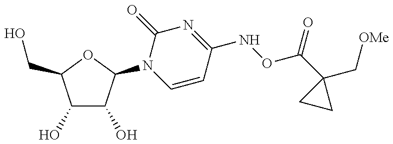 [SMILES: COCC1(C(=O)ONc2ccn([C@@H]3O[C@H](CO)[C@@H](O)[C@H]3O)c(=O)n2)CC1] |
| EX-91 | 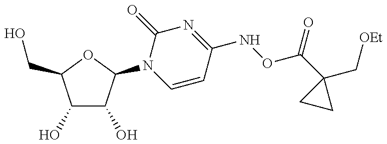 [SMILES: CCOCC1(C(=O)ONc2ccn([C@@H]3O[C@H](CO)[C@@H](O)[C@H]3O)c(=O)n2)CC1] |
| EX-92 | 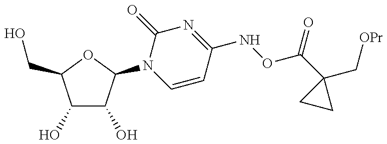 |
| EX-93 | 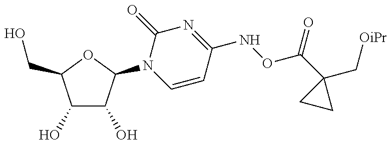 [SMILES: CC(C)OCC1(C(=O)ONc2ccn([C@@H]3O[C@H](CO)[C@@H](O)[C@H]3O)c(=O)n2)CC1] |
| EX-94 | 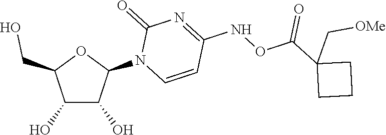 [SMILES: COCC1(C(=O)ONc2ccn([C@@H]3O[C@H](CO)[C@@H](O)[C@H]3O)c(=O)n2)CCC1] |
| EX-95 | 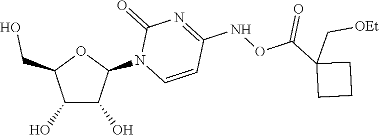 [SMILES: CCOCC1(C(=O)ONc2ccn([C@@H]3O[C@H](CO)[C@@H](O)[C@H]3O)c(=O)n2)CCC1] |
| EX-96 | 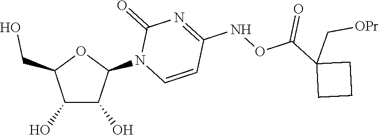 |
| EX-97 | 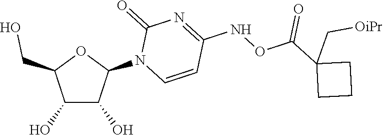 [SMILES: CC(C)OCC1(C(=O)ONc2ccn([C@@H]3O[C@H](CO)[C@@H](O)[C@H]3O)c(=O)n2)CCC1] |
| EX-98 | 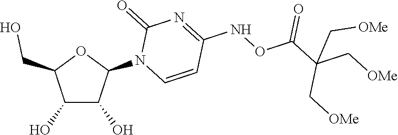 [SMILES: COCC(COC)(COC)C(=O)ONc1ccn([C@@H]2O[C@H](CO)[C@@H](O)[C@H]2O)c(=O)n1] |
| EX-99 | 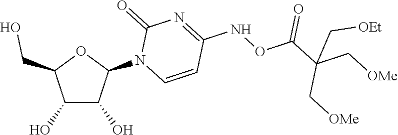 [SMILES: CCOCC(COC)(COC)C(=O)ONc1ccn([C@@H]2O[C@H](CO)[C@@H](O)[C@H]2O)c(=O)n1] |
| EX-100 | 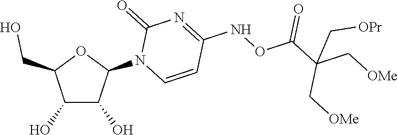 |
| EX-102 | 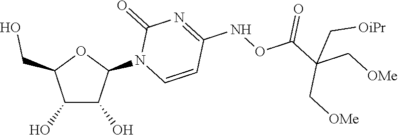 [SMILES: COCC(COC)(COC(C)C)C(=O)ONc1ccn([C@@H]2O[C@H](CO)[C@@H](O)[C@H]2O)c(=O)n1] |
| EX-103 | 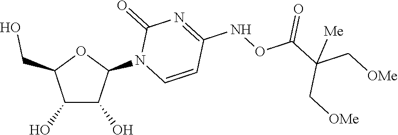 [SMILES: COCC(C)(COC)C(=O)ONc1ccn([C@@H]2O[C@H](CO)[C@@H](O)[C@H]2O)c(=O)n1] |
| EX-104 | 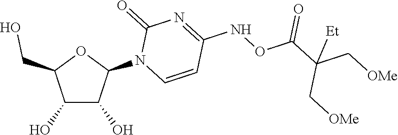 [SMILES: CCC(COC)(COC)C(=O)ONc1ccn([C@@H]2O[C@H](CO)[C@@H](O)[C@H]2O)c(=O)n1] |
| EX-105 | 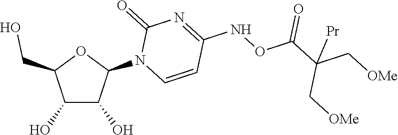 |
| EX-106 | 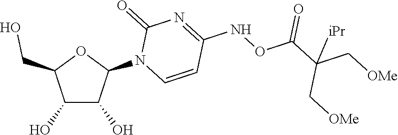 [SMILES: COCC(COC)(C(=O)ONc1ccn([C@@H]2O[C@H](CO)[C@@H](O)[C@H]2O)c(=O)n1)C(C)C] |
| EX-107 | 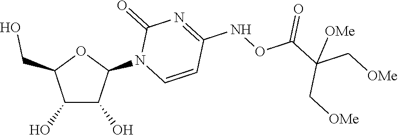 [SMILES: COCC(COC)(OC)C(=O)ONc1ccn([C@@H]2O[C@H](CO)[C@@H](O)[C@H]2O)c(=O)n1] |
| EX-108 | 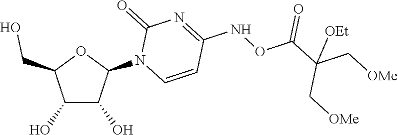 [SMILES: CCOC(COC)(COC)C(=O)ONc1ccn([C@@H]2O[C@H](CO)[C@@H](O)[C@H]2O)c(=O)n1] |
| EX-109 | 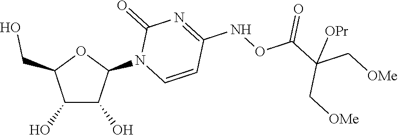 |
| EX-110 | 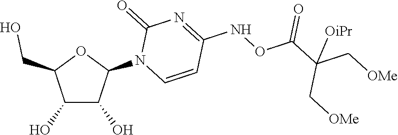 [SMILES: COCC(COC)(OC(C)C)C(=O)ONc1ccn([C@@H]2O[C@H](CO)[C@@H](O)[C@H]2O)c(=O)n1] |
| EX-111 | 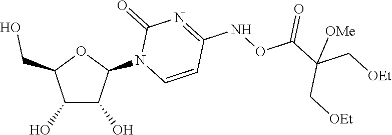 [SMILES: CCOCC(COCC)(OC)C(=O)ONc1ccn([C@@H]2O[C@H](CO)[C@@H](O)[C@H]2O)c(=O)n1] |
| EX-112 | 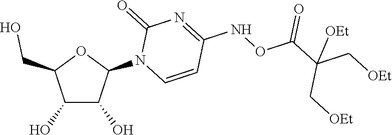 [SMILES: CCOCC(COCC)(OCC)C(=O)ONc1ccn([C@@H]2O[C@H](CO)[C@@H](O)[C@H]2O)c(=O)n1] |
| EX-113 | 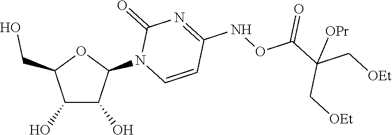 |
| EX-114 | 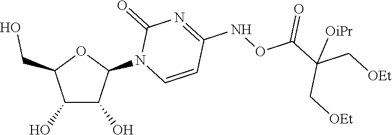 [SMILES: CCOCC(COCC)(OC(C)C)C(=O)ONc1ccn([C@@H]2O[C@H](CO)[C@@H](O)[C@H]2O)c(=O)n1] |
| EX-115 | 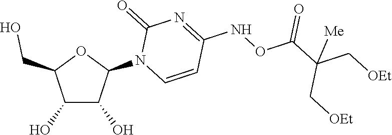 [SMILES: CCOCC(C)(COCC)C(=O)ONc1ccn([C@@H]2O[C@H](CO)[C@@H](O)[C@H]2O)c(=O)n1] |
| EX-116 | 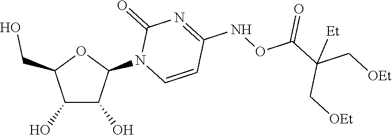 [SMILES: CCOCC(CC)(COCC)C(=O)ONc1ccn([C@@H]2O[C@H](CO)[C@@H](O)[C@H]2O)c(=O)n1] |
| EX-117 | 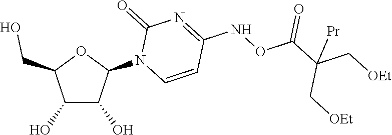 |
| EX-118 | 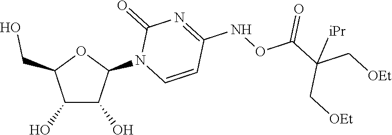 [SMILES: CCOCC(COCC)(C(=O)ONc1ccn([C@@H]2O[C@H](CO)[C@@H](O)[C@H]2O)c(=O)n1)C(C)C] |
| EX-119 | 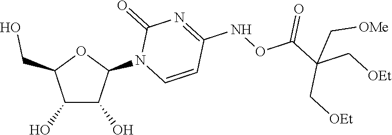 [SMILES: CCOCC(COC)(COCC)C(=O)ONc1ccn([C@@H]2O[C@H](CO)[C@@H](O)[C@H]2O)c(=O)n1] |
| EX-120 | 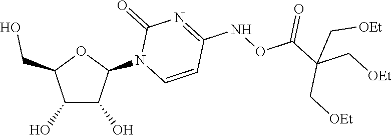 [SMILES: CCOCC(COCC)(COCC)C(=O)ONc1ccn([C@@H]2O[C@H](CO)[C@@H](O)[C@H]2O)c(=O)n1] |
| EX-121 | 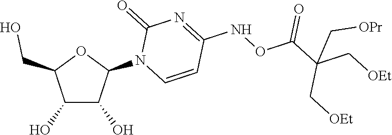 |
| EX-122 | 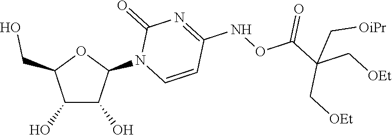 [SMILES: CCOCC(COCC)(COC(C)C)C(=O)ONc1ccn([C@@H]2O[C@H](CO)[C@@H](O)[C@H]2O)c(=O)n1] |
| EX-123 | 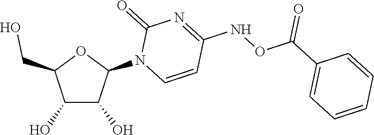 [SMILES: O=C(ONc1ccn([C@@H]2O[C@H](CO)[C@@H](O)[C@H]2O)c(=O)n1)c1ccccc1] |
| EX-124 | 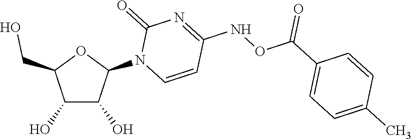 [SMILES: Cc1ccc(C(=O)ONc2ccn([C@@H]3O[C@H](CO)[C@@H](O)[C@H]3O)c(=O)n2)cc1] |
| EX-125 | 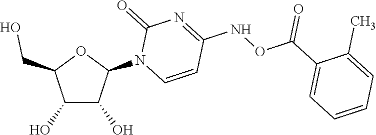 [SMILES: Cc1ccccc1C(=O)ONc1ccn([C@@H]2O[C@H](CO)[C@@H](O)[C@H]2O)c(=O)n1] |
| EX-126 | 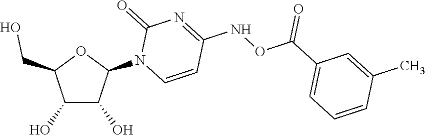 [SMILES: Cc1cccc(C(=O)ONc2ccn([C@@H]3O[C@H](CO)[C@@H](O)[C@H]3O)c(=O)n2)c1] |
| EX-127 | 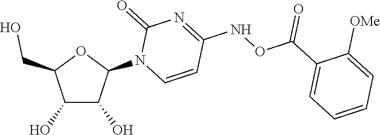 [SMILES: COc1ccccc1C(=O)ONc1ccn([C@@H]2O[C@H](CO)[C@@H](O)[C@H]2O)c(=O)n1] |
| EX-128 | 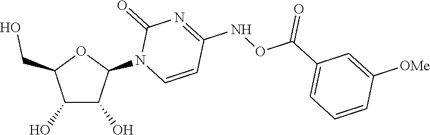 [SMILES: COc1cccc(C(=O)ONc2ccn([C@@H]3O[C@H](CO)[C@@H](O)[C@H]3O)c(=O)n2)c1] |
| EX-129 | 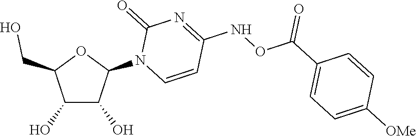 [SMILES: COc1ccc(C(=O)ONc2ccn([C@@H]3O[C@H](CO)[C@@H](O)[C@H]3O)c(=O)n2)cc1] |
| EX-130 | 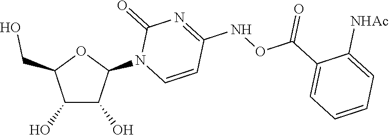 [SMILES: CC(=O)Nc1ccccc1C(=O)ONc1ccn([C@@H]2O[C@H](CO)[C@@H](O)[C@H]2O)c(=O)n1] |
| EX-131 | 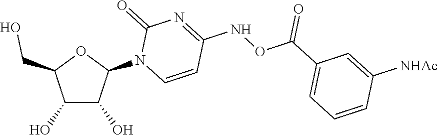 [SMILES: CC(=O)Nc1cccc(C(=O)ONc2ccn([C@@H]3O[C@H](CO)[C@@H](O)[C@H]3O)c(=O)n2)c1] |
| EX-132 | 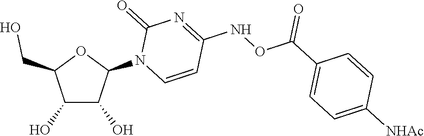 [SMILES: CC(=O)Nc1ccc(C(=O)ONc2ccn([C@@H]3O[C@H](CO)[C@@H](O)[C@H]3O)c(=O)n2)cc1] |
| EX-133 | 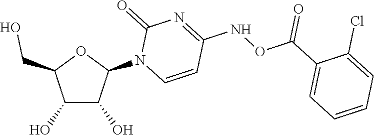 [SMILES: O=C(ONc1ccn([C@@H]2O[C@H](CO)[C@@H](O)[C@H]2O)c(=O)n1)c1ccccc1Cl] |
| EX-134 | 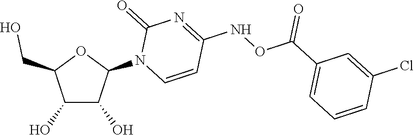 [SMILES: O=C(ONc1ccn([C@@H]2O[C@H](CO)[C@@H](O)[C@H]2O)c(=O)n1)c1cccc(Cl)c1] |
| EX-135 | 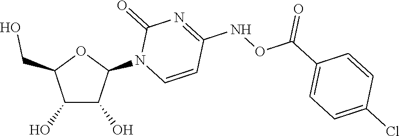 [SMILES: O=C(ONc1ccn([C@@H]2O[C@H](CO)[C@@H](O)[C@H]2O)c(=O)n1)c1ccc(Cl)cc1] |
| EX-136 | 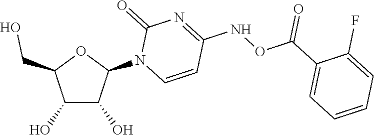 [SMILES: O=C(ONc1ccn([C@@H]2O[C@H](CO)[C@@H](O)[C@H]2O)c(=O)n1)c1ccccc1F] |
| EX-137 | 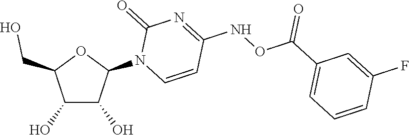 [SMILES: O=C(ONc1ccn([C@@H]2O[C@H](CO)[C@@H](O)[C@H]2O)c(=O)n1)c1cccc(F)c1] |
| EX-138 | 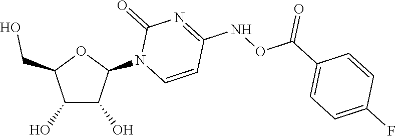 [SMILES: O=C(ONc1ccn([C@@H]2O[C@H](CO)[C@@H](O)[C@H]2O)c(=O)n1)c1ccc(F)cc1] |
| EX-139 | 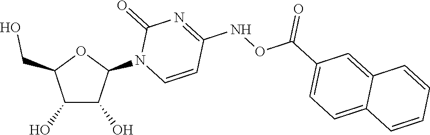 [SMILES: O=C(ONc1ccn([C@@H]2O[C@H](CO)[C@@H](O)[C@H]2O)c(=O)n1)c1ccc2ccccc2c1] |
| EX-140 | 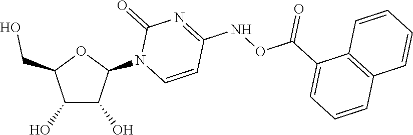 [SMILES: O=C(ONc1ccn([C@@H]2O[C@H](CO)[C@@H](O)[C@H]2O)c(=O)n1)c1cccc2ccccc12] |
| EX-141 | 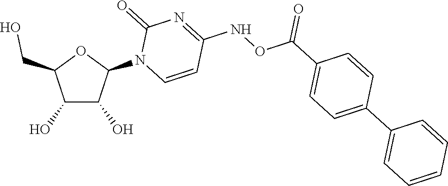 [SMILES: O=C(ONc1ccn([C@@H]2O[C@H](CO)[C@@H](O)[C@H]2O)c(=O)n1)c1ccc(-c2ccccc2)cc1] |
| EX-142 | 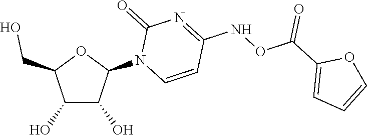 [SMILES: O=C(ONc1ccn([C@@H]2O[C@H](CO)[C@@H](O)[C@H]2O)c(=O)n1)c1ccco1] |
| EX-143 | 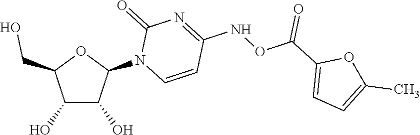 [SMILES: Cc1ccc(C(=O)ONc2ccn([C@@H]3O[C@H](CO)[C@@H](O)[C@H]3O)c(=O)n2)o1] |
| EX-144 | 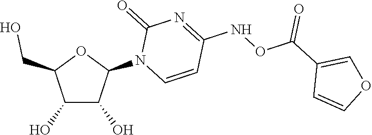 [SMILES: O=C(ONc1ccn([C@@H]2O[C@H](CO)[C@@H](O)[C@H]2O)c(=O)n1)c1ccoc1] |
| EX-145 | 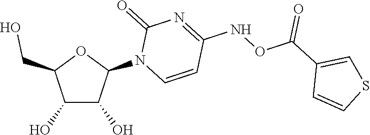 [SMILES: O=C(ONc1ccn([C@@H]2O[C@H](CO)[C@@H](O)[C@H]2O)c(=O)n1)c1ccsc1] |
| EX-146 | 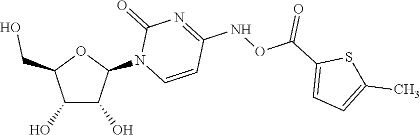 [SMILES: Cc1ccc(C(=O)ONc2ccn([C@@H]3O[C@H](CO)[C@@H](O)[C@H]3O)c(=O)n2)s1] |
| EX-147 | 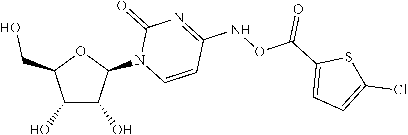 [SMILES: O=C(ONc1ccn([C@@H]2O[C@H](CO)[C@@H](O)[C@H]2O)c(=O)n1)c1ccc(Cl)s1] |
| EX-148 | 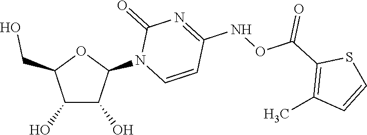 [SMILES: Cc1ccsc1C(=O)ONc1ccn([C@@H]2O[C@H](CO)[C@@H](O)[C@H]2O)c(=O)n1] |
| EX-149 | 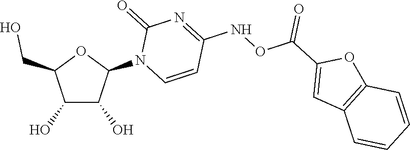 [SMILES: O=C(ONc1ccn([C@@H]2O[C@H](CO)[C@@H](O)[C@H]2O)c(=O)n1)c1cc2ccccc2o1] |
| EX-150 | 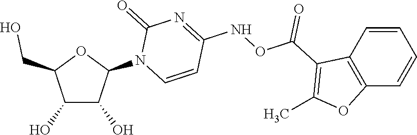 [SMILES: Cc1oc2ccccc2c1C(=O)ONc1ccn([C@@H]2O[C@H](CO)[C@@H](O)[C@H]2O)c(=O)n1] |
| EX-151 | 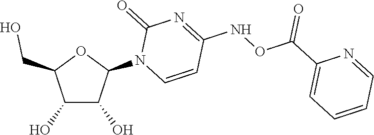 [SMILES: O=C(ONc1ccn([C@@H]2O[C@H](CO)[C@@H](O)[C@H]2O)c(=O)n1)c1ccccn1] |
| EX-152 | 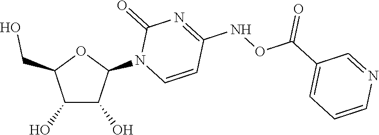 [SMILES: O=C(ONc1ccn([C@@H]2O[C@H](CO)[C@@H](O)[C@H]2O)c(=O)n1)c1cccnc1] |
| EX-153 | 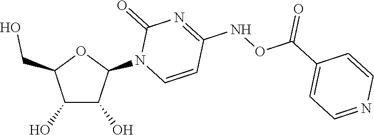 [SMILES: O=C(ONc1ccn([C@@H]2O[C@H](CO)[C@@H](O)[C@H]2O)c(=O)n1)c1ccncc1] |
| EX-154 | 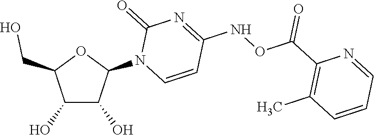 [SMILES: Cc1cccnc1C(=O)ONc1ccn([C@@H]2O[C@H](CO)[C@@H](O)[C@H]2O)c(=O)n1] |
| EX-155 | 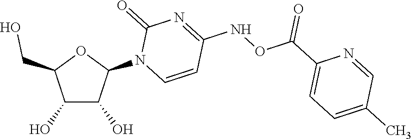 [SMILES: Cc1ccc(C(=O)ONc2ccn([C@@H]3O[C@H](CO)[C@@H](O)[C@H]3O)c(=O)n2)nc1] |
| EX-156 | 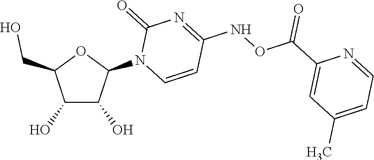 [SMILES: Cc1ccnc(C(=O)ONc2ccn([C@@H]3O[C@H](CO)[C@@H](O)[C@H]3O)c(=O)n2)c1] |
| EX-157 | 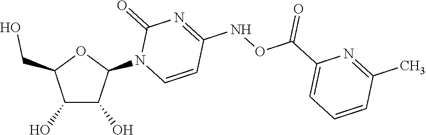 [SMILES: Cc1cccc(C(=O)ONc2ccn([C@@H]3O[C@H](CO)[C@@H](O)[C@H]3O)c(=O)n2)n1] |
| EX-158 | 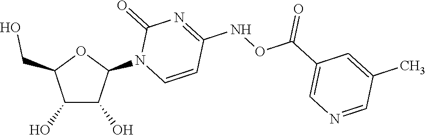 [SMILES: Cc1cncc(C(=O)ONc2ccn([C@@H]3O[C@H](CO)[C@@H](O)[C@H]3O)c(=O)n2)c1] |
| EX-159 | 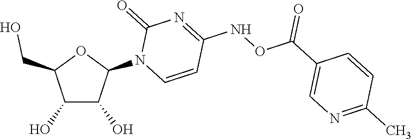 [SMILES: Cc1ccc(C(=O)ONc2ccn([C@@H]3O[C@H](CO)[C@@H](O)[C@H]3O)c(=O)n2)cn1] |
| EX-160 | 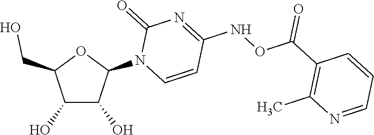 [SMILES: Cc1ncccc1C(=O)ONc1ccn([C@@H]2O[C@H](CO)[C@@H](O)[C@H]2O)c(=O)n1] |
| EX-161 | 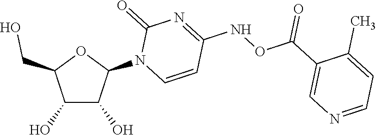 [SMILES: Cc1ccncc1C(=O)ONc1ccn([C@@H]2O[C@H](CO)[C@@H](O)[C@H]2O)c(=O)n1] |
| EX-162 | 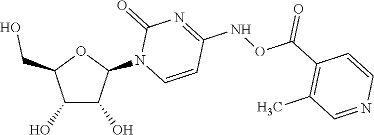 [SMILES: Cc1cnccc1C(=O)ONc1ccn([C@@H]2O[C@H](CO)[C@@H](O)[C@H]2O)c(=O)n1] |
| EX-163 | 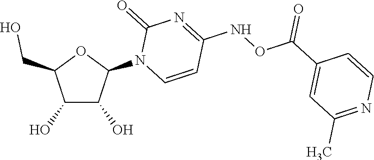 [SMILES: Cc1cc(C(=O)ONc2ccn([C@@H]3O[C@H](CO)[C@@H](O)[C@H]3O)c(=O)n2)ccn1] |
| EX-164 | 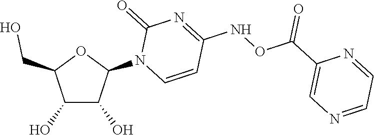 [SMILES: O=C(ONc1ccn([C@@H]2O[C@H](CO)[C@@H](O)[C@H]2O)c(=O)n1)c1cnccn1] |
| EX-165 | 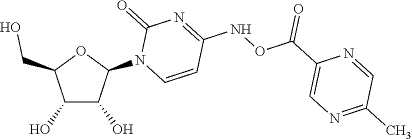 [SMILES: Cc1cnc(C(=O)ONc2ccn([C@@H]3O[C@H](CO)[C@@H](O)[C@H]3O)c(=O)n2)cn1] |
| EX-166 | 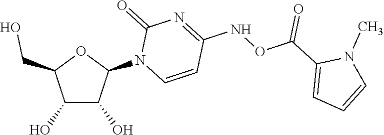 [SMILES: Cn1cccc1C(=O)ONc1ccn([C@@H]2O[C@H](CO)[C@@H](O)[C@H]2O)c(=O)n1] |
| EX-167 | 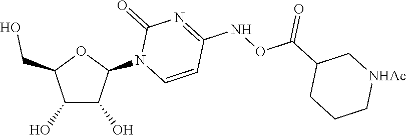 |
| EX-168 | 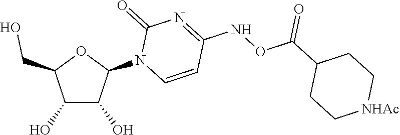 |
| EX-169 | 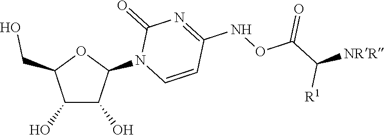 |

Embodiment 28. A process for the production of compounds of Formula I according to any one of Embodiments 1-27, comprising the following step:
reacting NHC with the acid anhydride of formula II to yield the compound of formula I.

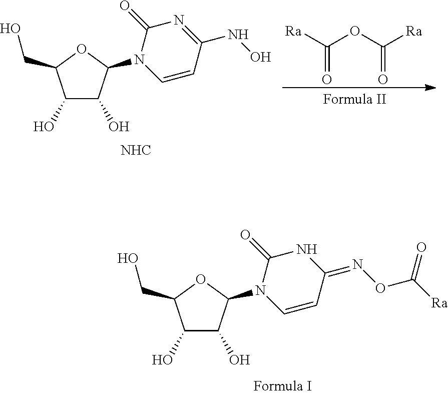

wherein Ra is defined as any one of embodiments 1-27.

Embodiment 29. The process according to Embodiment 28, wherein the reaction is carried out in water or a mixture of water and organic solvent(s), preferably the reaction solvent is selected from pure water, methanol, ethanol, propanol, isopropanol, other lower aliphatic alcohol or a mixture of aliphatic alcohols. DMF, DMSO, NMP, water-methanol mixture, water-ethanol mixture, water-propanol mixture, water-isopropanol mixture, water-nbutanol mixture, water-secbutanol mixture, water-isobutanol mixture, water-THF mixture, water-ACN mixture, water-DMF mixture, water-DMSO mixture, water/2-methyl THF mixture, or any mixture of water with organic solvent(s) that can fully or partially dissolve NHC; more preferably water, lower aliphatic alcohol, water-lower aliphatic alcohol(s) mixtures, water-THF mixture, water/2-methyl THF mixture, water/ACN mixture.

Embodiment 30. The process according to Embodiment 28 or 29, wherein the reaction is carried out without adding any inorganic or organic base (or catalyst), for example, alkali metal hydroxide, carbonate, bicarbonate, alkoxide or hydride, such as sodium bicarbonate, potassium bicarbonate, sodium carbonate, potassium carbonate, sodium hydroxide, potassium hydroxide, lithium hydroxide, sodium methoxide, sodium ethoxide, or sodium hydride, or organic tertiary amines, such as tri-C₁₋₄alkylamines, e.g., TEA, diisopropylethylamine, tripropylamine, tributylamine, or heterocyclic bases, such as pyridine, picolines, lutidines, DMAP, DBU etc.

Embodiment 31. The process according to Embodiment 28 or 29, wherein the reaction is carried out in the presence of inorganic or organic base (or catalyst), for example, alkali metal hydroxide, carbonate, bicarbonate, alkoxide or hydride, such as sodium bicarbonate, potassium bicarbonate, sodium carbonate, potassium carbonate, sodium hydroxide, potassium hydroxide, lithium hydroxide, sodium methoxide, sodium ethoxide, or sodium hydride, or organic tertiary amines, such as tri-C₁₋₄alkylamines, e.g., TEA, diisopropylethylamine, tripropylamine, tributylamine, or heterocyclic bases, such as pyridine, picolines, lutidines, DMAP, DBU etc.

Embodiment 32. The process according to any one of Embodiments 28-31, wherein the product is obtained in solid crystalline form, by cooling the reaction mixture without adding anti-solvent.

Embodiment 33. The process according to any one of Embodiments 28-32, wherein the product is obtained in solid crystalline form, without subjecting to any chromatography purification.

Embodiment 34. The process according to any one of Embodiments 28-33, wherein the purity of the product produced in the reaction solution is about 90%-98%.

Embodiment 35. The process according to any one of Embodiments 28-33, wherein the purity of the product produced in the reaction solution is over 98%.

Embodiment 36. A pharmaceutical composition, comprising the compound of any one of embodiments 1-27 or a tautomer, stereoisomer or racemate thereof or a pharmaceutically acceptable salt thereof, and optionally comprising a pharmaceutically acceptable excipient.

Embodiment 37. Use of the compound of any one of embodiments 1-27 or a tautomer, stereoisomer or racemate thereof or a pharmaceutically acceptable salt thereof in the manufacture of a medicament for treating or preventing a RNA viral infection.

Embodiment 38. The use according to Embodiment 29, wherein the RNA virus is coronavirus, e.g., a human coronavirus, SARS coronavirus or MERS coronavirus, alphavirus, e.g., Eastern equine encephalitis virus, Western equine encephalitis virus, Venezuelan equine encephalitis virus, Chikungunya virus, Ross River virus or Barmah Forest virus, filoviridae virus, e.g., ebola virus, orthomyxoviridae virus, e.g., influenza virus, influenza A virus or influenza B virus, paramyxoviridae virus, e.g., respiratory Syncytial Virus (RSV), flavivirus, e.g., Zika virus or virus; preferably, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

Embodiment 39. A method of treating or preventing a RNA viral infection in a subject, comprising administering to the subject in need thereof an effective amount of the compound of any one of embodiments 1-27 or a tautomer, stereoisomer or racemate thereof or a pharmaceutically acceptable salt thereof.

Embodiment 40. The method according to Embodiment 31, wherein the RNA virus is coronavirus, e.g., a human coronavirus, SARS coronavirus or MERS coronavirus, alphavirus, e.g., Eastern equine encephalitis virus, Western equine encephalitis virus, Venezuelan equine encephalitis virus, Chikungunya virus or Ross River virus, filoviridae virus, e.g., ebola virus, orthomyxoviridae virus, e.g., influenza virus, influenza A virus or influenza B virus, paramyxoviridae virus, e.g., respiratory Syncytial Virus (RSV), flavivirus, e.g., Zika virus; preferably, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

Embodiment 41. The compound of any one of embodiments 1-27 or a tautomer, stereoisomer or racemate thereof or a pharmaceutically acceptable salt thereof, for use as a medicament.

Embodiment 42. The compound of any one of embodiments 1-27 or a tautomer, stereoisomer or racemate thereof or a pharmaceutically acceptable salt thereof, for use in treating or preventing a RNA viral infection.

Embodiment 43. The compound for use according to Embodiment 34, wherein the RNA virus is coronavirus, e.g., a human coronavirus, SARS coronavirus or MERS coronavirus, alphavirus, e.g., Eastern equine encephalitis virus, Western equine encephalitis virus, Venezuelan equine encephalitis virus, Chikungunya virus or Ross River virus, filoviridae virus, e.g., ebola virus, orthomyxoviridae virus, e.g., influenza virus, influenza A virus or influenza B virus, paramyxoviridae virus, e.g., respiratory Syncytial Virus (RSV), flavivirus, e.g., Zika virus; preferably, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

Embodiment 44. A method for increasing bioavailability of N4-hydroxycytidine for treating or preventing a RNA viral infection comprising administering to the subject in need thereof an effective amount of the compound of any one of embodiments 1-27 or a tautomer, stereoisomer or racemate thereof or a pharmaceutically acceptable salt thereof.

Embodiment 45. A pharmaceutical combination, comprising the compound of any one of embodiments 1-27 or a tautomer, stereoisomer or racemate thereof or a pharmaceutically acceptable salt thereof, and at least one additional therapeutic agent.

Embodiment 46. The pharmaceutical combination according to Embodiment 37, wherein additional therapeutic agent is selected from the group consisting of:

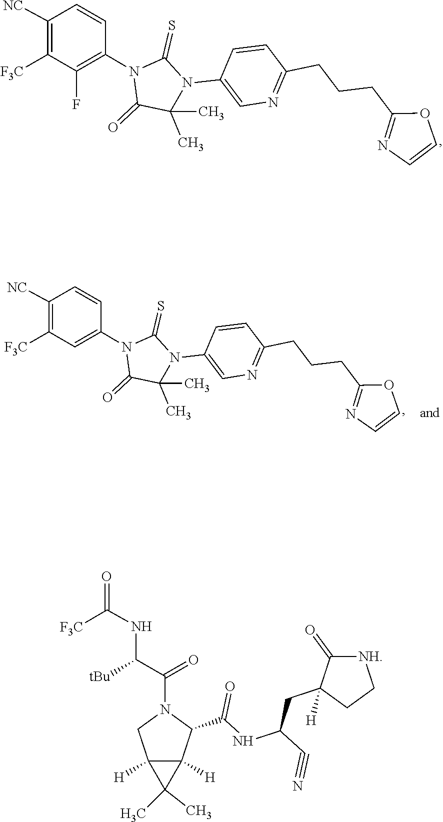 [SMILES: CC1(C)C(=O)N(c2ccc(C#N)c(C(F)(F)F)c2F)C(=S)N1c1ccc(CCCc2ncco2)nc1 ; CC1(C)C(=O)N(c2ccc(C#N)c(C(F)(F)F)c2)C(=S)N1c1ccc(CCCc2ncco2)nc1 ; CCC1(C)[C@@H]2[C@@H](C(=O)N[C@H](C#N)C[C@@H]3CCNC3=O)N(C(=O)[C@@H](NC(=O)C(F)(F)F)C(C)(C)C)C[C@@H]21]

### Methods of Use

According to the present disclosure, the RNA virus is coronavirus, e.g., a human coronavirus, SARS coronavirus or MERS coronavirus, alphavirus, e.g., Eastern equine encephalitis virus, Western equine encephalitis virus, Venezuelan equine encephalitis virus, Chikungunya virus or Ross River virus, filoviridae virus, e.g., ebola virus, orthomyxoviridae virus, e.g., influenza virus, influenza A virus (including subtype H1N1, H3N2, H7N9, or H5N1), influenza B virus or influenza C, paramyxoviridae virus, e.g., respiratory Syncytial Virus (RSV), flavivirus, e.g., Zika virus, rotavirus, e.g., rotavirus A, rotavirus B, rotavirus C. rotavirus D, rotavirus E; preferably, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

Preferably, according to the present disclosure, the RNA virus is a human coronavirus, SARS coronavirus, MERS coronavirus, Eastern equine encephalitis virus, Western equine encephalitis virus, Venezuelan equine encephalitis virus, Chikungunya virus, Ross River virus, orthomyxoviridae virus, paramyxoviridae virus, RSV virus, influenza A virus, influenza B virus, filoviridae virus, or Ebola virus.

More preferably, according to the present disclosure, the RNA virus is a human coronavirus, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

According to the present disclosure, the subject is at risk of, exhibiting symptoms of, or diagnosed with SARS-CoV-2/COVID-19 virus, influenza A virus including subtype H1N1, H3N2, H7N9, or H5N1, influenza B virus, influenza C virus, rotavirus A, rotavirus B, rotavirus C, rotavirus D, rotavirus E, human coronavirus, SARS coronavirus, MERS coronavirus, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), Dengue virus, Zika virus, chikungunya, Eastern equine encephalitis virus (EEEV), Western equine encephalitis virus (WEEV), Venezuelan equine encephalitis virus (VEEV), Ross River virus, Barmah Forest virus, yellow fever virus, measles virus, mumps virus, respiratory syncytial virus, rinderpest virus, California encephalitis virus, hantavirus, rabies virus, ebola virus, marburg virus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D, hepatitis E or human immunodeficiency virus (HIV), The Human T-lymphotropic virus Type I (HTLV-1), Friend spleen focus-forming virus (SFFV) or Xenotropic MuLVRelated Virus (XMRV). In some embodiments, the subject is at risk of, exhibiting symptoms of, or diagnosed with a Zika virus infection.

According to the present invention, the subject is diagnosed with SARS-CoV-2/COVID-19 virus including an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any variant SARS-CoV-2/COVID-19 virus that could be treated by formula (I) compound or pharmaceuticals contain formula (1) compound.

According to the present invention, the subject is diagnosed with influenza A virus including subtypes H1N1, H3N2, H7N9. H5N1 (low path), and H5N1 (high path) influenza B virus, influenza C virus, rotavirus A, rotavirus B, rotavirus C, rotavirus D, rotavirus E, SARS coronavirus, MERS-COV, human adenovirus types (HAdV-1 to 55), human papillomavirus (HPV) Types 16, 18, 31, 33, 35, 39, 45, 51, 52, 56, 58, and 59, parvovirus B19, molluscum contagiosum virus, JC virus (JCV), BK virus, Merkel cell polyomavirus, coxsackie A virus, norovirus, Rubella virus, lymphocytic choriomeningitis virus (LCMV), yellow fever virus, measles virus, mumps virus, respiratory syncytial virus, parainfluenza viruses 1 and 3, rinderpest virus, chikungunya, eastern equine encephalitis virus (EEEV). Venezuelan equine encephalitis virus (VEEV), western equine encephalitis virus (WEEV), California encephalitis virus, Japanese encephalitis virus, Rift Valley fever virus (RVFV), hantavirus, Dengue virus serotypes 1, 2, 3 and 4, Zika virus, West Nile virus, Tacaribe virus, Junin, rabies virus, ebola virus, marburg virus, adenovirus, herpes simplex virus-1 (HSV-1), herpes simplex virus-2 (HSV-2), varicella zoster virus (VZV), Epstein-Barr virus (EBV), cytomegalovirus (CMV), herpes lymphotropic virus, roseolovirus, or Kaposi's sarcoma-associated herpesvirus, hepatitis A, hepatitis B, hepatitis C, hepatitis D. hepatitis E or human immunodeficiency virus (HIV). In certain embodiments, the subject is diagnosed with a Zika virus infection.

According to the present invention, the subject is diagnosed with gastroenteritis, acute respiratory disease, severe acute respiratory syndrome, post-viral fatigue syndrome, viral hemorrhagic fevers, acquired immunodeficiency syndrome or hepatitis.

### Pharmaceutical Composition and Administration

The compounds of the present invention (such as any of the compounds of the Examples herein) alone or in combination with one or more additional therapeutic agents can be formulated into a pharmaceutical composition. The pharmaceutical composition includes: (a) an effective amount of the compound of the present invention; (b) a pharmaceutically acceptable excipient (for example, one or more pharmaceutically acceptable carriers); and optionally (c) at least one additional therapeutic agent.

A pharmaceutically acceptable excipient refers to an excipient that is compatible with the active ingredient(s) in the composition (in some embodiments, can stabilize the active ingredient) and is not harmful to the subject being treated. Suitable pharmaceutically acceptable excipients are disclosed in standard reference books in the art (e.g., Remington's Pharmaceutical Sciences, Remington: The Science and Practice of Pharmacy.), including one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., the compounds of the present invention or pharmaceutical composition thereof) or aid in the manufacturing of the pharmaceutical product (i.e., medicament).

The compound of the invention can be administered in various known manners, such as orally, parenterally, by inhalation, or through the lungs, i.e., pulmonary administration, nasal, sublingual, lingual, buccal, rectal, dermal, transdermal, conjunctival, otic route, or as an implant or stent. The term “parenterally” as used herein includes subcutaneous, intracutaneous, intravenous, intramuscular, intraarticular, intraarterial, intrasynovial, intrasternal, intrathecal, intralesional and intracranial injection or infusion.

Oral or parenteral administration is preferred, especially oral administration.

The compound of the invention may be administered in any convenient formulation, e.g., tablets, powders, capsules, pills, solutions, dispersions, suspensions, syrups, sprays, suppositories, gels, emulsions, patches, aqueous buffer, such as a saline or phosphate buffer etc. Such compositions may contain components conventional in pharmaceutical preparations, e.g., diluents, carriers, pH modifiers, sweeteners, bulking agents, and an additional active agent.

In general, it has been found to be advantageous in the case of parenteral administration to administer amounts of about 0.001 to 20 mg/kg, preferably about 0.01 to 10 mg/kg, of body weight to achieve effective results. In the case of oral administration, the dosage is about 0.01 to 100 mg/kg, preferably about 0.01 to 20 mg/kg and most preferably 0.1 to 15 mg/kg of body weight.

### Combination Therapies

The compound described herein can be administered adjunctively with at least one additional therapeutic agent.

The additional therapeutic agents include but are not limited to analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti-asthma drugs, cardiovascular drugs, corticosteroids, dopaminergics, electrolytes, gastro-intestinal drugs, muscle relaxants, nutritional agents, vitamins, parasympathomimetics, stimulants, anorectics, anti-narcoleptics, and antiviral agents. In a particular embodiment, the antiviral agent is a non-CNS targeting antiviral compound. “Adjunctive administration”, as used herein, means the compound can be administered in the same dosage form or in separate dosage forms with one or more other active agents. The additional therapeutic agent(s) can be formulated for immediate release, controlled release, or combinations thereof.

The compound of the present invention and pharmaceutical compositions can be administered in combination with at least one additional therapeutic agent, such as antiviral agent(s) such as abacavir, acyclovir, acyclovir, adefovir, amantadine, amprenavir, ampligen, arbidol, atazanavir, atripla, balapiravir, BCX4430, boceprevir, cidofovir, combivir, daclatasvir, darunavir, dasabuvir, delavirdine, didanosine, docosanol, edoxudine, efavirenz, emtricitabine, enfuvirtide, entecavir, famciclovir, favipiravir, fomivirsen, fosamprenavir, foscarnet, fosfonet, ganciclovir, GS-5734, ibacitabine, imunovir, idoxuridine, imiquimod, indinavir, inosine, interferon type III, interferon type II, interferon type I, lamivudine, ledipasvir, lopinavir, loviride, maraviroc, moroxydine, methisazone, nelfinavir, nevirapine, nexavir, NITD008, ombitasvir, oseltamivir, paritaprevir, peginterferon alfa-2a, penciclovir, peramivir, pleconaril, podophyllotoxin, raltegravir, ribavirin, rimantadine, ritonavir, pyramidine, saquinavir, simeprevir, sofosbuvir, stavudine, telaprevir, telbivudine, tenofovir, tenofovir disoproxil, Tenofovir Exalidex, tipranavir, trifluridine, trizivir, tromantadine, truvada, valaciclovir, valganciclovir, vicriviroc, vidarabine, viramidine zalcitabine, zanamivir, molnupiravir or zidovudine and combinations thereof.

The compound of the present invention and pharmaceutical compositions disclosed herein can be administered in combination with any of the compounds disclosed in WO2012119559 for the treatment of SARS-CoV-2/COVID-19 infection.

The compound of the present invention and pharmaceutical compositions disclosed herein can be administered in combination with any of the compounds disclosed in WO2012119559 for the prevention of SARS-CoV-2/COVID-19 infection.

The compound of the present invention and pharmaceutical compositions disclosed herein can be administered in combination with proxalutamide for the treatment of SARS-CoV-2/COVID-19 infection.

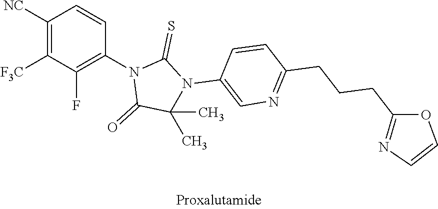 [SMILES: CC1(C)C(=O)N(c2ccc(C#N)c(C(F)(F)F)c2F)C(=S)N1c1ccc(CCCc2ncco2)nc1]

The compound of the present invention and pharmaceutical compositions disclosed herein can be administered in combination with proxalutamide for the prevention of SARS-CoV-2/COVID-19 infection.

The compound of the present invention and pharmaceutical compositions disclosed herein can be administered in combination with compound-X for the treatment of SARS-CoV-2/COVID-19 infection.

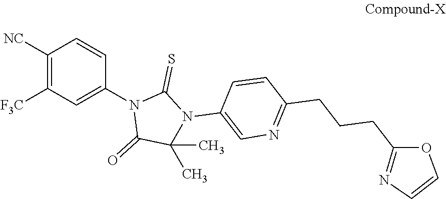 [SMILES: CC1(C)C(=O)N(c2ccc(C#N)c(C(F)(F)F)c2)C(=S)N1c1ccc(CCCc2ncco2)nc1]

The compound of the present invention and pharmaceutical compositions disclosed herein can be administered in combination with compound-X for the prevention of SARS-CoV-2/COVID-19 infection.

The compound of the present invention and pharmaceutical compositions disclosed herein can be administered in combination with PF-07321332 for the treatment of SARS-CoV-2/COVID-19 infection.

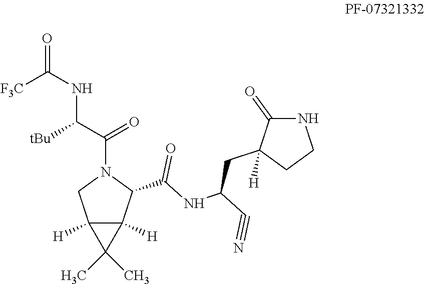 [SMILES: CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

The compound of the present invention and pharmaceutical compositions disclosed herein can be administered in combination with PF-07321332 for the prevention of SARS-CoV-2/COVID-19 infection.

Accordingly, the present disclosure also provides a pharmaceutical combination, comprising the compound of the present invention and at least one additional therapeutic agent. Examples of the additional therapeutic agent include, but are not limited to, those agents mentioned above, preferably proxalutamide, Compound-X and PF-07321332.

The percentages in the tests and examples which follow are, unless indicated otherwise, percentages by weight; parts are parts by weight. Solvent ratios, dilution ratios and concentration data for liquid/liquid solutions are based in each case on volume.

Each embodiment and technical solution described in the present disclosure and the features in each embodiment and technical solution should be understood as being capable of combining with each other in any manner, and those technical solutions obtained by such combination(s) are all included in the scope of the present disclosure the same as if each and every technical solution obtained by such combination(s) were specifically and individually listed, unless the context clearly shows otherwise.

All patents, patent applications, publications, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant material or prior art. The right to challenge the accuracy and pertinence of any assertion of such patents, patent applications, publications, and other references as relevant material or prior art is specifically reserved.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

All reagents and starting materials used in this invention are commercially available or prepared according to the prior art, unless specified otherwise.

¹H NMR spectra were measured on a Bruker 400 MHz instrument, Chemical shifts were measured relative to the appropriate solvent peak: CDCl3 (δ 7.27), DMSO-d6 (δ 2.50), CD3OD (δ 3.31), D2O (δ 4.79). The following abbreviations were used to describe coupling: s=singlet, d=doublet, t=triplet, q=quartet, quin=quintet, m=multiplet, br=broad. ¹³C NMR spectra were measured on a Bruker instrument at 100 MHz with chemical shifts relative to the appropriate solvent peak: CDCl3 (δ 77.0), DMSO d6 (δ 39.5), CD3OD (δ 49.0).

### Abbreviations and Acronyms

aq. aqueous solution
calc. calculated
br s broad singlet (in NMR)
DCI direct chemical ionization (in MS)
dec. decomposition point
DMF dimethylformamide
DMSO dimethyl sulphoxide
DSC dynamic differential calorimetry
eq. equivalent(s)
ESI electrospray ionization (in MS)
Et ethyl
fnd. found
h hour(s)
HPLC high-pressure high-performance liquid chromatography
HRMS high-resolution mass spectrometry
conc. concentrated
LC-MS liquid chromatography-coupled mass spectrometry
LiHMDS lithium hexamethyldisilazide
Me methyl
min minute(s)
MS mass spectrometry
NMR nuclear magnetic resonance spectrometry
Pd2 dba3 tris(dibenzylideneacetone) dipalladium
Ph phenyl
PLM polarized light microscope
RT room temperature
Rt retention time (in HPLC)
TGA thermogravimetric analysis
THF tetrahydrofuran
UV ultraviolet spectrometry
v/v volume to volume ratio (of a solution)

### Preparations of Starting Materials and Intermediates

### Preparation 1: Synthesis of Alkoxyl Substituted Propionic Acids and Anhydrides

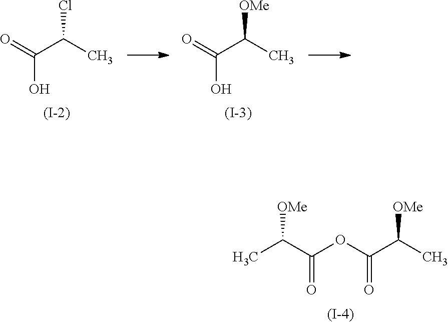

### Procedure for Synthesis of (R)-2-methoxypropanic acid (I-3) and the anhydride (I-4)

(S)-2-Choropropanoic acid (80.0 g, 738 mmol, 1 equiv., 98%) was added to a two-neck round-bottom flask under nitrogen. 25 wt % Sodium methoxide in methanol (506 mL, 2.212 mol, 3 equiv.) was added slowly. The reaction was heated to 60° C. for 16 hours and conversion was monitored until <2% starting material remained. When sufficient conversion was achieved, the reaction vessel was cooled to room temperature, and pH was adjusted with 4M HCl in dioxane (200 mL, 99%) to the point at which pH just changes from >12 to 7, indicating neutralization of excess sodium methoxide without protonating the sodium salt of the carboxylate. The reaction mixture was filtered to remove salts, and the salt cake was washed twice with 5 mL of methanol. The filtrate was concentrated, redissolved in water, acidified to pH=˜2 with 6M HCl and extracted with EtOAc. The organic layer were dried with sodium sulfate and concentrated to afford compound (I-3) as a liquid (73 g, 95%) which was of sufficient purity to be used without purification. ¹H NMR (CD₃OD) δ 3.67 (q, 1H), 3.33 (s, 3H), and 1.33 ppm (d, 3H).

In a 2-liter, four-necked glass reactor provided with a thermometer and a stirrer were placed, in a nitrogen atmosphere, 500 g of methylene chloride, 104.1 g (1.0 mol) of (R)-2-methoxypropanic acid (3) and 57.3 g (0.5 mol) of methanesulfonyl chloride. The mixture was cooled to 5° C.

Then, 101.3 g (1.0 mol, 1 equivalent relative to the acids generated from methanesulfonyl chloride) of triethylamine was added dropwise in 2 hours with the temperature of the reaction mixture being controlled at 30° C. or lower. After the completion of the dropwise addition, stirring for 1 hour with the same temperature being kept. The reaction mixture was analyzed by a gas chromatograph (GC), which indicated that the conversion of (R)-2-methoxypropanic acid (3) was >95%.

After the completion of the reaction, 200 g of water was added to the reaction mixture to wash the reaction mixture. The reaction mixture was further washed twice each time with 200 g of water, after which distillation was conducted to remove methylene chloride. 85.6 g of the (R)-2-methoxypropanic acid anhydride (I-4) obtained as a yellow liquid and it was used in the acylation step without further purification.

In the same manner as described in preparation 1, the following 2-alkoxylsubstituted propionic acid anhydrides were prepared:

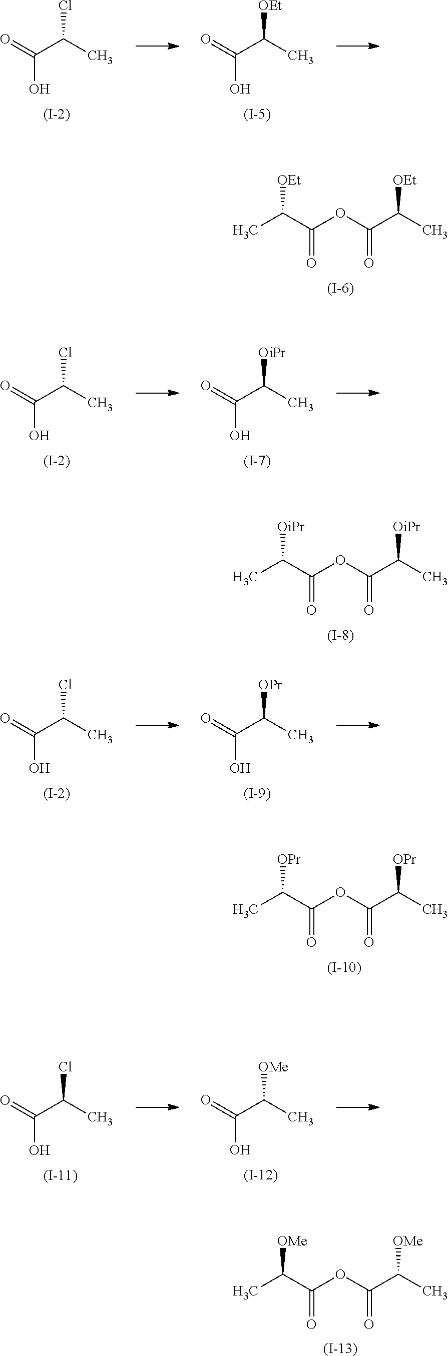

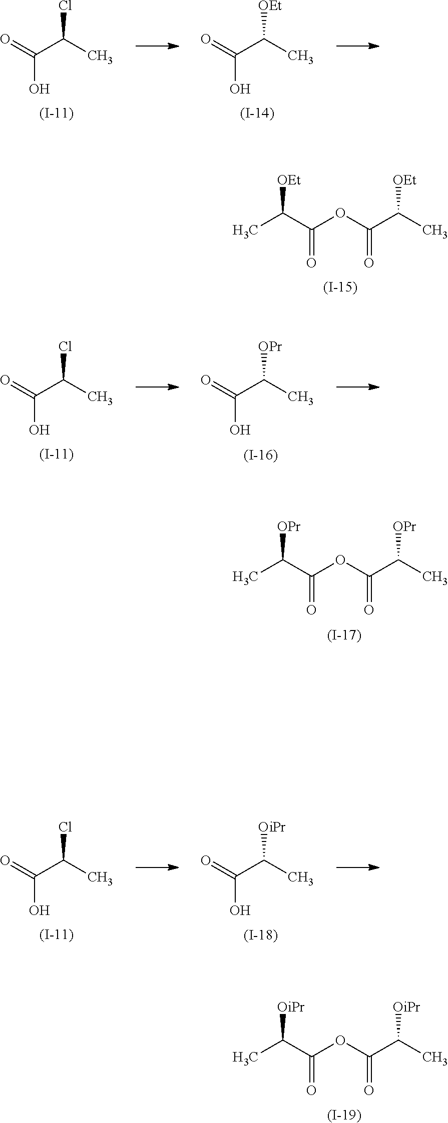

### Preparation 2: Synthesis of Alkoxyl Substituted Isobutyric Acid and Anhydrides

### Procedure for Synthesis of 2-ethoxyisobutyric acid/2-ethoxy-2-methylpropanoic acid (I-21) and the anhydride (I-22)

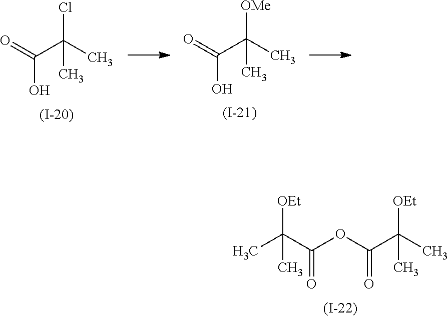

2-Ethoxyisobutyric acid was prepared according to reference (Ragan, John A.; Ide, Nathan D.; Cai, Weiling; Cawley, James J.; Colon-Cruz, Roberto. Kumar, Rajesh; Peng, Zhihui; Vanderplas, Brian C. [Organic process research and development, 2010, vol. 14, #6, p. 1402-1406]): In a 500 mL 3 neck round bottomed flask was dissolved 2-bromo-2-methylpropanoic acid (I-20) (40 g, 239.5 mmole) in ethanol (320 mL) and cooled to 0 to 5° C. followed by dropwise addition of DIPEA (87.4 mL, 502.9 mmole) at 0 to 5° C. and the reaction mixture was stirred at 0° C. for 30 min. The reaction mixture was warmed to 40° C. for 16 h. After 16 h, the reaction mixture was cooled to room temperature, ethanol was removed in vacuo, leaving a thick white slurry. Diethyl ether and water was added to the slurry and cooled to 0° C. The mixture was acidified with 10% HCl (50 mL) and the organic layer was separated and washed with brine. To the organic phase was added 10% aq NaHSO₃ (and the mixture was stirred at room temperature for 6 h. The biphasic mixture was acidified with 10% HCl (50 mL) to the pH 1.0 t 0.5. The organic phase was washed with brine (100 mL), dried over sodium sulfate, filtered and concentrated to give 30 g of 2-ethoxy-2-methylpropanoic acid (I-21). The product, 2-ethoxy-2-methylpropanoic acid (I-21), was carried forwarded to the next step without further purification.

In a 2-liter, four-necked glass reactor equipped with a thermometer and a stirrer were placed, in a nitrogen atmosphere, 300 g of methylene chloride, 66.1 g (0.5 mol) of 2-ethoxy-2-methylpropanoic acid (I-21) and 28.65 g (0.25 mol) of methanesulfonyl chloride. The mixture was cooled to 5° C. Then, 50.65 g (0.5 mol, 1 equivalent relative to the acids generated from methanesulfonyl chloride) of triethylamine was added dropwise in 2 hours with the temperature of the reaction mixture being controlled at 30° C. or lower. After the completion of the dropwise addition, the mixture was stirred for 1 hour with the same temperature being kept. The reaction mixture was analyzed by a gas chromatograph (GC), which indicated that the conversion of 2-ethoxy-2-methylpropanoic acid (I-21) was >95%.

After the completion of the reaction, 100 g of water was added to the reaction mixture to wash the reaction mixture. The reaction mixture was further washed twice each time with 100 g of water, after which distillation was conducted to remove methylene chloride. 51 g of the 2-ethoxy-2-methylpropanoic acid anhydride (I-22) obtained as a yellow liquid and it was used in the acylation step without further purification

In the same manner as described in preparation 1, the following 2-alkoxylsubstituted 2-methylpropionic acid/2-alkoxylsubstituted isobutyric acid anhydrides were prepared:

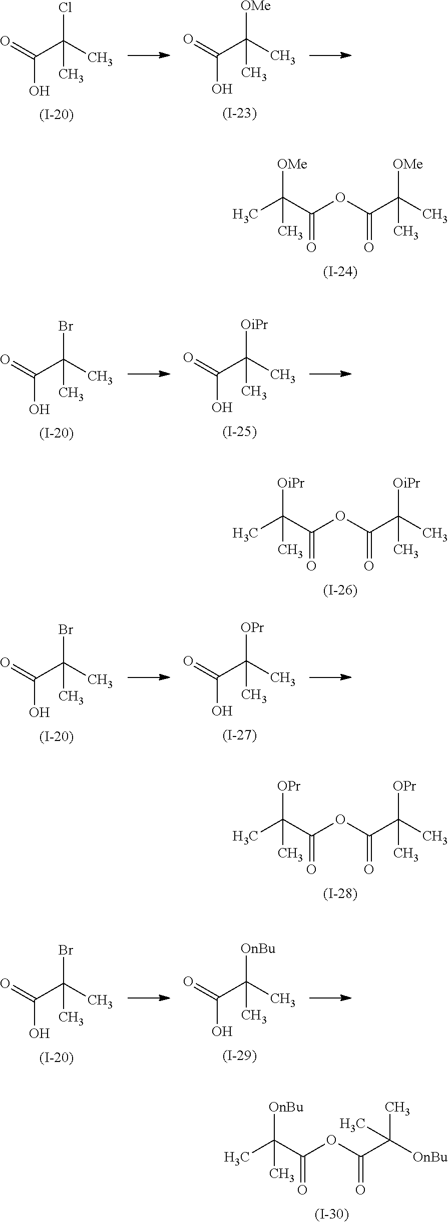

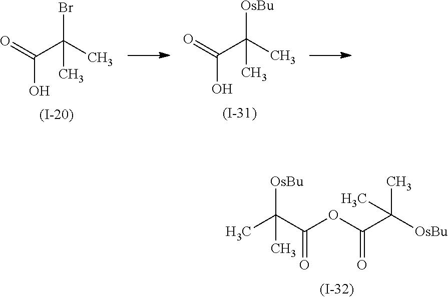

### Preparation 3 Synthesis of 4-alkoxytetrahydro-2H-pyran-4-carboxylic acid and anhydrides

### Procedure for Synthesis of 4-methoxytetrahydro-2H-pyran-4-carboxylic acid (I-36) and the anhydride (I-37)

Commercially available methyl tetrahydro-2H-pyran-4-carboxylate was brominated according to the method described in Organic Letters, 2020, vol. 22, #10, p. 3922-3925. The ester is then hydrolyzed to the corresponding alpha bromoacid (I-35). The the alpha bromoacid (I-35) is then converted to the corresponding acid (I-36) and the anhydride (I-37) according to the process of Preparation 2.

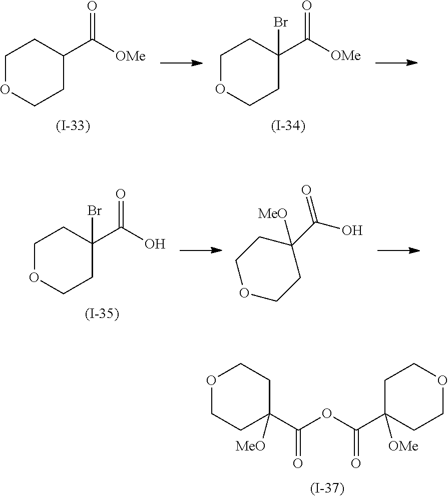

The following 4-alkoxytetrahydro-2H-pyran-4-carboxylic acids and anhydrides were prepared similarly.

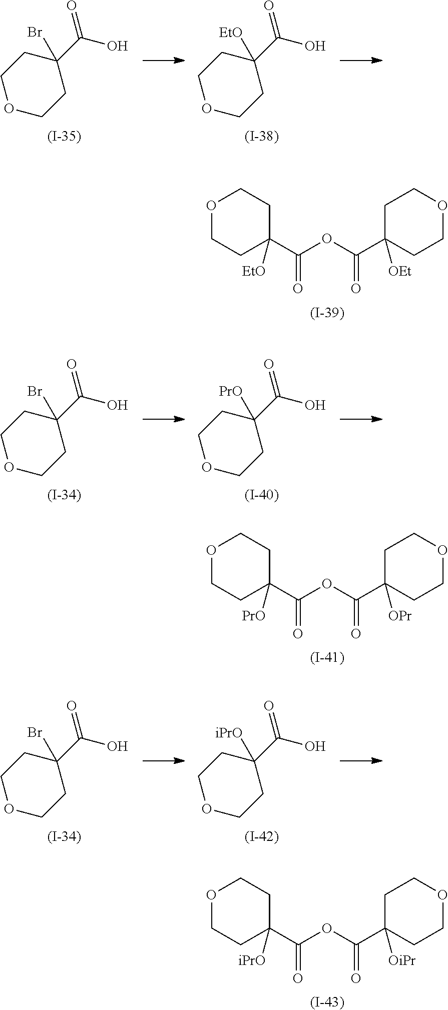

### Preparation 4 Synthesis of 4-alkyltetrahydro-2H-pyran-4-carboxylic acid and anhydrides

### Procedure for Synthesis of 4-methyltetrahydro-2H-pyran-4-carboxylic acid (I-46) and the anhydride (I-47)

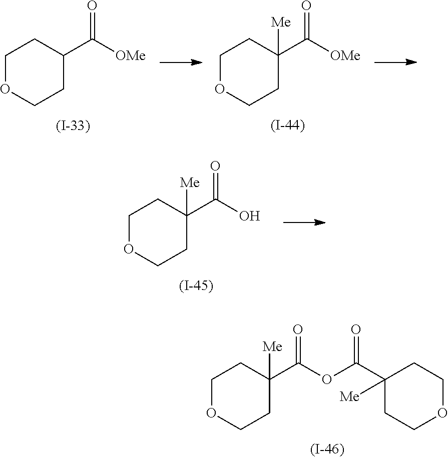

Commercially available tetrahydro-2H-pyran-4-carboxylic acid methyl ester (I-33) was methylated in the same manner as described in Example 64.1A in U.S. Pat. No. 9,434,690. The methyl ester was then hydrolyzed with aqueous NaOH and acidified with HCl to afford 4-methyltetrahydro-2H-pyran-4-carboxylic acid (I-46) as an off white solid.

4-methyltetrahydro-2H-pyran-4-carboxylic acid anhydride (I-47) was prepared according to the process of Preparation 2 as a yellowish oil.

The following 4-alkyltetrahydro-2H-pyran-4-carboxylic acid anhydrides were prepared similarly.

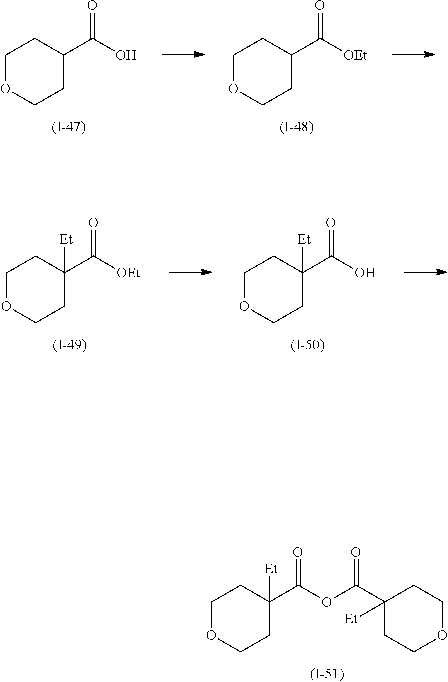

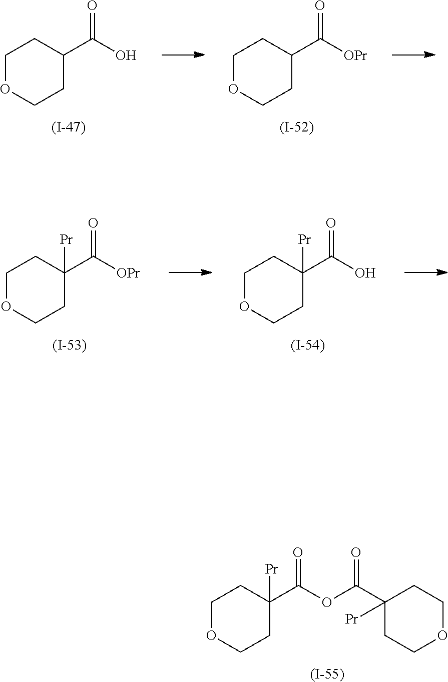

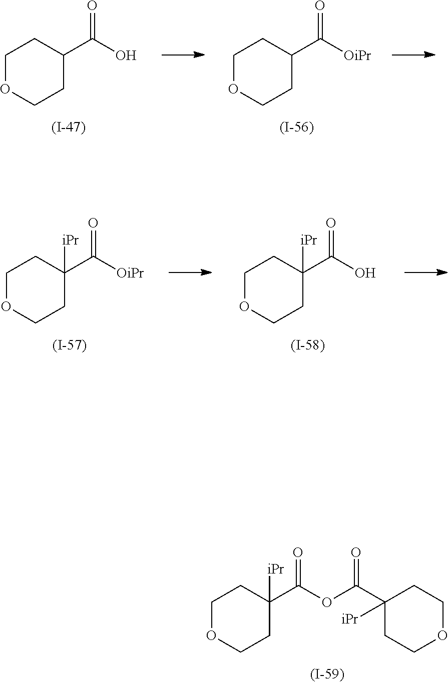

### Preparation 5 Synthesis of 2-ethyl-2-alkoxyl-butyric acid and anhydrides

### Procedure for Synthesis of 2-ethyl-2-methoxyl-butyric acid (I-62) and the anhydride (I-63)

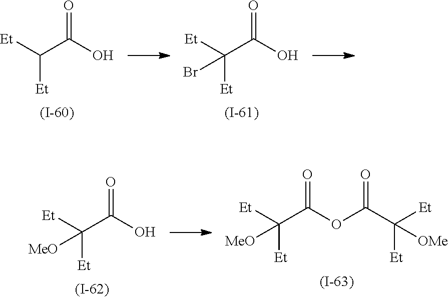

2-Ethyl-2-bromo-butyric acid (I-61) is commercially available or can be prepared according to the procedure described by Doran; Shonle in Journal of Organic Chemistry, 1938, vol. 3, p. 195.

2-Ethyl-2-bromo-butyric acid (I-61) was first converted to Ethyl-2-methoxy-butyric acid (I-62) and then to Ethyl-2-methoxy-butyric acid anhdride (I-63) as a light yellowish oil as described in Preparation 2.

The following s and anhydrides were prepared similarly.

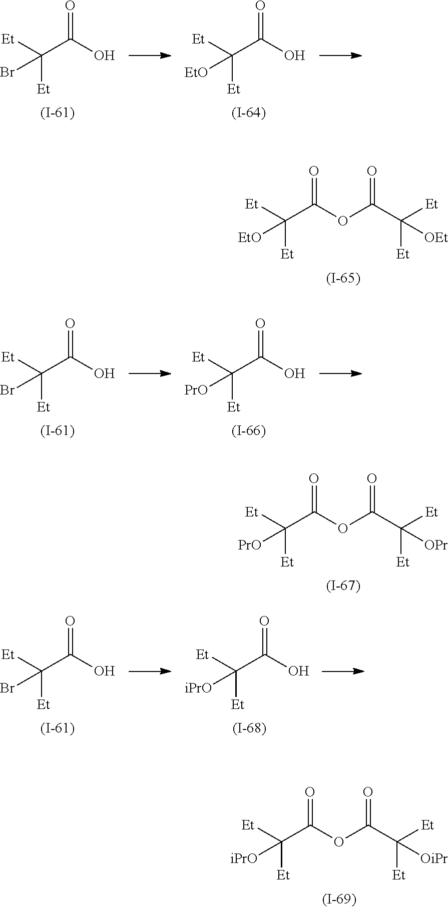

### Preparation 6 Synthesis of 2-methyl-2-alkoxyl-butyric acid and anhydrides

### Procedure for Synthesis of 2-methyl-2-methoxyl-butyric acid (I-72) and the anhydride (I-73)

Commercially available (R,S)-2-hydroxy-2-methylbutyric acid (I-70) was resolved into enatiomerically pure R and S isomers (I-71) and then esterified to the methyl ester (I-72) according to the method described in Preparation 74 of US2008114005.

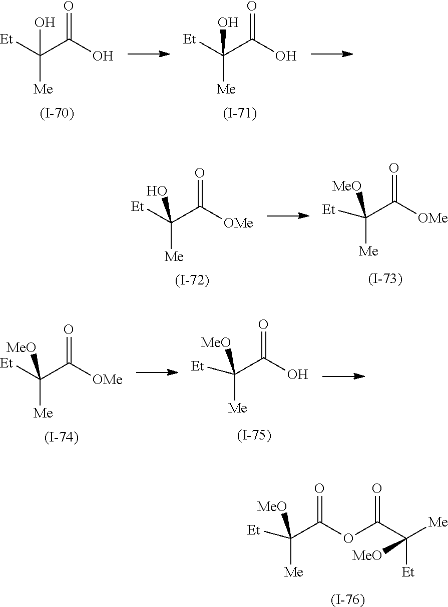

Alternatively commercially available 2-bromo-2-methylbutanoic acid was transformed to (R,S)-2-methoxy-2-methylbutyric acid (I-78) according to the method disclosed in preparation 2, (I-78) was resolved to the enatiomers (I-80) and (I-77) according to the method described in Preparation 74 of US2008114005. The chiral acid (I-75) was then transformed to the anhydride (I-76 as an oil as described in Preparation 2.

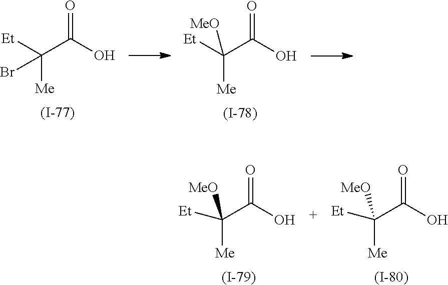

The following s and anhydrides were prepared similarly.

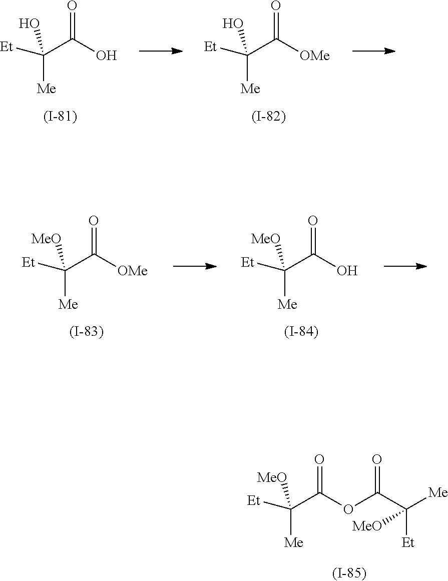

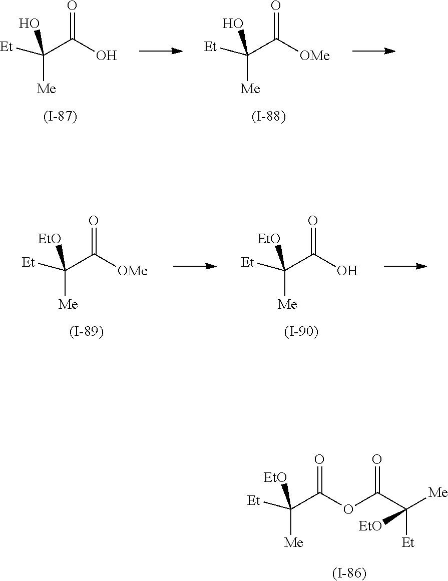

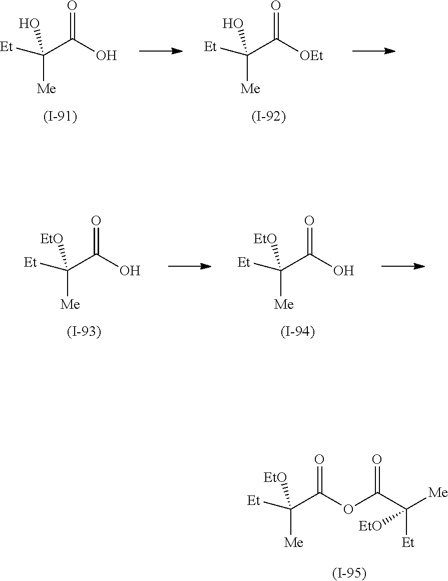

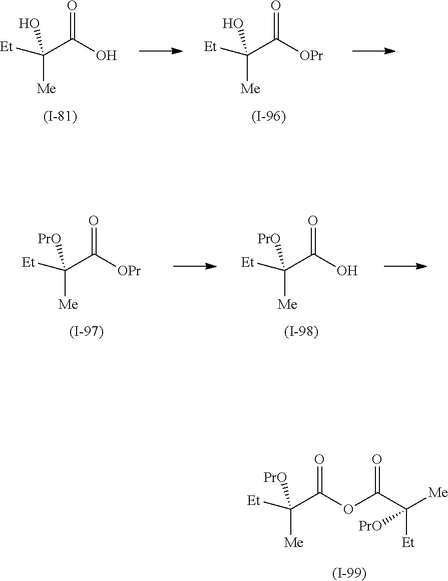

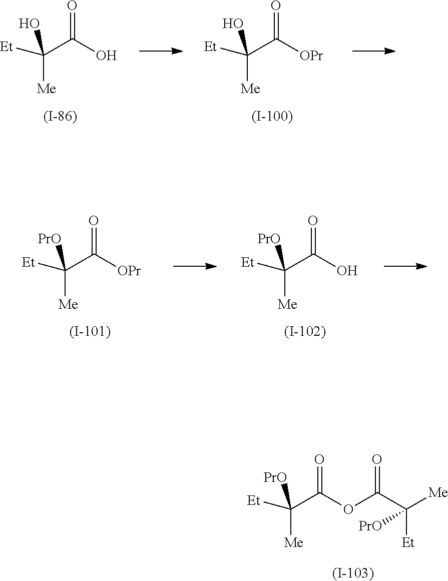

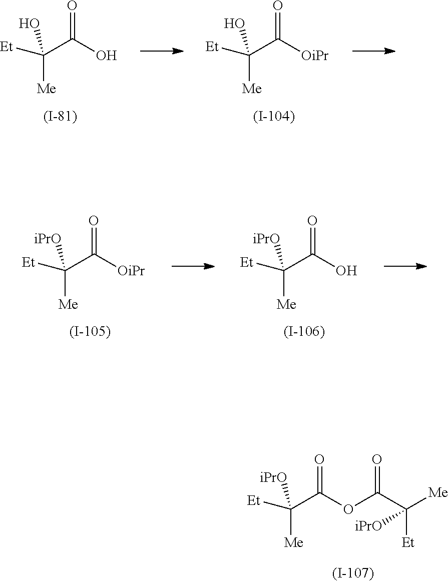

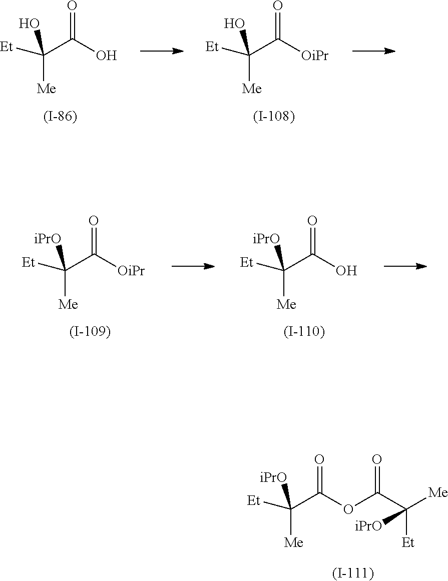

### Preparation 7 Synthesis of 2-alkyl tetrahydrofuran-2-carboxylic acids and anhydrides

Procedure for Synthesis of 2-methyl tetrahydrofuran-2-carboxylic acids (I-112), (I-114) and the anhydride (I-113), (I-115): Enatiomerically pure 2-methyl tetrahydrofuran-2-carboxylic acids (I-112) and (I-114) were prepared according to the process described by Pohl; Wollweber in European Journal of Medicinal Chemistry, 1976, vol. 11, p. 163,168,169. The acids are then converted to the corresponding anhydrides (I-113) and (I-115) in the similar manner as that described in preparation 2.

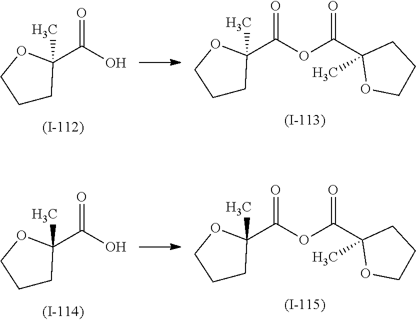

The following 2-alkyl tetrahydrofuran-2-carboxylic acids and the anhydrides were prepared similarly:

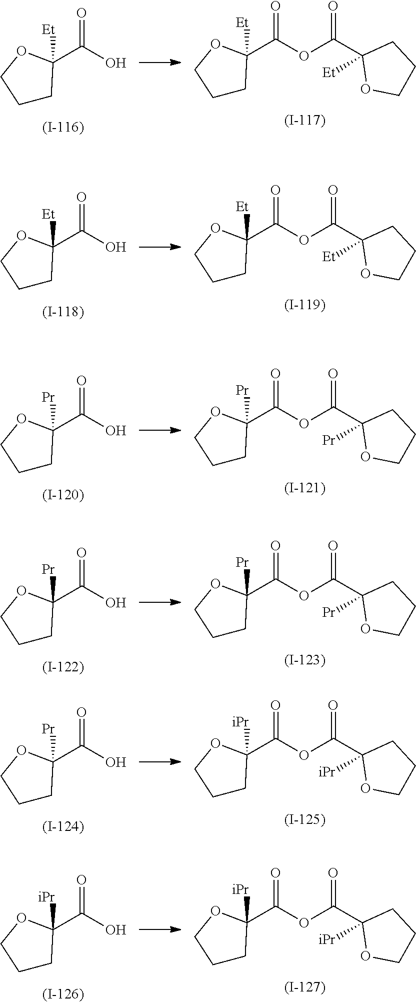

### Preparation 8 Synthesis of 2-methyl-2-alkoxymethylpropionic acid and anhydrides

### Procedure for Synthesis of 2-methyl-2-methoxymethylpropionic acid (I-130) and the anhydride (I-131)

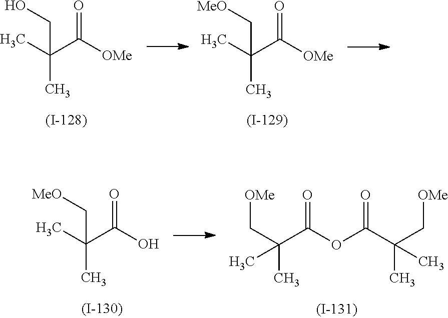

Commercially available 2-methyl-2-hydroxymethylpropionic acid methyl ester (I-128) was first methylated and then the ester hydrolyzed to afford the 2-methyl-2-methoxymethylpropionic acid (I-130) using the processes described in Example 55 and 56 in WO2009/77608, 2009.

2-methyl-2-methoxymethylpropionic acid (I-130) was then converted to the anhydride (I-131) according to the process of Preparation 2 to afford it as an oil.

The following 2-methyl-2-alkoxymethylpropionic acid anhydrides were prepared similarly.

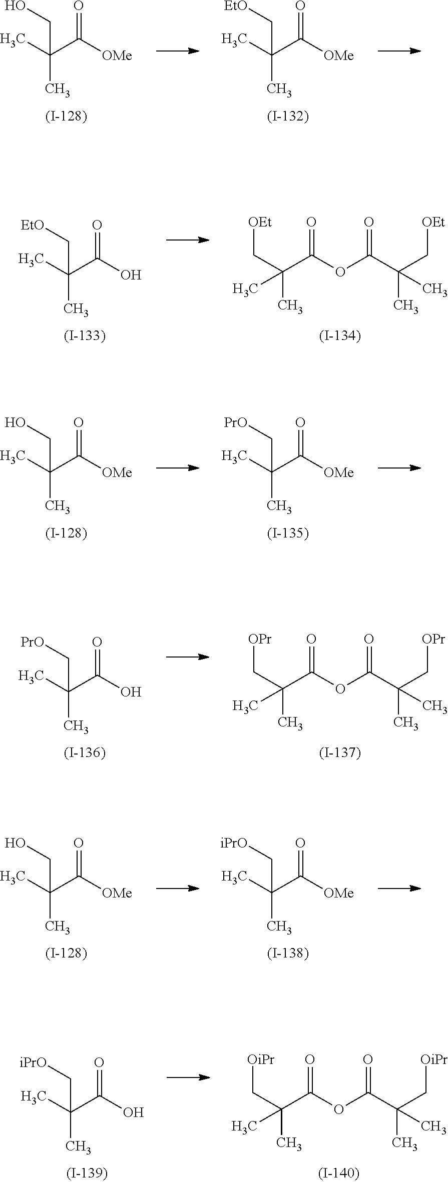

### Preparation 9 Synthesis of 1-alkyl,2,2-dialkoxy-isobutyric acids and anhydrides

### Procedure for Synthesis of 1-Methyl-2,2-Dimethoxy-isobutyric acids (I-142) and the anhydride (I-143)

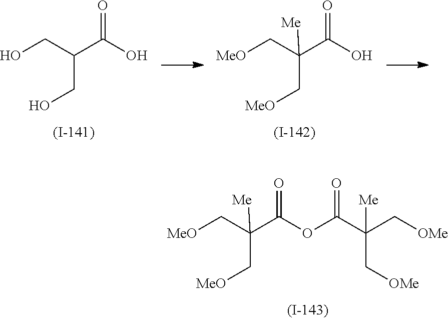 [SMILES: O=C(O)C(CO)CO ; COCC(C)(COC)C(=O)O ; COCC(C)(COC)C(=O)OC(=O)C(C)(COC)COC]

1-Methyl-2,2-Dimethoxy-isobutyric acid (I-142) was prepared according to the procedure described in Reference example 14 of US2004248941.

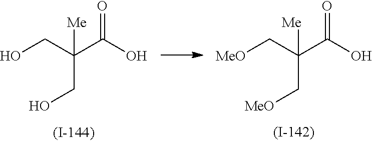 [SMILES: CC(CO)(CO)C(=O)O ; COCC(C)(COC)C(=O)O]

Alternatively, 1-Methyl-2,2-Dimethoxy-isobutyric acid (I-142) was prepared from commercially available 2,2-bis(hydroxymethyl)propanoic acid according to Reference Example 14 of EP1437352.

1-Methyl-2,2-Dimethoxy-isobutyric acid (I-142) was then converted to the anhydride (I-143) according to the process of Preparation 2 to afford it as an oil.

The following 1-Alkyl-2,2-Dialkoxy-isobutyric acids and anhydrides were prepared similarly.

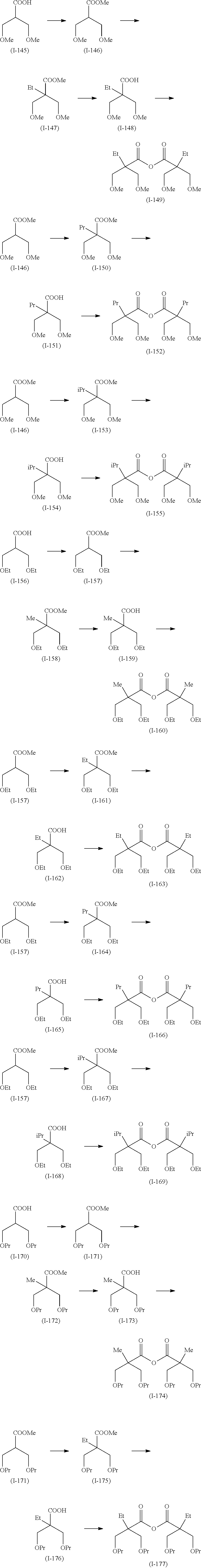

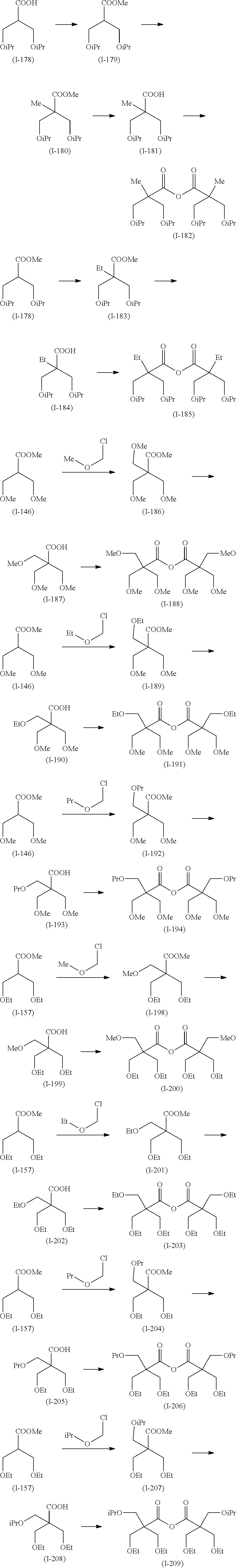

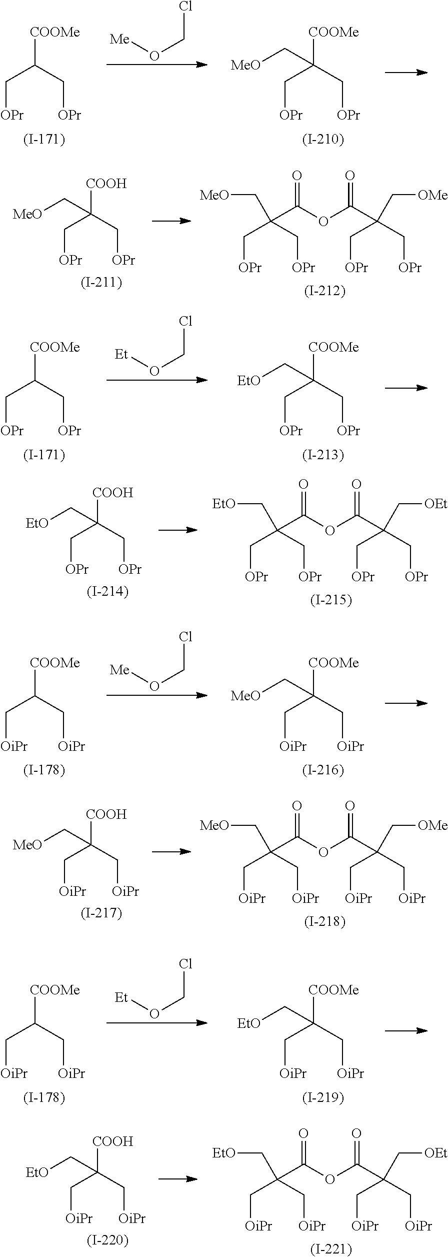

### Preparation 10 Synthesis of 1-(alkoxymethyl)cyclopropane-1-carboxylic acid and anhydrides

### Procedure for Synthesis of 1-(methoxymethyl)cyclopropane-1-carboxylic acid (I-225) and the anhydride (I-226)

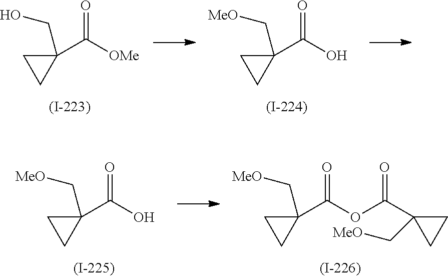 [SMILES: COC(=O)C1(CO)CC1 ; COCC1(C(=O)O)CC1 ; COCC1(C(=O)O)CC1 ; COCC1(C(=O)OC(=O)C2(COC)CC2)CC1]

1-(hydroxymethyl)cyclopropane-1-carboxylic acid methyl ester (I-223) was prepared according to the procedure described in Reference example 22-1 of U.S. Pat. No. 9,546,155. The hydroxy group was then alkylated with Methyl iodide using the similar process described by Shen, Peng-Xiang; et al., in Journal of the American Chemical Society, 2018, vol. 140, #21, p. 6545-6549. The ester is then hydrolyzed to afford 1-(methoxymethyl)cyclopropane-1-carboxylic acid (I-225).

1-(methoxymethyl)cyclopropane-1-carboxylic acid (I-225) was then converted to the anhydride (I-226) according to the process of Preparation 2 to afford (I-226) as an oil.

The following 1-(alkoxymethyl)cyclopropane-1-carboxylic acids and anhydrides were prepared similarly.

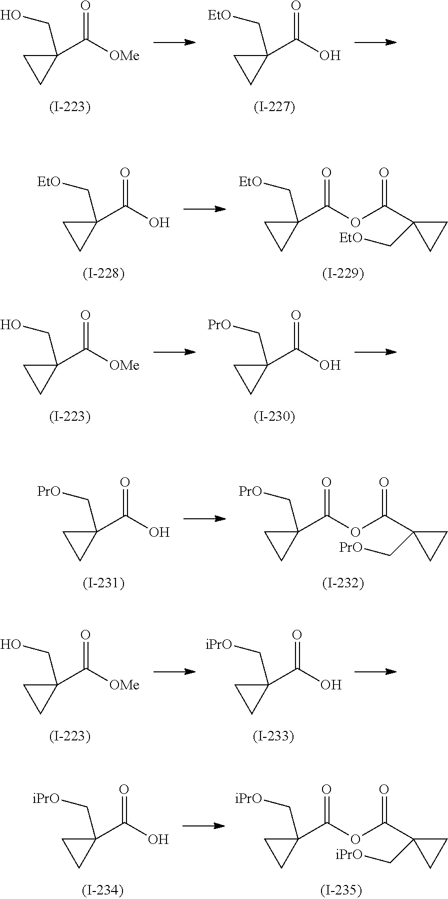

### Preparation 11 Synthesis of 1-(alkoxymethyl)cyclobutane-1-carboxylic acid and anhydrides

### Procedure for Synthesis of 1-(Methoxymethyl)cyclobutane-1-carboxylic acid (I-238) and the anhydride (I-239)

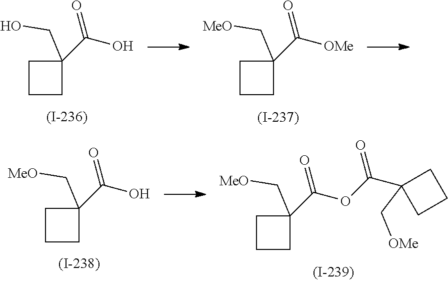 [SMILES: O=C(O)C1(CO)CCC1 ; COCC1(C(=O)OC)CCC1 ; COCC1(C(=O)O)CCC1 ; COCC1(C(=O)OC(=O)C2(COC)CCC2)CCC1]

1-(hydroxymethyl)cyclobutane-1-carboxylic acid methyl ester (I-236) was prepared according to the procedure described in Reference example 22-4 of U.S. Pat. No. 9,546,155. The hydroxy group was then alkylated with Methyl iodide followed by ester hydrolysis using the similar process described in Reference example K-19 in U.S. Ser. No. 10/040,791 to afford 1-(Metoxymethyl)cyclobutane-1-carboxylic acid (I-238).

1-(Metoxymethyl)cyclobutane-1-carboxylic acid (I-238) was then converted to the anhydride (I-239) according to the process of Preparation 2 to afford it as an oil.

The following 1-(alkoxymethyl)cyclobutane-1-carboxylic acids and anhydrides were prepared similarly.

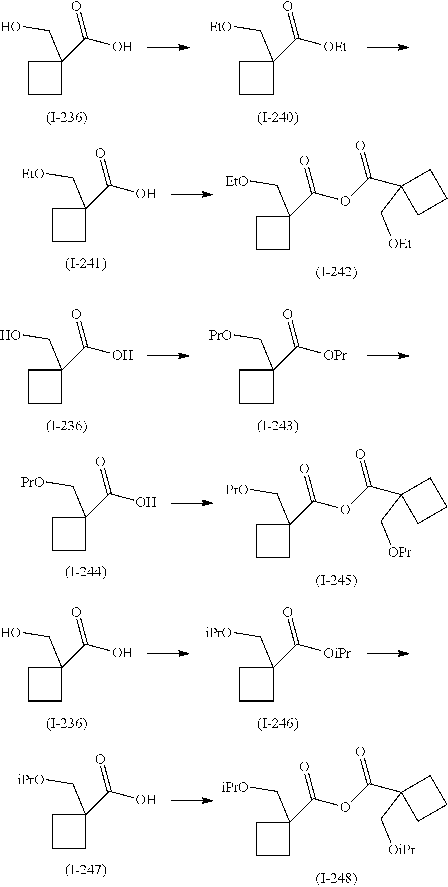

### Preparation 12 Synthesis of 1,2,2-trialkoxy-isobutyric acids and anhydrides

### Procedure for Synthesis of 1-Methoxy-2,2-Diethoxy-isobutyric acids (223) and the anhydride (224)

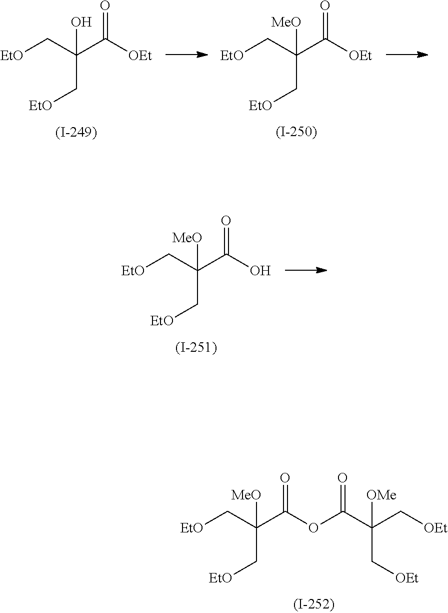 [SMILES: CCOCC(O)(COCC)C(=O)OCC ; CCOCC(COCC)(OC)C(=O)OCC ; CCOCC(COCC)(OC)C(=O)O ; CCOCC(COCC)(OC)C(=O)OC(=O)C(COCC)(COCC)OC]

1-Hydroxy-2,2-Diethoxy-isobutyric acids ethyl ester (I-249) was prepared according to the procedure described by Bernardon, C. et al. In Comptes Rendus des Seances de l'Academie des Sciences, Serie C: Sciences Chimiques, 1968, vol. 266, p. 1502-1505. The hydroxy group was then alkylated with Methyl iodide followed by ester hydrolysis using the similar process described in Reference example K-19 in U.S. Ser. No. 10/040,791 to afford 1-Methoxy-2,2-Diethoxy-isobutyric acid (I-251).

1-Methoxy-2,2-Diethoxy-isobutyric acid (I-251) was then converted to the anhydride (I-252) according to the process of Preparation 2 to afford it as an oil.

The following 1-Alkoxy-2,2-Dialkoxy-isobutyric acid and anhydride were prepared similarly.

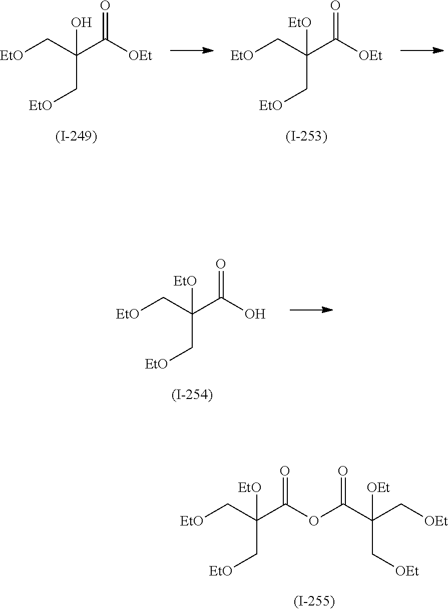 [SMILES: CCOCC(O)(COCC)C(=O)OCC ; CCOCC(COCC)(OCC)C(=O)OCC ; CCOCC(COCC)(OCC)C(=O)O ; CCOCC(COCC)(OCC)C(=O)OC(=O)C(COCC)(COCC)OCC]

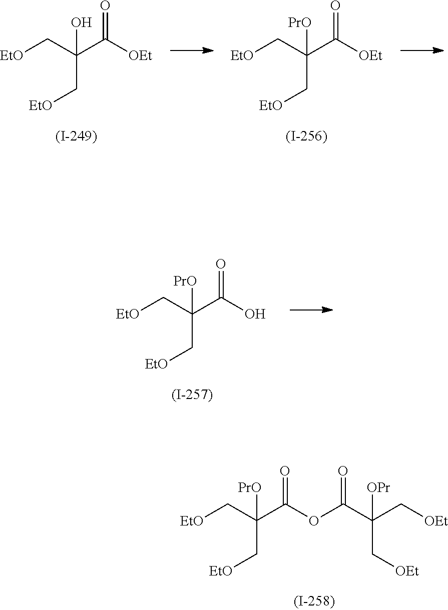

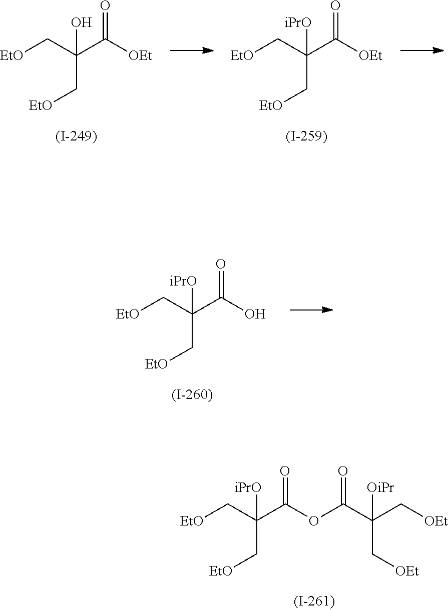 [SMILES: CCOCC(O)(COCC)C(=O)OCC ; CCOCC(COCC)(OC(C)C)C(=O)OCC ; CCOCC(COCC)(OC(C)C)C(=O)O ; CCOCC(COCC)(OC(C)C)C(=O)OC(=O)C(COCC)(COCC)OC(C)C]

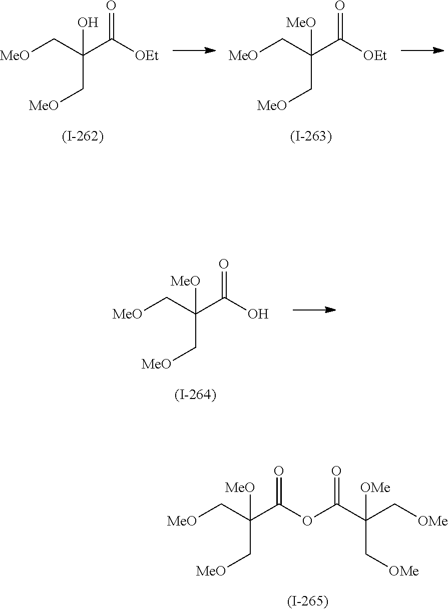

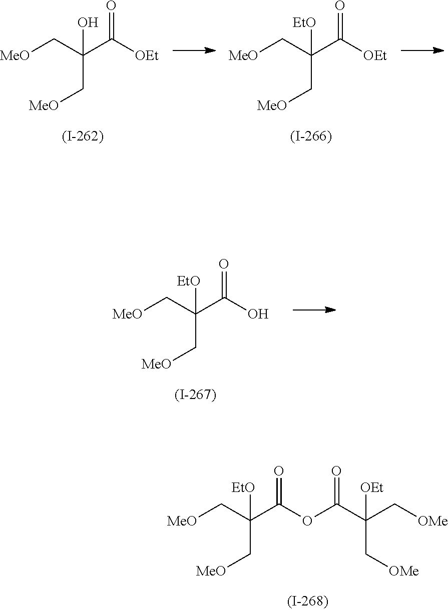

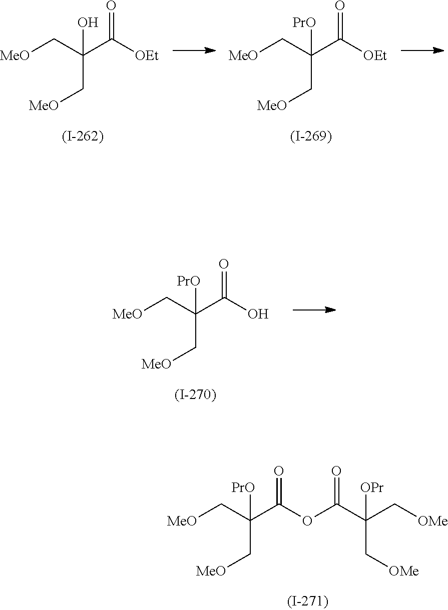

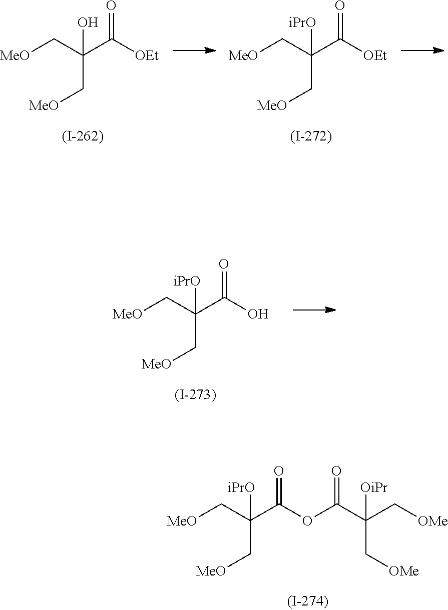

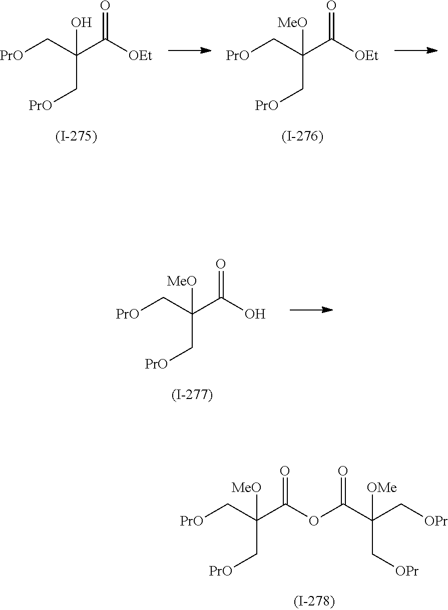

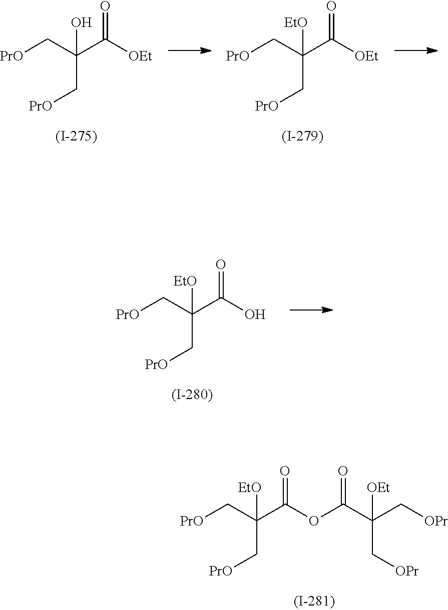

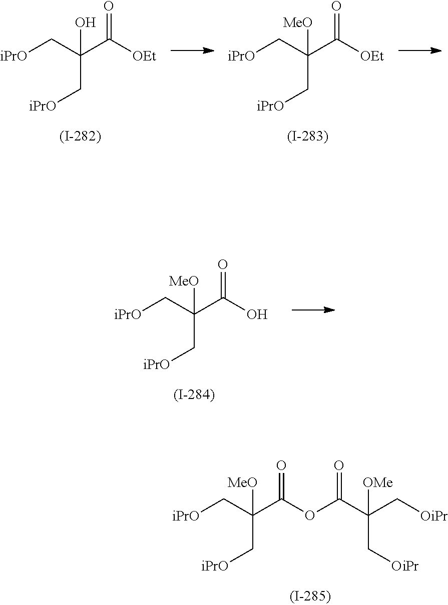

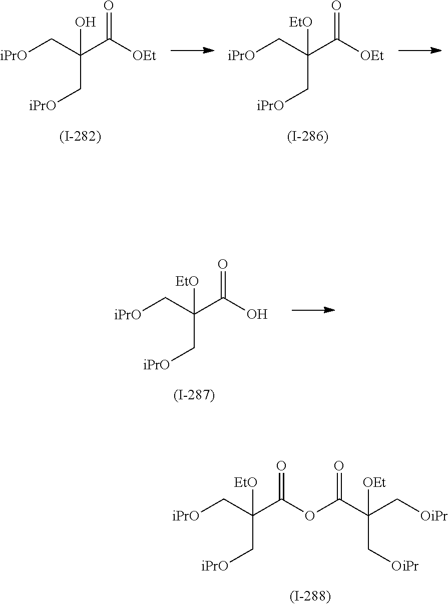

### Preparation 13: Synthesis of 1-alkoxycyclobutanecarboxylic acids and anhydrides

### Procedure for Synthesis of 1-methoxycyclopropanecarboxylic acid (I-291) and the anhydride (I-292)

Commercially available methyl 2-methoxylacetate (I-289) was alkylated with dibromoethane in the same manner as those described in Example 26 3A of U.S. Ser. No. 10/464,914 to afforded the 1-methoxycyclopropanecarboxylic acid methyl ester which was then hydrolyzed under basic condition to afford the corresponding acid (I-291). The acid (I-291) was then transformed to the corresponding anhydride (I-292) according to the process of Preparation 2 to afford it as an oil.

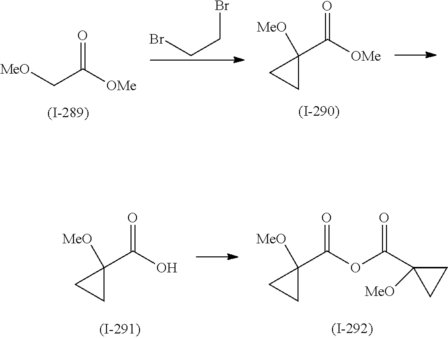

In the same manner as described in the preparation above, the following 1-methoxycyclopropanecarboxylic acid and the anhydride as well as 1-alkoxycyclobutanecarboxylic acids and anhydrides can be prepared:

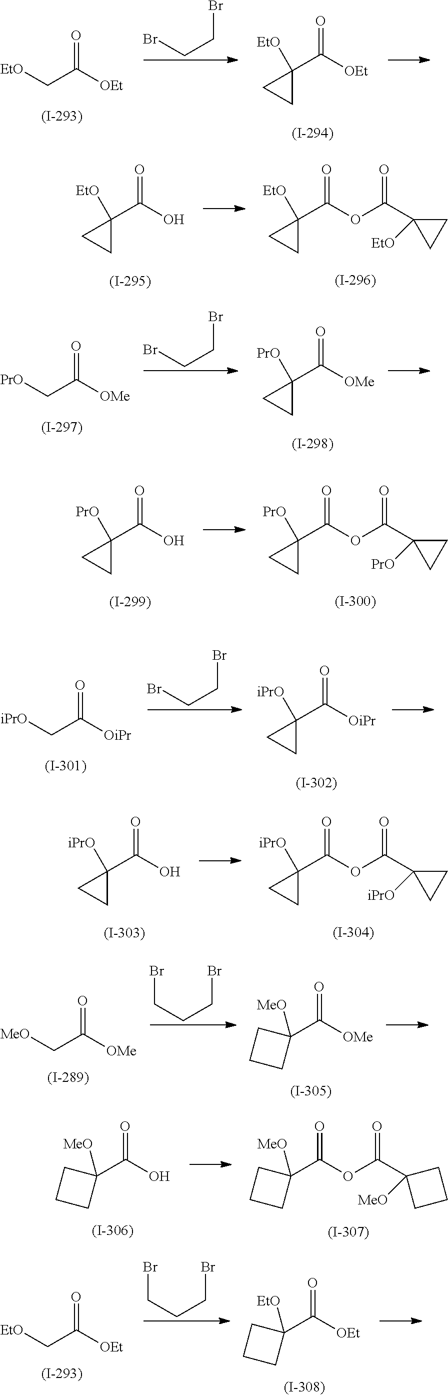

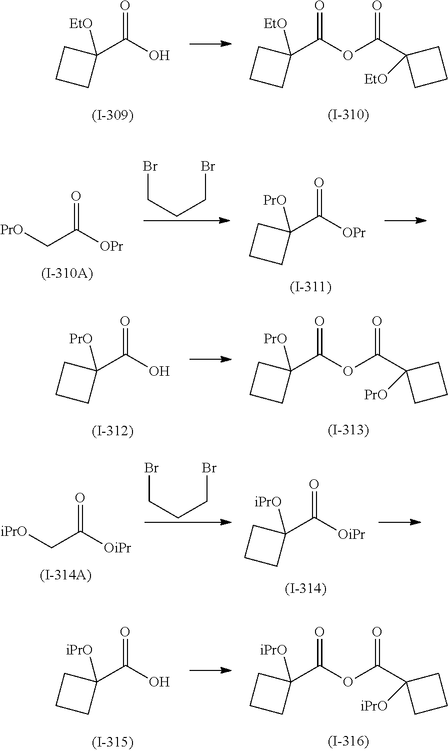

### Example 1: Synthesis of N4-hydroxycytidine (NHC) or 1-(3,4-dihydroxy-5-(hydroxymethyl) tetrahydrofuran-2-yl)-4-(hydroxyamino)pyrimidin-2-one

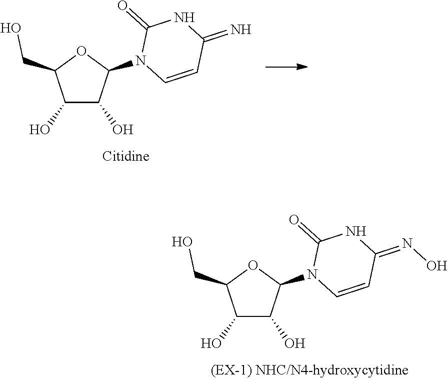 [SMILES: N=c1ccn([C@@H]2O[C@H](CO)[C@@H](O)[C@H]2O)c(=O)[nH]1 ; O=c1[nH]/c(=N/O)ccn1[C@@H]1O[C@H](CO)[C@@H](O)[C@H]1O]

A mixture of cytidine (20.0 g, 82.24 mmol, 1.0 eq) and NH₂OH·AcOH (23 g, 246.7 mmol, 3.0 eq) in H₂O (350 mL) was stirred for 48 hrs at 40° C. Reaction was monitored by HPLC and after completion of reaction, water was evaporated in rotavapor under vacuum to give a thick syrup, which was then suspended in 100 mL of water and placed in refrigerator for crystallization for 24 hrs. The solid thus crystallized was filtered, washed with cold H₂O (˜15.0 mL), dried under vacuum to yield the desired N4-hydroxycytidine (NHC/EX-1) as white solid (8.48 g, 40% yield). 1H NMR (400 MHz, DMSO) δ 9.97 (br. s, 1H), 9.45 (bs, 1H), 7.04 (d, 1H), 5.74 (d, 1H), 5.57 (d, 1H), 5.52 (m, 2H), 4.98-5.03 (m, 2H) 3.91-4.00 (m, 2H), 3.78 (dd, 1H), 3.56 (m 2H); Purity: 98% (assessed by HPLC).

### Example 2: Preparation of Compound (EX-2)

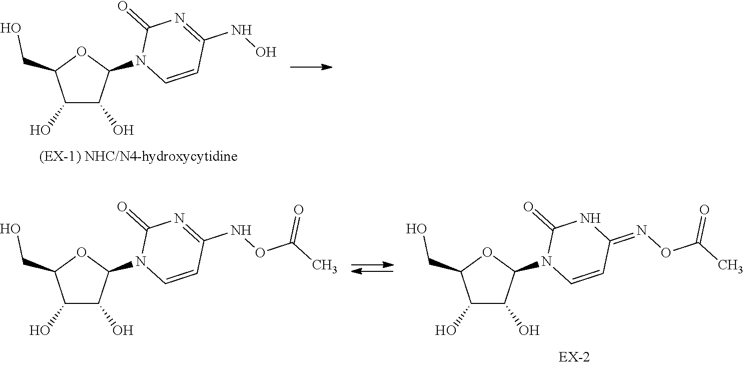 [SMILES: O=c1nc(NO)ccn1[C@@H]1O[C@H](CO)[C@@H](O)[C@H]1O ; CC(=O)ONc1ccn([C@@H]2O[C@H](CO)[C@@H](O)[C@H]2O)c(=O)n1 ; CC(=O)O/N=c1\ccn([C@@H]2O[C@H](CO)[C@@H](O)[C@H]2O)c(=O)[nH]1]

In a 250 mL round bottom flask with magnetic stirring was added N4-hydroxycytidine (20 g, 72.2 mmol) in 30 mL water under stirring and heated to 40° C., then acetic anhydride (8.1 g, 0.794 mmol) was added drop wise. The reaction was stirred at this temperature for 2 hours until HPLC indicated the completion of the reaction. The reaction mixture was slowly cooled to 0° C., and the solid formed was filtered, and washed with methanol to afford EX-2-1 as a white solid. 1H NMR (400 MHz, DMSO), two sets of peaks were observed in NMR due to tautomerization of the NH═CNHO bonds δ 10.9 (br. s, 1H), 7.4 (d, 1H), 5.7 (m, 2H), 5.3 (br.s, 1H), 5.0 (s, 2H), 4.0 (m, 2H), 3.8 (s, 1H), 3.6 (m, 2H), 2.10 (s, 3H); Purity: 99% (assessed by HPLC).

The following examples illustrate the novel process of the current selective acylation reaction with acetic anhydride. Single solvent or mixture of solvents (binary, trinary or quartnary mixtures etc.) can be used for the acylation with varying ratios. Acylated product is generally formed in >95% b HPLC in the reaction solution.

| | Solvent(s) | Anhydride | Reaction | EX2 purity in reaction |
|---|---|---|---|---|
| EX # | (Solvent(s): NHC = 8-12 w/w) | mol. Eq. | temperature | solution by HPLC |
| 2A | Water | 1.2 | 40-45° C. | 98.8% |
| 2B | nPropanol | 1.2 | 40-45° C. | 98.0% |
| 2C | Acetonitril | 1.2 | 40-45° C. | 98.4% |
| 2D | THF | 1.2 | 40-45° C. | 95.9% |
| 2E | Methanol | 1.2 | 40-45° C. | 99.5% |
| 2F | Ethanol | 1.2 | 40-45° C. | 99.2% |
| 2G | ACN:water (6:4, v/v) | 1.2 | 40-45° C. | 98.4% |
| 2H | THF:water (7:3, v/v) | 1.2 | 40-45° C. | 97.8% |
| 2I | MeOH:water (7:3, v/v) | 1.2 | 40-45° C. | 98.2% |
| 2J | EtOH:water (6:4, v/v) | 1.2 | 40-45° C. | 98.6% |
| 2K | nPrOH water (5:5, v/v) | 1.2 | 40-45° C. | 99.0% |
| 2L | iPrOH:water (5:5, v/v) | 1.2 | 40-45° C. | 98.9% |

The following is an example illustrating the industrial feasibility of the novel process:

In a 2 Liter four-neck round bottom flask equipped with stirrer and thermometer was added 160 g of NHC/N4-hydroxycytidine followed by 1.52 kg of methanol. The reaction mixture was heated to 45-50° C. and acetic anhydride (65 g) was added dropwise at this temperature with the dropping addition time of 10-15 min. About 50% of the methanol was distilled off under vacuum at 40-45° C. The reaction mixture was cooled to 30° C. and another 5 g of acetic anhydride was added dropwise. The reaction mixture was further cooled to below 10° C. and stirred at this temperature for 2 hours. The formed solid was filtered and washed with 100-150 g of methanol. The solid was dried to afford 160 g of compound EX-2 as a white solid with purity of 99.5%.

### Example 3: Preparation of Compound (EX-3)

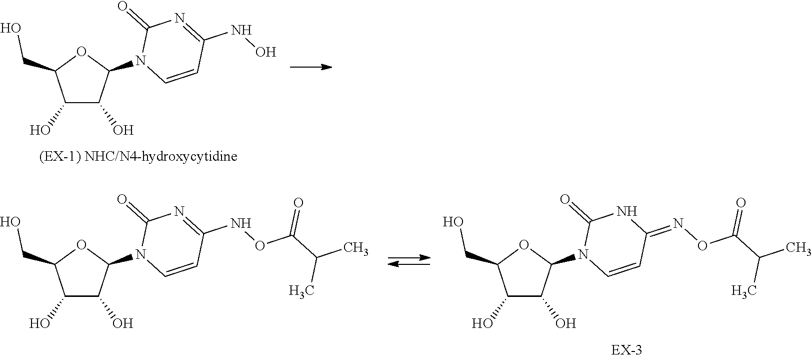 [SMILES: O=c1nc(NO)ccn1[C@@H]1O[C@H](CO)[C@@H](O)[C@H]1O ; CC(C)C(=O)ONc1ccn([C@@H]2O[C@H](CO)[C@@H](O)[C@H]2O)c(=O)n1 ; CC(C)C(=O)O/N=c1\ccn([C@@H]2O[C@H](CO)[C@@H](O)[C@H]2O)c(=O)[nH]1]

In a 100 mL round bottom flask with magnetic stirring was added N4-hydroxycytidine (20 g, 72. mmol) in 36 mL water under stirring and heated to 45° C., then isobutyric anhydride (12.5 g, 0.79 mmol) was added drop wise for 5-10 min. The reaction was stirred at this temperature for 1-2 hours until HPLC indicated the completion of the reaction. The water in the reaction flask was evaporated to dryness and methanol (20 mL) was then added. The reaction mixture was heated to 50-60° C. to fully disolve the solid and it was then slowly cooled to 0° C., and the solid formed was filtered, and washed with methanol to afford EX-3-1 as a white solid (20.5 g) with a purity of 99.3% in 86% yield. 1H NMR (400 MHz, DMSO), two sets of peaks were observed in NMR due to tautomerization of the NH═CNHO bonds δ 10.9 (br. s, 1H), 7.4 (d, 1H), 5.7 (m, 2H), 5.3 (br.s, 1H), 5.0 (s, 2H), 4.0 (m, 2H), 3.8 (s, 1H), 3.6 (m, 2H), 2.8 (m, 1H), 1.1 (s, 6H).

The following examples illustrate the novel process of the current selective acylation reaction with isobutyric anhydride. Single solvent or mixture of solvents (binary, trinary or quartnary mixtures etc.) can be used for the acylation with varying ratios. Acylated product is generally formed in >95% by HPLC in the reaction solution.

| | Solvent(s) | Anhydride | Reaction | EX2 purity in reaction |
|---|---|---|---|---|
| EX -# | (Solvent(s): NHC = 8-12 w/w) | mol. Eq. | temperature | solution by HPLC |
| 3A | Water | 1.2 | 40-45° C. | 99.6% |
| 2B | Methanol | 1.2 | 40-45° C. | 98.9% |
| 3C | ACN:water (3:2, v/v) | 1.2 | 40-45° C. | 99.3% |
| 3D | THF:water (3:2, v/v) | 1.2 | 40-45° C. | 98.7% |
| 3E | EtOH:water (4:1, v/v) | 1.2 | 40-45° C. | 99.1% |
| 3F | nPrOH water (7:3, v/v) | 1.2 | 40-45° C. | 99.4% |
| 3G | iPrOH:water (7:3, v/v) | 1.2 | 40-45° C. | 99.3% |

### Example 4: Preparation of Compound (EX-4)

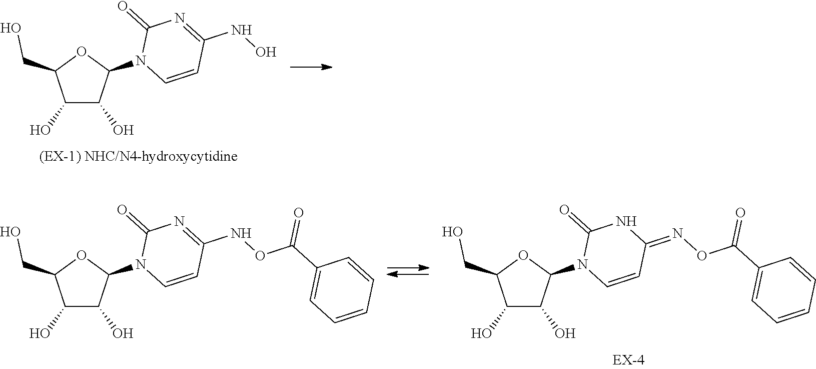 [SMILES: O=c1nc(NO)ccn1[C@@H]1O[C@H](CO)[C@@H](O)[C@H]1O ; O=C(ONc1ccn([C@@H]2O[C@H](CO)[C@@H](O)[C@H]2O)c(=O)n1)c1ccccc1 ; O=C(O/N=c1\ccn([C@@H]2O[C@H](CO)[C@@H](O)[C@H]2O)c(=O)[nH]1)c1ccccc1]

In a 100 mL round bottom flask with magnetic stirring was added N4-hydroxycytidine (20 g, 72. mmol) in 40 mL pyridine under stirring at room temperature, then benzoyl chloride (11.1 g, 0.79 mmol) was added drop wise for 5-10 min. The reaction was stirred at 40-50° C. for overnight until HPLC indicated the completion of the reaction. The excess pyridine was removed under vacuum, and the reaction residue was disolved in EtOAc, the organic layer was washed with saturated sodium chloride solution. The organic layer was dried, concentrated and purified on silica gel column (DCM and MeOH, gradients) to afford EX-4-1 as a white solid. 1H NMR (400 MHz, DMSO), two sets of peaks were observed in NMR due to tautomerization of the NH═CNHO bonds δ 11.14 (s, 1H), 8.22 (d, 1H), 7.96 (dd, 1H), 7.4-7.6 (m, 5H), 5.80 (m, 1H), 5.3 (br.s, 1H), 5.0 (br.s, 2H), 3.83-4.03 (m, 2H), 3.8 (s, 1H), 3.5 (m, 2H).

The following exemplary compounds were prepared similarly as described in Example 2, 3, or 4 using commercially available anhydrides or acyl chlorides. For those carboxylic anhydrides and acyl chlorides that are not commercially available, they can be easily prepared by well known standard procedures.

| Example | | |
|---|---|---|
| # | Structure | NMR & LC-MS data |
| EX-2 | 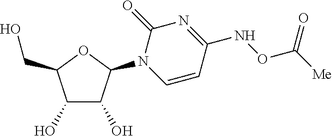 [SMILES: CC(=O)ONc1ccn([C@@H]2O[C@H](CO)[C@@H](O)[C@H]2O)c(=O)n1] | 1H NMR(400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.10 (s, 3H) |
| EX-3 | 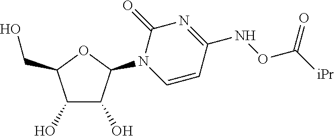 [SMILES: CC(C)C(=O)ONc1ccn([C@@H]2O[C@H](CO)[C@@H](O)[C@H]2O)c(=O)n1] | 1H NMR(400 MHz, DMSO) δ 10.87 (br. s, 1H), 7.51 (d, 1H), 5.7 (m, 2H), 5.3 (br. s, 1H), 5.0 (s, 2H), 4.0 (m, 2H), 3.8 (s, 1H), 3.58 (m, 2H), 2.81 (m, 1H), 1.1 (s, 6H) |
| EX-4 | 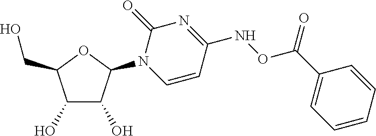 [SMILES: O=C(ONc1ccn([C@@H]2O[C@H](CO)[C@@H](O)[C@H]2O)c(=O)n1)c1ccccc1] | 1H NMR(400 MHz, DMSO) δ 11.14 (s, 1H), 8.22 (d, 1H), 7.96 (dd, 1H), 7.4-7.6 (m, 5H), 5.80 (m, 1H), 5.3 (br. s, 1H), 5.0 (br. s, 2H), 3.83-4.03 (m, 2H), 3.8 (s, 1H), 3.5 (m, 2H). |
| EX-5 | 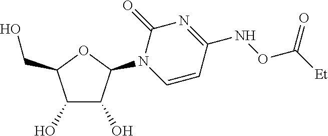 [SMILES: CCC(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.5 (m, 2H), 1.1 (m, 3H). |
| EX-4a | 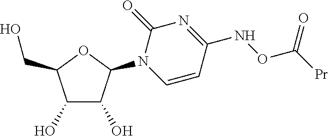 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.4 (m, 2H), 1.5 (m, 2H), 0.8 (m, 3H). |
| EX-5a | 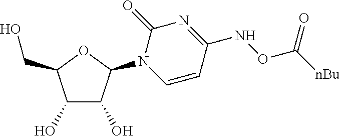 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.5 (m, 2H), 1.5 (m, 2H), 1.3 (m, 2H), 0.8 (tr, 3H). |
| EX-6 | 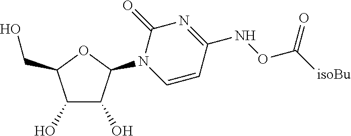 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.3 (m, 2H), 2.0 (m, 1H), 0.9 (d, 3H). |
| EX-7 | 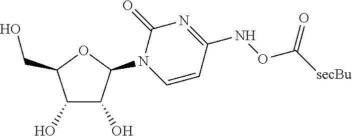 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.8 (m, 1H), 1.6 (m, 1H), 1.4 (m, 1H), 1.1 (d, 3H), 0.8 (tr, 3H). |
| EX-8 | 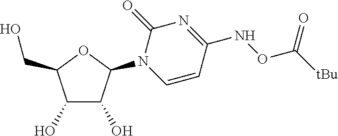 [SMILES: CC(C)(C)C(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 1.2 (tr, 9H). |
| EX-9 | 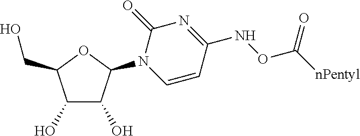 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.5 (m, 2H), 1.65 (m, 2H), 1.3 (m, 4H), 0.8 (tr, 3H). |
| EX-11 | 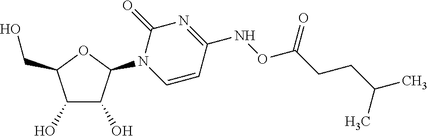 [SMILES: CC(C)CCC(=O)ONc1ccn([C@@H]2O[C@H](CO)[C@@H](O)[C@H]2O)c(=O)n1] | 1H NMR (400 MHz, DMSO) δ 10.9 (s, 1H), 7.5 (d, 1H), 5.7 (m, 2H), 5.3 (d, 1H), 5.0 (s, 2H), 4.0 (m, 2H), 3.8 (d, 1H), 3.6 (m, 2H), 2.5 (m, 2H), 1.5 (m, 3H) 0.9 (t, 6H). |
| EX-12 | 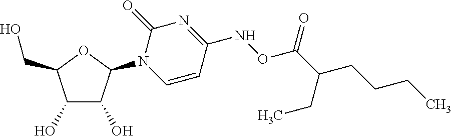 [SMILES: CCCCC(CC)C(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.4 & 2.7 (m, 1H), 1.5 (m, 2H), 1.4 (m, 2H), 1.2 (m, 4H), 0.8 (tr, 6H). |
| EX-13 | 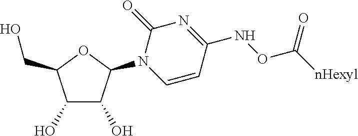 | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.5 (m, 2H), 1.55 (m, 2H), 1.3 (m, 6H), 0.8 (tr, 3H). |
| EX-15 | 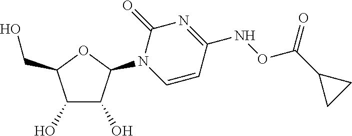 [SMILES: O=C(ONc1ccn([C@@H]2O[C@H](CO)[C@@H](O)[C@H]2O)c(=O)n1)C1CC1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.0 (m, 1H), 0.8 (m, 4H). |
| EX-16 | 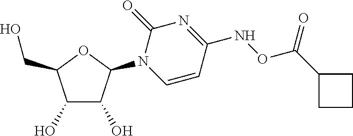 [SMILES: O=C(ONc1ccn([C@@H]2O[C@H](CO)[C@@H](O)[C@H]2O)c(=O)n1)C1CCC1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 3.3 (m, 1H), 2.2 (m, 4H), 1.9 (m, 1H), 1.8 (m, 1H). |
| EX-17 | 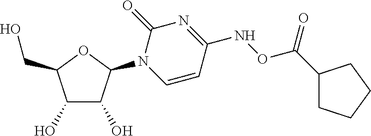 [SMILES: O=C(ONc1ccn([C@@H]2O[C@H](CO)[C@@H](O)[C@H]2O)c(=O)n1)C1CCCC1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 3.0 (m, 1H), 1.8 (m, 2H), 1.5-1.7 (m, 6H). |
| EX-18 | 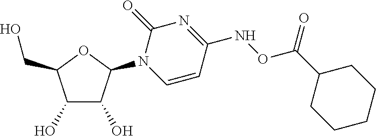 [SMILES: O=C(ONc1ccn([C@@H]2O[C@H](CO)[C@@H](O)[C@H]2O)c(=O)n1)C1CCCCC1] | 1H NMR (400 MHz, DMSO) δ 10.87 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 3.45 (m, 2H), 3.34 (m, 1H), 2.51 (m, 1H) 1.84 (m, 2H), 1.22-1.36 (m, 5H). |
| EX-24 | 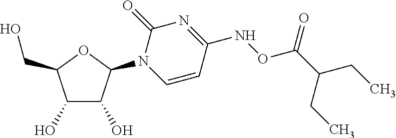 [SMILES: CCC(CC)C(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.5 (m, 1H), 1.5 (m, 4H), 0.8 (tr, 6H). |
| EX-25 | 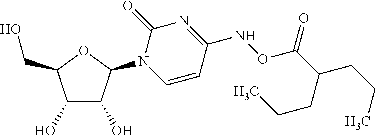 [SMILES: CCCC(CCC)C(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.4 & 2.6 (m, 1H), 1.5 (m, 4H), 1.2 (m, 4H), 0.8 (tr, 6H). |
| EX-26 | 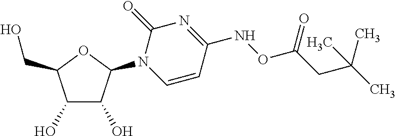 [SMILES: CC(C)(C)CC(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.45 & 2.6 (s, 2H), 1.0 (s, 9H). |
| EX-27 | 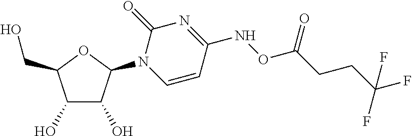 [SMILES: O=C(CCC(F)(F)F)ONc1ccn([C@@H]2O[C@H](CO)[C@@H](O)[C@H]2O)c(=O)n1] | 1H NMR (400 MHz, DMSO) δ 11.0 (br. s, 1H), 7.9 (s, 1H), 7.4 (d, 1H), 5.7 (m, 2H), 5.3 (br. s, 1H), 5.0 (s, 2H), 4.0 (m, 2H), 3.8 (s, 1H), 3.6 (m, 2H), 2.45 & 2.6 (s, 6H), 2.5 (s, 2H). |
| EX-39 | 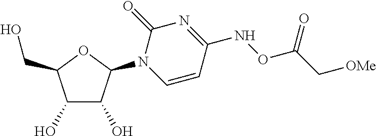 [SMILES: COCC(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.2 (s, 2H), 4.0 (m, 2H), 3.8 (s, 1H), 3.6 (m, 2H), 3.3 (s, 3H). |
| EX-40 | 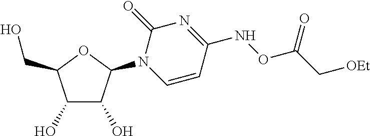 [SMILES: CCOCC(=O)ONc1ccn([C@@H]2O[C@H](CO)[C@@H](O)[C@H]2O)c(=O)n1] | 1H NMR (400 MHz, DMSO) δ 10.9 (br. s, 1H), 7.4 (d, 1H), 5.7 (m, 2H), 5.3 (br. s, 1H), 5.0 (s, 2H), 4.2 (s, 2H), 4.0 (m, 2H), 3.8 (s, 1H), 3.5 (m, 4H), 1.1 (m, 3H). |
| EX-41 | 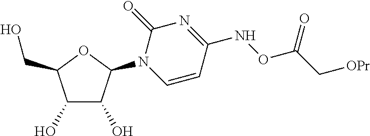 | 1H NMR (400 MHz, DMSO) δ 10.9 (br. s, 1H), 7.5 (d, 1H), 5.7 (m, 2H), 5.3 (br. s, 1H), 5.0 (s, 2H), 4.2 (s, 2H), 4.0 (m, 2H), 3.8 (s, 1H), 3.5 (m, 4H), 1.5 (m, 2H), 0.8 (t, 3H) |
| EX-42 | 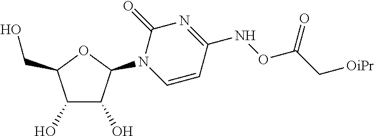 [SMILES: CC(C)OCC(=O)ONc1ccn([C@@H]2O[C@H](CO)[C@@H](O)[C@H]2O)c(=O)n1] | 1H NMR (400 MHz, DMSO) δ 10.9 (br. s, 1H), 7.5 (d, 1H), 5.7 (m, 2H), 5.3 (br. s, 1H), 5.0 (s, 2H), 4.2 (s, 2H), 4.0 (m, 2H), 3.8 (s, 1H), 3.5 (m, 3H), 1.1 (m, 6H) |
| EX-51 | 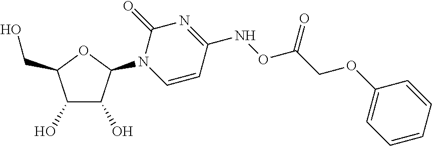 [SMILES: O=C(COc1ccccc1)ONc1ccn([C@@H]2O[C@H](CO)[C@@H](O)[C@H]2O)c(=O)n1] | 1H NMR(400 MHz, DMSO) δ 11.0 (s, 1H), 7.5 (s, 1H), 7.3 (m, 2H), 7.0 (m, 3H), 5.8 (dd , 2H), 5.3 (d, 1H), 5.0 (dd, 2H), 4.9 (s, 2H), 4.0 (m, 1H), 3.8 (s, 1H) 3.6 (m, 2H). |
| EX-52 | 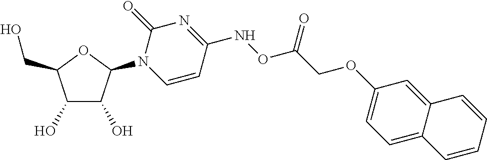 [SMILES: O=C(COc1ccc2ccccc2c1)ONc1ccn([C@@H]2O[C@H](CO)[C@@H](O)[C@H]2O)c(=O)n1] | 1H NMR(400 MHz, DMSO) 8 11.0 (s, 1H), 7.8-7.9 (m, 3H), 7.2-7.5 (m, 5H), , 5.8 (dd, 1H), 5.3 (d, 1H), 5.0 (dd, 3H), 4.0 (m, 2H), 3.8 (d, 1H), 3.6 (m, 2H) 3.6 (m, 2H). |
| EX-53 | 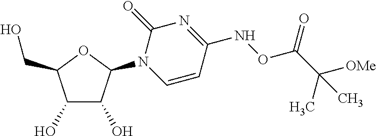 [SMILES: COC(C)(C)C(=O)ONc1ccn([C@@H]2O[C@H](CO)[C@@H](O)[C@H]2O)c(=O)n1] | two sets of peaks are observed in NMR due to tautomerization 1H NMR (400 MHz, DMSO) δ 11.2 & 10.7 (br. s, 1H), 7.55 & 7.45 (d, 1H), 6.0 & 5.7 (m, 2H), 5.3 (br. s, 1H), 5.0 (s, 2H), 4.0 (m, 2H), 3.8 (s, 1H), 3.6 (m, 2H), 3.2 (s, 3H), 1.45 & 1.35 (s, 6H) |
| EX-54 | 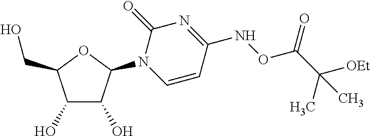 [SMILES: CCOC(C)(C)C(=O)ONc1ccn([C@@H]2O[C@H](CO)[C@@H](O)[C@H]2O)c(=O)n1] | two sets of peaks are observed in NMR due to tautomerization 1H NMR (400 MHz, DMSO) δ 11.2 & 10.6 (br. s, 1H), 7.55 & 7.45 (d, 1H), 6.0 & 5.7 (m, 2H), 5.3 (br. s, 1H), 5.0 (s, 2H), 4.0 (m, 2H), 3.8 (s, 1H), 3.6 (m, 2H), 3.4 (m, 2H), 1.45 & 1.35 (s, 6H), 1.1 (tr, 3H) |
| EX-55 | 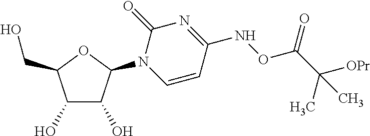 | two sets of peaks are observed in NMR due to tautomerization 1H NMR (400 MHz, DMSO) δ 11.2 & 10.6 (br. s, 1H), 7.55 & 7.45 (d, 1H), 6.0 & 5.7 (m, 2H), 5.0-5.3 (br. s, 3H), 4.0 (m, 2H), 3.9 (s, 1H), 3.5 (m, 2H), 3.3 (m, 2H), 1.5 (m, 8H), 0.8 (t, 3H) |
| EX-56 | 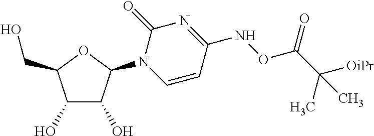 [SMILES: CC(C)OC(C)(C)C(=O)ONc1ccn([C@@H]2O[C@H](CO)[C@@H](O)[C@H]2O)c(=O)n1] | two sets of peaks are observed in NMR due to tautomerization 1H NMR (400 MHz, DMSO) δ 11.2 & 10.6 (br. s, 1H), 7.55 & 7.45 (d, 1H), 6.0 & 5.7 (m, 2H), 5.3 (br. s, 1H), 5.0 (s, 2H), 4.0 (m, 2H), 3.8 (s, 1H), 3.6 (m, 1H), 3.5 (m, 2H), 1.4 (m, 6H). 1.0 (tr, 6H) |
| EX-57 | 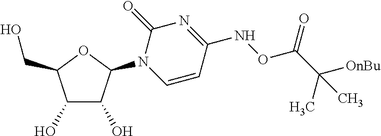 | two sets of peaks are observed in NMR due to tautomerization 1H NMR (400 MHz, DMSO) δ 11.2 & 10.6 (br. s, 1H), 7.55 & 7.45 (d, 1H), 6.0 & 5.7 (m, 2H), 5.3 (br. s, 1H), 5.0 (s, 2H), 4.0 (m, 2H), 3.8 (s, 1H), 3.6 (m, 2H), 3.3 (m, 2H), 1.3 (m, 4H), 0.8 (tr, 3H) |
| EX-58 | 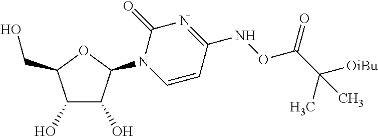 | two sets of peaks are observed in NMR due to tautomerization 1H NMR (400 MHz, DMSO) δ 11.2 & 10.6 (br. s, 1H), 7.55 & 7.45 (d, 1H), 6.0 & 5.7 (m, 2H), 5.3 (br. s, 1H), 5.0 (s, 2H), 4.0 (m, 2H), 3.8 (s, 1H), 3.6 (m, 2H), 3.1 (m, 2H), 1.6 (m, 1H), 1.4 (m, 6H), 0.8 (s, 6H) |
| EX-67 | 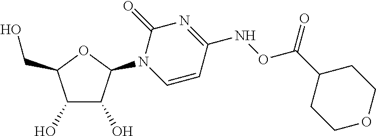 [SMILES: O=C(ONc1ccn([C@@H]2O[C@H](CO)[C@@H](O)[C@H]2O)c(=O)n1)C1CCOCC1] | 1H NMR (400 MHz, DMSO) δ 10.9 (br. s, 1H), 7.4 (s, 1H), 5.7 (m, 2H), , 5.3 (s, 1H), 5.0 (s, 2H), 3.8-4.0 (m, 5H), 3.6 (m, 2H), 3.4 (m, 2H), 2.8 (m, 1H), 1.7 (m, 2H), 1.6 (m, 2H). |
| EX-79 | 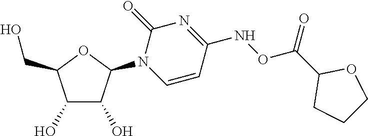 [SMILES: O=C(ONc1ccn([C@@H]2O[C@H](CO)[C@@H](O)[C@H]2O)c(=O)n1)C1CCCO1] | 1H NMR (400 MHz, DMSO) multiple sets of peaks: δ 10.8 (br. s, 1H), 7.4 (d, 1H), 5.7 (m, 2H), 5.3 (br. s, 1H), 5.0 (s, 2H), 3.8-4.0 (m, 5H), 3.6 (m, 2H), 1.8 (m, 2H), 1.6 (m, 2H). |
| EX-123 | 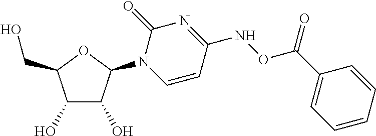 [SMILES: O=C(ONc1ccn([C@@H]2O[C@H](CO)[C@@H](O)[C@H]2O)c(=O)n1)c1ccccc1] | 1H NMR(400 MHz, DMSO) δ 11.14 (s, 1H), 8.22 (d, 1H), 7.96 (dd, 1H), 7.4-7.6 (m, 5H), 5.80 (m, 1H), 5.3 (br. s, 1H), 5.0 (br. s, 2H), 3.83-4.03 (m, 2H), 3.8 (s, 1H), 3.5 (m, 2H). |
| EX-139 | 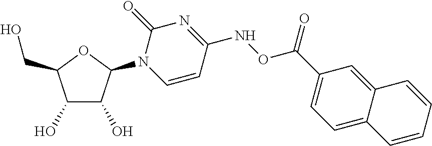 [SMILES: O=C(ONc1ccn([C@@H]2O[C@H](CO)[C@@H](O)[C@H]2O)c(=O)n1)c1ccc2ccccc2c1] | 1H NMR (400 MHz, DMSO) δ 11.1 (s, 1H), 8.9 (s, 1H), 8.0-8.2 (m, 4H), 7.5-7.7 (m, 3H), 5.8 (m, 2H), 5.3 (d, 1H), 5.1 (d, 2H), 4.1 (d, 2H), 3.9 (s, 1H), 3.6 (m, 2H). |
| EX-140 | 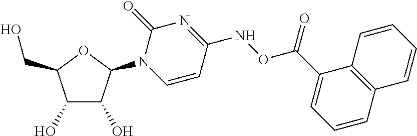 [SMILES: O=C(ONc1ccn([C@@H]2O[C@H](CO)[C@@H](O)[C@H]2O)c(=O)n1)c1cccc2ccccc12] | 1H NMR(400 MHz, DMSO) δ 11.1 (s, 1H), 8.7 (dd, 1H), 8.4 (d, 1H), 8.2 (dd, 1H), 8.0 (d, 1H), 7.5-7.7 (m, 4H), 5.9 (d, 1H), 5.8 (dd, 1H), 5.4 (d, 1H), 5.1 (dd, 2H), 4.0 (m, 1H), 3.9 (s, 1H), 3.8 (s, 1H), 3.5 (m, 2H) |
| EX-144 | 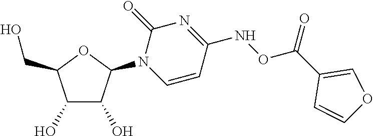 [SMILES: O=C(ONc1ccn([C@@H]2O[C@H](CO)[C@@H](O)[C@H]2O)c(=O)n1)c1ccoc1] | 1H NMR(400 MHz, DMSO) δ 10.98 (s, 1H), 8.82 (s, 1H), 7.83 (s, 1H), 7.55 (d, 1H), 7.02 (s, 1H), 5.76 (m, 2H), 5.34 (d, 1H), 5.08 (s, 2H), 3.96-4.11 (m, 3H), 3.83 (d, 1H), 3.55-3.60 (m, 2H), 3.17 (d, 1H). |
| EX-145 | 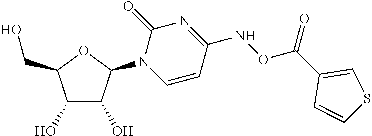 [SMILES: O=C(ONc1ccn([C@@H]2O[C@H](CO)[C@@H](O)[C@H]2O)c(=O)n1)c1ccsc1] | 1H NMR(400 MHz, DMSO) δ 11.02 (s, 1H), 8.86 (s, 1H), 7.46-7.71 (m, 3H), 8.84 (d, 2H), 5.35 (s, 1H), 5.08 (d, 1H), 5.34 (d, 1H), 5.08 (s, 2H), 9.95-4.02 (m, 2H), 3.83 (s, 1H), 3.54 (m, 2H) |

The following exemplary compounds can be prepared with the methods similar to the methods described in the above Examples using commercially available anhydrides or acyl chlorides. For those carboxylic anhydrides and acyl chlorides that are not commercially available, they can be easily prepared by well known standard procedures.

| Example # | Structure |
|---|---|
| EX-10 | 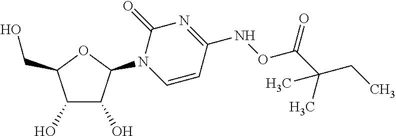 [SMILES: CCC(C)(C)C(=O)ONc1ccn([C@@H]2O[C@H](CO)[C@@H](O)[C@H]2O)c(=O)n1] |
| EX-14 | 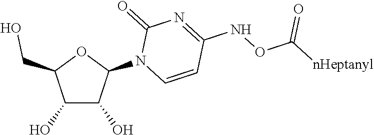 |
| EX-19 | 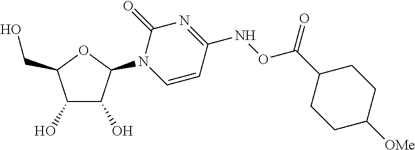 [SMILES: COC1CCC(C(=O)ONc2ccn([C@@H]3O[C@H](CO)[C@@H](O)[C@H]3O)c(=O)n2)CC1] |
| EX-20 | 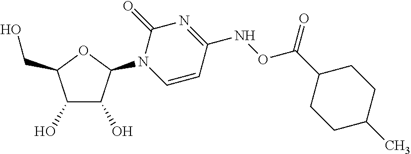 [SMILES: CC1CCC(C(=O)ONc2ccn([C@@H]3O[C@H](CO)[C@@H](O)[C@H]3O)c(=O)n2)CC1] |
| EX-21 | 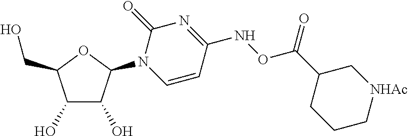 |
| EX-22 | 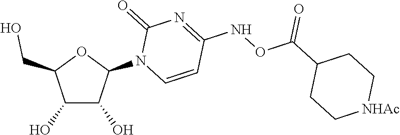 |
| EX-23 | 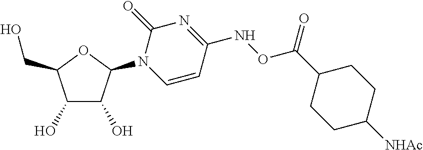 [SMILES: CC(=O)NC1CCC(C(=O)ONc2ccn([C@@H]3O[C@H](CO)[C@@H](O)[C@H]3O)c(=O)n2)CC1] |
| EX-28 | 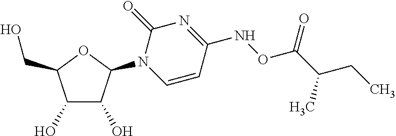 [SMILES: CC[C@H](C)C(=O)ONc1ccn([C@@H]2O[C@H](CO)[C@@H](O)[C@H]2O)c(=O)n1] |
| EX-29 | 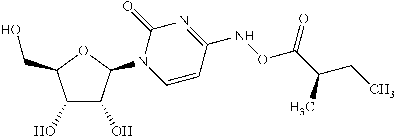 [SMILES: CC[C@@H](C)C(=O)ONc1ccn([C@@H]2O[C@H](CO)[C@@H](O)[C@H]2O)c(=O)n1] |
| EX-30 | 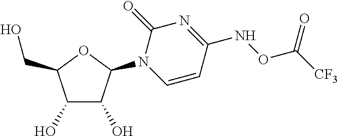 [SMILES: O=C(ONc1ccn([C@@H]2O[C@H](CO)[C@@H](O)[C@H]2O)c(=O)n1)C(F)(F)F] |
| EX-31 | 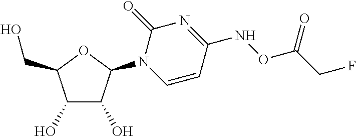 [SMILES: O=C(CF)ONc1ccn([C@@H]2O[C@H](CO)[C@@H](O)[C@H]2O)c(=O)n1] |
| EX-32 | 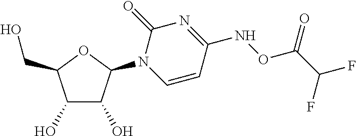 [SMILES: O=C(ONc1ccn([C@@H]2O[C@H](CO)[C@@H](O)[C@H]2O)c(=O)n1)C(F)F] |
| EX-33 | 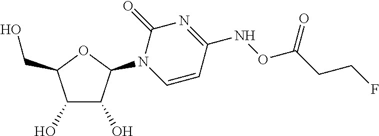 [SMILES: O=C(CCF)ONc1ccn([C@@H]2O[C@H](CO)[C@@H](O)[C@H]2O)c(=O)n1] |
| EX-34 | 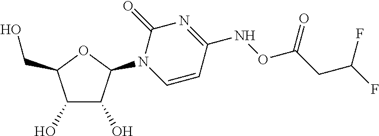 [SMILES: O=C(CC(F)F)ONc1ccn([C@@H]2O[C@H](CO)[C@@H](O)[C@H]2O)c(=O)n1] |
| EX-35 | 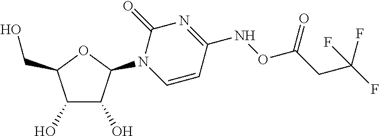 [SMILES: O=C(CC(F)(F)F)ONc1ccn([C@@H]2O[C@H](CO)[C@@H](O)[C@H]2O)c(=O)n1] |
| EX-36 | 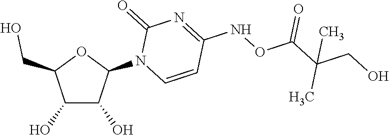 [SMILES: CC(C)(CO)C(=O)ONc1ccn([C@@H]2O[C@H](CO)[C@@H](O)[C@H]2O)c(=O)n1] |
| EX-37 | 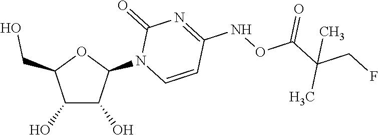 [SMILES: CC(C)(CF)C(=O)ONc1ccn([C@@H]2O[C@H](CO)[C@@H](O)[C@H]2O)c(=O)n1] |
| EX-38 | 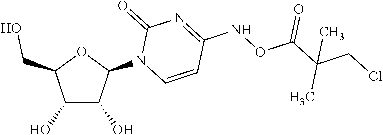 [SMILES: CC(C)(CCl)C(=O)ONc1ccn([C@@H]2O[C@H](CO)[C@@H](O)[C@H]2O)c(=O)n1] |
| EX-43 | 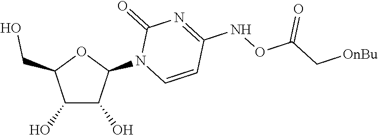 |
| EX-44 | 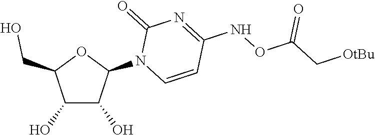 [SMILES: CC(C)(C)OCC(=O)ONc1ccn([C@@H]2O[C@H](CO)[C@@H](O)[C@H]2O)c(=O)n1] |
| EX-45 | 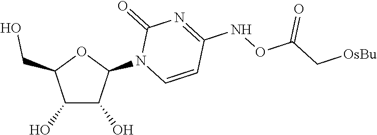 |
| EX-46 | 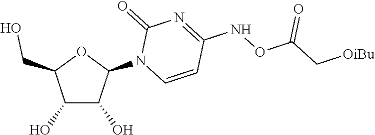 |
| EX-47 | 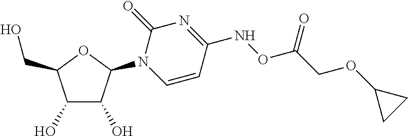 [SMILES: O=C(COC1CC1)ONc1ccn([C@@H]2O[C@H](CO)[C@@H](O)[C@H]2O)c(=O)n1] |
| EX-48 | 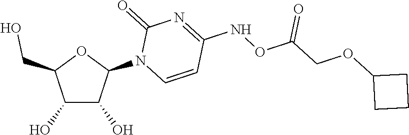 [SMILES: O=C(COC1CCC1)ONc1ccn([C@@H]2O[C@H](CO)[C@@H](O)[C@H]2O)c(=O)n1] |
| EX-49 | 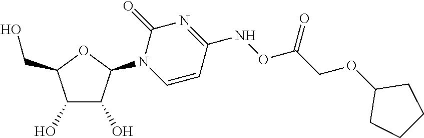 [SMILES: O=C(COC1CCCC1)ONc1ccn([C@@H]2O[C@H](CO)[C@@H](O)[C@H]2O)c(=O)n1] |
| EX-50 | 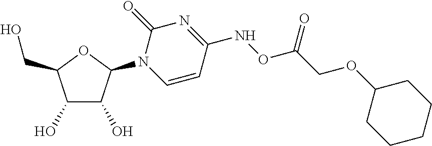 [SMILES: O=C(COC1CCCCC1)ONc1ccn([C@@H]2O[C@H](CO)[C@@H](O)[C@H]2O)c(=O)n1] |
| EX-59 | 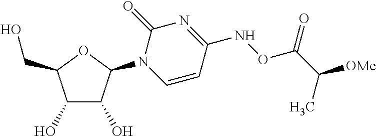 [SMILES: CO[C@@H](C)C(=O)ONc1ccn([C@@H]2O[C@H](CO)[C@@H](O)[C@H]2O)c(=O)n1] |
| EX-60 | 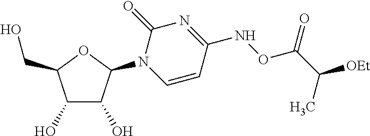 [SMILES: CCO[C@@H](C)C(=O)ONc1ccn([C@@H]2O[C@H](CO)[C@@H](O)[C@H]2O)c(=O)n1] |
| EX-61 | 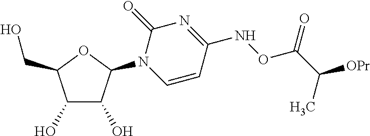 |
| EX-62 | 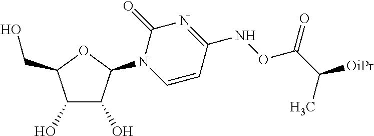 [SMILES: CC(C)O[C@@H](C)C(=O)ONc1ccn([C@@H]2O[C@H](CO)[C@@H](O)[C@H]2O)c(=O)n1] |
| EX-63 | 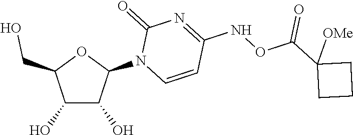 [SMILES: COC1(C(=O)ONc2ccn([C@@H]3O[C@H](CO)[C@@H](O)[C@H]3O)c(=O)n2)CCC1] |
| EX-64 | 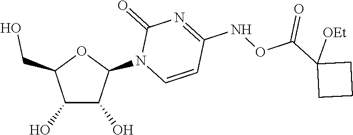 [SMILES: CCOC1(C(=O)ONc2ccn([C@@H]3O[C@H](CO)[C@@H](O)[C@H]3O)c(=O)n2)CCC1] |
| EX-65 | 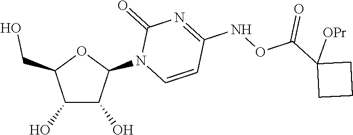 |
| EX-66 | 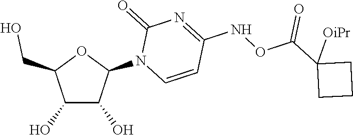 [SMILES: CC(C)OC1(C(=O)ONc2ccn([C@@H]3O[C@H](CO)[C@@H](O)[C@H]3O)c(=O)n2)CCC1] |
| EX-68 | 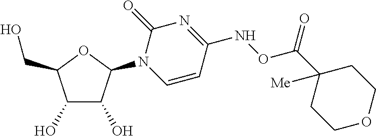 [SMILES: CC1(C(=O)ONc2ccn([C@@H]3O[C@H](CO)[C@@H](O)[C@H]3O)c(=O)n2)CCOCC1] |
| EX-69 | 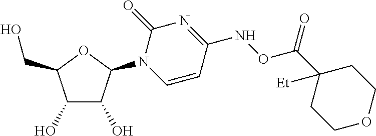 [SMILES: CCC1(C(=O)ONc2ccn([C@@H]3O[C@H](CO)[C@@H](O)[C@H]3O)c(=O)n2)CCOCC1] |
| EX-70 | 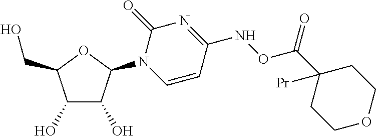 |
| EX-71 | 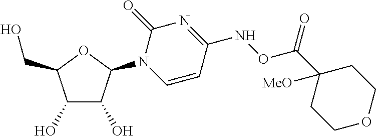 [SMILES: COC1(C(=O)ONc2ccn([C@@H]3O[C@H](CO)[C@@H](O)[C@H]3O)c(=O)n2)CCOCC1] |
| EX-72 | 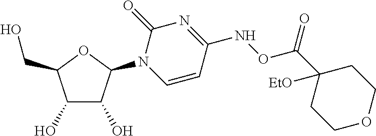 [SMILES: CCOC1(C(=O)ONc2ccn([C@@H]3O[C@H](CO)[C@@H](O)[C@H]3O)c(=O)n2)CCOCC1] |
| EX-73 | 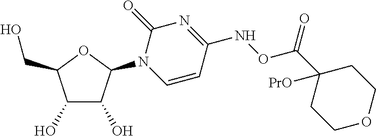 |
| EX-74 | 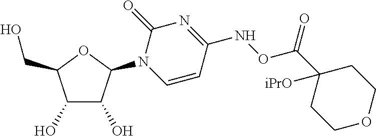 [SMILES: CC(C)OC1(C(=O)ONc2ccn([C@@H]3O[C@H](CO)[C@@H](O)[C@H]3O)c(=O)n2)CCOCC1] |
| EX-75 | 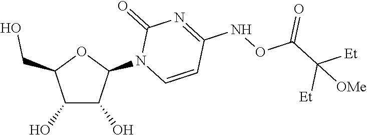 [SMILES: CCC(CC)(OC)C(=O)ONc1ccn([C@@H]2O[C@H](CO)[C@@H](O)[C@H]2O)c(=O)n1] |
| EX-76 | 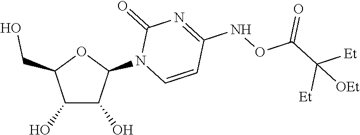 [SMILES: CCOC(CC)(CC)C(=O)ONc1ccn([C@@H]2O[C@H](CO)[C@@H](O)[C@H]2O)c(=O)n1] |
| EX-77 | 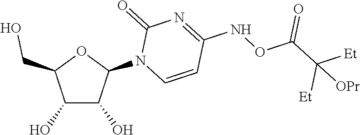 |
| EX-78 | 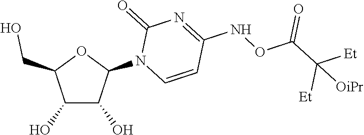 [SMILES: CCC(CC)(OC(C)C)C(=O)ONc1ccn([C@@H]2O[C@H](CO)[C@@H](O)[C@H]2O)c(=O)n1] |
| EX-80 | 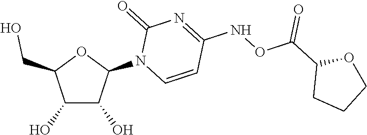 |
| EX-81 | 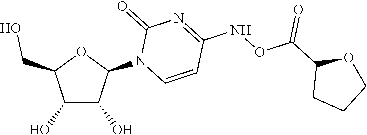 |
| EX-82 | 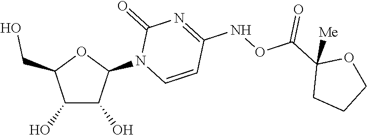 [SMILES: C[C@]1(C(=O)ONc2ccn([C@@H]3O[C@H](CO)[C@@H](O)[C@H]3O)c(=O)n2)CCCO1] |
| EX-83 | 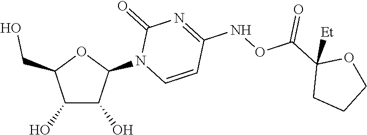 [SMILES: CC[C@]1(C(=O)ONc2ccn([C@@H]3O[C@H](CO)[C@@H](O)[C@H]3O)c(=O)n2)CCCO1] |
| EX-84 | 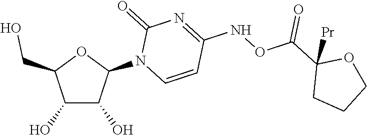 |
| EX-85 | 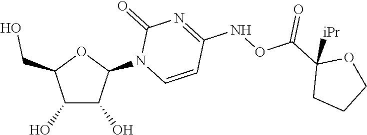 [SMILES: CC(C)[C@]1(C(=O)ONc2ccn([C@@H]3O[C@H](CO)[C@@H](O)[C@H]3O)c(=O)n2)CCCO1] |
| EX-86 | 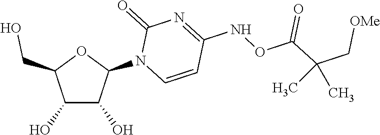 [SMILES: COCC(C)(C)C(=O)ONc1ccn([C@@H]2O[C@H](CO)[C@@H](O)[C@H]2O)c(=O)n1] |
| EX-87 | 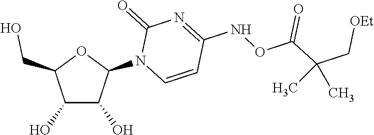 [SMILES: CCOCC(C)(C)C(=O)ONc1ccn([C@@H]2O[C@H](CO)[C@@H](O)[C@H]2O)c(=O)n1] |
| EX-88 | 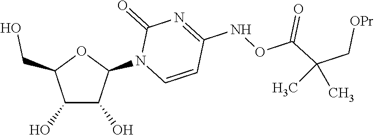 |
| EX-89 | 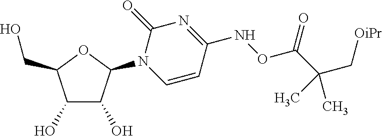 [SMILES: CC(C)OCC(C)(C)C(=O)ONc1ccn([C@@H]2O[C@H](CO)[C@@H](O)[C@H]2O)c(=O)n1] |
| EX-90 | 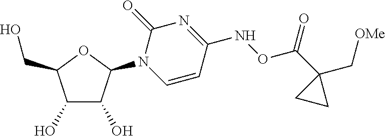 [SMILES: COCC1(C(=O)ONc2ccn([C@@H]3O[C@H](CO)[C@@H](O)[C@H]3O)c(=O)n2)CC1] |
| EX-91 | 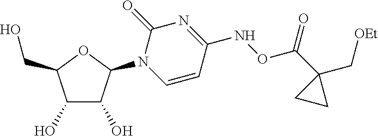 [SMILES: CCOCC1(C(=O)ONc2ccn([C@@H]3O[C@H](CO)[C@@H](O)[C@H]3O)c(=O)n2)CC1] |
| EX-92 | 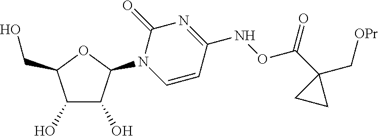 |
| EX-93 | 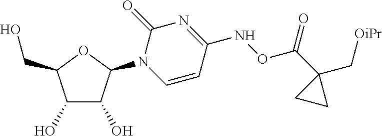 [SMILES: CC(C)OCC1(C(=O)ONc2ccn([C@@H]3O[C@H](CO)[C@@H](O)[C@H]3O)c(=O)n2)CC1] |
| EX-94 | 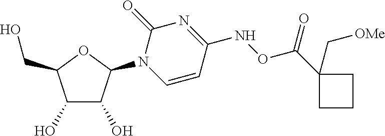 [SMILES: COCC1(C(=O)ONc2ccn([C@@H]3O[C@H](CO)[C@@H](O)[C@H]3O)c(=O)n2)CCC1] |
| EX-95 | 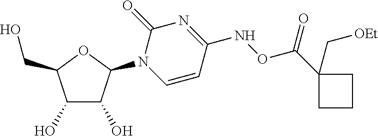 [SMILES: CCOCC1(C(=O)ONc2ccn([C@@H]3O[C@H](CO)[C@@H](O)[C@H]3O)c(=O)n2)CCC1] |
| EX-96 | 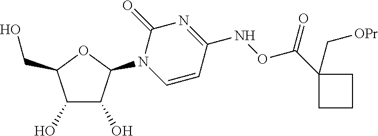 |
| EX-97 | 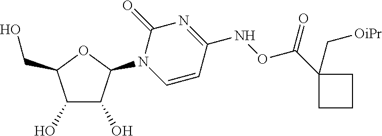 [SMILES: CC(C)OCC1(C(=O)ONc2ccn([C@@H]3O[C@H](CO)[C@@H](O)[C@H]3O)c(=O)n2)CCC1] |
| EX-98 | 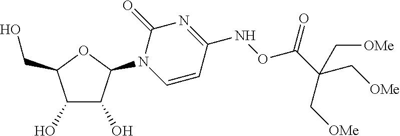 [SMILES: COCC(COC)(COC)C(=O)ONc1ccn([C@@H]2O[C@H](CO)[C@@H](O)[C@H]2O)c(=O)n1] |
| EX-99 | 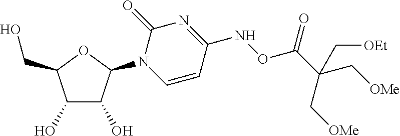 [SMILES: CCOCC(COC)(COC)C(=O)ONc1ccn([C@@H]2O[C@H](CO)[C@@H](O)[C@H]2O)c(=O)n1] |
| EX-100 | 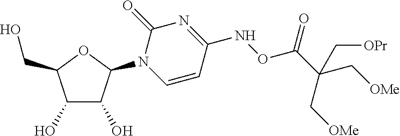 |
| EX-102 | 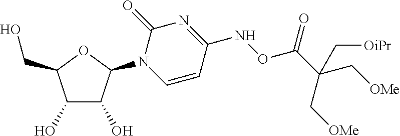 [SMILES: COCC(COC)(COC(C)C)C(=O)ONc1ccn([C@@H]2O[C@H](CO)[C@@H](O)[C@H]2O)c(=O)n1] |
| EX-103 | 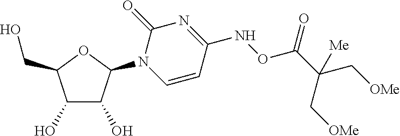 [SMILES: COCC(C)(COC)C(=O)ONc1ccn([C@@H]2O[C@H](CO)[C@@H](O)[C@H]2O)c(=O)n1] |
| EX-104 | 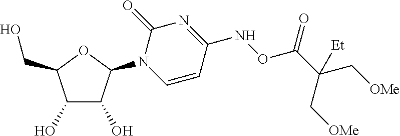 [SMILES: CCC(COC)(COC)C(=O)ONc1ccn([C@@H]2O[C@H](CO)[C@@H](O)[C@H]2O)c(=O)n1] |
| EX-105 | 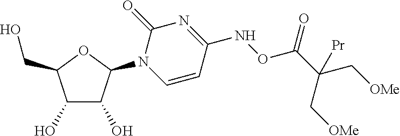 |
| EX-106 | 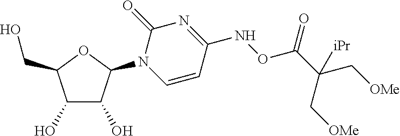 [SMILES: COCC(COC)(C(=O)ONc1ccn([C@@H]2O[C@H](CO)[C@@H](O)[C@H]2O)c(=O)n1)C(C)C] |
| EX-107 | 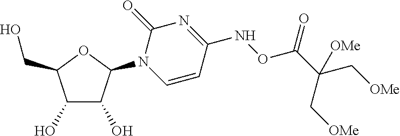 [SMILES: COCC(COC)(OC)C(=O)ONc1ccn([C@@H]2O[C@H](CO)[C@@H](O)[C@H]2O)c(=O)n1] |
| EX-108 | 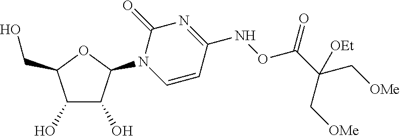 [SMILES: CCOC(COC)(COC)C(=O)ONc1ccn([C@@H]2O[C@H](CO)[C@@H](O)[C@H]2O)c(=O)n1] |
| EX-109 | 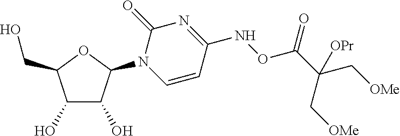 |
| EX-110 | 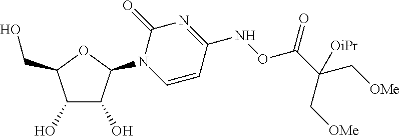 [SMILES: COCC(COC)(OC(C)C)C(=O)ONc1ccn([C@@H]2O[C@H](CO)[C@@H](O)[C@H]2O)c(=O)n1] |
| EX-111 | 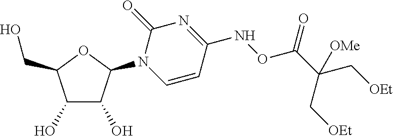 [SMILES: CCOCC(COCC)(OC)C(=O)ONc1ccn([C@@H]2O[C@H](CO)[C@@H](O)[C@H]2O)c(=O)n1] |
| EX-112 | 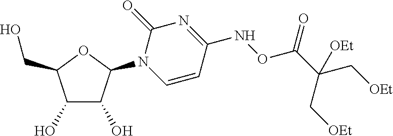 [SMILES: CCOCC(COCC)(OCC)C(=O)ONc1ccn([C@@H]2O[C@H](CO)[C@@H](O)[C@H]2O)c(=O)n1] |
| EX-113 | 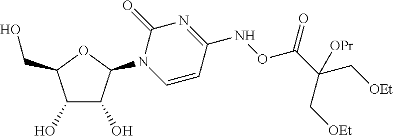 |
| EX-114 | 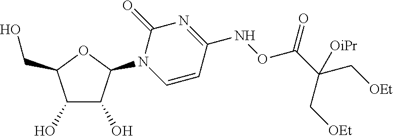 [SMILES: CCOCC(COCC)(OC(C)C)C(=O)ONc1ccn([C@@H]2O[C@H](CO)[C@@H](O)[C@H]2O)c(=O)n1] |
| EX-115 | 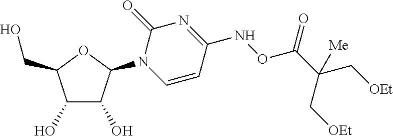 [SMILES: CCOCC(C)(COCC)C(=O)ONc1ccn([C@@H]2O[C@H](CO)[C@@H](O)[C@H]2O)c(=O)n1] |
| EX-116 | 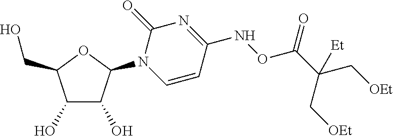 [SMILES: CCOCC(CC)(COCC)C(=O)ONc1ccn([C@@H]2O[C@H](CO)[C@@H](O)[C@H]2O)c(=O)n1] |
| EX-117 | 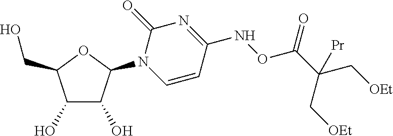 |
| EX-118 | 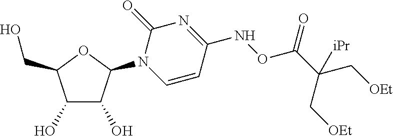 [SMILES: CCOCC(COCC)(C(=O)ONc1ccn([C@@H]2O[C@H](CO)[C@@H](O)[C@H]2O)c(=O)n1)C(C)C] |
| EX-119 | 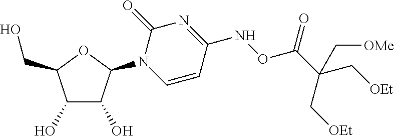 [SMILES: CCOCC(COC)(COCC)C(=O)ONc1ccn([C@@H]2O[C@H](CO)[C@@H](O)[C@H]2O)c(=O)n1] |
| EX-120 | 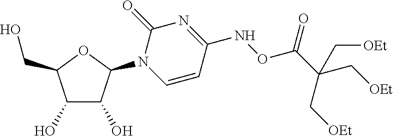 [SMILES: CCOCC(COCC)(COCC)C(=O)ONc1ccn([C@@H]2O[C@H](CO)[C@@H](O)[C@H]2O)c(=O)n1] |
| EX-121 | 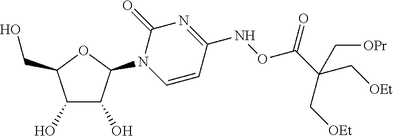 |
| EX-122 | 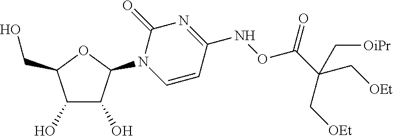 [SMILES: CCOCC(COCC)(COC(C)C)C(=O)ONc1ccn([C@@H]2O[C@H](CO)[C@@H](O)[C@H]2O)c(=O)n1] |
| EX-124 | 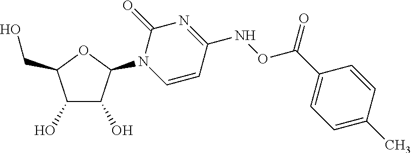 [SMILES: Cc1ccc(C(=O)ONc2ccn([C@@H]3O[C@H](CO)[C@@H](O)[C@H]3O)c(=O)n2)cc1] |
| EX-125 | 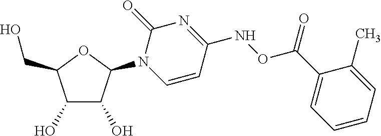 [SMILES: Cc1ccccc1C(=O)ONc1ccn([C@@H]2O[C@H](CO)[C@@H](O)[C@H]2O)c(=O)n1] |
| EX-126 | 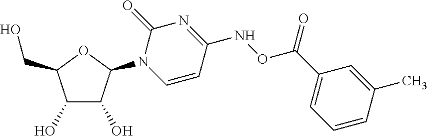 [SMILES: Cc1cccc(C(=O)ONc2ccn([C@@H]3O[C@H](CO)[C@@H](O)[C@H]3O)c(=O)n2)c1] |
| EX-127 | 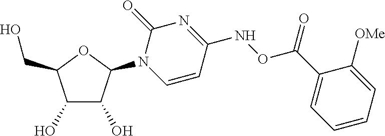 [SMILES: COc1ccccc1C(=O)ONc1ccn([C@@H]2O[C@H](CO)[C@@H](O)[C@H]2O)c(=O)n1] |
| EX-128 | 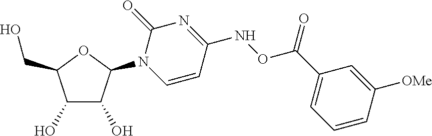 [SMILES: COc1cccc(C(=O)ONc2ccn([C@@H]3O[C@H](CO)[C@@H](O)[C@H]3O)c(=O)n2)c1] |
| EX-129 | 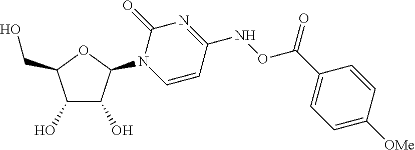 [SMILES: COc1ccc(C(=O)ONc2ccn([C@@H]3O[C@H](CO)[C@@H](O)[C@H]3O)c(=O)n2)cc1] |
| EX-130 | 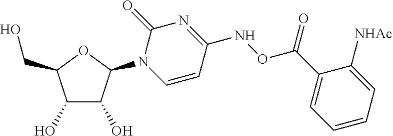 [SMILES: CC(=O)Nc1ccccc1C(=O)ONc1ccn([C@@H]2O[C@H](CO)[C@@H](O)[C@H]2O)c(=O)n1] |
| EX-131 | 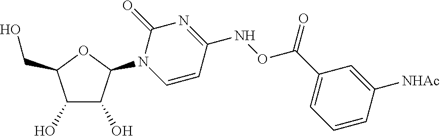 [SMILES: CC(=O)Nc1cccc(C(=O)ONc2ccn([C@@H]3O[C@H](CO)[C@@H](O)[C@H]3O)c(=O)n2)c1] |
| EX-132 | 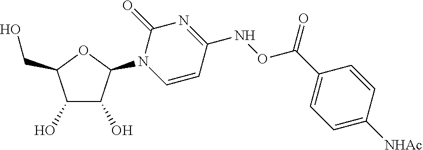 [SMILES: CC(=O)Nc1ccc(C(=O)ONc2ccn([C@@H]3O[C@H](CO)[C@@H](O)[C@H]3O)c(=O)n2)cc1] |
| EX-133 | 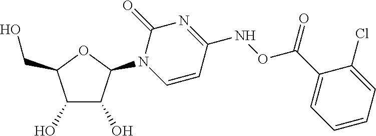 [SMILES: O=C(ONc1ccn([C@@H]2O[C@H](CO)[C@@H](O)[C@H]2O)c(=O)n1)c1ccccc1Cl] |
| EX-134 | 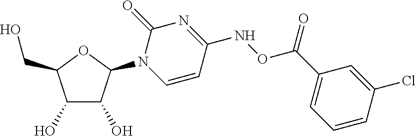 [SMILES: O=C(ONc1ccn([C@@H]2O[C@H](CO)[C@@H](O)[C@H]2O)c(=O)n1)c1cccc(Cl)c1] |
| EX-135 | 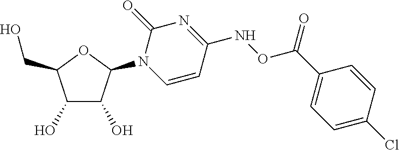 [SMILES: O=C(ONc1ccn([C@@H]2O[C@H](CO)[C@@H](O)[C@H]2O)c(=O)n1)c1ccc(Cl)cc1] |
| EX-136 | 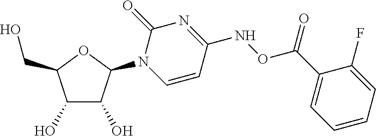 [SMILES: O=C(ONc1ccn([C@@H]2O[C@H](CO)[C@@H](O)[C@H]2O)c(=O)n1)c1ccccc1F] |
| EX-137 | 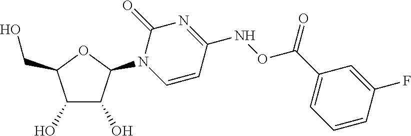 [SMILES: O=C(ONc1ccn([C@@H]2O[C@H](CO)[C@@H](O)[C@H]2O)c(=O)n1)c1cccc(F)c1] |
| EX-138 | 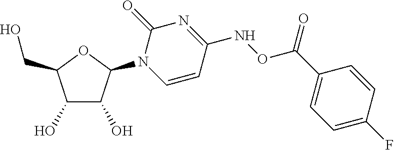 [SMILES: O=C(ONc1ccn([C@@H]2O[C@H](CO)[C@@H](O)[C@H]2O)c(=O)n1)c1ccc(F)cc1] |
| EX-141 | 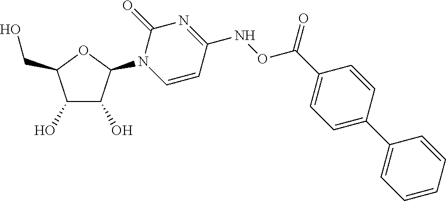 [SMILES: O=C(ONc1ccn([C@@H]2O[C@H](CO)[C@@H](O)[C@H]2O)c(=O)n1)c1ccc(-c2ccccc2)cc1] |
| EX-142 | 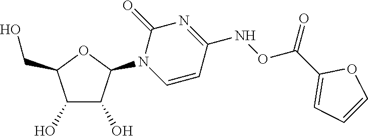 [SMILES: O=C(ONc1ccn([C@@H]2O[C@H](CO)[C@@H](O)[C@H]2O)c(=O)n1)c1ccco1] |
| EX-143 | 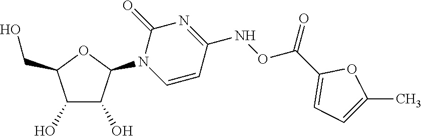 [SMILES: Cc1ccc(C(=O)ONc2ccn([C@@H]3O[C@H](CO)[C@@H](O)[C@H]3O)c(=O)n2)o1] |
| EX-146 | 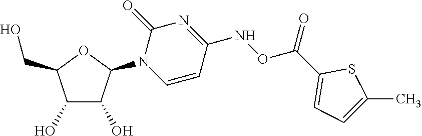 [SMILES: Cc1ccc(C(=O)ONc2ccn([C@@H]3O[C@H](CO)[C@@H](O)[C@H]3O)c(=O)n2)s1] |
| EX-147 | 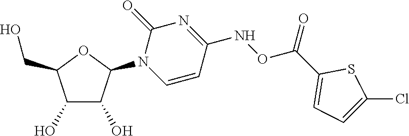 [SMILES: O=C(ONc1ccn([C@@H]2O[C@H](CO)[C@@H](O)[C@H]2O)c(=O)n1)c1ccc(Cl)s1] |
| EX-148 | 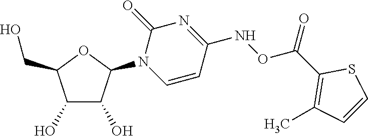 [SMILES: Cc1ccsc1C(=O)ONc1ccn([C@@H]2O[C@H](CO)[C@@H](O)[C@H]2O)c(=O)n1] |
| EX-149 | 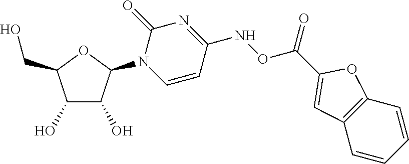 [SMILES: O=C(ONc1ccn([C@@H]2O[C@H](CO)[C@@H](O)[C@H]2O)c(=O)n1)c1cc2ccccc2o1] |
| EX-150 | 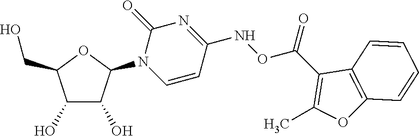 [SMILES: Cc1oc2ccccc2c1C(=O)ONc1ccn([C@@H]2O[C@H](CO)[C@@H](O)[C@H]2O)c(=O)n1] |
| EX-151 | 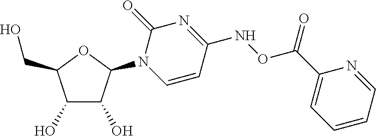 [SMILES: O=C(ONc1ccn([C@@H]2O[C@H](CO)[C@@H](O)[C@H]2O)c(=O)n1)c1ccccn1] |
| EX-152 | 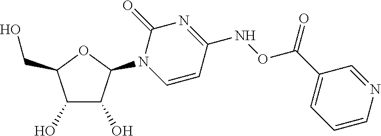 [SMILES: O=C(ONc1ccn([C@@H]2O[C@H](CO)[C@@H](O)[C@H]2O)c(=O)n1)c1cccnc1] |
| EX-153 | 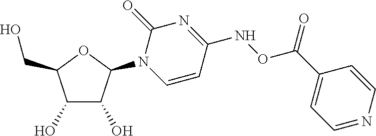 [SMILES: O=C(ONc1ccn([C@@H]2O[C@H](CO)[C@@H](O)[C@H]2O)c(=O)n1)c1ccncc1] |
| EX-154 | 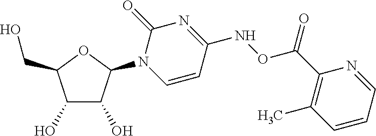 [SMILES: Cc1cccnc1C(=O)ONc1ccn([C@@H]2O[C@H](CO)[C@@H](O)[C@H]2O)c(=O)n1] |
| EX-155 | 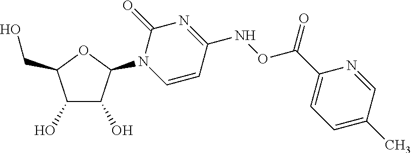 [SMILES: Cc1ccc(C(=O)ONc2ccn([C@@H]3O[C@H](CO)[C@@H](O)[C@H]3O)c(=O)n2)nc1] |
| EX-156 | 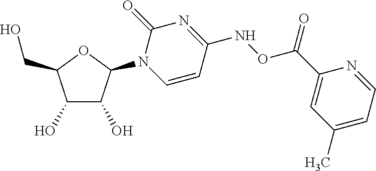 [SMILES: Cc1ccnc(C(=O)ONc2ccn([C@@H]3O[C@H](CO)[C@@H](O)[C@H]3O)c(=O)n2)c1] |
| EX-157 | 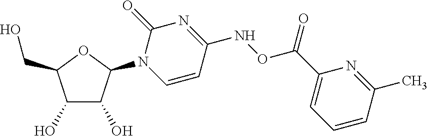 [SMILES: Cc1cccc(C(=O)ONc2ccn([C@@H]3O[C@H](CO)[C@@H](O)[C@H]3O)c(=O)n2)n1] |
| EX-158 | 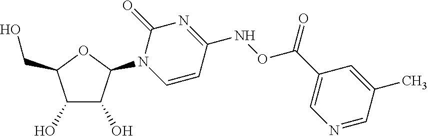 [SMILES: Cc1cncc(C(=O)ONc2ccn([C@@H]3O[C@H](CO)[C@@H](O)[C@H]3O)c(=O)n2)c1] |
| EX-159 | 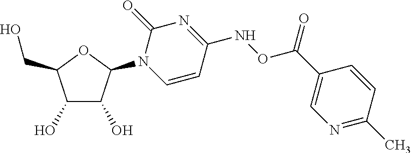 [SMILES: Cc1ccc(C(=O)ONc2ccn([C@@H]3O[C@H](CO)[C@@H](O)[C@H]3O)c(=O)n2)cn1] |
| EX-160 | 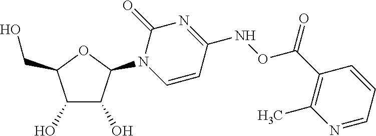 [SMILES: Cc1ncccc1C(=O)ONc1ccn([C@@H]2O[C@H](CO)[C@@H](O)[C@H]2O)c(=O)n1] |
| EX-161 | 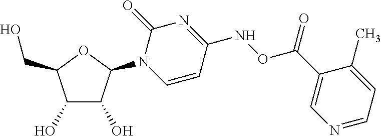 [SMILES: Cc1ccncc1C(=O)ONc1ccn([C@@H]2O[C@H](CO)[C@@H](O)[C@H]2O)c(=O)n1] |
| EX-162 | 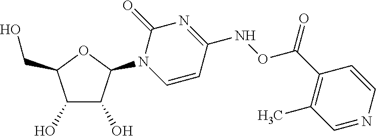 [SMILES: Cc1cnccc1C(=O)ONc1ccn([C@@H]2O[C@H](CO)[C@@H](O)[C@H]2O)c(=O)n1] |
| EX-163 | 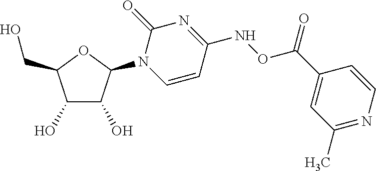 [SMILES: Cc1cc(C(=O)ONc2ccn([C@@H]3O[C@H](CO)[C@@H](O)[C@H]3O)c(=O)n2)ccn1] |
| EX-164 | 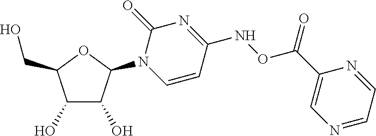 [SMILES: O=C(ONc1ccn([C@@H]2O[C@H](CO)[C@@H](O)[C@H]2O)c(=O)n1)c1cnccn1] |
| EX-165 | 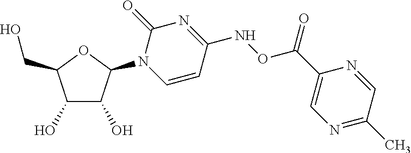 [SMILES: Cc1cnc(C(=O)ONc2ccn([C@@H]3O[C@H](CO)[C@@H](O)[C@H]3O)c(=O)n2)cn1] |
| EX-166 | 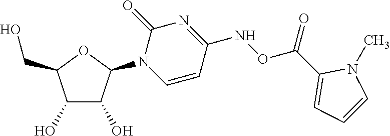 [SMILES: Cn1cccc1C(=O)ONc1ccn([C@@H]2O[C@H](CO)[C@@H](O)[C@H]2O)c(=O)n1] |
| EX-167 | 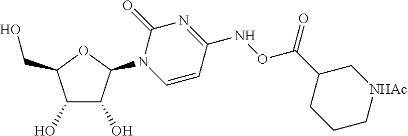 |
| EX-168 | 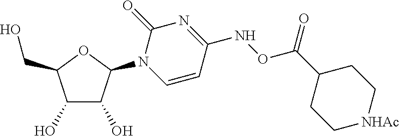 |
| EX-169 | 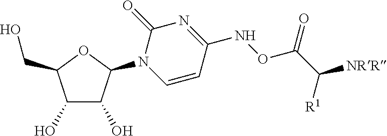 |

### Example 170: Plasma Stability

T½ (min)

| Compound | Mouse | Rat | Dog | Monkey | Human |
|---|---|---|---|---|---|
| EX-2 | 7.4 | 87.8 | 79.1 | 29.3 | 11.6 |
| EX-3 | 0.6 | 25.9 | 21.2 | 3.2 | 2.8 |
| EX-15 | 5.2 | 88.9 | 109.4 | 16.5 | 5.8 |
| EX-53 | 3.1 | 94.3 | 156.4 | 9.8 | 4.8 |
| Molnupiravir | 25.8 | 3.3 | 39.4 | 25.8 | 66.2 |

Preparation of Solutions: Stock solution (10 mM) of each of test compounds was prepared in DMSO. The stock solution for each compound was then diluted into 100 μM with acetonitrile.

Plasma Incubations: Plasma incubations were conducted in duplicate in 96-well plate at 37° C. Plasma was prewarmed in a total volume of 198 μL for 5 min at 37° C., then added 2 μL of 100 μM test compound into an incubation well containing plasma, pipette-mixed to achieve a homogenous suspension and immediately transferred 20 μL incubate as a 0 min sample to wells in a “Quenching” plate followed by adding 200 μL of acetonitrile with metolazone as internal standard (IS) and pipette-mixing. At 2, 5, 60, and 90 min, pipette-mixed the incubate and serially transfer samples of 20 μL incubate per time point to wells in a separate “Quenching” plate followed by adding 200 μL of acetonitrile with metolazone as IS and pipette-mixing.

Sample Analysis: The 96-well plate was centrifuged at 6000 g for 10 min. The supernatant was injected onto the LC-MS/MS system for analysis.

### Example 171: Microsome Stability

T_{1/2} (min)

| Compound | Mouse | Rat | Dog | Monkey | Human |
|---|---|---|---|---|---|
| EX-2 | 16.2 | 287.8 | 76.3 | 50.8 | 33.5 |
| EX-3 | 6.8 | 46.6 | 33.8 | 4.4 | 13.5 |
| EX-15 | 15.4 | ∞ | 132.1 | 25.0 | 67.3 |
| EX-53 | 17.1 | 70.5 | 87.5 | 7.3 | 86.6 |
| Molnupiravir | 5.6 | 13.8 | 3.9 | 4.7 | 71.3 |

Preparation of Solutions: Stock solution (10 mM) of each of test compounds was prepared in DMSO. The stock solution for each compound was then diluted into 100 μM with acetonitrile.

Microsome Incubations: Incubation mixtures were prepared in a total volume of 200 μL with final component concentrations as follows: 0.1M PBS (pH 7.4), NADPH (2 mM) and liver microsomes (0.2 mg/mL) as well as test compound (1 μM) or Molnupiravir (1 μM) as a positive control, wherein the NADPH was added after a 5-min preincubation of all other components at 37° C. Pipette-mixed to achieve a homogenous suspension and immediately transferred 20 μL incubate as a 0 min sample to wells in a “Quenching” plate followed by adding 200 μL of acetonitrile with metolazone as IS and pipette-mixing. At 2, 5, 10, and 45 min, pipette-mixed the incubate and serially transfer samples of 20 μL incubate per time point to wells in a separate “Quenching” plate followed by adding 200 μL of acetonitrile with metolazone as IS and pipette-mixing.

Sample Analysis: The 96-well plate was centrifuged at 6000 g for 10 min. The supernatant was injected onto the LC-MS/MS system for analysis.

### Example 172: The In Vitro Activity of Example Compound EX-2 (CH2101) Vs. Molnupiravir Against SARS-CoV-2 Omicron B.1.1.529 Variant

This experiment was intended to study the antiviral activity of the compound of the present invention against SARS-CoV-2 Omicron B.1.1.529 variant in VERO E6 cells.

TABLE 1
Experimental methods

| | | Drug treatment time (Day)/ | Positive |
|---|---|---|---|
| Virus | Cell | Termination method | control |
| SARS-CoV-2 omicron | Vero | 4/CPE | Molnupiravir |
| B.1.1.529 | E6 | | |

Vero E6 cells was transferred and inoculated into a 96-well plate with a densicity of 10,000 cells per well. The plate was then incubated with 5% CO₂ at 37° C. overnight. Diluted test compound EX-2 (CH2101) and Molnupiravir (3-fold serial dilution, 8 concentrations with triplicates) and virus (MOI=0.1) were added, along with parallel blank control (Vero E6 cells without virus or test compound) and virus control (Vero E6 cells with virus but without test compound). The 96-well plate was incubated under 5% CO₂ at 37° C. for 4 days. The cell vitality was measured with Celltiter Glo for each cell. The cell vitality was used to calculate the anti viral activities of test compounds. If the cell activity is higher in the test compounds treated wells than those in the wells with virus treatment but no test compounds treatment, i.e. a weeker CPE, it means the test compounds have inhibitory activity against the virus.

EC₅₀ and CC₅₀ values of test compounds were calculated by using GraphPad Prism (version 8) with the method of log(inhibitor) vs. response—Variable slope. The equation for calculation of EC₉₀ value is: EC₉₀=EC₅₀×9\hat{ }(1/slope).

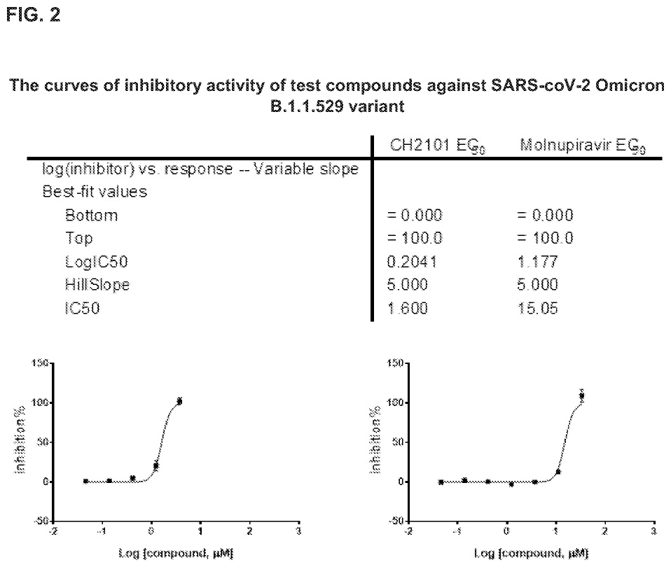

The experimental results were shown in Table 2 and FIG. 2.

TABLE 2
The activity of test compounds against
SARS-CoV-2 Omicron B.1.1.529 variant

| Test compound | EC₅₀ (μM) | EC₉₀ (μM) | CC₅₀ (μM) | SI (CC₅₀/EC₅₀) |
|---|---|---|---|---|
| EX-2/CH2101 | 1.60 | 2.483 | 18.20 | 11.38 |
| Molnupiravir | 15.05 | 23.355 | >100 | >6.65 |

### Example 173: In Vivo Efficacy Evaluation of Test Compounds CH2101 (Compound EX-2) Against SARS-CoV-2 in the K18-hACE2 Transgenic Mouse Infection Model

The study was to evaluate in vivo efficacy of test compound CH2101 (EX-2) against SARS-CoV-2 in the K18-hACE2 transgenic mouse infection model, with the primary endpoint readouts of body weight changes, clinical symptom scores, death and lung viral titers.

1.1 Male and specific pathogen free K18 hACE2 transgenic mice (B6.Cg-Tg(K18-ACE2)2Primn/J mice) were purchased from the Jackson Laboratories. The mice were housed and cared following the IACUC approved protocol #20003. Qualified mice were used for the study after an acclimation of no less than 3 days.

1.2 Virus: SARS-CoV-2, Hongkong strain was sourced from BEI Resources (NR-52282) and amplified in house.

1.3 Cells: Vero E6 was purchased from ATCC and propagated in house.

Vehicle for EX-2/CH2101 is 10% (v/v) PEG400+0.5% (w/v) CMC in purified water. The compound EX-2/CH2101 is formulated in the vehicle mentioned above at the desired concentration of 45 mg/mL, with purity taken into consideration

Vehicle for Remdesivir is 5% DMSO+10% Solutol+85% Saline (0.9% sodium chloride) at the desired concentration of 2.5 mg/mL, with purity taken into consideration.

Animal Grouping: The qualified mice were evenly grouped randomly into 6 groups according to the study designs.

Virus inoculation: The mice were anesthetized and inoculated with SARS-CoV-2 virus on day 0 via intranasal route at the amount of 5,000 p.f.u./mouse/50 μL.

Compound/vehicle administration: Mice were treated with vehicle, or remdesivir or test compound EX-2/CH2101 from day 0 to day 6 or day 4 (groups 4-6), by PO or SC routes, twice daily with interval of 8-16 hrs apart. 1^{st} dose was given at 2 hrs post-infection. See details in Table 3.

Animal monitoring: Body weight and mortality were monitored daily during the whole study. Clinical symptoms were observed and 1 point was given if any of the positive sign appears: ruffled fur, hunched, lethargic/low mobility and respiratory distress.

Samples harvesting: Terminal samples harvesting: mice from group 4-6 were sacrificed at day 5, lung samples were collected in a container with 1 mL EMEM. Weigh the vial prior to and after the lung sample collection to calculate the net lung weight. Lung samples were frozen for viral titration by plaque assay.

Termination of the in vivo study: Day 14 was the scheduled terminal day, all surviving animals were sacrificed.

Humane endpoint: Any mouse suffered ≥20% or/and a score of 3, was euthanized and counted as mortality.

Design and schedule: A detailed study design of in vivo part is listed in Table 3.

TABLE 3
Study design and compound administration regimen

| | | | Treatment | | | | | | | |
|---|---|---|---|---|---|---|---|---|---|---|
| Group | Number | Virus | Test | Dosage | volume | | | | 1^{st} dose | observation |
| # | of mice | inoculation | compound | (mg/kg) | (mL/kg) | route | frequency | Time | time | and readout |
| 1 | 5 | Virus: | Vehicle | 0 | 10 | PO | Twice | Days | 2 hrs | 1. Body |
| | | SARS-CoV- | | | | | daily | 0-6 | post- | weight |
| 2 | 5 | 2 Hong | Remdesivir | 25 | 10 | SC | Twice | | infection | changes, |
| | | Kong | | | | | daily | | | survival rate |
| 3 | 5 | Isolate | CH2101 | 450 | 10 | PO | Twice | | | and clinical |
| | | Inoculation: | | | | | daily | | | symptom |
| 4 | 5 | Via | Vehicle | 0 | 10 | PO | Twice | Days | 2 hrs | scores |
| | | intranasal | | | | | daily | 0-4 | post- | 2. Lung virus |
| 5 | 5 | Dosage; | Remdesivir | 25 | 10 | SC | Twice | | infection | titers (groups |
| | | 5000 p.f.u. | | | | | daily | | | 4-6, day 5) |
| 6 | 5 | Volumne: | CH2101 | 450 | 10 | PO | Twice | | | |
| | | 50 μL | | | | | daily | | | |

Viral titration: The lung viral titer was determined by a plaque assay. The plaque assay was performed with the VERO E6 cells. Homogenize the lung samples with tissue lyser and then 10-fold serially dilute the supernatant of lung homogenate, and 0.2 mL was pipetted to the pre-seeded 6 well plate. After 3 days' incubation, the cells were fixed with 4% paraformaldehyde, and stained with crystal violet solution. The plaques were visually counted and the virus titer was calculated with the following formula: virus titer/g lung tissue = Log 10 (plaques/well/0.2 * dilution factor/lung weight * 1000).

Result: The in vivo efficacy of the test compounds were determined by the body weight changes, clinical symptom scores, survival rates and the lung viral titers.

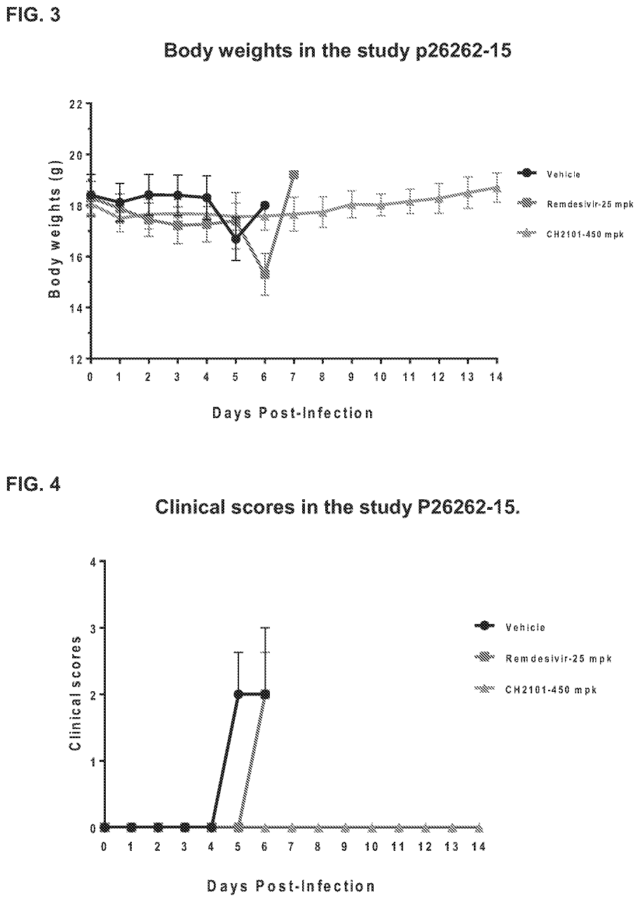

Protection to mice body weights: During the entire period of the in vivo study, the mice were monitored daily for body weights and body weight changes, which were normalized by the body weight at day 0, which are described as below and plotted in FIGS. 3 and 4.

Infection control group (Vehicle): Body weight loss was observed from day 5, and followed by continuous loss without sign of recovery, which was accompanied by death or euthanasia.

Remdesivir-25 mpk group: Body weight loss was observed from day 2, and followed by continuous loss and dropped by −13.6% on day 6, accompanied by death or euthanasia.

Test compound group (EX-2/CH2101-450 mpk) group: All mice maintained stable body weights, without notable body weight loss observed.

Clinical symptoms observation: During the entire period of the in vivo study, mice were monitored daily for clinical symptoms. Any positive sign of ruffled fur, hunched, lethargic and respiratory distress was scored as 1 point. The total score is described as below and plotted in FIG. 4.

Infection control group (Vehicle): Mice started to appear to show gross clinical symptoms from day 5, progressed rapidly and peaked at day 6, with a max clinical symptom score of 2 (mean value, similarly hereinafter).

Remdesivir group (25 mpk): Mice started to appear gross clinical symptoms from day 6, with a max clinical symptom scores of 2.

Test compound (CH2101-450 mpk) group: Mice were in good health condition, no infection-related clinical symptom was observed.

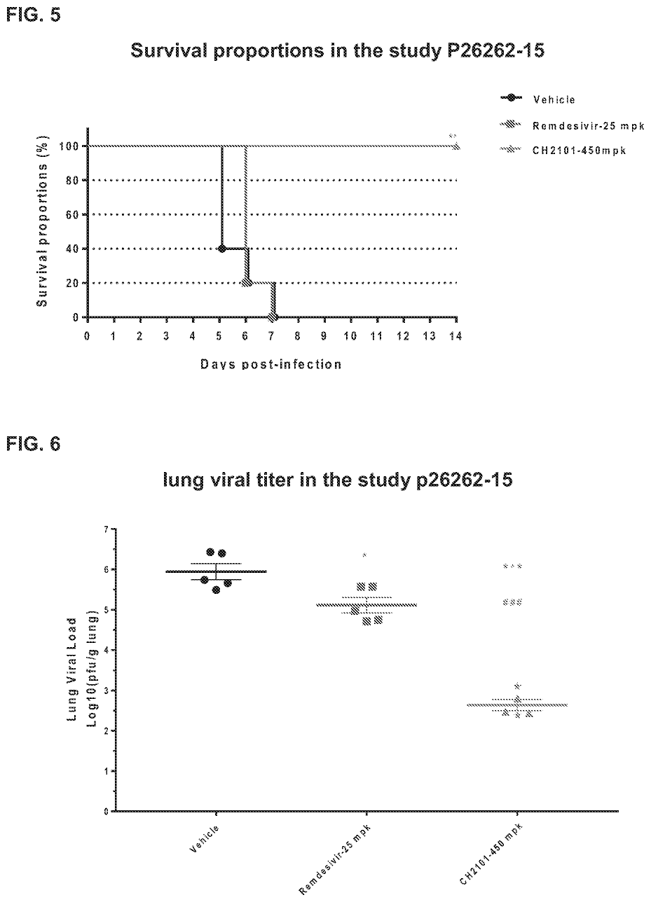

Survival rates: During the entire period of the in vivo study, mice were monitored daily for mortality. The surviving status of mice was described as below, plotted in FIG. 5 and summarized in Table 4.

Infection control group (Vehicle): Mortality was observed during days 5 and 7. Survival proportion was 0% and the median survival time was 5 days.

Remdesivir group (25 mpk): Mortality was observed during days 6 and 7. Survival proportion was 0% and the median survival time was 6 days.

Test compound (CH2101-450 mpk) group: No mortality was observed throughout the whole study, all mice survived to the end of the study, i.e., 100% survival rate.

TABLE 4
Surviving status

| | | | Survival | Median | Prolonged | |
|---|---|---|---|---|---|---|
| | Treatment | | pro- | survival | survival | |
| | Com- | Dosage | portions | time | time* | Log rank |
| Group | pound | (mg/kg) | (%) | (day) | (%) | P value* |
| 1 | Vehicle | 0 | 0% | 5 | Undefined | NA |
| 2 | Remdesivir | 25 | 0% | 6 | 20% | 0.2649 |
| 3 | CH2101 | 450 | 100% | Undefined | Undefined | 0.0015 |

*Compared with vehicle group (group 1)

Lung viral titers: For groups 4-6, lung samples were harvested at day 5 and the lung viral titer was determined by a plaque assay. The results are described as follows, plotted in FIG. 6 and summarized in Table 5.

Infection control group (Vehicle): Mean virus titer was 5.94 Log 10 (Plaques/g lung, similarly hereinafter), which met the study design and inclusion criteria, and was consistent with the historical data.

Remdesivir group (25 mpk): Mean virus titer was 5.11 Log, which was 0.83 Log lower than that of the vehicle group, with significant difference (p>0.05), indicating good antiviral efficacy in vivo.

Test compound group (CH2101-450 mpk): Mean virus titer was 2.64 Log, which was 3.31 Log lower than that of the vehicle group, and the difference was statistically significant (p<0.01), of which, 2 samples reached the lower limit of detection, which indicated optimal antiviral efficacy.

FIG. 6 shows lung viral titer in the study p26262-15. Mice were treated as indicated and sacrificed at day 5 for lung viral titration by plaque assay. The data was shown as Mean t SEM and analyzed by T test: *, p<0.05; ***, p<0.001, compared with vehicle group; and ###, p<0.001, compared with remdesivir group. Data formatted with star was below or equals to LLOD.

TABLE 5
Lung viral titer summary

| | | Virus titer | Comparison with | | Comparison | |
|---|---|---|---|---|---|---|
| | | in lung | Vehicle group | | with | |
| | | (Log 10 | Mean | | Remdesivir | |
| | | plaque#/g | difference | P value | group | |
| Group | Treatment | lung) | vs. group 1 | (T test ) | NA | NA |
| 4 | Vehicle | 5.94 | NA | NA | NA | NA |
| | 0 mpk | | | | | |
| 5 | Remdesivir | 5.11 | 0.83 | *, | 2.47 | ###, |
| | 25 mpk | | | p = 0.0165 | | p < |
| | | | | | | 0.001 |
| 6 | CH2101 | 2.64 | 3.31 | ***, | NA | NA |
| | 450 mpk | | | p < 0.001 | | |

### Conclusions

Data showed that the mice in the vehicle group appeared to have the designed infection symptoms, suffered from body weight loss and finally died from the infection. Furthermore, virus titer in lung samples of vehicle group was 5.94 Log, and remdesivir significantly reduced lung virus titers by 0.83 Log. All of these results demonstrated the successful establishment of SARS-COV-2 mouse infection model and providing of a platform for efficacy evaluation of test compounds in vivo.

With significant inhibition of virus replication in lungs and the protection for the infected mice on body weight loss and clinical symptoms, and increase of survival rate, the test compound EX-2 (CH2101) showed optimal antiviral efficacy in vivo in the current model, under the set conditions.

Example 174: Pharmacokinetic Study of EX-2 (CH2101), Molnupiravir (CH2017) and its Metabolite NHC (CH2018) Following a Single PO Administration of EX-2 (CH2101) or Molnupiravir (CH2017) in Male and Female Beagle Dogs

Both test compounds were prepared in 1% methyl cellulose (400 cps, SIGMA, SLCF9694) solution in purified water.

A total of 6 male+6 female Beagle Dogs, approximately 7-13 kg of bodyweight, were originally purchased from Marshall Biotechnology Co., Ltd. The animals were fasted overnight prior to dosing and food was re-provided after 4 hours post dosing.

Animal Assignment to Study

| | Dose | | | | | |
|---|---|---|---|---|---|---|
| | route- | | | | | |
| | com- | Blood | | | | |
| | pound | sam- | | | | |
| | name: | pling | | | | |
| | Dose | Time | Male | | Female | |
| Group | level | (hr) | Animal No. (Animal ID) | | | |
| 1 | PO, | 0.083 | 2001 | 2002 | 2501 | 2502 |
| | EX-2/ | 0.25 | (8373266) | (8386180) | (8616983) | (8356338) |
| | CH2101: | 0.5 | | | | |
| | 10 mg/ | 1 | | | | |
| | kg | 2 | | | | |
| | | 4 | | | | |
| | | 6 | | | | |
| | | 8 | | | | |
| | | 24 | | | | |
| 2 | PO | 0.083 | 3001 | 3002 | 3501 | 3502 |
| | EX-2/ | 0.25 | (8528596) | (8386635) | (8355705) | (8352064) |
| | CH2101: | 0.5 | | | | |
| | 20 mg/ | 1 | | | | |
| | kg | 2 | | | | |
| | | 4 | | | | |
| | | 6 | | | | |
| | | 8 | | | | |
| | | 24 | | | | |
| 3 | PO | 0.083 | 4001 | 4002 | 4501 | 4502 |
| | CH2107 | 0.25 | (8529712) | (8529479) | (8354491) | (8351335) |
| | (Molnu- | 0.5 | | | | |
| | piravir): | 1 | | | | |
| | 22 mg/ | 2 | | | | |
| | kg | 4 | | | | |
| | | 6 | | | | |
| | | 8 | | | | |
| | | 24 | | | | |

The animals were restrained manually, and approximately 1 mL of blood at each time point was collected into pre-cooled EDTA-K2 tubes via cephalic or saphenous veins. Blood samples were centrifuged at 4° C. (4000 rpm. 5 min) to obtain plasma within 30 min after sample collection. All samples were stored at approximately −80° C. until analysis. The backup samples will be discarded after the in vivo experiment has been completed for two months unless requested.

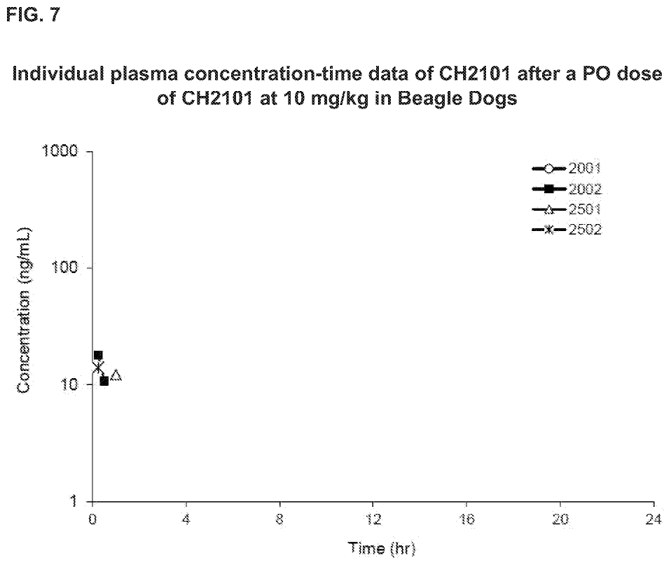

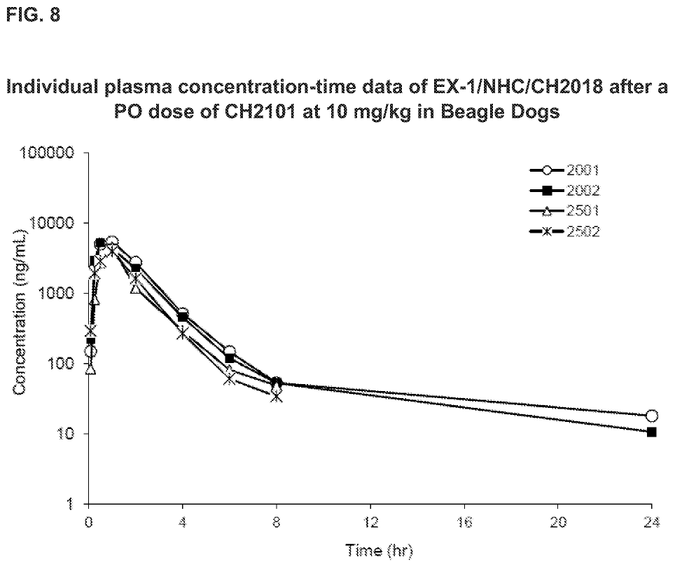

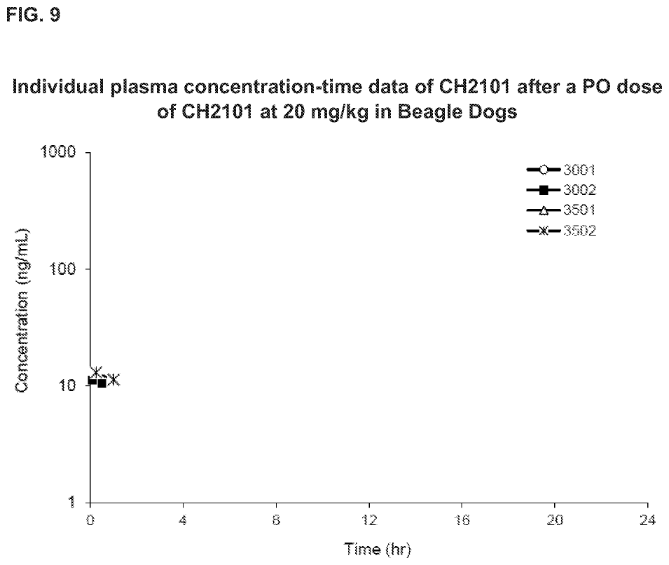

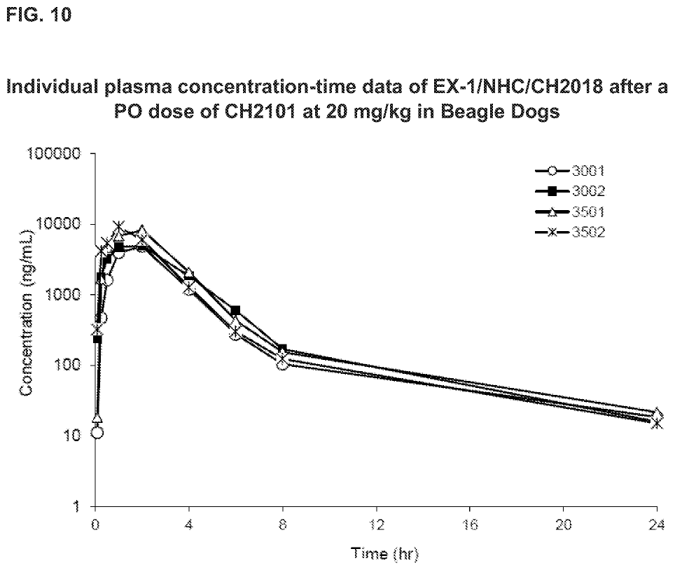

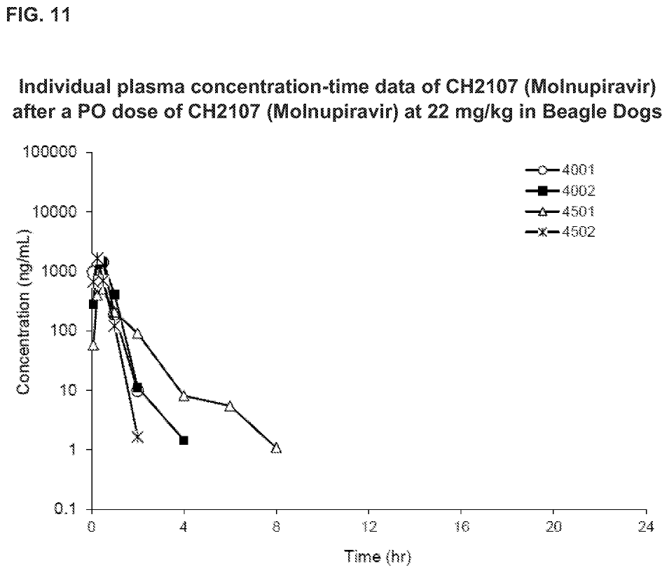

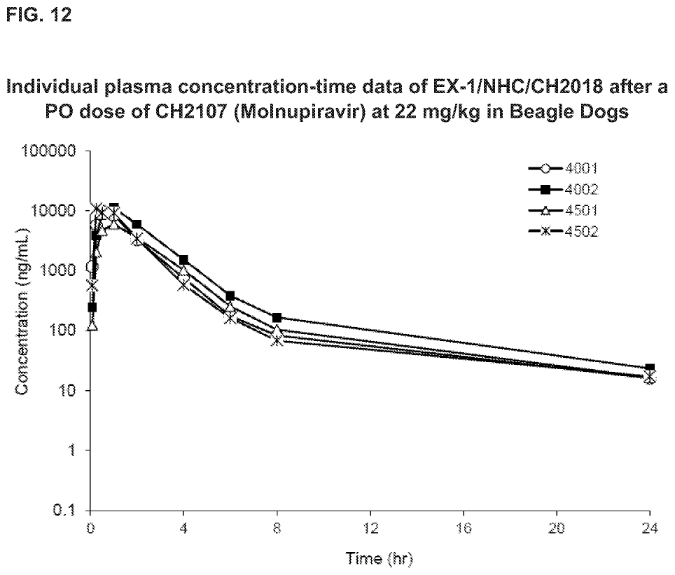

The experimental results were shown in Tables 6-11 and FIGS. 7-12.

TABLE 6
Plasma concentration-time data of EX-2/CH2101 and PK parameters after a PO dose at 10
mg/kg in Beagle Dogs

| | | Sampling | Concentration(ng/mL) | | | | | | | | |
|---|---|---|---|---|---|---|---|---|---|---|---|
| Dose | Dose | time | Male | | Female | | Male | Female | Male + Female | | |
| (mg/kg) | route | (br) | 2001 | 2002 | 2501 | 2502 | Mean | | Mean | SD | CV(%) |
| 10 | PO | 0.083 | BQL | BQL | BQL | BQL | BQL | BQL | BQL | NA | NA |
| | | 0.25 | BQL | 17.9 | BQL | 14.0 | 17.9 | 14.0 | 16.0 | NA | NA |
| | | 0.5 | BQL | 10.7 | BQL | BQL | 10.7 | BQL | 10.7 | NA | NA |
| | | 1 | BQL | BQL | 12.2 | BQL | BQL | 12.2 | 12.2 | NA | NA |
| | | 2 | BQL | BQL | BQL | BQL | BQL | BQL | BQL | NA | NA |
| | | 4 | BQL | BQL | BQL | BQL | BQL | BQL | BQL | NA | NA |
| | | 6 | BQL | BQL | BQL | BQL | BQL | BQL | BQL | NA | NA |
| | | 8 | BQL | BQL | BQL | BQL | BQL | BQL | BQL | NA | NA |
| | | 24 | BOL | BQL | BQL | BQL | BOL | BQL | BQL | NA | NA |

| | | | | | | Mean | Mean | | | |
|---|---|---|---|---|---|---|---|---|---|---|
| PK parameters | Unit | 2001 | 2002 | 2501 | 2502 | (male) | (female) | Mean | SD | CV(%) |
| Tₘₐₓ | hr | NC | 0.250 | 1.00 | 0.250 | 0.250 | [symbol] 0.625 | 0.438 | 0.433 | 99.0 |
| Cₘₐₓ | ng/ml | NC | 17.9 | 12.2 | 14.0 | 17.9 | [symbol] 13.1 | 15.5 | 2.91 | 18.8 |
| T_{1/2} | hr | NC | NC | NC | NO | NA | NA | NA | NA | NA |
| Regression Points | hr | NC | NC | NC | NC | NA | NA | NA | NA | NA |
| AUCₗₐₛₜ | hr*ng/mL | NC | 5.07 | NC | NC | 5.07 | NA | 5.07 | NA | NA |
| AUC_{INF} | hr*ng/ml | NC | NC | NC | NC | NA | NA | NA | NA | NA |
| MRT_{INF} | hr | NC | NC | NC | NC | NA | NA | NA | NA | NA |

| | | |
|---|---|---|
| AUCmale/female | % | NA |

TABLE 7
Plasma concentration-time data of EX-1/NHC/CH2018 and PK parameters after a PO dose
of CH2101 at 10 mg/kg in Beagle Dogs

| | | Sampling | Concentration(ng/mL) | | | | | | | | |
|---|---|---|---|---|---|---|---|---|---|---|---|
| Dose | Dose | time | Male | | Female | | Male | Female | Male + Female | | |
| (mg/kg) | route | (hr) | 2001 | 2002 | 2501 | 2502 | Mean | | Mean | SD | CV(%) |
| 10 | PO | 0.083 | 150 | 225 | 84.6 | 290 | [symbol] 188 | [symbol] 187 | 187 | [symbol] 89.3 | 47.6 |
| | | 0.25 | 2240 | 2930 | 819 | 1930 | [symbol] 2585 | [symbol] 1375 | 1980 | [symbol] 879 | 44.4 |
| | | 0.5 | 5010 | 5240 | 2780 | 2890 | [symbol] 5125 | [symbol] 2835 | 3980 | [symbol] 1326 | 33.3 |
| | | 1 | 5380 | 4330 | 4550 | 3940 | [symbol] 4855 | [symbol] 4245 | 4550 | [symbol] 608 | 13.4 |
| | | 2 | 2770 | 2260 | 1190 | 1640 | [symbol] 2515 | [symbol] 1415 | 1965 | [symbol] 693 | 35.3 |
| | | 4 | 514 | 457 | 292 | 269 | [symbol] 486 | [symbol] 281 | 383 | [symbol] 121 | 31.6 |
| | | 6 | 148 | 120 | 81.5 | 61.1 | [symbol] 134 | [symbol] 71.3 | 103 | [symbol] 38.9 | 37.9 |
| | | 8 | 53.3 | 52.9 | 48.7 | 34.0 | [symbol] 53.1 | [symbol] 41.4 | 47.2 | [symbol] 9.06 | 19.2 |
| | | 24 | 18.1 | 10.7 | BQL | BQL | [symbol] 14.4 | BQL | 14.4 | 5.23 | 36.3 |

| | | | | | | Mean | Mean | | | |
|---|---|---|---|---|---|---|---|---|---|---|
| PK parameters | Unit | 2001 | 2002 | 2501 | 2502 | (male) | (female) | Mean | SD | CV(%) |
| Tₘₐₓ | hr | 1.00 | 0.500 | 1.00 | 1.00 | [symbol] 0.750 | [symbol] 1.00 | 0.875 | 0.250 | 28.6 |
| Cₘₐₓ | ng/mL | 5380 | 5240 | 4550 | 3940 | [symbol] 5310 | [symbol] 4245 | 4778 | 666 | 13.9 |
| T_{1/2} | hr | 7.08 | 5.72 | 1.28 | 1.06 | [symbol] 6.40 | [symbol] 1.17 | 3.79 | 3.08 | 81.2 |
| Regression Points | hr | 6-24 | 6-24 | 2-8 | 2-8 | NA | NA | NA | NA | NA |
| AUCₗₐₛₜ | hr*ng/mL | 12503 | 10957 | 7217 | 7632 | [symbol] 11730 | [symbol] 7424 | 9577 | 2570 | 26.8 |
| AUC_{INF} | hr*ng/ml | 12688 | 11046 | 7307 | 7683 | [symbol] 11867 | [symbol] 7495 | 9681 | 2616 | 27.0 |
| MART_{INF} | hr | 2.60 | 2.26 | 1.66 | 1.58 | [symbol] 2.43 | [symbol] 1.62 | 2.03 | 0.486 | 24.0 |

| | | |
|---|---|---|
| AUCmale/female | % | 158 |

TABLE 8
Plasma concentration-time data of EX-2/CH2101 and PK parameters after a PO dose at 20
mg/kg in Beagle Dogs

| | | Sampling | Concentration(ng/mL) | | | | | | | | |
|---|---|---|---|---|---|---|---|---|---|---|---|
| Dose | Dose | time | Male | | Female | | Male | Female | Male + Female | | |
| (mg/kg) | route | (hr) | 3001 | 3002 | 3501 | 3502 | Mean | | Mean | SD | CV(%) |
| 20 | PO | 0.083 | BQL | 11.2 | BQL | BQL | 11.2 | BQL | 11.2 | NA | NA |
| | | 0.25 | BQL | 12.5 | BQL | 13.0 | 12.5 | 13.0 | 12.8 | NA | NA |
| | | 0.5 | BQL | 10.5 | BQL | BQL | 10.5 | BQL | 10.5 | NA | NA |
| | | 1 | BQL | BQL | BQL | 11.3 | BQL | 11.3 | 11.3 | NA | NA |
| | | 2 | BQL | BQL | BQL | BQL | BQL | BQL | BQL | NA | NA |
| | | 4 | BQL | BQL | BQL | BQL | BQL | BQL | BQL | NA | NA |
| | | 6 | BQL | BQL | BQL | BQL | BQL | BQL | BQL | NA | NA |
| | | 8 | BQL | BQL | BQL | BQL | BQL | BQL | BQL | NA | NA |
| | | 24 | BQL | BQL | BOL | BQL | BQL | BQL | BOL | NA | NA |

| | | | | | | Mean | Mean | | | |
|---|---|---|---|---|---|---|---|---|---|---|
| PK parameters | Unit | 3001 | 3002 | 3501 | 3502 | (male) | (female) | Mean | SD | CV(%) |
| Tₘₐₓ | hr | NC | 0.250 | NC | 0.250 | 0.250 | 0.250 | 0.250 | NA | NA |
| Cₘₐₓ | ng/mL | NC | 12.5 | NC | 13.0 | 12.5 | 13.0 | 12.8 | NA | NA |
| T_{1/2} | hr | NC | NC | NC | NC | NA | NA | NA | NA | NA |
| Regression Points | hr | NC | NC | NC | NC | NA | NA | NA | NA | NA |
| AUCₗₐₛₜ | hr*ng/ml | NC | 5.32 | NC | 5.54 | 5.32 | 5.54 | 5.43 | NA | NA |
| AUC_{INF} | hr*ng/ml | NC | NC | NC | NC | NA | NA | NA | NA | NA |
| MRT_{INF} | hr | NC | NC | NC | NC | NA | NA | NA | NA | NA |

| | | |
|---|---|---|
| AUCmale/female | % | 96.1 |

TABLE 9
Plasma concentration-time data of EX-1/NHC/CH2018 and PK parameters after a PO dose
of CH2101 at 20 mg/kg in Beagle Dogs

| | | Sampling | Concentration(ng/ml) | | | | | | | | |
|---|---|---|---|---|---|---|---|---|---|---|---|
| | Dose | time | Male | | Female | | Male | Female | Male + Female | | |
| (mg/kg) | route | (hr) | 3001 | 3002 | 3501 | 3502 | Mean | | Mean | SD | CV(%) |
| 20 | PC | 0.083 | 11.1 | 241 | 18.4 | 321 | [symbol] 126 | [symbol] 170 | 148 | [symbol] 157 | 106 |
| | | 0.25 | 465 | 1800 | 1680 | 4110 | [symbol] 1133 | [symbol] 2895 | 2014 | [symbol] 1522 | 75.6 |
| | | 0.5 | 1600 | 3220 | 4680 | 5310 | [symbol] 2410 | [symbol] 4995 | 3703 | [symbol] 1653 | 44.6 |
| | | 1 | 3930 | 4700 | 6770 | 9070 | [symbol] 4315 | [symbol] 7920 | 6118 | [symbol] 2305 | 37.7 |
| | | 2 | 4810 | 4890 | 8170 | 5960 | [symbol] 4850 | [symbol] 7065 | 5958 | [symbol] 1565 | 26.3 |
| | | 4 | 1190 | 1840 | 2090 | 1280 | [symbol] 1515 | [symbol] 1685 | 1600 | [symbol] 435 | 27.2 |
| | | 6 | 275 | 598 | 430 | 301 | [symbol] 437 | [symbol] 366 | 401 | [symbol] 148 | 36.9 |
| | | 8 | 104 | 170 | 154 | 123 | [symbol] 137 | [symbol] 139 | 138 | [symbol] 29.8 | 21.6 |
| | | 24 | 18.4 | 15.8 | 21.5 | 15.0 | [symbol] 17.1 | [symbol] 18.3 | 17.7 | [symbol] 2.93 | 16.6 |

| | | | | | | Mean | Mean | | | |
|---|---|---|---|---|---|---|---|---|---|---|
| PK parameters | Unit | 3001 | 3002 | 3501 | 3502 | (male) | (female) | Mean | SD | CV(%) |
| Tₘₐₓ | hr | 2.00 | 2.00 | 2.00 | 1.00 | [symbol] 2.00 | [symbol] 1.50 | 1.75 | 0.500 | 28.6 |
| Cₘₐₓ | ng/ml | 4810 | 4890 | 8170 | 9070 | [symbol] 4850 | [symbol] 8620 | 6735 | 2208 | 32.8 |
| T_{1/2} | hr | 5.17 | 3.82 | 4.63 | 4.53 | [symbol] 4.49 | [symbol] 4.58 | 4.54 | 0.553 | 12.2 |
| Regression Points | hr | 6-24 | 6-24 | 6-24 | 6-24 | NA | NA | NA | NA | NA |
| AUCₗₐₛₜ | br*ng/mL | 14874 | 19005 | 26038 | 23020 | [symbol] 16940 | [symbol] 24529 | 20734 | 4854 | 23.4 |
| AUC_{INF} | hr*ng/mL | 15011 | 19092 | 26182 | 23118 | [symbol] 17052 | [symbol] 24650 | 20851 | 4856 | 23.3 |
| MRT_{INF} | hr | 3.05 | 3.07 | 2.75 | 2.36 | [symbol] 3.06 | [symbol] 2.55 | 2.81 | 0.335 | 11.9 |
| AUCmale/female | % | | | | | 69.2 | | | | |

TABLE 10
Plasma concentration-time data of CH2107 (Molnupiravir) and PK parameters after a PO
dose of CH2107 (Molnupiravir) at 22 mg/kg in Beagle Dogs

| | | Sampling | Concentration(ng/ml) | | | | | | | | |
|---|---|---|---|---|---|---|---|---|---|---|---|
| Dose | Dose | time | Male | | Female | | Male | Female | Male + Female | | |
| (mg/kg) | route | (hr) | 4001 | 4002 | 4501 | 4502 | Mean | | Mean | SD | CV(%) |
| 22 | PO | 0.083 | 978 | 283 | 57.5 | 664 | [symbol] 631 | [symbol] 361 | 496 | [symbol] 408 | 82.2 |
| | | 0.25 | 1450 | 1680 | 398 | 1650 | [symbol] 1565 | [symbol] 1024 | 1295 | [symbol] 606 | 46.8 |
| | | 0.5 | 1420 | 1430 | 499 | 693 | [symbol] 1425 | [symbol] 596 | 1011 | [symbol] 485 | 48.0 |
| | | 1 | 187 | 407 | 207 | 120 | [symbol] 297 | [symbol] 164 | 230 | [symbol] 124 | 53.7 |
| | | 2 | 9.81 | 11.1 | 90.4 | 1.63 | [symbol] 10.5 | [symbol] 46.0 | 28.2 | [symbol] 41.7 | 148 |
| | | 4 | BQL | 1.44 | 8.06 | BQL | 1.44 | 8.06 | 4.75 | NA | NA |
| | | 6 | BQL | BQL | 5.45 | BQL | BQL | 5.45 | 5.45 | NA | NA |
| | | 8 | BQL | BQL | 1.08 | BQL | BQL | 1.08 | 1.08 | NA | NA |
| | | 24 | BQL | BQL | BQL | BQL | BQL | BQL | BQL | NA | NA |

| | | | | | | Mean | Mean | | | |
|---|---|---|---|---|---|---|---|---|---|---|
| PK parameters | Unit | 4001 | 4002 | 4501 | 4502 | (male) | (female) | Mean | SD | CV(%) |
| Tₘₐₓ | hr | 0.250 | 0.250 | 0.500 | 0.250 | [symbol] 0.250 | [symbol] 0.375 | 0.313 | 0.125 | 40.0 |
| Cₘₐₓ | ng/ml | 1450 | 1680 | 499 | 1650 | [symbol] 1565 | [symbol] 1075 | 1320 | 557 | 42.2 |
| T_{1/2} | hr | 0.212 | 0.352 | 0.945 | 0.170 | [symbol] 0.282 | [symbol] 0.558 | 0.420 | 0.359 | 85.5 |
| Regression Points | hr | 0.5-2 | 0.5-4 | 1-8 | 0.5-2 | NA | NA | NA | NA | NA |
| AUCₗₐₛₜ | hr*ng/ml | 1102 | 1245 | 596 | 778 | [symbol] 1174 | [symbol] 687 | 930 | 296 | 31.9 |
| AUC_{INF} | hr*ng/ml | 1105 | 1246 | 598 | 778 | [symbol] 1176 | [symbol] 688 | 932 | 297 | 31.8 |
| MRT_{INF} | hr | 0.461 | 0.567 | 1.16 | 0.405 | [symbol] 0.514 | [symbol] 0.783 | 0.648 | 0.348 | 53.7 |
| AUCmale/female | % | | | | | 171 | | | | |

TABLE 11
Plasma concentration-time data of EX-1/NHC/CH2018 and PK parameters after a PO
dose of CH2107 (Molnupiravir) at 22 mg/kg in Beagle Dogs

| | | | Concentration(ng/ml) | | | | | | | | |
|---|---|---|---|---|---|---|---|---|---|---|---|
| Dose | Dose | Sampling | Male | | Female | | Male | Female | Male + Female | | |
| (mg/kg) | route | time | 4001 | 4002 | 4501 | 4502 | Mean | | Mean | SD | CV(%) |
| 22 | PO | 0.083 | 1170 | 247 | 123 | 568 | [symbol] 709 | [symbol] 346 | 527 | [symbol] 468 | 88.8 |
| | | 0.25 | 5770 | 3860 | 2080 | 10900 | [symbol] 4815 | [symbol] 6490 | 5653 | [symbol] 3809 | 67.4 |
| | | 0.5 | 9840 | 8390 | 4720 | 9210 | [symbol] 9115 | [symbol] 6965 | 8040 | [symbol] 2292 | 28.5 |
| | | 1 | 8390 | 11100 | 5960 | 9140 | [symbol] 9745 | [symbol] 7550 | 8648 | [symbol] 2125 | 24.6 |
| | | 2 | 3290 | 5910 | 3530 | 3420 | [symbol] 4600 | [symbol] 3475 | 4038 | [symbol] 1252 | 31.0 |
| | | 4 | 749 | 1530 | 1020 | 580 | [symbol] 1140 | [symbol] 800 | 970 | [symbol] 415 | 42.8 |
| | | 6 | 176 | 384 | 254 | 162 | [symbol] 280 | [symbol] 208 | 244 | [symbol] 102 | 41.7 |
| | | 8 | 82.9 | 166 | 104 | 67.9 | [symbol] 124 | [symbol] 86.0 | 105 | [symbol] 43.2 | 41.0 |
| | | 24 | 16.1 | 23.6 | 16.5 | 17.2 | [symbol] 19.9 | [symbol] 16.9 | 18.4 | [symbol] 3.53 | 19.2 |

| | | | | | | Mean | Mean | | | |
|---|---|---|---|---|---|---|---|---|---|---|
| PK parameters | Unit | 4001 | 4002 | 4501 | 4502 | (male) | (female) | Mean | SD | CV(%) |
| Tₘₐₓ | hr | 0.500 | 1.00 | 1.00 | 0.250 | [symbol] 0.750 | [symbol] 0.625 | 0.688 | 0.375 | 54.5 |
| Cₘₐₓ | ng/ml | 9840 | 11100 | 5960 | 10900 | [symbol] 10470 | [symbol] 8430 | 9450 | 2391 | 25.3 |
| T_{1/2} | hr | 5.72 | 4.87 | 5.03 | 6.32 | [symbol] 5.30 | [symbol] 5.67 | 5.48 | 0.666 | 12.1 |
| Regression Points | hr | 6-24 | 6-24 | 6-24 | 6-24 | NA | NA | NA | NA | NA |
| AUCₗₐₛₜ | hr*ng/ml | 18992 | 26683 | 15600 | 20015 | [symbol] 22837 | [symbol] 17808 | 20322 | 4641 | 22.8 |
| AUC_{INF} | hr*ng/ml | 19125 | 26849 | 15720 | 20172 | [symbol] 22987 | [symbol] 17946 | 20466 | 4660 | 22.8 |
| MRT_{INF} | hr | 2.08 | 2.47 | 2.65 | 1.95 | [symbol] 2.27 | [symbol] 2.30 | 2.29 | 0.327 | 14.3 |

| | | |
|---|---|---|
| AUCmale/female | % | 128 |

As can be seen from Tables 6-11 and FIGS. 7-12, the AUC values of EX-1/NHC/CH2018 after administration of a PO dose of CH2101 at 20 mg/kg in Beagle Dogs were very similar to the AUC values of EX-1/NHC/CH2018 after administration of a PO dose of CH2107 (Molnupiravir) at 22 mg/kg in Beagle Dogs. Therefore, single administration of CH2101 (EX-2) has comparable pharmacokinetic profile to single administration of CH2107 (Molnupiravir) in Beagle Dogs.

1. A compound of Formula (I): 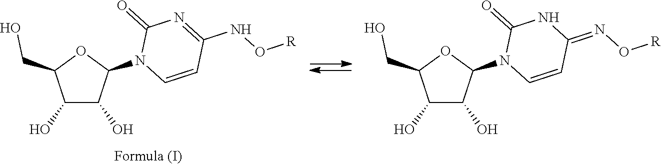
or a tautomer, stereoisomer or racemate thereof or a pharmaceutically acceptable salt thereof, wherein
R is Ra—(C═O)—;
wherein Ra is selected from the group consisting of C₁₋₇ alkyl, C₂₋₆ alkenyl, C₂₋₆ alkynyl, C₃₋₈ cycloalkyl, C₁₋₇ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl, wherein each of said alkyl, cycloalkyl, aryl, heteroaryl and heterocycloalkyl is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, aryloxy, heteroaryloxy, halo-C₁₋₇ alkyl, halo-C₁₋₇ alkoxy, halo-C₂₋₆ alkenyl, halo-C₂₋₆ alkynyl, hydroxy-C₁₋₇ alkyl, C₁₋₇ alkoxy-C₁₋₇ alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₈ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

2. The compound of Formula (I) according to claim 1, wherein
Ra is selected from the group consisting of C₁₋₇ alkyl, C₃₋₈ cycloalkyl, C₆₋₁₀ aryl, 5 to 10 membered heteroaryl, and 3 to 12 membered heterocycloalkyl, each of which is optionally substituted with one or more substituents selected from the following groups: halogen, acyl, hydroxy, cyano, nitro, amino, —NH(C₁₋₇ alkyl), —N(C₁₋₇ alkyl)₂, —CO—NH₂, —CO—NH(C₁₋₇ alkyl), —CO—N(C₁₋₇ alkyl)₂, —NH(acyl), —N(acyl)₂, NH₂-acyl, NHRy-acyl, N(Ry)₂-acyl, C₁₋₇ alkyl, C₂₋₆ alkenyl, C₂₋₆ alkynyl, C₁₋₇ alkoxy, halo-C₁₋₇ alkyl, halo-C₁₋₇ alkoxy, aryloxy, heteroaryloxy, halo-C₂₋₆ alkenyl, halo-C₂₋₆ alkynyl, hydroxy-C₁₋₇alkyl, C₁₋₇ alkoxy-C₁₋₇alkyl, halo-C₁₋₇ alkoxy-C₁₋₇ alkyl, halo-C₃₋₈ cycloalkyl, C₃₋₈ cycloalkyl, C₆₋₁₀ aryl, 5 to 10 membered heteroaryl, 3 to 12 membered heterocycloalkyl, C₃₋₈ cycloalkyloxy or 3 to 12 membered heterocycloalkyloxy,
wherein Ry is independently selected from C₁₋₇ alkyl, C₃₋₈ cycloalkyl, C₆₋₁₀ aryl, 5 to 10 membered heteroaryl, 3 to 12 membered heterocycloalkyl, C₃₋₈ cycloalkyl-C₁₋₇ alkyl, C₆₋₁₀ aryl-C₁₋₇ alkyl, 5 to 10 membered heteroaryl-C₁₋₇ alkyl, and 3 to 12 membered heterocycloalkyl-C₁₋₇ alkyl.

3. The compound of Formula (I) according to claim 1, or a tautomer, stereoisomer or racemate thereof or a pharmaceutically acceptable salt thereof, wherein R is selected from the following groups:

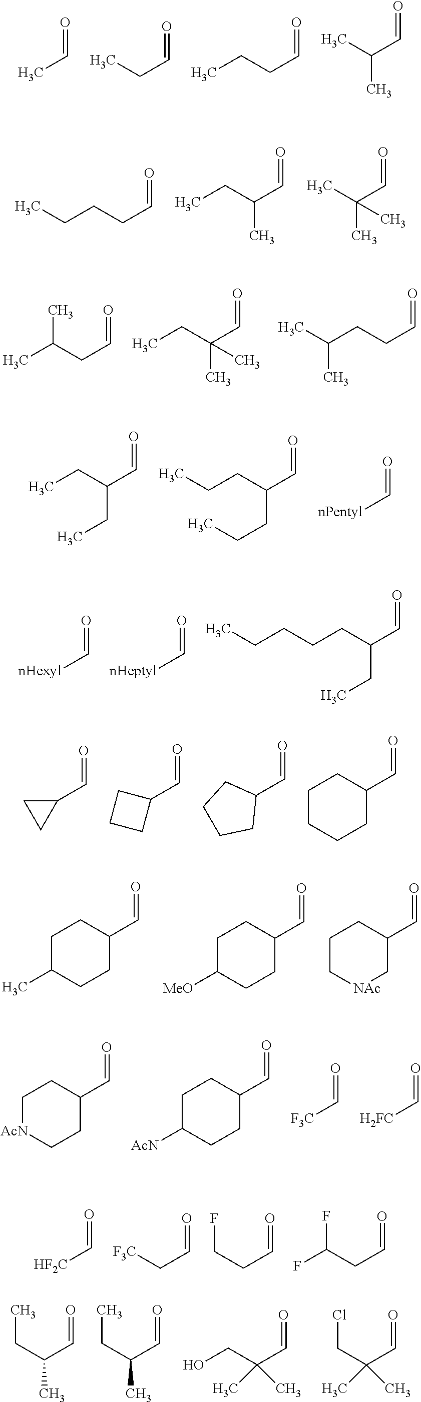

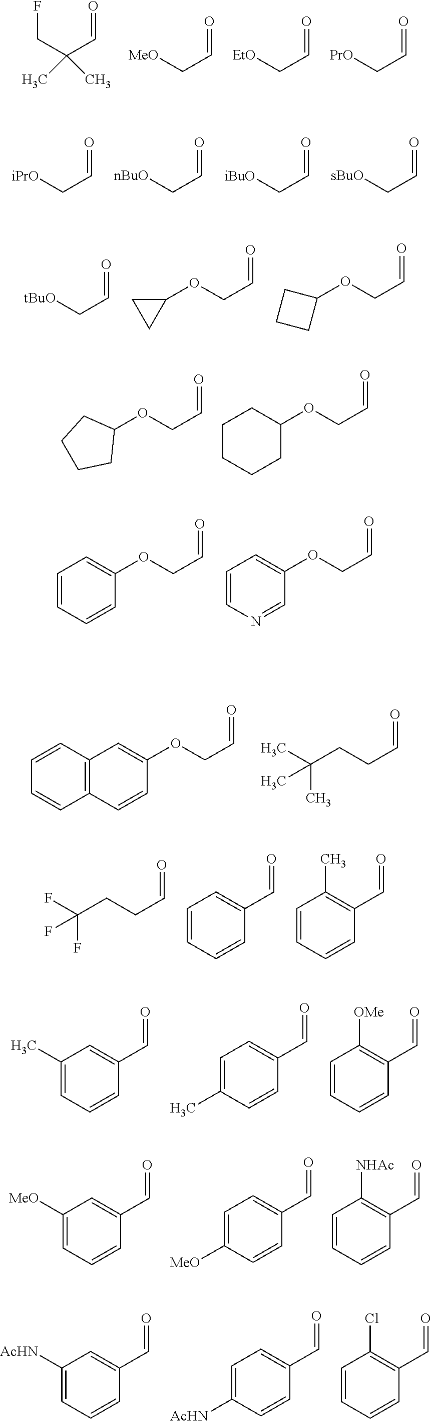

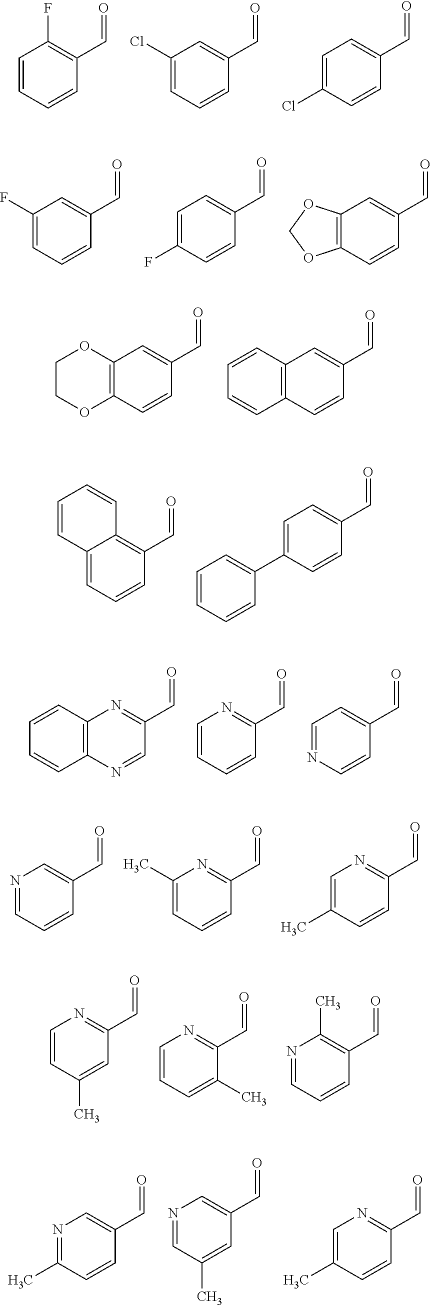

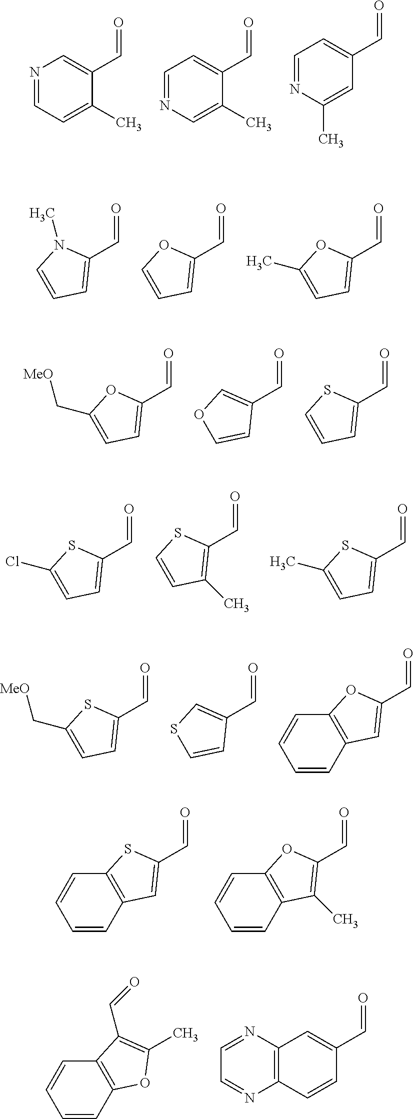

4. A compound according to claim 1, or a tautomer, stereoisomer or racemate thereof or a pharmaceutically acceptable salt thereof, wherein
R is Ra—(C═O)—,
Ra is methyl substituted with Ra1, Ra2 and Ra3;
wherein each of Ra1, Ra2 and Ra3 is independently selected from the group consisting of H, C₁₋₆alkyl, C₁₋₆alkyl-O—C₁₋₆alkyl, C₃₋₆cycloalkyl, 3-6 membered heterocycloalkyl, C₁₋₆alkyl-O—(CH₂)ₙ—, C₁₋₇alkyl-O-aryl, C₁₋₇alkyl-O-heteroaryl, C₁₋₆alkyl-O—C₁₋₆alkyl-O—(CH₂)ₙ—, C₁₋₆haloalkyl-O—(CH₂)ₙ—, C₃₋₆cycloalkyl-O—(CH₂)ₙ— and 3-6 membered heterocycloalkyl-O—(CH₂)ₙ—, wherein each of said alkyl, cycloalkyl and heterocycloalkyl is optionally substituted with one or more substituents selected from the following groups: halogen, acyl, hydroxy, cyano, nitro, amino, —NH(C₁₋₇ alkyl), —N(C₁₋₇ alkyl)₂, C₁₋₇ alkyl, C₁₋₆ alkoxy, halo-C₁₋₇ alkyl, or halo-C₁₋₇ alkoxy; and
n is 0 or 1.

5. A compound according to claim 1 or a tautomer, stereoisomer or racemate thereof or a pharmaceutically acceptable salt thereof, wherein
R is Ra—(C═O)—,
wherein Ra—(C═O)— is selected from the group consisting of: 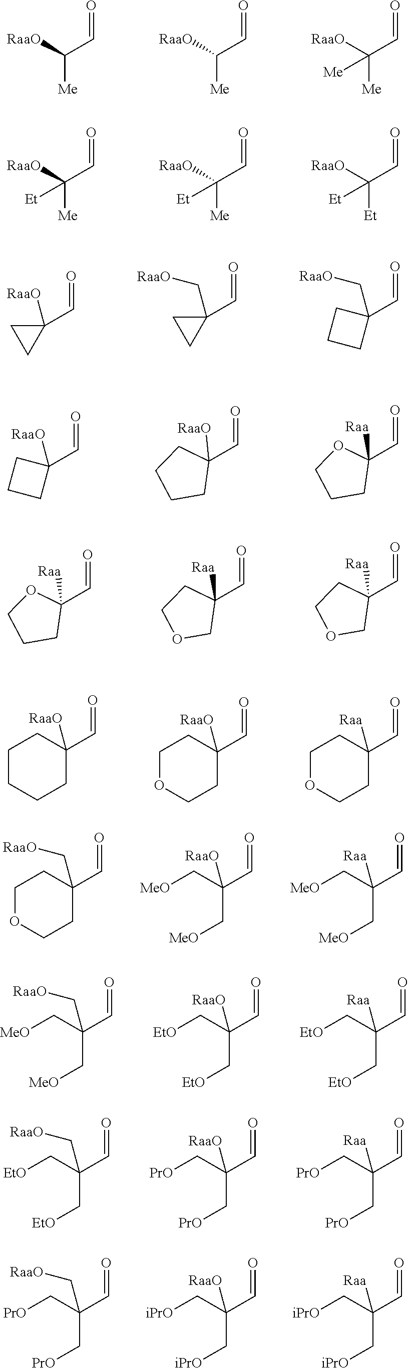 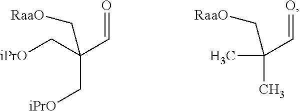
Raa is selected from the group consisting of C₁₋₆alkyl, C₁₋₆haloalkyl, C₁₋₆alkyl-O—C₁₋₆alkyl-, C₃₋₆cycloalkyl and 3-6 membered heterocycloalkyl; preferably C₁₋₆alkyl.

6. A compound according to claim 5, or a tautomer, stereoisomer or racemate thereof or a pharmaceutically acceptable salt thereof, wherein Ra—(C═O)— is selected from the group consisting of:

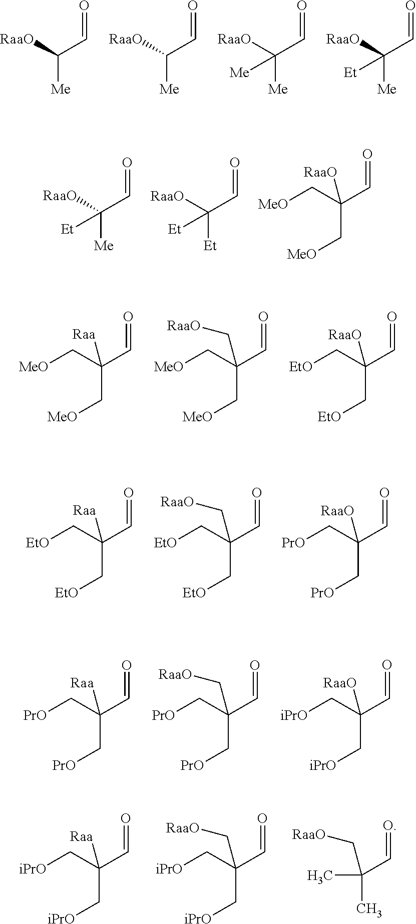

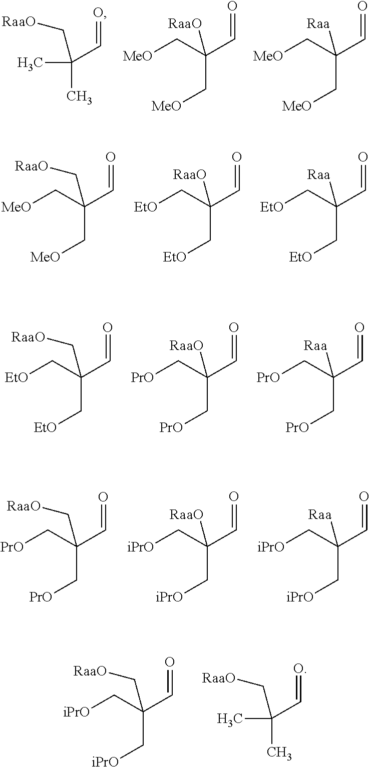

7. A compound according to claim 6, or a tautomer, stereoisomer or racemate thereof or a pharmaceutically acceptable salt thereof, wherein RaC═O is selected from the group consisting of:

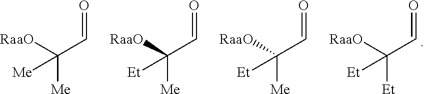

8. A compound according to any of the preceding claims, or a tautomer, stereoisomer or racemate thereof or a pharmaceutically acceptable salt thereof, wherein Raa is selected from the group consisting of methyl, ethyl, propyl, isopropyl, n-butyl, sec-butyl, 2-methoxyethyl, fluorosubstitued ethyl, flurosubstituted propyl, cyclopropyl, cyclobutyl, cyclopentyl, oxetanyl, tetrahydro-2-furanyl, tetrahydro-3-furanyl or tetrahydro-2H-pyran-4-yl; preferably methyl, ethyl, propyl, isopropyl, oxetanyl and tetrahydro-2H-pyran-4-yl.

9. A compound according to any of the preceding claims, or a tautomer, stereoisomer or racemate thereof or a pharmaceutically acceptable salt thereof, wherein Raa is selected from the group consisting of methyl, ethyl, propyl, isopropyl, n-butyl and sec-butyl.

10. A compound according to claim 4, or a tautomer, stereoisomer or racemate thereof or a pharmaceutically acceptable salt thereof, wherein
Ra1 and Ra3 is independently selected from the group consisting of H, C₁₋₆alkyl, C₁₋₆alkyl-O— and C₁₋₆alkyl-O—CH₂—;
Ra2 is selected from the group consisting of C₁₋₆alkyl, C₁₋₆alkyl-O and C₁₋₆alkyl-O—CH₂—; or
Ra2 and Ra3 are taken together with the carbon they attached to form C₃₋₆cycloalkyl, or 5-6 membered haloheterocycloalkyl comprising 1 ring heteroatom selected from O;
provided that when Ra3 is either H or C₁₋₆alkyl, Ra1 is not H or C₁₋₆alkyl.

11. A compound according to any of the preceding claims, or a tautomer, stereoisomer or racemate thereof or a pharmaceutically acceptable salt thereof, wherein
Ra1 is C₁₋₆alkyl-O—, and
Ra2 and Ra3 is independently C₁₋₃alkyl, preferably Ra2 and Ra3 are the same.

12. A compound according to any of the preceding claims, or a tautomer, stereoisomer or racemate thereof or a pharmaceutically acceptable salt thereof, wherein R¹ is RaC═O, and each of R² and R³ is H.

13. A compound according to any of the preceding claims, or a tautomer, stereoisomer or racemate thereof or a tautomer, stereoisomer or racemate thereof or a pharmaceutically acceptable salt thereof, wherein Ra1 is C₁₋₆alkyl-O—.

14. A compound according to claim 1, or a tautomer, stereoisomer or racemate thereof or a tautomer, stereoisomer or racemate thereof or a pharmaceutically acceptable salt thereof, wherein the compound is selected from the group consisting of:

| Example # | Structure |
|---|---|
| EX-2 | 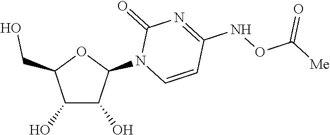 [SMILES: CC(=O)ONc1ccn([C@@H]2O[C@H](CO)[C@@H](O)[C@H]2O)c(=O)n1] |
| EX-3 | 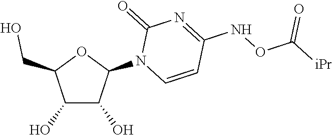 [SMILES: CC(C)C(=O)ONc1ccn([C@@H]2O[C@H](CO)[C@@H](O)[C@H]2O)c(=O)n1] |
| EX-4 | 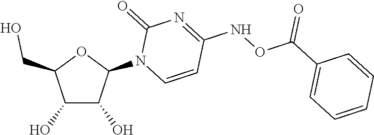 [SMILES: O=C(ONc1ccn([C@@H]2O[C@H](CO)[C@@H](O)[C@H]2O)c(=O)n1)c1ccccc1] |
| EX-5 | 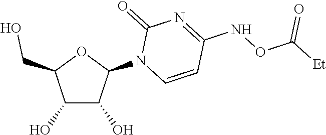 [SMILES: CCC(=O)ONc1ccn([C@@H]2O[C@H](CO)[C@@H](O)[C@H]2O)c(=O)n1] |
| EX-4a | 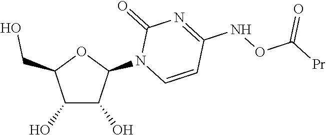 |
| EX-5a | 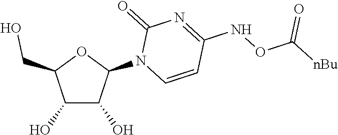 |
| EX-6 | 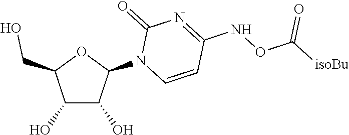 |
| EX-7 | 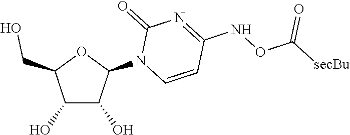 |
| EX-8 | 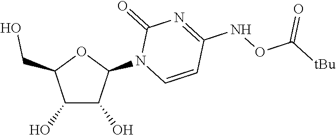 [SMILES: CC(C)(C)C(=O)ONc1ccn([C@@H]2O[C@H](CO)[C@@H](O)[C@H]2O)c(=O)n1] |
| EX-9 | 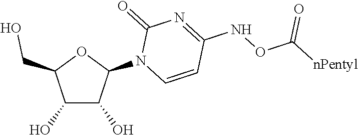 |
| EX-10 | 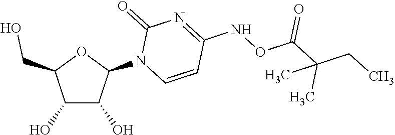 [SMILES: CCC(C)(C)C(=O)ONc1ccn([C@@H]2O[C@H](CO)[C@@H](O)[C@H]2O)c(=O)n1] |
| EX-11 | 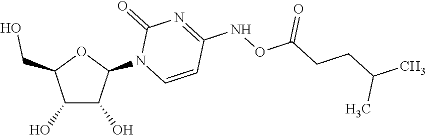 [SMILES: CC(C)CCC(=O)ONc1ccn([C@@H]2O[C@H](CO)[C@@H](O)[C@H]2O)c(=O)n1] |
| EX-12 | 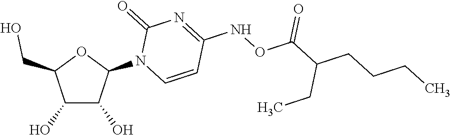 [SMILES: CCCCC(CC)C(=O)ONc1ccn([C@@H]2O[C@H](CO)[C@@H](O)[C@H]2O)c(=O)n1] |
| EX-13 | 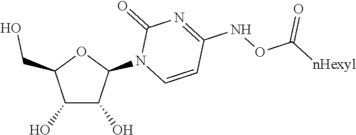 |
| EX-14 | 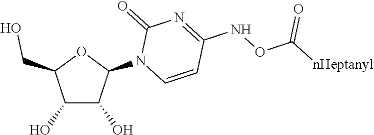 |
| EX-15 | 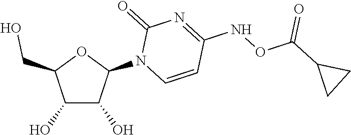 [SMILES: O=C(ONc1ccn([C@@H]2O[C@H](CO)[C@@H](O)[C@H]2O)c(=O)n1)C1CC1] |
| EX-16 | 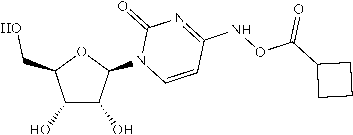 [SMILES: O=C(ONc1ccn([C@@H]2O[C@H](CO)[C@@H](O)[C@H]2O)c(=O)n1)C1CCC1] |
| EX-17 | 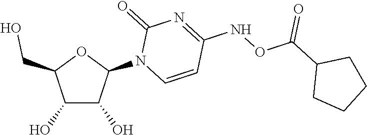 [SMILES: O=C(ONc1ccn([C@@H]2O[C@H](CO)[C@@H](O)[C@H]2O)c(=O)n1)C1CCCC1] |
| EX-18 | 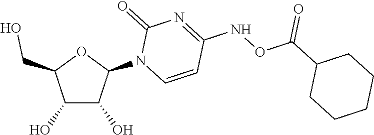 [SMILES: O=C(ONc1ccn([C@@H]2O[C@H](CO)[C@@H](O)[C@H]2O)c(=O)n1)C1CCCCC1] |
| EX-19 | 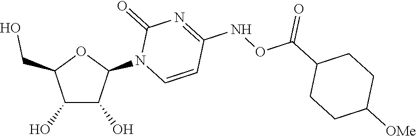 [SMILES: COC1CCC(C(=O)ONc2ccn([C@@H]3O[C@H](CO)[C@@H](O)[C@H]3O)c(=O)n2)CC1] |
| EX-20 | 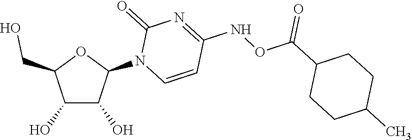 [SMILES: CC1CCC(C(=O)ONc2ccn([C@@H]3O[C@H](CO)[C@@H](O)[C@H]3O)c(=O)n2)CC1] |
| EX-21 | 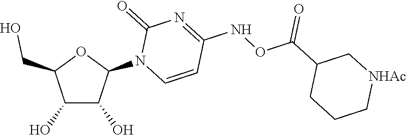 |
| EX-22 | 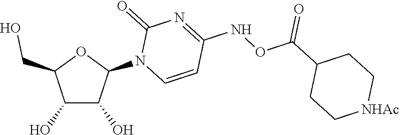 |
| EX-23 | 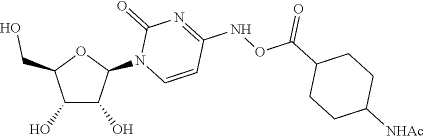 [SMILES: CC(=O)NC1CCC(C(=O)ONc2ccn([C@@H]3O[C@H](CO)[C@@H](O)[C@H]3O)c(=O)n2)CC1] |
| EX-24 | 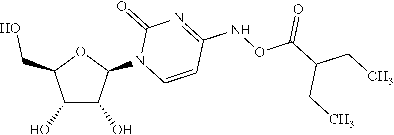 [SMILES: CCC(CC)C(=O)ONc1ccn([C@@H]2O[C@H](CO)[C@@H](O)[C@H]2O)c(=O)n1] |
| EX-25 | 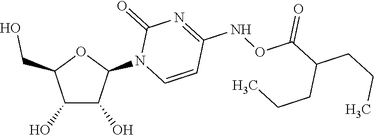 [SMILES: CCCC(CCC)C(=O)ONc1ccn([C@@H]2O[C@H](CO)[C@@H](O)[C@H]2O)c(=O)n1] |
| EX-26 | 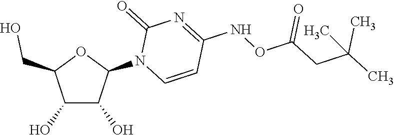 [SMILES: CC(C)(C)CC(=O)ONc1ccn([C@@H]2O[C@H](CO)[C@@H](O)[C@H]2O)c(=O)n1] |
| EX-27 | 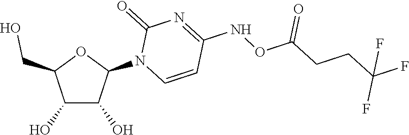 [SMILES: O=C(CCC(F)(F)F)ONc1ccn([C@@H]2O[C@H](CO)[C@@H](O)[C@H]2O)c(=O)n1] |
| EX-28 | 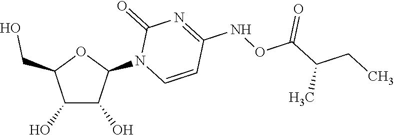 [SMILES: CC[C@H](C)C(=O)ONc1ccn([C@@H]2O[C@H](CO)[C@@H](O)[C@H]2O)c(=O)n1] |
| EX-29 | 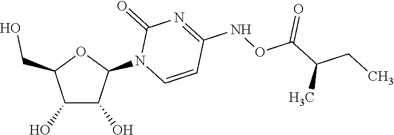 [SMILES: CC[C@@H](C)C(=O)ONc1ccn([C@@H]2O[C@H](CO)[C@@H](O)[C@H]2O)c(=O)n1] |
| EX-30 | 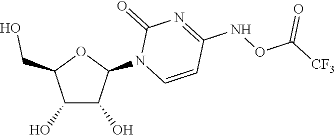 [SMILES: O=C(ONc1ccn([C@@H]2O[C@H](CO)[C@@H](O)[C@H]2O)c(=O)n1)C(F)(F)F] |
| EX-31 | 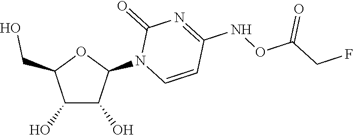 [SMILES: O=C(CF)ONc1ccn([C@@H]2O[C@H](CO)[C@@H](O)[C@H]2O)c(=O)n1] |
| EX-32 | 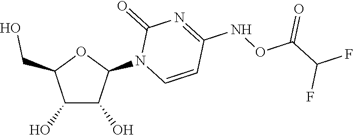 [SMILES: O=C(ONc1ccn([C@@H]2O[C@H](CO)[C@@H](O)[C@H]2O)c(=O)n1)C(F)F] |
| EX-33 | 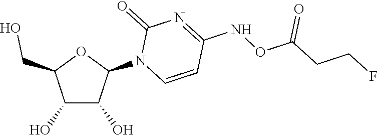 [SMILES: O=C(CCF)ONc1ccn([C@@H]2O[C@H](CO)[C@@H](O)[C@H]2O)c(=O)n1] |
| EX-34 | 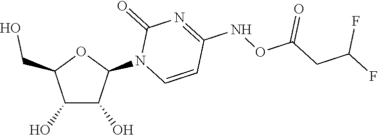 [SMILES: O=C(CC(F)F)ONc1ccn([C@@H]2O[C@H](CO)[C@@H](O)[C@H]2O)c(=O)n1] |
| EX-35 | 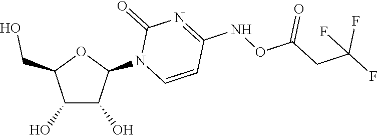 [SMILES: O=C(CC(F)(F)F)ONc1ccn([C@@H]2O[C@H](CO)[C@@H](O)[C@H]2O)c(=O)n1] |
| EX-36 | 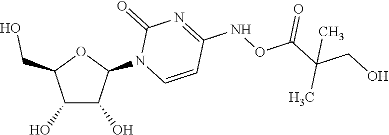 [SMILES: CC(C)(CO)C(=O)ONc1ccn([C@@H]2O[C@H](CO)[C@@H](O)[C@H]2O)c(=O)n1] |
| EX-37 | 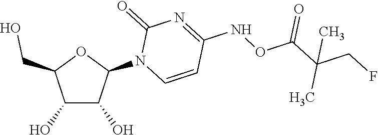 [SMILES: CC(C)(CF)C(=O)ONc1ccn([C@@H]2O[C@H](CO)[C@@H](O)[C@H]2O)c(=O)n1] |
| EX-38 | 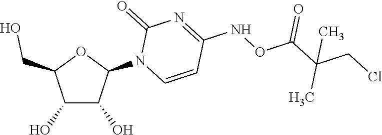 [SMILES: CC(C)(CCl)C(=O)ONc1ccn([C@@H]2O[C@H](CO)[C@@H](O)[C@H]2O)c(=O)n1] |
| EX-39 | 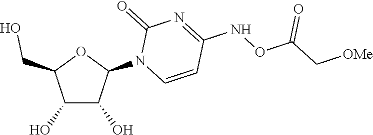 [SMILES: COCC(=O)ONc1ccn([C@@H]2O[C@H](CO)[C@@H](O)[C@H]2O)c(=O)n1] |
| EX-40 | 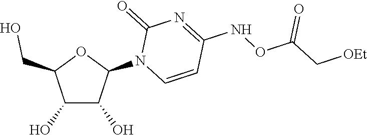 [SMILES: CCOCC(=O)ONc1ccn([C@@H]2O[C@H](CO)[C@@H](O)[C@H]2O)c(=O)n1] |
| EX-41 | 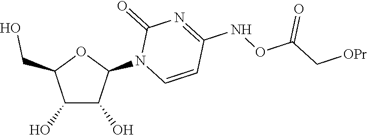 |
| EX-42 | 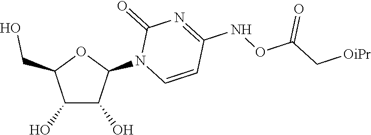 [SMILES: CC(C)OCC(=O)ONc1ccn([C@@H]2O[C@H](CO)[C@@H](O)[C@H]2O)c(=O)n1] |
| EX-43 | 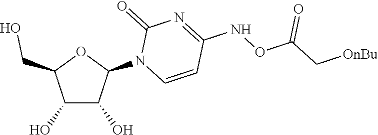 |
| EX-44 | 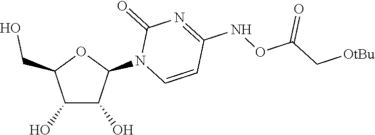 [SMILES: CC(C)(C)OCC(=O)ONc1ccn([C@@H]2O[C@H](CO)[C@@H](O)[C@H]2O)c(=O)n1] |
| EX-45 | 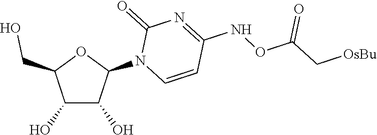 |
| EX-46 | 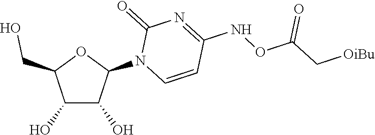 |
| EX-47 | 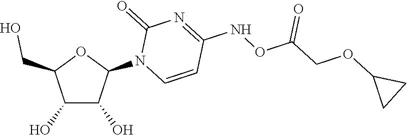 [SMILES: O=C(COC1CC1)ONc1ccn([C@@H]2O[C@H](CO)[C@@H](O)[C@H]2O)c(=O)n1] |
| EX-48 | 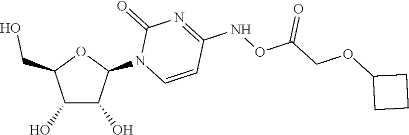 [SMILES: O=C(COC1CCC1)ONc1ccn([C@@H]2O[C@H](CO)[C@@H](O)[C@H]2O)c(=O)n1] |
| EX-49 | 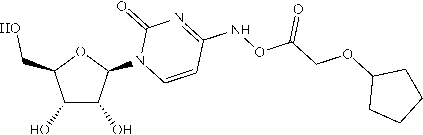 [SMILES: O=C(COC1CCCC1)ONc1ccn([C@@H]2O[C@H](CO)[C@@H](O)[C@H]2O)c(=O)n1] |
| EX-50 | 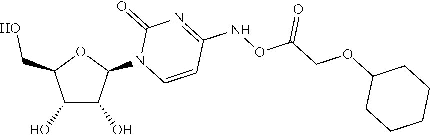 [SMILES: O=C(COC1CCCCC1)ONc1ccn([C@@H]2O[C@H](CO)[C@@H](O)[C@H]2O)c(=O)n1] |
| EX-51 | 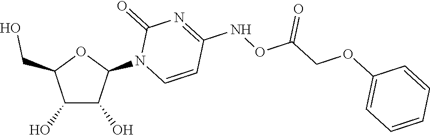 [SMILES: O=C(COc1ccccc1)ONc1ccn([C@@H]2O[C@H](CO)[C@@H](O)[C@H]2O)c(=O)n1] |
| EX-52 | 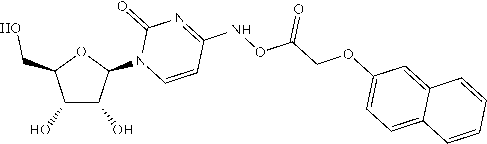 [SMILES: O=C(COc1ccc2ccccc2c1)ONc1ccn([C@@H]2O[C@H](CO)[C@@H](O)[C@H]2O)c(=O)n1] |
| EX-53 | 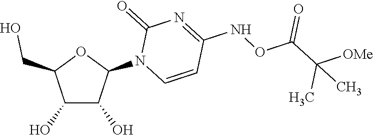 [SMILES: COC(C)(C)C(=O)ONc1ccn([C@@H]2O[C@H](CO)[C@@H](O)[C@H]2O)c(=O)n1] |
| EX-54 | 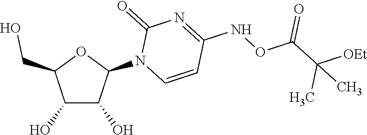 [SMILES: CCOC(C)(C)C(=O)ONc1ccn([C@@H]2O[C@H](CO)[C@@H](O)[C@H]2O)c(=O)n1] |
| EX-55 | 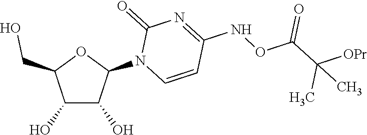 |
| EX-56 | 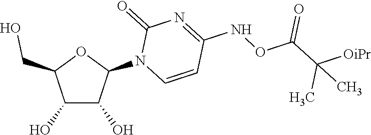 [SMILES: CC(C)OC(C)(C)C(=O)ONc1ccn([C@@H]2O[C@H](CO)[C@@H](O)[C@H]2O)c(=O)n1] |
| EX-57 | 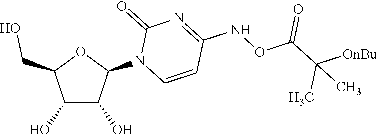 |
| EX-58 | 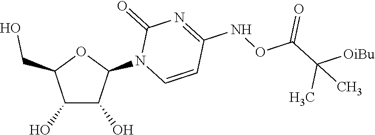 |
| EX-59 | 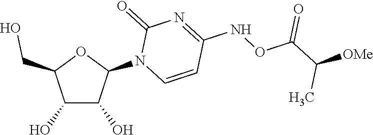 [SMILES: CO[C@@H](C)C(=O)ONc1ccn([C@@H]2O[C@H](CO)[C@@H](O)[C@H]2O)c(=O)n1] |
| EX-60 | 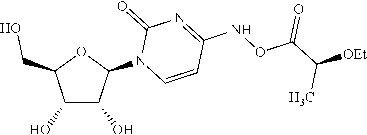 [SMILES: CCO[C@@H](C)C(=O)ONc1ccn([C@@H]2O[C@H](CO)[C@@H](O)[C@H]2O)c(=O)n1] |
| EX-61 | 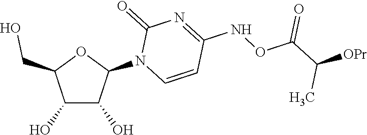 |
| EX-62 | 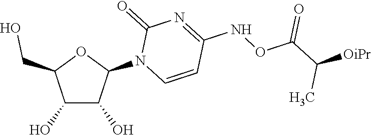 [SMILES: CC(C)O[C@@H](C)C(=O)ONc1ccn([C@@H]2O[C@H](CO)[C@@H](O)[C@H]2O)c(=O)n1] |
| EX-63 | 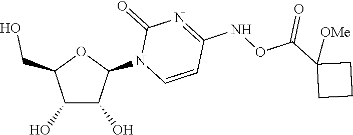 [SMILES: COC1(C(=O)ONc2ccn([C@@H]3O[C@H](CO)[C@@H](O)[C@H]3O)c(=O)n2)CCC1] |
| EX-64 | 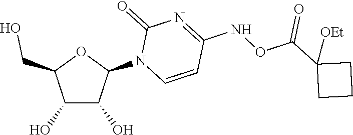 [SMILES: CCOC1(C(=O)ONc2ccn([C@@H]3O[C@H](CO)[C@@H](O)[C@H]3O)c(=O)n2)CCC1] |
| EX-65 | 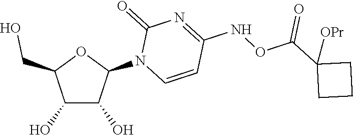 |
| EX-66 | 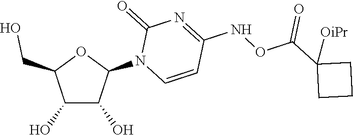 [SMILES: CC(C)OC1(C(=O)ONc2ccn([C@@H]3O[C@H](CO)[C@@H](O)[C@H]3O)c(=O)n2)CCC1] |
| EX-67 | 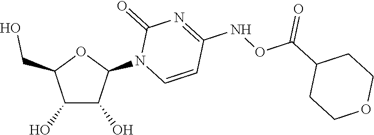 [SMILES: O=C(ONc1ccn([C@@H]2O[C@H](CO)[C@@H](O)[C@H]2O)c(=O)n1)C1CCOCC1] |
| EX-68 | 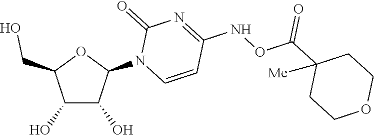 [SMILES: CC1(C(=O)ONc2ccn([C@@H]3O[C@H](CO)[C@@H](O)[C@H]3O)c(=O)n2)CCOCC1] |
| EX-69 | 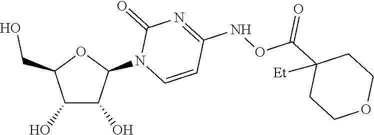 [SMILES: CCC1(C(=O)ONc2ccn([C@@H]3O[C@H](CO)[C@@H](O)[C@H]3O)c(=O)n2)CCOCC1] |
| EX-70 | 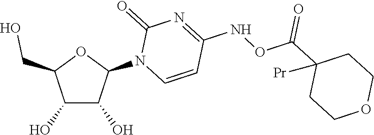 |
| EX-71 | 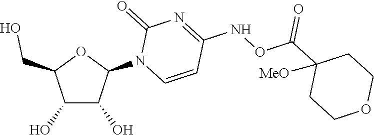 [SMILES: COC1(C(=O)ONc2ccn([C@@H]3O[C@H](CO)[C@@H](O)[C@H]3O)c(=O)n2)CCOCC1] |
| EX-72 | 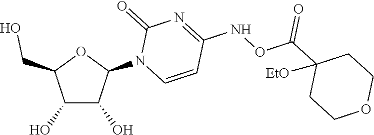 [SMILES: CCOC1(C(=O)ONc2ccn([C@@H]3O[C@H](CO)[C@@H](O)[C@H]3O)c(=O)n2)CCOCC1] |
| EX-73 | 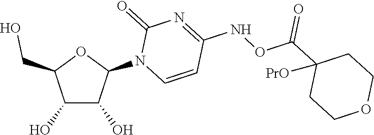 |
| EX-74 | 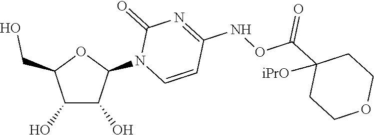 [SMILES: CC(C)OC1(C(=O)ONc2ccn([C@@H]3O[C@H](CO)[C@@H](O)[C@H]3O)c(=O)n2)CCOCC1] |
| EX-75 | 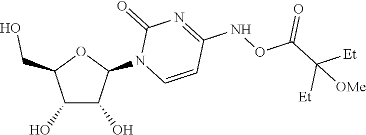 [SMILES: CCC(CC)(OC)C(=O)ONc1ccn([C@@H]2O[C@H](CO)[C@@H](O)[C@H]2O)c(=O)n1] |
| EX-76 | 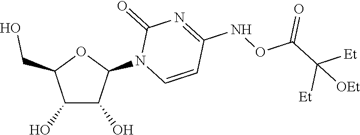 [SMILES: CCOC(CC)(CC)C(=O)ONc1ccn([C@@H]2O[C@H](CO)[C@@H](O)[C@H]2O)c(=O)n1] |
| EX-77 | 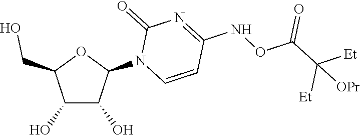 |
| EX-78 | 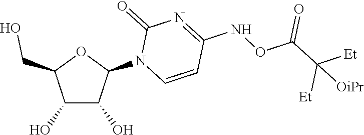 [SMILES: CCC(CC)(OC(C)C)C(=O)ONc1ccn([C@@H]2O[C@H](CO)[C@@H](O)[C@H]2O)c(=O)n1] |
| EX-79 | 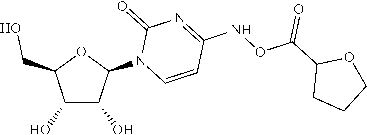 [SMILES: O=C(ONc1ccn([C@@H]2O[C@H](CO)[C@@H](O)[C@H]2O)c(=O)n1)C1CCCO1] |
| EX-80 | 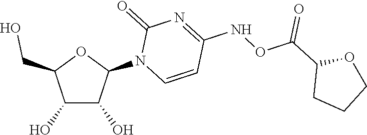 |
| EX-81 | 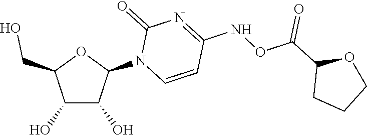 |
| EX-82 | 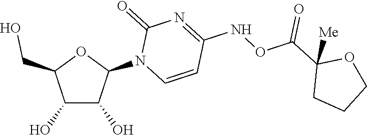 [SMILES: C[C@]1(C(=O)ONc2ccn([C@@H]3O[C@H](CO)[C@@H](O)[C@H]3O)c(=O)n2)CCCO1] |
| EX-83 | 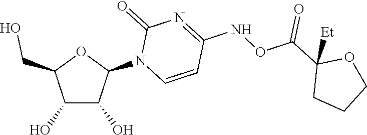 [SMILES: CC[C@]1(C(=O)ONc2ccn([C@@H]3O[C@H](CO)[C@@H](O)[C@H]3O)c(=O)n2)CCCO1] |
| EX-84 | 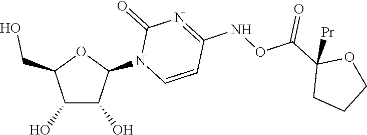 |
| EX-85 | 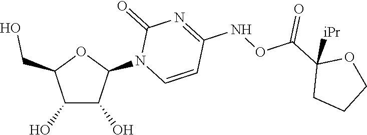 [SMILES: CC(C)[C@]1(C(=O)ONc2ccn([C@@H]3O[C@H](CO)[C@@H](O)[C@H]3O)c(=O)n2)CCCO1] |
| EX-86 | 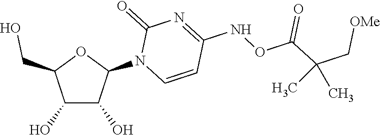 [SMILES: COCC(C)(C)C(=O)ONc1ccn([C@@H]2O[C@H](CO)[C@@H](O)[C@H]2O)c(=O)n1] |
| EX-87 | 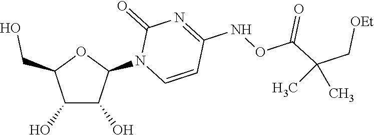 [SMILES: CCOCC(C)(C)C(=O)ONc1ccn([C@@H]2O[C@H](CO)[C@@H](O)[C@H]2O)c(=O)n1] |
| EX-88 | 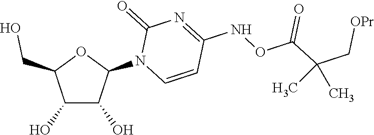 |
| EX-89 | 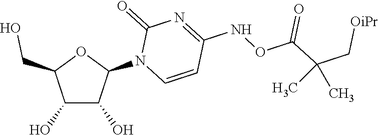 [SMILES: CC(C)OCC(C)(C)C(=O)ONc1ccn([C@@H]2O[C@H](CO)[C@@H](O)[C@H]2O)c(=O)n1] |
| EX-90 | 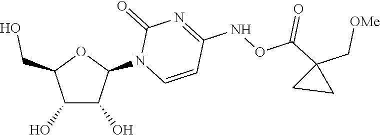 [SMILES: COCC1(C(=O)ONc2ccn([C@@H]3O[C@H](CO)[C@@H](O)[C@H]3O)c(=O)n2)CC1] |
| EX-91 | 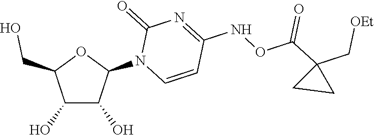 [SMILES: CCOCC1(C(=O)ONc2ccn([C@@H]3O[C@H](CO)[C@@H](O)[C@H]3O)c(=O)n2)CC1] |
| EX-92 | 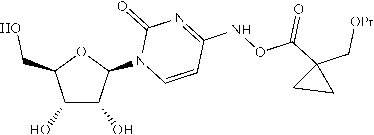 |
| EX-93 | 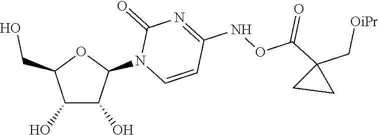 [SMILES: CC(C)OCC1(C(=O)ONc2ccn([C@@H]3O[C@H](CO)[C@@H](O)[C@H]3O)c(=O)n2)CC1] |
| EX-94 | 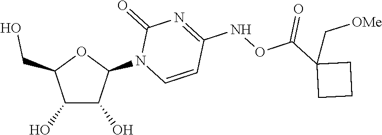 [SMILES: COCC1(C(=O)ONc2ccn([C@@H]3O[C@H](CO)[C@@H](O)[C@H]3O)c(=O)n2)CCC1] |
| EX-95 | 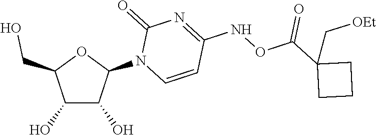 [SMILES: CCOCC1(C(=O)ONc2ccn([C@@H]3O[C@H](CO)[C@@H](O)[C@H]3O)c(=O)n2)CCC1] |
| EX-96 | 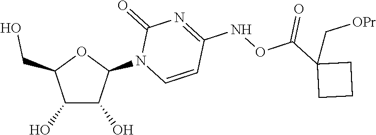 |
| EX-97 | 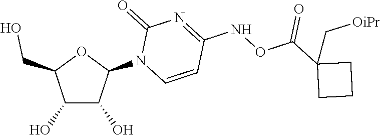 [SMILES: CC(C)OCC1(C(=O)ONc2ccn([C@@H]3O[C@H](CO)[C@@H](O)[C@H]3O)c(=O)n2)CCC1] |
| EX-98 | 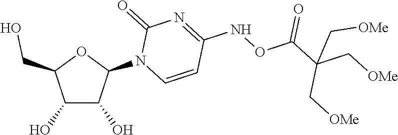 [SMILES: COCC(COC)(COC)C(=O)ONc1ccn([C@@H]2O[C@H](CO)[C@@H](O)[C@H]2O)c(=O)n1] |
| EX-99 | 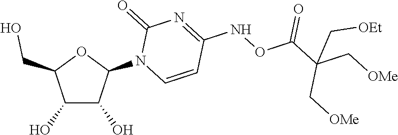 [SMILES: CCOCC(COC)(COC)C(=O)ONc1ccn([C@@H]2O[C@H](CO)[C@@H](O)[C@H]2O)c(=O)n1] |
| EX-100 | 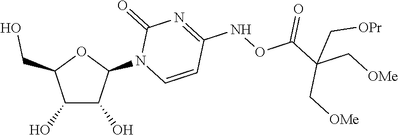 |
| EX-102 | 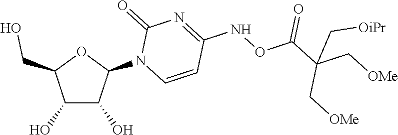 [SMILES: COCC(COC)(COC(C)C)C(=O)ONc1ccn([C@@H]2O[C@H](CO)[C@@H](O)[C@H]2O)c(=O)n1] |
| EX-103 | 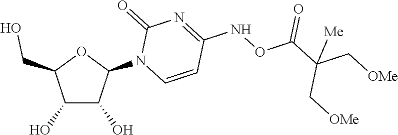 [SMILES: COCC(C)(COC)C(=O)ONc1ccn([C@@H]2O[C@H](CO)[C@@H](O)[C@H]2O)c(=O)n1] |
| EX-104 | 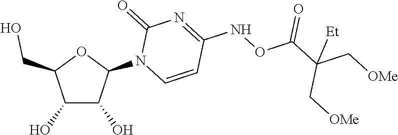 [SMILES: CCC(COC)(COC)C(=O)ONc1ccn([C@@H]2O[C@H](CO)[C@@H](O)[C@H]2O)c(=O)n1] |
| EX-105 | 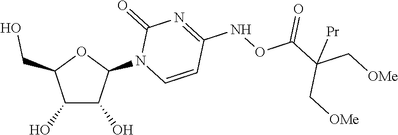 |
| EX-106 | 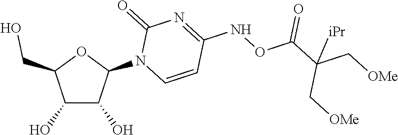 [SMILES: COCC(COC)(C(=O)ONc1ccn([C@@H]2O[C@H](CO)[C@@H](O)[C@H]2O)c(=O)n1)C(C)C] |
| EX-107 | 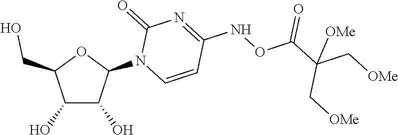 [SMILES: COCC(COC)(OC)C(=O)ONc1ccn([C@@H]2O[C@H](CO)[C@@H](O)[C@H]2O)c(=O)n1] |
| EX-108 | 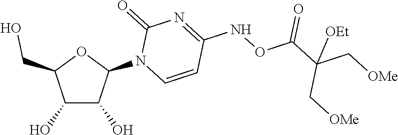 [SMILES: CCOC(COC)(COC)C(=O)ONc1ccn([C@@H]2O[C@H](CO)[C@@H](O)[C@H]2O)c(=O)n1] |
| EX-109 | 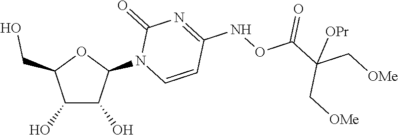 |
| EX-110 | 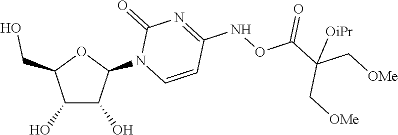 [SMILES: COCC(COC)(OC(C)C)C(=O)ONc1ccn([C@@H]2O[C@H](CO)[C@@H](O)[C@H]2O)c(=O)n1] |
| EX-111 | 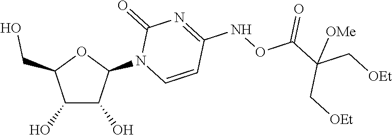 [SMILES: CCOCC(COCC)(OC)C(=O)ONc1ccn([C@@H]2O[C@H](CO)[C@@H](O)[C@H]2O)c(=O)n1] |
| EX-112 | 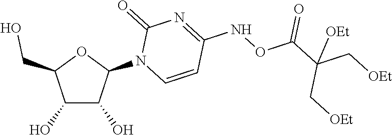 [SMILES: CCOCC(COCC)(OCC)C(=O)ONc1ccn([C@@H]2O[C@H](CO)[C@@H](O)[C@H]2O)c(=O)n1] |
| EX-113 | 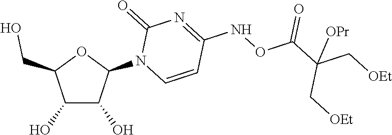 |
| EX-114 | 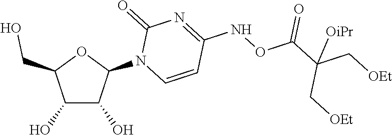 [SMILES: CCOCC(COCC)(OC(C)C)C(=O)ONc1ccn([C@@H]2O[C@H](CO)[C@@H](O)[C@H]2O)c(=O)n1] |
| EX-115 | 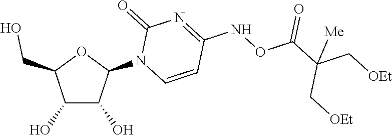 [SMILES: CCOCC(C)(COCC)C(=O)ONc1ccn([C@@H]2O[C@H](CO)[C@@H](O)[C@H]2O)c(=O)n1] |
| EX-116 | 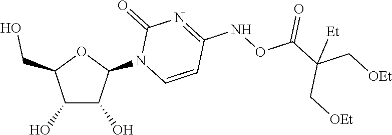 [SMILES: CCOCC(CC)(COCC)C(=O)ONc1ccn([C@@H]2O[C@H](CO)[C@@H](O)[C@H]2O)c(=O)n1] |
| EX-117 | 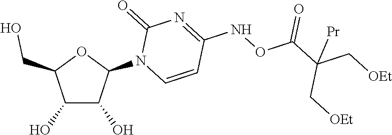 |
| EX-118 | 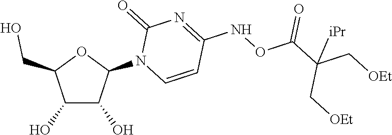 [SMILES: CCOCC(COCC)(C(=O)ONc1ccn([C@@H]2O[C@H](CO)[C@@H](O)[C@H]2O)c(=O)n1)C(C)C] |
| EX-119 | 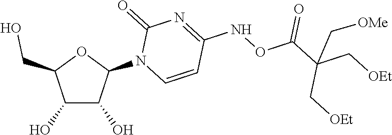 [SMILES: CCOCC(COC)(COCC)C(=O)ONc1ccn([C@@H]2O[C@H](CO)[C@@H](O)[C@H]2O)c(=O)n1] |
| EX-120 | 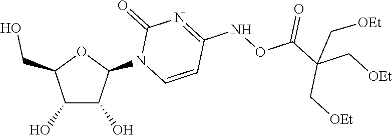 [SMILES: CCOCC(COCC)(COCC)C(=O)ONc1ccn([C@@H]2O[C@H](CO)[C@@H](O)[C@H]2O)c(=O)n1] |
| EX-121 | 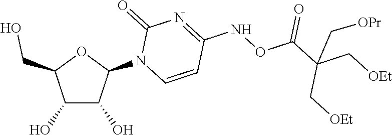 |
| EX-122 | 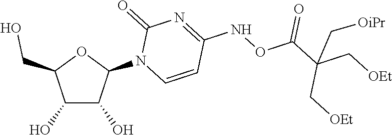 [SMILES: CCOCC(COCC)(COC(C)C)C(=O)ONc1ccn([C@@H]2O[C@H](CO)[C@@H](O)[C@H]2O)c(=O)n1] |
| EX-123 | 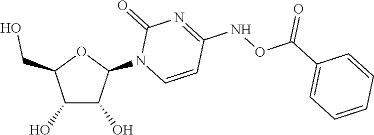 [SMILES: O=C(ONc1ccn([C@@H]2O[C@H](CO)[C@@H](O)[C@H]2O)c(=O)n1)c1ccccc1] |
| EX-124 | 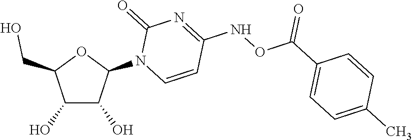 [SMILES: Cc1ccc(C(=O)ONc2ccn([C@@H]3O[C@H](CO)[C@@H](O)[C@H]3O)c(=O)n2)cc1] |
| EX-125 | 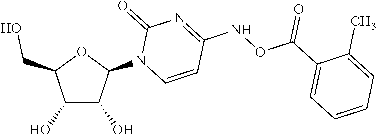 [SMILES: Cc1ccccc1C(=O)ONc1ccn([C@@H]2O[C@H](CO)[C@@H](O)[C@H]2O)c(=O)n1] |
| EX-126 | 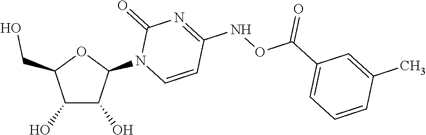 [SMILES: Cc1cccc(C(=O)ONc2ccn([C@@H]3O[C@H](CO)[C@@H](O)[C@H]3O)c(=O)n2)c1] |
| EX-127 | 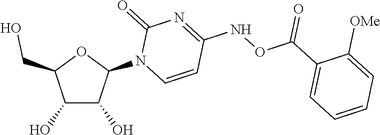 [SMILES: COc1ccccc1C(=O)ONc1ccn([C@@H]2O[C@H](CO)[C@@H](O)[C@H]2O)c(=O)n1] |
| EX-128 | 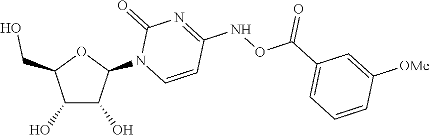 [SMILES: COc1cccc(C(=O)ONc2ccn([C@@H]3O[C@H](CO)[C@@H](O)[C@H]3O)c(=O)n2)c1] |
| EX-129 | 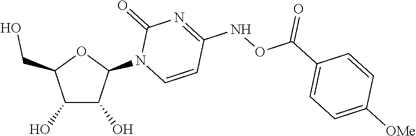 [SMILES: COc1ccc(C(=O)ONc2ccn([C@@H]3O[C@H](CO)[C@@H](O)[C@H]3O)c(=O)n2)cc1] |
| EX-130 | 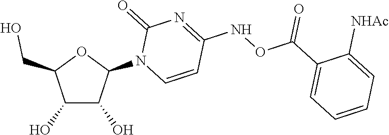 [SMILES: CC(=O)Nc1ccccc1C(=O)ONc1ccn([C@@H]2O[C@H](CO)[C@@H](O)[C@H]2O)c(=O)n1] |
| EX-131 | 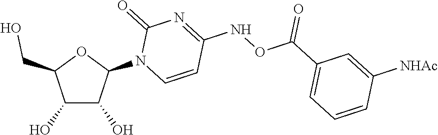 [SMILES: CC(=O)Nc1cccc(C(=O)ONc2ccn([C@@H]3O[C@H](CO)[C@@H](O)[C@H]3O)c(=O)n2)c1] |
| EX-132 | 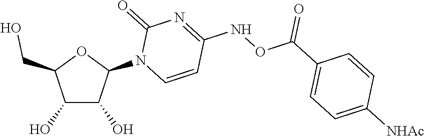 [SMILES: CC(=O)Nc1ccc(C(=O)ONc2ccn([C@@H]3O[C@H](CO)[C@@H](O)[C@H]3O)c(=O)n2)cc1] |
| EX-133 | 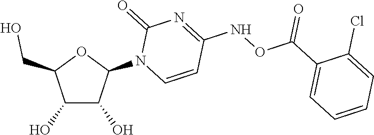 [SMILES: O=C(ONc1ccn([C@@H]2O[C@H](CO)[C@@H](O)[C@H]2O)c(=O)n1)c1ccccc1Cl] |
| EX-134 | 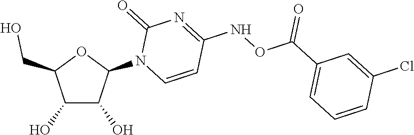 [SMILES: O=C(ONc1ccn([C@@H]2O[C@H](CO)[C@@H](O)[C@H]2O)c(=O)n1)c1cccc(Cl)c1] |
| EX-135 | 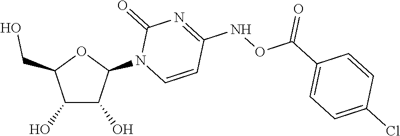 [SMILES: O=C(ONc1ccn([C@@H]2O[C@H](CO)[C@@H](O)[C@H]2O)c(=O)n1)c1ccc(Cl)cc1] |
| EX-136 | 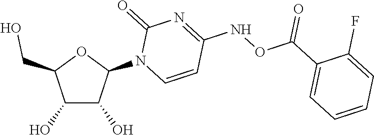 [SMILES: O=C(ONc1ccn([C@@H]2O[C@H](CO)[C@@H](O)[C@H]2O)c(=O)n1)c1ccccc1F] |
| EX-137 | 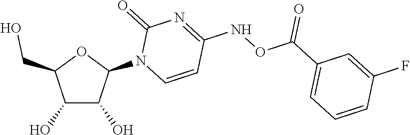 [SMILES: O=C(ONc1ccn([C@@H]2O[C@H](CO)[C@@H](O)[C@H]2O)c(=O)n1)c1cccc(F)c1] |
| EX-138 | 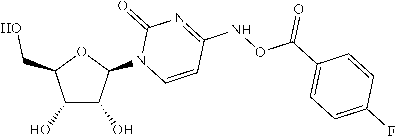 [SMILES: O=C(ONc1ccn([C@@H]2O[C@H](CO)[C@@H](O)[C@H]2O)c(=O)n1)c1ccc(F)cc1] |
| EX-139 | 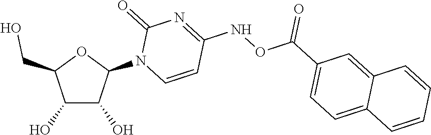 [SMILES: O=C(ONc1ccn([C@@H]2O[C@H](CO)[C@@H](O)[C@H]2O)c(=O)n1)c1ccc2ccccc2c1] |
| EX-140 | 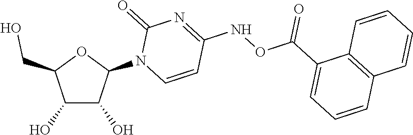 [SMILES: O=C(ONc1ccn([C@@H]2O[C@H](CO)[C@@H](O)[C@H]2O)c(=O)n1)c1cccc2ccccc12] |
| EX-141 | 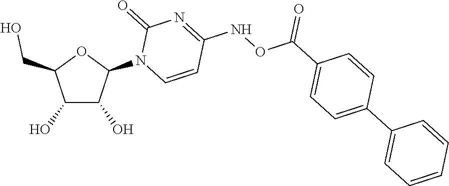 [SMILES: O=C(ONc1ccn([C@@H]2O[C@H](CO)[C@@H](O)[C@H]2O)c(=O)n1)c1ccc(-c2ccccc2)cc1] |
| EX-142 | 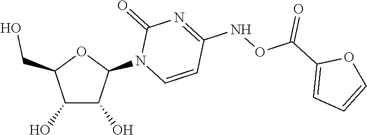 [SMILES: O=C(ONc1ccn([C@@H]2O[C@H](CO)[C@@H](O)[C@H]2O)c(=O)n1)c1ccco1] |
| EX-143 | 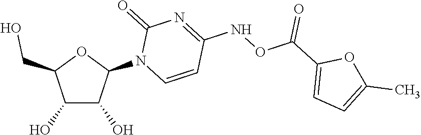 [SMILES: Cc1ccc(C(=O)ONc2ccn([C@@H]3O[C@H](CO)[C@@H](O)[C@H]3O)c(=O)n2)o1] |
| EX-144 | 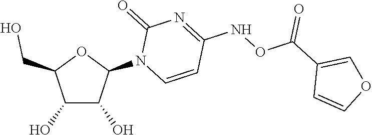 [SMILES: O=C(ONc1ccn([C@@H]2O[C@H](CO)[C@@H](O)[C@H]2O)c(=O)n1)c1ccoc1] |
| EX-145 | 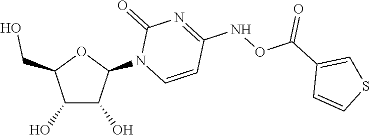 [SMILES: O=C(ONc1ccn([C@@H]2O[C@H](CO)[C@@H](O)[C@H]2O)c(=O)n1)c1ccsc1] |
| EX-146 | 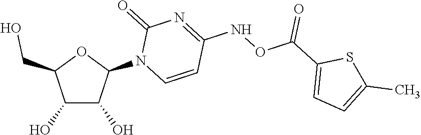 [SMILES: Cc1ccc(C(=O)ONc2ccn([C@@H]3O[C@H](CO)[C@@H](O)[C@H]3O)c(=O)n2)s1] |
| EX-147 | 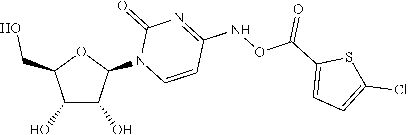 [SMILES: O=C(ONc1ccn([C@@H]2O[C@H](CO)[C@@H](O)[C@H]2O)c(=O)n1)c1ccc(Cl)s1] |
| EX-148 | 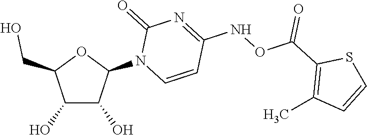 [SMILES: Cc1ccsc1C(=O)ONc1ccn([C@@H]2O[C@H](CO)[C@@H](O)[C@H]2O)c(=O)n1] |
| EX-149 | 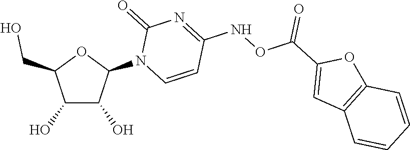 [SMILES: O=C(ONc1ccn([C@@H]2O[C@H](CO)[C@@H](O)[C@H]2O)c(=O)n1)c1cc2ccccc2o1] |
| EX-150 | 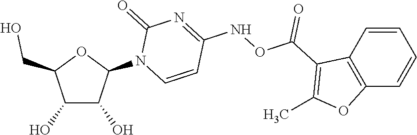 [SMILES: Cc1oc2ccccc2c1C(=O)ONc1ccn([C@@H]2O[C@H](CO)[C@@H](O)[C@H]2O)c(=O)n1] |
| EX-151 | 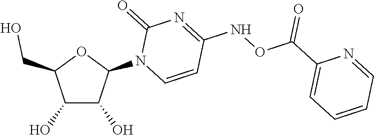 [SMILES: O=C(ONc1ccn([C@@H]2O[C@H](CO)[C@@H](O)[C@H]2O)c(=O)n1)c1ccccn1] |
| EX-152 | 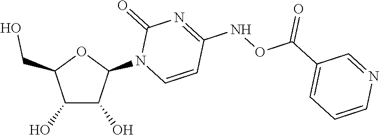 [SMILES: O=C(ONc1ccn([C@@H]2O[C@H](CO)[C@@H](O)[C@H]2O)c(=O)n1)c1cccnc1] |
| EX-153 | 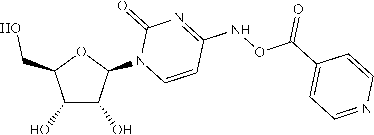 [SMILES: O=C(ONc1ccn([C@@H]2O[C@H](CO)[C@@H](O)[C@H]2O)c(=O)n1)c1ccncc1] |
| EX-154 | 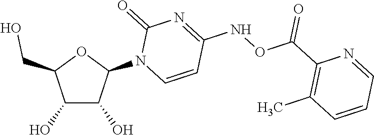 [SMILES: Cc1cccnc1C(=O)ONc1ccn([C@@H]2O[C@H](CO)[C@@H](O)[C@H]2O)c(=O)n1] |
| EX-155 | 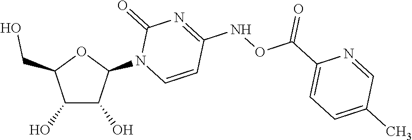 [SMILES: Cc1ccc(C(=O)ONc2ccn([C@@H]3O[C@H](CO)[C@@H](O)[C@H]3O)c(=O)n2)nc1] |
| EX-156 | 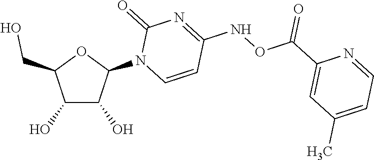 [SMILES: Cc1ccnc(C(=O)ONc2ccn([C@@H]3O[C@H](CO)[C@@H](O)[C@H]3O)c(=O)n2)c1] |
| EX-157 | 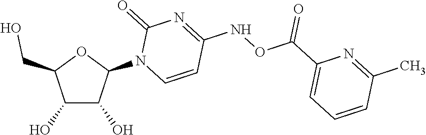 [SMILES: Cc1cccc(C(=O)ONc2ccn([C@@H]3O[C@H](CO)[C@@H](O)[C@H]3O)c(=O)n2)n1] |
| EX-158 | 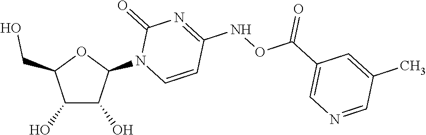 [SMILES: Cc1cncc(C(=O)ONc2ccn([C@@H]3O[C@H](CO)[C@@H](O)[C@H]3O)c(=O)n2)c1] |
| EX-159 | 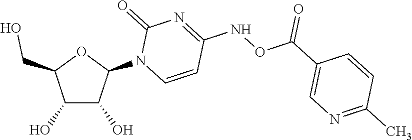 [SMILES: Cc1ccc(C(=O)ONc2ccn([C@@H]3O[C@H](CO)[C@@H](O)[C@H]3O)c(=O)n2)cn1] |
| EX-160 | 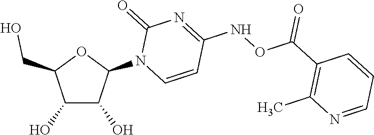 [SMILES: Cc1ncccc1C(=O)ONc1ccn([C@@H]2O[C@H](CO)[C@@H](O)[C@H]2O)c(=O)n1] |
| EX-161 | 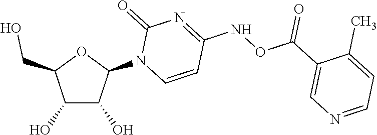 [SMILES: Cc1ccncc1C(=O)ONc1ccn([C@@H]2O[C@H](CO)[C@@H](O)[C@H]2O)c(=O)n1] |
| EX-162 | 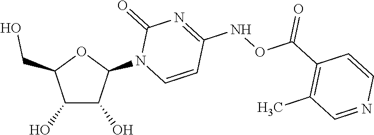 [SMILES: Cc1cnccc1C(=O)ONc1ccn([C@@H]2O[C@H](CO)[C@@H](O)[C@H]2O)c(=O)n1] |
| EX-163 | 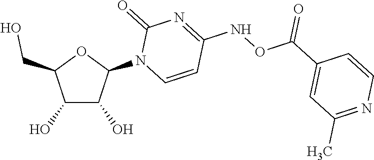 [SMILES: Cc1cc(C(=O)ONc2ccn([C@@H]3O[C@H](CO)[C@@H](O)[C@H]3O)c(=O)n2)ccn1] |
| EX-164 | 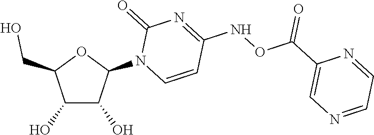 [SMILES: O=C(ONc1ccn([C@@H]2O[C@H](CO)[C@@H](O)[C@H]2O)c(=O)n1)c1cnccn1] |
| EX-165 | 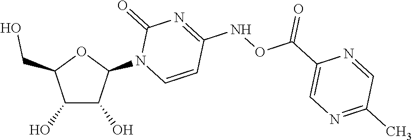 [SMILES: Cc1cnc(C(=O)ONc2ccn([C@@H]3O[C@H](CO)[C@@H](O)[C@H]3O)c(=O)n2)cn1] |
| EX-166 | 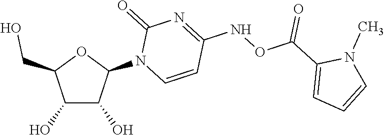 [SMILES: Cn1cccc1C(=O)ONc1ccn([C@@H]2O[C@H](CO)[C@@H](O)[C@H]2O)c(=O)n1] |
| EX-167 | 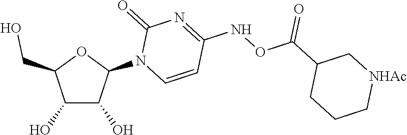 |
| EX-168 | 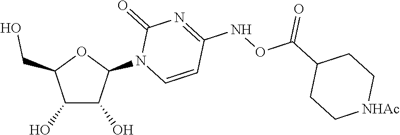 |
| EX-169 | 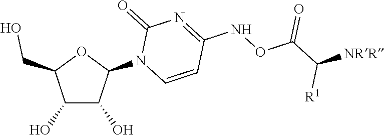 |

15. A pharmaceutical composition, comprising the compound of any one of claims 1-14 or a tautomer, stereoisomer or racemate thereof or a pharmaceutically acceptable salt thereof, and optionally comprising a pharmaceutically acceptable excipient.

16. Use of the compound of any one of claims 1-14 or a tautomer, stereoisomer or racemate thereof or a pharmaceutically acceptable salt thereof in the manufacture of a medicament for treating or preventing a RNA viral infection.

17. A method of treating or preventing a RNA viral infection in a subject, comprising administering to the subject in need thereof an effective amount of the compound of any one of claims 1-14 or a tautomer, stereoisomer or racemate thereof or a pharmaceutically acceptable salt thereof.

18. The compound of any one of claims 1-14 or a tautomer, stereoisomer or racemate thereof or a pharmaceutically acceptable salt thereof, for use as a medicament.

19. The compound of any one of claims 1-14 or a tautomer, stereoisomer or racemate thereof or a pharmaceutically acceptable salt thereof, for use in treating or preventing a RNA viral infection.

20. The use according to claim 16, the method according to claim 17 or the compound for use according to any one of claims 18-19, wherein the RNA virus is coronavirus, e.g., a human coronavirus, SARS coronavirus or MERS coronavirus, alphavirus, e.g., Eastern equine encephalitis virus, Western equine encephalitis virus, Venezuelan equine encephalitis virus, Chikungunya virus or Ross River virus, filoviridae virus, e.g., ebola virus, orthomyxoviridae virus, e.g., influenza virus, influenza A virus or influenza B virus, paramyxoviridae virus, e.g., respiratory Syncytial Virus (RSV), flavivirus, e.g., Zika virus; preferably, a SARS-CoV-2/COVID-19 virus, an alpha variant SARS-CoV-2/COVID-19 virus, a beta variant SARS-CoV-2/COVID-19 virus, a gamma variant SARS-CoV-2/COVID-19 virus, a delta variant SARS-CoV-2/COVID-19 virus, or any other variant SARS-CoV-2/COVID-19 virus.

21. A pharmaceutical combination, comprising the compound of any one of claims 1-14 or a tautomer, stereoisomer or racemate thereof or a pharmaceutically acceptable salt thereof, and at least one additional therapeutic agent.

22. The pharmaceutical combination according to claim 21, wherein additional therapeutic agent is selected from the group consisting of:

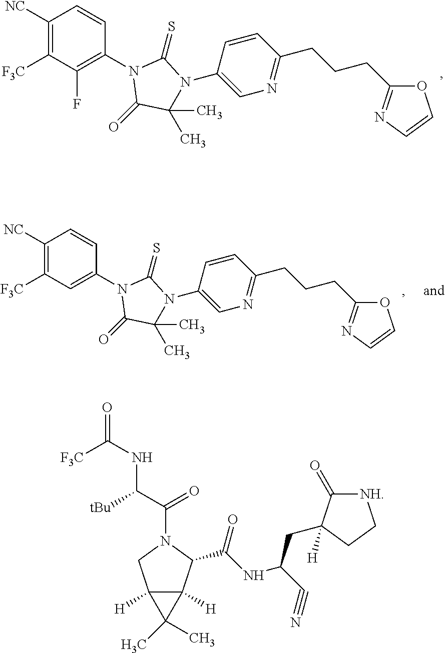 [SMILES: CC1(C)C(=O)N(c2ccc(C#N)c(C(F)(F)F)c2F)C(=S)N1c1ccc(CCCc2ncco2)nc1 ; CC1(C)C(=O)N(c2ccc(C#N)c(C(F)(F)F)c2)C(=S)N1c1ccc(CCCc2ncco2)nc1 ; CC(C)(C)[C@H](NC(=O)C(F)(F)F)C(=O)N1C[C@H]2[C@@H]([C@H]1C(=O)N[C@H](C#N)C[C@@H]1CCNC1=O)C2(C)C]

23. A pharmaceutical composition for the treatment of 2019_nCoV/SARS-CoV-2 infection, comprising a pharmaceutically acceptable excipient and a compound of Formula I, or a tautomer thereof, or a pharmaceutically acceptable or physiologically acceptable salt thereof, wherein the compound of Formula I is defined as in any one of claims 1-14.

24. A process for the production of compounds of Formula I according to any one of claims 1-14, comprising the following step:
reacting NHC with the acid anhydride of formula II to yield the compound of formula I, 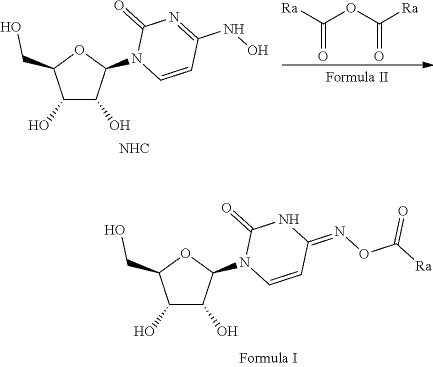
wherein Ra is defined as any one of claims 1-14.

25. The process according to claim 24, wherein the reaction is carried out in water or a mixture of water and organic solvent(s), preferably the reaction solvent is selected from pure water, methanol, ethanol, propanol, isopropanol, other lower aliphatic alcohol or a mixture of aliphatic alcohols, DMF, DMSO, NMP, water-methanol mixture, water-ethanol mixture, water-propanol mixture, water-isopropanol mixture, water-nbutanol mixture, water-secbutanol mixture, water-isobutanol mixture, water-THF mixture, water-ACN mixture, water-DMF mixture, water-DMSO mixture, water/2-methyl THF mixture, or any mixture of water with organic solvent(s) that can fully or partially dissolve NHC; more preferably water, lower aliphatic alcohol, water-THF mixture, water/2-methyl THF mixture, water/ACN mixture or water-lower aliphatic alcohol(s) mixtures.

26. The process according to claim 24 or 25, wherein the reaction is carried out without adding any inorganic or organic base (or catalyst), for example, alkali metal hydroxide, carbonate, bicarbonate, alkoxide or hydride, such as sodium bicarbonate, potassium bicarbonate, sodium carbonate, potassium carbonate, sodium hydroxide, potassium hydroxide, lithium hydroxide, sodium methoxide, sodium ethoxide, or sodium hydride, or organic tertiary amines, such as tri-C₁₋₄alkylamines, e.g., TEA, diisopropylethylamine, tripropylamine, tributylamine, or heterocyclic bases, such as pyridine, picolines, lutidines, DMAP, DBU etc.

27. The process according to claim 24 or 25, wherein the reaction is carried out in the presence of inorganic or organic base (or catalyst), for example, alkali metal hydroxide, carbonate, bicarbonate, alkoxide or hydride, such as sodium bicarbonate, potassium bicarbonate, sodium carbonate, potassium carbonate, sodium hydroxide, potassium hydroxide, lithium hydroxide, sodium methoxide, sodium ethoxide, or sodium hydride, or organic tertiary amines, such as tri-C₁₋₄alkylamines, e.g., TEA, diisopropylethylamine, tripropylamine, tributylamine, or heterocyclic bases, such as pyridine, picolines, lutidines, DMAP, DBU etc.

28. The process according to any one of claims 24-27, wherein the product is obtained in solid crystalline form, by cooling the reaction mixture without adding anti-solvent.

29. The process according to any one of claims 24-28, wherein the product is obtained in solid crystalline form, without subjecting to any chromatography purification.

30. The process according to any one of claims 24-29, wherein the purity of the product obtained is about 90%-98%.

31. The process according to any one of claims 24-29, wherein the purity of the product obtained is over 98%.
